(19) United States
(12) Patent Application Publication
(10) Pub. No.: US 2026/0035709 A1
(43) Pub. Date: Feb. 5, 2026

(54) POD SHATTER TOLERANCE IN BRASSICA PLANTS

(71) Applicant: PIONEER HI-BRED INTERNATIONAL, INC., Johnston, IA (US)

(72) Inventors: Sarah Atwood, Ankeny, IA (US); Norbert Brugiere, Johnston, IA (US); Igor FALAK, Guelph (CA); Kevin A Fengler, Clive, IA (US); Siva S. Ammiraju Jetty, Johnston, IA (US); Jonathan Myrvold, SACRAMENTO, CA (US)

(73) Assignee: PIONEER HI-BRED INTERNATIONAL, INC., Johnston, IA (US)

(21) Appl. No.: 19/355,403

(22) Filed: Oct. 10, 2025

Related U.S. Application Data
(63) Continuation of application No. 17/617,724, now Pat. No. 12,442,010.
(60) Provisional application No. 62/863,551, filed on Jun. 19, 2019.

(51) Int. Cl. C12N 15/82; C07K 14/415; C12N 9/22; C12N 9/24; C12N 15/11; C12Q 1/6895

(52) U.S. Cl. CPC C12N 15/8213; C07K 14/415; C12N 9/22; C12N 9/24; C12N 15/111; C12Q 1/6895; C12N 2310/20

(57) ABSTRACT

This disclosure provides methods and compositions for identifying Brassica plants that have a native deletion of the INDEHISCENT gene (BnIND-A) located on chromosome A of B. napus. Also provided are methods of improving one or more agronomic characteristics such as pod shatter and breeding methods for introducing a pod shatter tolerant phenotype in Brassica plants and/or their progeny.

### CROSS-REFERENCE TO RELATED APPLICATIONS

This application is a continuation of U.S. patent application Ser. No. 17/617,724, filed on Dec. 9, 2021, which is a 35 U.S.C. § 371 national stage application of PCT International Application No. PCT/US2020/038124, filed on Jun. 17, 2020, which claims the benefit of U.S. Provisional Application No. 62/863,551, filed Jun. 19, 2019, the disclosures of which are incorporated by reference in their entirety.

### REFERENCE TO SEQUENCE LISTING SUBMITTED ELECTRONICALLY

The official copy of the sequence listing is submitted electronically via EFS-Web as an xml-formatted sequence listing file named 107558-US-CON-1_ST26.xml created on Oct. 6, 2025 and having a size of 404,703 bytes, which is filed concurrently with the specification. The sequence listing comprised in this xml-formatted document is part of the specification and is herein incorporated by reference in its entirety.

### FIELD

This disclosure relates to compositions and methods, including sequences, markers, assays and the use of marker assisted selection for improving agronomic traits in plants, specifically improving pod shatter tolerance in Brassica plants.

### BACKGROUND

Brassica napus (also referred to as canola or oilseed rape) is one of the most important vegetable oilseed crops in the world, especially in China, Canada, the European Union and Australia, where the oils are used extensively in the food industry and for biodiesel production. Oilseed rape is a recently domesticated plant and retains some of the traits of its wild ancestors which were useful in the wild but are not useful in commercial crop plants. One example of such a trait is fruit dehiscence, which refers to the natural opening of reproductive structures to disperse seeds. In species that disperse their fruit through dehiscense, siliques or pods are composed of two carpels that are held together by a central replum via a valve margin. Where the valve margin connects to the replum is called the dehiscence zone (DZ). When the pod is ripe, the valve margin detaches from the replum and the pod splits open, releasing the seeds inside. The DZ demarcates the precise location where the valves detach.

During crop domestication, farmers and breeders have selected for Brassica plants that avoid releasing their seeds early, before the crop is harvested. However, such early pod dehiscence (also known as “pod shatter”, “seed shatter” or “seed shedding”) has not been fully eliminated. Therefore, B. napus plants remain prone to seed losses due to pod shatter prior to harvest. Pod shatter poses significant problems for commercial production of canola seeds and adverse weather conditions can exacerbate the process resulting in an increase in shatter-related losses of 25% or more. This loss of seed not only has a dramatic effect on yield but can also result in the emergence of the crop as a weed in the subsequent growing season.

In addition to direct losses of income from reduced seed yield, increased input costs and reduced price paid for low oil content seeds, pod shatter also results in additional indirect costs to the grower. The shed seed results in self-sown or volunteer B. napus plants growing in the next year's crop, which creates further expense due to the need for increased herbicide use. Such self-sown B. napus plants cause losses due to competition with subsequent crop and can cause problems for farmers using reduced-tillage strategies such as no-till, zone-till, and strip tillage.

Resistance to pod shatter (indehiscent phenotype) is a key trait that has been selected during crop domestication. Plants have been also generated using Ethyl Methane Sulfonate (EMS) mutagenesis or through single guide gene editing. Rajani and Sundaresan, 2001, Current Biology, 11 (24), 1914-1922; Liljegren et al., 2004, Cell, 116 (6), 843-853; Braatz et al., 2017, Plant Physiology, 174 (2), 935-942; Braatz et al., 2018, Euphytica, 214 (2), 29; Braatz et al., 2018, Theor. Applied Genetics, 131 (4), 959-971. However, some of these approaches have produced plants with “huge background mutations” and plants that are otherwise “unsuitable for agronomic purposes” (Zhai et al., 2019, Theor. Applied Genetics, 132:2111-2123 at 2112 and 2121). Thus, there remain varieties of B. napus that are still dehiscent and prone to pod shatter. In view of the foregoing, there is a need for more B. napus lines having a pod shatter tolerance, i.e., indehiscent phenotype, and new approaches for generating such plants. There is also a need for pod-shatter phenotypes that permit plant seeds to be collected at harvest by threshing pods, e.g., using a combine harvester, with minimal damage to the seed.

### SUMMARY

Provided herein are methods, assays and molecular markers based, at least in part, on the discovery of an unexpected deletion of genomic sequence affecting the INDEHISCENT gene (BnIND-A) located on chromosome A of B. napus. Also disclosed herein is the discovery that this deletion confers a pod shatter tolerant phenotype in B. napus plants and/or their progeny. Generally, a plant with pod shatter tolerance is one having increased pod shatter tolerance relative to an otherwise isogenic plant lacking the BIND-A deletion disclosed herein. Therefore, the methods and markers disclosed herein can be used to identify (i) a plant having a pod shatter tolerant phenotype and/or (ii) a plant suitable for use as a parent plant in a breeding program to generate progeny plants having a pod shatter tolerant phenotype.

The methods, assays, and molecular markers can be used with a Brassica crop plant. As used herein, Brassica preferably refers to Brassica napus, Brassica juncea, Brassica carinata, Brassica rapa or Brassica oleracea.

In one aspect, this disclosure provides a method of identifying a Brassica plant, cell, or germplasm thereof comprising a BnIND-A genomic deletion that contributes to a pod shatter tolerance phenotype. The method comprises obtaining a nucleic acid sample from a Brassica plant cell, or germplasm; and screening the sample for genomic sequence comprising a deletion of the BnIND-A gene on chromosome N03. This BnIND-A deletion allele is missing a genomic segment that is from about 200 kb to about 310 kb in length, depending on the reference genome used for comparison. The deletion segment start breakpoint is located at about position 13,300,000 to 14,915,000 of an N03 wildtype reference genome and the deletion segment end breakpoint corresponds to a position located at about position 13,500,000 to 15,250,000 of a N03 wildtype reference genome. See Example 1 and Table 6 herein. The absence of this deleted genomic segment of BnIND-A contributes to a pod shatter tolerance phenotype in Brassica.

For example, the method of identifying a Brassica plant, cell, or germplasm thereof comprising a BnIND-A genomic deletion can include screening the sample for the absence of the deleted genomic segment at the breakpoint locus corresponding to positions 14,989,780 to 14,989,781 of Brassica napus line G00010BC N03 genome, e.g., the breakpoint locus corresponding to positions 10,002-10,003 of SEQ ID NO:2. Screening the sample can be done using any suitable method for detecting a genetic polymorphism, including any method disclosed herein.

When screening the plant sample for genomic sequence comprising the BnIND-A genomic deletion, the disclosed methods can include amplifying the genomic sequence to produce an amplicon. The amplicon comprises amplified genomic sequence which is generated using a nucleic acid amplification such as polymerase chain reaction (PCR). Thus, for example, the method can include amplifying genomic DNA to produce an amplicon that includes the breakpoint locus sequence corresponding to positions 14,989,780 to 14,989,781 of Brassica napus line G00010BC N03 genome or positions 10,002-10,003 of SEQ ID NO:2. The amplicon can be sequenced to confirm the presence of the breakpoint and/or the size of the amplicon produced is diagnostic for the BnIND-A genomic deletion.

In some examples, sequencing or amplification of a BnIND-A deletion allele can produce a sequencing product or amplicon, respectively, comprising the following start and end breakpoint locus (shown in bold and underlined) and flanking sequence corresponding to SEQ ID NO: 2 (positions 9995-1011): ATTTCTCTATTTGTTTT. Such a sequencing product or amplicon comprising the breakpoint locus is diagnostic for the BnIND-A genomic deletion. Thus, in particular examples, detecting the BnIND-A deletion can include DNA sequencing or amplification of the breakpoint locus and 5 bp or more, 10 bp or more, 15 bp or more, 20 bp or more, 30 bp or more, 40 bp or more, 50 bp or more, 60 bp or more, 70 bp or more, 80 bp or more, 90 bp or more, 100 bp or more, 110 bp or more, 120 bp or more, 130 bp or more, 140 bp or more, 150 bp or more, 175 bp or more, 200 bp or more, 250 bp or more, 300 bp or more, 350 bp or more, 400 bp or more, 450 bp or more, 500 bp or more, 550 bp or more, or 600 bp or more of flanking sequence that is (i) upstream of (i.e., located 5′ to) the deletion start breakpoint at position 10,002 of SEQ ID NO:2 and/or (ii) downstream of (i.e., located 3′ to) the deletion end breakpoint at position 10,003 of SEQ ID NO:2. Further, in particular examples, the BnIND-A deletion disclosed herein can be detected amplifying genomic sequence to produce an amplicon comprising the BnIND-A deletion allele sequence indicated in Table 1 below. Additionally, the BnIND-A deletion disclosed herein can be detected by nucleotide sequencing to detect the presence of the genomic sequence (e.g., in amplified genomic sequence) comprising any one or more of the BnIND-A deletion allele sequences indicated in Table 1 below.

TABLE 1

| SEQ ID NO: 2 | SEQ ID NO: 2 | SEQ ID NO: 2 | SEQ ID NO: 2 |
|---|---|---|---|
| positions | positions | positions | positions |
| 9997 to 10008 | 9952 to 10053 | 9882 to 10123 | 9702 to 10303 |
| 9992 to 10013 | 9942 to 10063 | 9872 to 10133 | 9652 to 10353 |
| 9987 to 10018 | 9932 to 10073 | 9862 to 10143 | 9602 to 10403 |
| 9982 to 10023 | 9922 to 10083 | 9852 to 10153 | 9552 to 10453 |
| 9977 to 10028 | 9912 to 10093 | 9827 to 10178 | 9502 to 10503 |
| 9972 to 10033 | 9902 to 10103 | 9802 to 10203 | 9452 to 10553 |
| 9962 to 10043 | 9892 to 10113 | 9752 to 10253 | 9402 to 10603 |

The disclosure also provides an amplification, e.g., PCR assay method that comprises obtaining a nucleic acid sample from a Brassica plant, cell, or germplasm thereof, isolating genomic DNA from the sample and screening the isolated DNA for genomic sequence comprising the BnIND-A deletion disclosed herein by contacting the isolated genomic DNA with a deletion forward primer and deletion reverse primer to selectively produce an amplicon comprising the BnIND-A deletion breakpoint locus at positions 10,002-10,003 of SEQ ID NO:2. Selective amplification of the BnIND-A deletion amplicon can be achieved using a first deletion primer that anneals upstream of the deletion breakpoint BnIND-A deletion breakpoint and a second deletion primer that anneals downstream of the deletion breakpoint. The method can further, optionally, include contacting the isolated genomic DNA with a wildtype forward primer and wildtype reverse primer capable of selectively producing a second amplicon of wildtype genomic BnIND-A that includes sequence from the deleted genomic segment. Selective amplification of the wildtype amplicon can be achieved using at least one wildtype primer that anneals within the deleted genomic segment disclosed herein. The primers used in such a PCR assay can be labeled, e.g., with a radioactive or fluorescent label for detection of amplified product. If both deletion and wildtype labeled primers are used, the label on a deletion primer is preferably different from the label on a wildtype primer. Examples of forward and reverse primers for amplification of BnIND-A deletion allele sequence and wildtype genomic BnIND-A sequence, respectively, are provided in Table 2.

TABLE 2

| SEQ ID NO | Name | Description |
|---|---|---|
| SEQ ID NO: 34 | BP_F001 | Deletion Forward Primer |
| SEQ ID NO: 35 | BP_R001 | Deletion Reverse Primer |
| SEQ ID NO: 51 | BP_F002 | Deletion Forward Primer |
| SEQ ID NO: 52 | BP_R002 | Deletion Reverse Primer |
| SEQ ID NO: 53 | BP_F003 | Deletion Forward Primer |
| SEQ ID NO: 54 | BP_R003 | Deletion Reverse Primer |
| SEQ ID NO: 55 | BP_R004 | Deletion Reverse Primer |
| SEQ ID NO: 56 | BP_R005 | Deletion Reverse Primer |
| SEQ ID NO: 57 | BP_R006 | Deletion Reverse Primer |
| SEQ ID NO: 32 | IND_A_001_F001 | WT Forward Primer |
| SEQ ID NO: 33 | IND_A_001_R001 | WT Reverse Primer |
| SEQ ID NO: 58 | IND_A_001_R002 | WT Reverse Primer |

A disclosed amplification or PCR assay can include obtaining a nucleic acid sample from a Brassica plant, cell, or germplasm thereof, isolating genomic DNA from the sample and screening for genomic sequence comprising the BnIND-A deletion disclosed herein by contacting the isolated genomic DNA with a deletion forward primer and deletion reverse primer to produce an amplicon comprising the BnIND-A deletion breakpoint locus at positions 10,002-10,003 of SEQ ID NO:2, and then contacting a labeled probe (deletion probe) to the deletion amplicon comprising the deletion breakpoint, and thereby detecting the BnIND-A deletion amplicon. The method can further, optionally, include contacting the isolated genomic DNA with a wildtype forward primer and wildtype reverse primer capable of producing a second amplicon of wildtype genomic BnIND-A that includes sequence from the deleted genomic segment, and then adding a labeled wildtype probe which is capable of detecting the wildtype amplicon. The deletion probe and wildtype probe are preferably differently labeled to permit, which can enable the use of both probes in the same reaction mix or in a high throughput amplification assay method. Examples of forward primers, reverse primers, and probes for the detection of BnIND-A deletion allele and wildtype genomic BnIND-A, respectively, are provided in Table 3.

TABLE 3

| SEQ ID NO | Name | Description |
|---|---|---|
| SEQ ID NO: 39 | N101T10-F001 | Deletion Forward Primer |
| SEQ ID NO: 40 | N101T10-R001 | Deletion Reverse Primer |
| SEQ ID NO: 41 | N101T10-001-X001 | Deletion Probe |
| SEQ ID NO: 42 | N101T10-F002 | Deletion Forward Primer |
| SEQ ID NO: 43 | N101T10-R002 | Deletion Reverse Primer |
| SEQ ID NO: 44 | N101T10-001-X002 | Deletion Probe |
| SEQ ID NO: 46 | N101T10-R003 | Deletion Reverse Primer |
| SEQ ID NO: 47 | N101T10-001-X003 | Deletion Probe |
| SEQ ID NO: 36 | N101T11-F001 | WT Forward Primer |
| SEQ ID NO: 37 | N101T11-R001 | WT Reverse Primer |
| SEQ ID NO: 38 | N101T11-001-X001 | WT Probe |
| SEQ ID NO: 48 | N101T11-R002 | WT Reverse Primer |
| SEQ ID NO: 49 | N101T11-001-X002 | WT Probe |
| SEQ ID NO: 50 | N101T11-001-X003 | WT Probe |

Each of the methods disclosed herein for identifying a Brassica plant, cell, or germplasm thereof comprising the disclosed BnIND-A genomic deletion can further include selecting such a Brassica plant, cell, or germplasm thereof comprising the disclosed BnIND-A genomic deletion that contributes to a pod shatter tolerance phenotype. This method of selection can be used advantageously in methods of introducing the BnIND-A deletion into a Brassica variety and thereby generate new plant lines comprising the BnIND-A deletion.

In one aspect, provided herein is a method of introducing the native BnIND-A deletion into a new Brassica plant, e.g., a B. napus plant. The method can include crossing a first parent Brassica plant comprising a native deletion in the BnIND-A gene on chromosome N03 with a second parent Brassica plant that does not have the deletion to produce progeny plants (e.g. hybrid progeny), obtaining a nucleic acid sample from one or more of the progeny plants, and identifying one or more of the progeny plants that has the BIND-A deletion. Progeny plants can be identified using one or more of the methods disclosed herein (which include, but are not limited to, whole genome sequencing, coupled genomic DNA amplification and sequencing, DNA amplification methods that include the use of labeled primers and/or labeled probes, marker assisted selection, primer extension etc.) to identify a Brassica plant, cell, or germplasm thereof comprising the disclosed BnIND-A genomic deletion. The method can further include selecting the hybrid progeny plant identified as having the BnIND-A genomic deletion. This method can thus be used to create progeny plants having the BnIND-A genomic deletion that provides the pod shatter tolerance trait disclosed herein.

In certain examples, the foregoing method steps can be repeated by crossing the one or more selected progeny plants with the first or second parent Brassica plant (the recurrent parent plant) to produce backcross progeny plants. Nucleic acid samples are obtained from one or more backcross progeny plants; and backcross progeny plants comprising the disclosed BnIND-A genomic deletion are identified. The method further includes selecting the one or more backcross progeny plants having the BnIND-A deletion to produce another generation of backcross progeny plants. This process can be further repeated two, three, four, five, six, or seven times, i.e., by crossing the latest generation of selected backcross progeny plants having the BnIND-A deletion with the recurrent parent plant, and each time identifying and selecting additional backcross progeny plants having the BnIND-A deletion. Repeated backcrossing to the recurrent parent plant can be used to create Brassica plant lines that combine the BIND-A deletion shatter tolerance trait with the agronomic characteristics of the recurrent parent plant, when grown in the same environmental conditions.

Further provided is the use of gene editing technology to create a targeted genomic modification of the BnIND-A gene in a Brassica genomic locus. The modification produces a deletion of from about 200 kb to about 310 kb in length, wherein the deletion segment start breakpoint corresponds to about position 13,300,000 to 14,915,000 of an N03 wildtype reference genome and the deletion end breakpoint corresponds to about position 13,500,000 to 15,250,000 of an N03 wildtype reference genome. The resulting modified Brassica plant, cell, or germplasm comprises BnIND-A sequence that includes the breakpoint locus corresponding to positions 14,989,780 to 14,989,781 of Brassica napus line G00010BC N03 genome or positions 10,002-10,003 of SEQ ID NO:2 and sequence flanking thereof. Methods for creating such gene edited plants dropouts comprise inducing a first and second double strand break in genomic DNA using a TALE-nuclease (TALEN), a meganuclease, a zinc finger nuclease, or a CRISPR-associated nuclease. In a preferred aspect, the method comprises introducing a CRISPR-associated nuclease and guide RNAs into a B. napus plant cell.

The disclosure can be more fully understood from the following detailed description and the accompanying drawings and Sequence Listing, which form a part of this application.

### BRIEF DESCRIPTION OF THE DRAWINGS AND SEQUENCE LISTING

FIG. 1 is a schematic illustrating a KASPar™ assay designed to detect a native BnIND-A deletion on chromosome N03 (SEQ ID NO: 2). In FIG. 1, “1” indicates a wildtype allele-specific forward primer, “2” indicates a wildtype specific common or reverse primer, “3” indicates a natural deletion allele-specific forward primer, and “4” indicates a natural deletion allele-specific common or reverse primer.

FIG. 2 is a schematic illustrating a TAQMAN™ assay designed to detect a native BnIND-A deletion on chromosome N03. In FIG. 2, star “1” indicates a wildtype specific probe; star “2” indicates a natural deletion specific probe; “3” indicates a wildtype and mutant common forward primer, “4” indicates a wildtype allele-specific reverse primer; and “5” indicates a natural deletion allele-specific reverse primer.

FIG. 3 is a scatterplot showing the results of using a TAQMAN™ assay to screen B. napus germplasm segregating in a mapping population for the native BnIND-A deletion disclosed herein.

FIG. 4A is a scatterplot showing the results of using a KASPar™ assay to interrogate global elite B. napus germplasm for the native BnIND-A deletion disclosed herein. FIG. 4B is a scatterplot showing the results of using a TAQMAN™ assay to interrogate global elite B. napus germplasm for the native BnIND-A deletion disclosed herein.

FIG. 5 is a bar graph showing the average percentage shattered pods of B. napus inbreds G00010BC, NS1822BC, and G00555MC, which were evaluated using a method for laboratory phenotyping pod shatter tolerance as disclosed herein.

FIG. 6 is a bar graph showing the average percentage shattered pods of G00010BC plants segregating for an IND-C dropout determined using a laboratory method for phenotyping pod shatter tolerance. Asterisk indicates a significant difference (T-test, p<0.05) as compared to wild-type (WT) plants. N=number of 15 pods replications for each zygosity category.

FIG. 7 is a bar graph showing the average percentage of shattered pods of a commercial pod-shatter tolerant line (PST Check 1), G00010BC plants (2 KO), and modified G00010BC plants that are either homozygous (4 KO) or heterozygous (3 KO) for gene-edited deletions of IND-C gene allele. Pod shatter tolerance was determined using a laboratory phenotyping method.

FIG. 8 is a bar graph showing the SHTPC of unmodified G00010BC plants (2 KO), Recovered segregant with homozygous BnIND-A deletion (2 KO), and G00010BC plants that are either homozygous (4 KO) or heterozygous (3 KO) for gene-edited deletions of IND-C gene allele. Pod shatter tolerance was determined using a field phenotyping method.

FIG. 9 is a bar graph showing the average percentage of shattered pods Shatter tolerance of G00555MC×G00010BC gene edited hybrids with indicated dropout allele combinations and hybrid checks calculated as average percent shattered pods +/−SE. Pod shatter tolerance was determined using a lab phenotyping method. A and C indicate functional IND-A and IND-C alleles, respectively and lower case a and c indicate deletions of IND-A and IND-C alleles, respectively. Single asterisk indicates a significant difference (T-test, p<0.01) and double asterisk indicates a significant difference (T-test, p<0.05).

Sequence listings are described in the following Table 4. Nucleic acid sequences listed in the accompanying sequence listing and referenced herein are shown using standard letter abbreviations for nucleotide bases. Only one strand of each nucleic acid sequence is shown, but the complementary strand is understood to be included by any reference to the displayed strand.

TABLE 4

| SEQ ID NO: | DESCRIPTION |
|---|---|
| SEQ ID NO: 1 | BnIND A genome protein |
| SEQ ID NO: 2 | G00010BC IND genomic seq A genome (Native Deletion) |
| SEQ ID NO: 3 | G00010BC BnIND genomic seq C genome |
| SEQ ID NO: 4 | G00010BC BnIND C genome dropout |
| SEQ ID NO: 5 | G00010BC BnALC genomic seq A genome |
| SEQ ID NO: 6 | G00010BC BnALC genomic seq C genome |
| SEQ ID NO: 7 | G00010BC BnPGAZ genomic seq A genome |
| SEQ ID NO: 8 | G00010BC BnPGAZ A genome dropout |
| SEQ ID NO: 9 | G00010BC BnPGAZ genomic seq C genome |
| SEQ ID NO: 10 | G00010BC BnPGAZ C genome dropout |
| SEQ ID NO: 11 | NS1822BC BnIND genomic seq A genome |
| SEQ ID NO: 12 | NS1822BC BnIND A genome dropout |
| SEQ ID NO: 13 | NS1822BC BnIND genomic seq C genome |
| SEQ ID NO: 14 | NS1822BC BnIND C genome dropout |
| SEQ ID NO: 15 | NS1822BC BnPGAZ genomic seq A genome |
| SEQ ID NO: 16 | NS1822BC BnPGAZ A genome dropout 1 |
| SEQ ID NO: 17 | NS1822BC BnPGAZ A genome dropout 2 |
| SEQ ID NO: 18 | NS1822BC BnPGAZ genomic seq C genome |
| SEQ ID NO: 19 | NS1822BC BnPGAZ C genome dropout 1 |
| SEQ ID NO: 20 | NS1822BC BnPGAZ C genome dropout 2 |
| SEQ ID NO: 21 | NS1822BC BnPGAZ C genome dropout 3 |
| SEQ ID NO: 22 | G00555MC BnIND genomic seq A genome |
| SEQ ID NO: 23 | G00555MC BnIND A genome dropout |
| SEQ ID NO: 24 | G00555MC BnIND genomic seq C genome |
| SEQ ID NO: 25 | G00555MC BnIND C genome dropout |
| SEQ ID NO: 26 | G00555MC BnALC genomic seq A genome |
| SEQ ID NO: 27 | G00555MC BnALC genomic seq C genome |
| SEQ ID NO: 28 | G00555MC BnPGAZ genomic seq A genome |
| SEQ ID NO: 29 | G00555MC BnPGAZ A genome dropout |
| SEQ ID NO: 30 | G00555MC BnPGAZ genomic seq C genome |
| SEQ ID NO: 31 | G00555MC BnPGAZ C genome dropout |
| SEQ ID NO: 32 | IND_A_001_F001 WT Forward Primer |
| SEQ ID NO: 33 | IND_A_001_R001 WT Reverse Primer |
| SEQ ID NO: 34 | BP_F003 Deletion Forward Primer |
| SEQ ID NO: 35 | BP_R003 Deletion Reverse Primer |
| SEQ ID NO: 36 | N101T11-F001 WT Forward Primer |
| SEQ ID NO: 37 | N101T11-R001 WT Reverse Primer |
| SEQ ID NO: 38 | N101T11-001-X001 WT Probe |
| SEQ ID NO: 39 | N101T10-F001 Deletion F Primer |
| SEQ ID NO: 40 | N101T10-R001 Deletion R Primer |
| SEQ ID NO: 41 | N101T10-001-X001 Deletion Probe |
| SEQ ID NO: 42 | N101T10-F002 Deletion Forward Primer |
| SEQ ID NO: 43 | N101T10-R002 Deletion Reverse Primer |
| SEQ ID NO: 44 | N101T10-001-X002 Deletion Probe |
| SEQ ID NO: 45 | Probe Sequence for BnIND-A locus SNPs (see Table 5 herein) |
| SEQ ID NO: 46 | N101T10-R003 Deletion Reverse Primer |
| SEQ ID NO: 47 | N101T10-001-X003 Deletion Probe |
| SEQ ID NO: 48 | N101T11-R002 WT Forward Primer |
| SEQ ID NO: 49 | N101T11-001-X002 WT Probe |
| SEQ ID NO: 50 | N101T11-001-X003 WT Probe |
| SEQ ID NO: 51 | BP_F002 Deletion Forward Primer |
| SEQ ID NO: 52 | BP_R002 Deletion Reverse Primer |
| SEQ ID NO: 53 | BP_F003 Deletion Forward Primer |
| SEQ ID NO: 54 | BP_R003 Deletion Reverse Primer |
| SEQ ID NO: 55 | BP_R004 Deletion Reverse Primer |
| SEQ ID NO: 56 | BP_R005 Deletion Reverse Primer |
| SEQ ID NO: 57 | BP_R006 Deletion Reverse Primer |
| SEQ ID NO: 58 | IND_A_001_R002 WT Reverse Primer |
| SEQ ID NOs: 59-160 | Probe Sequences for BnIND-A locus SNPs (see Tables 4 and 5 herein) |

### DETAILED DESCRIPTION

### Terms and Definitions

“ALCATRAZ gene”, “ALC gene”, “ALCATRAZ allele” or “ALC allele” refers herein to a gene that can contribute to pod shatter resistance in B. napus and A. thaliana. ALC gene plays a role in cell separation during fruit dehiscence by promoting the differentiation of a cell layer that is the site of separation between the valves and the replum within the dehiscence zone. Examples of ALC gene sequences include BnALC-A (e.g. SEQ ID NO:5 or 26) and BnALC-C(SEQ ID NO:6 or 27).

An “allele” is one of several alternative forms of a gene occupying a given locus on a chromosome. When all the alleles present at a given locus on a chromosome are the same, that plant is “homozygous” at that locus. If the alleles present at a given locus on a chromosome differ, that plant is “heterozygous” at that locus. In B. napus, a plant can be homozygous wildtype for the IND gene in the A genome, but heterozygous mutant for the IND gene in the C genome.

An “amplicon” is amplified nucleic acid, e.g., a nucleic acid that is produced by amplifying a template nucleic acid by any available amplification method (e.g., PCR, LCR, transcription, or the like).

“Backcrossing” refers to the process whereby hybrid progeny plants are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. Backcrossing has been widely used to introduce new traits into plants. See e.g., Jensen, N., Ed. Plant Breeding Methodology, John Wiley & Sons, Inc., 1988. In a typical backcross protocol, the original variety of interest (recurrent parent) is crossed to a second variety (non-recurrent parent) that carries a gene of interest to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred gene from the nonrecurrent parent.

“Brassica” refers to any one of Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16).

A “Cas protein” refers to a polypeptide encoded by a Cas (CRISPR-associated) gene. A Cas protein includes but is not limited to: a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence.

A “Cas endonuclease” may comprise domains that enable it to function as a double-strand-break-inducing agent. A “Cas endonuclease” may also comprise one or more modifications or mutations that abolish or reduce its ability to cleave a double-strand polynucleotide (dCas). In some aspects, the Cas endonuclease molecule may retain the ability to nick a single-strand polynucleotide (for example, a D10A mutation in a Cas9 endonuclease molecule) (nCas9). When complexed with a guide polynucleotide, the guide polynucleotide/Cas endonuclease complex”, (or “guide polynucleotide/Cas endonuclease system”, “guide polynucleotide/Cas complex”, “guide polynucleotide/Cas system” and “guided Cas system” or “Polynucleotide-guided endonuclease”, “PGEN”” are capable of directing the Cas endonuclease to a DNA target site, enabling the Cas endonuclease to recognize, bind to, and nick or cleave (introduce a single or double-strand break) the DNA target site. A guided Cas system referred to herein can comprise Cas protein(s) and suitable polynucleotide component(s) of any known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

As used herein, the term “commercially useful” refers to plant lines and hybrids that have sufficient plant vigor and fertility, such that a crop of the plant line or hybrid can be produced by farmers using conventional farming equipment. In particular embodiments, plant commodity products with described components and/or qualities may be extracted from plants or plant materials of the commercially useful variety. For example, oil comprising desired oil components may be extracted from the seed of a commercially useful plant line or hybrid utilizing conventional crushing and extraction equipment. In another example, canola meal may be prepared from the crushed seed of commercially useful plant lines which are provided by the invention and which have one or more BnIND-A deletion allele disclosed herein. In certain embodiments, a commercially useful plant line is an inbred line or a hybrid line. “Agronomically elite” lines and hybrids typically have desirable agronomic characteristics; for example and without limitation: improved yield of at least one plant commodity product; maturity; disease resistance; and standability.

The term “cross” (or “crossed”) refers to the fusion of gametes via pollination to produce progeny (e.g., cells, seeds, and plants). This term encompasses both sexual crosses (i.e., the pollination of one plant by another) and selfing (i.e., self-pollination, for example, using pollen and ovule from the same plant).

The terms “dropout”, “gene dropout”, “knockout” and “gene knockout” refer to a DNA sequence of a cell (e.g. the BnIND-C gene or BnALC gene) that has been excised from the genome by targeted deletion mediated by a Cas protein.

The term “elite line” means any line that has resulted from breeding and selection for superior agronomic performance. An elite plant is any plant from an elite line.

The term “gene” (or “genetic element”) may refer to a heritable genomic DNA sequence with functional significance. A gene includes a nucleic acid fragment that expresses a functional molecule such as, but not limited to, a specific protein, including regulatory sequences preceding (5′ non-coding sequences) and following (3′ non-coding sequences) the coding sequence, as well as intervening intron sequences. The term “gene” may also be used to refer to, for example and without limitation, a cDNA and/or an mRNA encoded by a heritable genomic DNA sequence.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

A “genomic sequence” or “genomic region” is a segment of a chromosome in the genome of a cell that is present on either side of the target site or, alternatively, also comprises the target site or a portion thereof. An “endogenous genomic sequence” refers to genomic sequence within a plant cell, (e.g. an endogenous genomic sequence of an IND gene present within the genome of a Brassica plant cell).

A “genomic locus” as used herein refers to the genetic or physical location on a chromosome of a gene. As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

The term “genotype” refers to the physical components, i.e., the actual nucleic acid sequence at one or more loci in an individual plant.

The term “germplasm” refers to genetic material of or from an individual plant or group of plants (e.g., a plant line, variety, and family), and a clone derived from a plant or group of plants. A germplasm may be part of an organism or cell, or it may be separate (e.g., isolated) from the organism or cell. In general, germplasm provides genetic material with a specific molecular makeup that is the basis for hereditary qualities of the plant. As used herein, “germplasm” refers to cells of a specific plant; seed; tissue of the specific plant (e.g., tissue from which new plants may be grown); and non-seed parts of the specific plant (e.g., leaf, stem, pollen, and cells).

The term “germplasm” is synonymous with “genetic material,” and it may be used to refer to seed (or other plant material) from which a plant may be propagated. A “germplasm bank” may refer to an organized collection of different seed or other genetic material (wherein each genotype is uniquely identified) from which a known cultivar may be cultivated, and from which a new cultivar may be generated. In embodiments, a germplasm utilized in a method or plant as described herein is from a canola line or variety. In particular examples, a germplasm is seed of the canola line or variety. In particular examples, a germplasm is a nucleic acid sample from the canola line or variety.

A “haplotype” is the genotype of an individual at a plurality of genetic loci. In some examples, the genetic loci described by a haplotype may be physically and genetically linked; i.e., the loci may be positioned on the same chromosome segment.

The terms “increased” or “improved” in connection with “pod shatter tolerance” or “pod shatter resistance” as well as “reduced seed shattering” are used herein to reference decreased seed shatter tendency and/or a delay in the timing of seed shattering, in particular until harvest, of Brassica plants, the fruits of which normally do not mature synchronously, but sequentially, so that some pods burst open and shatter their seeds before or during harvest.

The term “INDEHISCENT gene”, “IND gene”, “INDEHISCENT allele” or “IND allele” refers herein to a gene that can contribute to pod shatter resistance in B. napus and A. thaliana. IND encodes a member of an atypical class of eukaryotic bHLH proteins which are required for seed dispersal. IND genes are involved in the differentiation of all three cell types required for fruit dehiscence and acts as the key regulator in a network that controls specification of the valve margin. Examples of IND gene sequences include BnIND-A (SEQ ID NOs: 2, 11, and 22) and BnIND-C(SEQ ID NOs: 3, 13, and 24).

In connection with pod shatter phenotypes evaluated herein, “fully shattered pods” are those with both valves detached from the replum and all seeds dispersed. “Half shattered pods” are those with one valve fully or partially detached from the replum, seeds dispersed, though the second valve is still attached and all or some seeds remain between the attached valve and the septum. “Unshattered pods” have both valves attached to the replum and seeds are contained between both valves and the septum. The “Percent shattered pods” or “SHTPC” is used herein as a quantitative measure of seed pod integrity after a laboratory assay or field trial shatter inducing treatment. In laboratory assay results, SHTPC refers to the number of fully shattered+half shattered pods/total number of pods*100%. In field trial results, SHTPC refers to the number of fully shattered/total number of pods*100%.

As used herein, the term “introgression” refers to the transmission of an allele at a genetic locus into a genetic background. In some embodiments, introgression of a specific allele form at the locus may occur by transmitting the allele form to at least one progeny via a sexual cross between two parents of the same species, where at least one of the parents has the specific allele form in its genome. Progeny comprising the specific allele form may be repeatedly backcrossed to a line having a desired genetic background. Backcross progeny may be selected for the specific allele form, so as to produce a new variety wherein the specific allele form has been fixed in the genetic background. In some embodiments, introgression of a specific allele form may occur by recombination between two donor genomes (e.g., in a fused protoplast), where at least one of the donor genomes has the specific allele form in its genome. Introgression may involve transmission of a specific allele form that may be, for example and without limitation, a selected allele form of a marker allele, a QTL, and/or a transgene. In this disclosure, introgression may involve transmission of one or more alleles of the native BnIND-A deletion (provided by this disclosure) into a progeny plant.

As used herein an “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component. For example and without limitation, a nucleic acid may be isolated from a chromosome by breaking chemical bonds connecting the nucleic acid to the remaining DNA in the chromosome and/or the other material previously associated with the nucleic acid in its cellular milieu (e.g., the nucleus). Nucleic acid molecules and proteins that have been “isolated” include nucleic acid molecules and proteins that are enriched or purified. The term also embraces nucleic acids and proteins prepared by recombinant expression in a host cell, as well as chemically-synthesized nucleic acid molecules, proteins, and peptides.

Marker: Unlike DNA sequences that encode proteins, which are generally well-conserved within a species, other regions of DNA (e.g., non-coding DNA and introns) tend to develop and accumulate polymorphism, and therefore may be variable between individuals of the same species. The genomic variability can be of any origin, for example, the variability may be due to DNA insertions, deletions, duplications, repetitive DNA elements, point mutations, recombination events, and the presence and sequence of transposable elements. Such regions may contain useful molecular genetic markers. In general, any differentially inherited polymorphic trait (including nucleic acid polymorphisms) that segregates among progeny is a potential marker.

As used herein, the terms “marker” and “molecular marker” refer to a nucleic acid or encoded product thereof (e.g., a protein) used as a point of reference when identifying a linked locus. Thus, a marker may refer to a gene or nucleic acid that can be used to identify plants having a particular allele. A marker may be described as a variation at a given genomic locus. A genetic marker may be a short DNA sequence, such as a sequence surrounding a single base-pair change (single nucleotide polymorphism, or “SNP”), or a long one, for example, a microsatellite/simple sequence repeat (“SSR”). A “marker allele” or “marker allele form” refers to the version of the marker that is present in a particular individual. The term “marker” as used herein may refer to a cloned segment of chromosomal DNA, and may also or alternatively refer to a DNA molecule that is complementary to a cloned segment of chromosomal DNA. The term also refers to nucleic acid sequences complementary to genomic marker sequences, such as nucleic acid primers and probes.

A marker may be described, for example, as a specific polymorphic genetic element at a specific location in the genetic map of an organism. A genetic map may be a graphical representation of a genome (or a portion of a genome, such as a single chromosome) where the distances between landmarks on the chromosome are measured by the recombination frequencies between the landmarks. A genetic landmark can be any of a variety of known polymorphic markers, for example and without limitation: simple sequence repeat (SSR) markers; restriction fragment length polymorphism (RFLP) markers; and single nucleotide polymorphism (SNP) markers. As one example, SSR markers can be derived from genomic or expressed nucleic acids (e.g., expressed sequence tags (ESTs)).

Additional markers include, for example and without limitation, ESTs; amplified fragment length polymorphisms (AFLPs) (Vos et al., 1995, Nucl. Acids Res. 23:4407; Becker ct al., 1995, Mol. Gen. Genet. 249:65; Meksem et al., 1995, Mol. Gen. Genet. 249:74); randomly amplified polymorphic DNA (RAPD); and isozyme markers. Isozyme markers may be employed as genetic markers, for example, to track isozyme markers or other types of markers that are linked to a particular first marker. Isozymes are multiple forms of enzymes that differ from one another with respect to amino acid sequence (and therefore with respect to their encoding nucleic acid sequences). Some isozymes are multimeric enzymes containing slightly different subunits. Other isozymes are either multimeric or monomeric, but have been cleaved from a pro-enzyme at different sites in the pro-enzyme amino acid sequence. Isozymes may be characterized and analyzed at the protein level or at the nucleic acid level. Thus, any of the nucleic acid based methods described herein can be used to analyze isozyme markers in particular examples.

Accordingly, genetic marker alleles that are polymorphic in a population can be detected and distinguished by one or more analytic methods such as, PCR-based sequence specific amplification methods, RFLP analysis, AFLP analysis, isozyme marker analysis, SNP analysis, SSR analysis, allele specific hybridization (ASH) analysis, detection of amplified variable sequences of the plant genome, detection of self-sustained sequence replication, detection of simple sequence repeats (SSRs), randomly amplified polymorphic DNA (RAPD) analysis. Thus, in certain examples of the invention, such known methods can be used to detect the BnIND-A deletion breakpoint and flanking sequence(s) as well as the SNP markers for detecting the presence or absence of the BIND-A deletion allele which are disclosed herein. Sec, e.g., Tables 1, 4, and 5 herein.

Numerous statistical methods for determining whether markers are genetically linked to a QTL (or to another marker) are known to those of skill in the art and include, for example and without limitation, standard linear models (e.g., ANOVA or regression mapping; Haley and Knott, 1992, Heredity 69:315); and maximum likelihood methods (e.g., expectation-maximization algorithms; Lander and Botstein, 1989, Genetics 121:185-99; Jansen, 1992, Theor. Appl. Genet. 85:252-60; Jansen, 1993, Biometrics 49:227-31; Jansen, 1994, “Mapping of quantitative trait loci by using genetic markers: an overview of biometrical models,” In J. W. van Ooijen and J. Jansen (eds.), Biometrics in Plant breeding: applications of molecular markers, pp. 116-24 (CPRO-DLO Netherlands); Jansen, 1996, Genetics 142:305-11; and Jansen and Stam, 1994, Genetics 136:1447-55).

Exemplary statistical methods include single point marker analysis; interval mapping (Lander and Botstein, 1989, Genetics 121:185); composite interval mapping; penalized regression analysis; complex pedigree analysis; MCMC analysis; MQM analysis (Jansen, 1994, Genetics 138:871); HAPLO-IM+ analysis, HAPLO-MQM analysis, and HAPLO-MQM+ analysis; Bayesian MCMC; ridge regression; identity-by-descent analysis; and Haseman-Elston regression, any of which are suitable in the context of particular embodiments of the invention. Alternative statistical methods applicable to complex breeding populations that may be used to identify and localize QTLs in particular examples are described in U.S. Pat. No. 6,399,855 and PCT International Patent Publication No. WO0149104 A2. All of these approaches are computationally intensive and are usually performed with the assistance of a computer-based system comprising specialized software. Appropriate statistical packages are available from a variety of public and commercial sources, and are known to those of skill in the art.

“Marker-assisted selection” (MAS) is a process by which phenotypes are selected based on marker genotypes. Marker assisted selection includes the use of marker genotypes for identifying plants for inclusion in and/or removal from a breeding program or planting.

Molecular marker technologies generally increase the efficiency of plant breeding through MAS. A molecular marker allele that demonstrates linkage disequilibrium with a desired phenotypic trait (e.g., a QTL) provides a useful tool for the selection of the desired trait in a plant population. The key components to the implementation of an MAS approach are the creation of a dense (information rich) genetic map of molecular markers in the plant germplasm; the detection of at least one QTL based on statistical associations between marker and phenotypic variability; the definition of a set of particular useful marker alleles based on the results of the QTL analysis; and the use and/or extrapolation of this information to the current set of breeding germplasm to enable marker-based selection decisions to be made.

The closer a particular marker is to a gene that encodes a polypeptide that contributes to a particular phenotype (whether measured in terms of genetic or physical distance), the more tightly-linked is the particular marker to the phenotype. In view of the foregoing, it will be appreciated that the closer (whether measured in terms of genetic or physical distance) that a marker is linked to a particular gene, the more likely the marker is to segregate with that gene (e.g., the BnIND-A deletion disclosed herein) and its associated phenotype (e.g., the contribution to pod shatter tolerance of the BnIND-A deletion disclosed herein). Thus, the extremely tightly linked genetic markers of the BnIND-A deletion disclosed herein can be used in MAS programs to identity canola varieties that have or can generate progeny that have increased pod shatter tolerance (when compared to parental varieties and/or otherwise isogenic plants lacking the BnIND-A deletion), to identify individual canola plants comprising this increased pod shatter tolerance trait, and to breed this trait into other canola varieties to improve their pod shatter tolerance.

A “marker set” or a “set” of markers or probes refers to a specific collection of markers (or data derived therefrom) that may be used to identify individuals comprising a trait of interest. In some embodiments, a set of markers linked to a BnIND-A deletion may be used to identify a Brassica plant comprising one or more allele of the BnIND-A deletion disclosed herein. Data corresponding to a marker set (or data derived from the use of such markers) may be stored in an electronic medium. While each marker in a marker set may possess utility with respect to trait identification, individual markers selected from the set and subsets including some, but not all, of the markers may also be effective in identifying individuals comprising the trait of interest.

A “mutated gene” or “modified gene” is a gene that has been altered through human intervention. Such a “mutated” or “modified” gene has a sequence that differs from the sequence of the corresponding non-mutated gene by at least one nucleotide addition, deletion, or substitution. In certain embodiments of the disclosure, the mutated gene comprises an excision or deletion of a sequence of nucleotides within that results from two double strands break which are specifically targeted to a genomic sequence by guide polynucleotide/Cas endonuclease system as disclosed herein. A “mutated” or “modified” plant is a plant comprising a mutated gene or deletion. As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

As used herein the term “native gene” refers to a gene as found in its natural endogenous location with its own regulatory sequences. In the context of this disclosure, a “mutated” or “modified” gene is not a native gene.

As used herein, a ‘nucleic acid molecule” is a polymeric form of nucleotides, which can include both sense and anti-sense strands of RNA, cDNA, genomic DNA, and synthetic forms and mixed polymers of the above. A nucleotide refers to a ribonucleotide, deoxynucleotide, or a modified form of either type of nucleotide. A “nucleic acid molecule” as used herein is synonymous with “nucleic acid”, “nucleotide sequence”, “nucleic acid sequence”, and “polynucleotide.” The term includes single- and double-stranded forms of DNA. A nucleic acid molecule can include either or both naturally occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., peptides), intercalators (e.g., acridine, psoralen, etc.), chelators, alkylators, and modified linkages (e.g., alpha anomeric nucleic acids, etc.). The term “nucleic acid molecule” also includes any topological conformation, including single-stranded, double-stranded, partially duplexed, triplexed, hairpinned, circular, and padlocked conformations. An “endogenous nucleic acid sequence” refers to a nucleic acid sequence within a plant cell, (e.g. an endogenous allele of an IND gene present within the genome of a Brassica plant cell).

The term “single-nucleotide polymorphism” (SNP) refers to a DNA sequence variation occurring when a single nucleotide in the genome (or other shared sequence) differs between members of a species or paired chromosomes in an individual. In some examples, markers linked to a BnIND-A deletion disclosed herein are SNP markers. Recent high-throughput genotyping technologies such as GoldenGate® and INFINIUM® assays (Illumina, San Diego, CA) may be used in accurate and quick genotyping methods by multiplexing SNPs from 384-plex to >100,000-plex assays per sample.

As used herein, “phenotype” means the detectable characteristics (e.g. pod shatter tolerance) of a cell or organism which can be influenced by genotype.

As used herein, the term “plant material” refers to any processed or unprocessed material derived, in whole or in part, from a plant. For example, and without limitation, a plant material may be a plant part, a seed, a fruit, a leaf, a root, a plant tissue, a plant tissue culture, a plant explant, or a plant cell.

As used herein, the term “plant” may refer to a whole plant, a cell or tissue culture derived from a plant, and/or any part of any of the foregoing. Thus, the term “plant” encompasses, for example and without limitation, whole plants; plant components and/or organs (e.g., leaves, stems, and roots); plant tissue; seed; and a plant cell. A plant cell may be, for example and without limitation, a cell in and/or of a plant, a cell isolated from a plant, and a cell obtained through culturing of a cell isolated from a plant. Thus, the term Brassica “plant” may refer to, for example and without limitation, a whole Brassica plant; multiple Brassica plants; Brassica plant cell(s); Brassica plant protoplast; Brassica tissue culture (e.g., from which a canola plant can be regenerated); Brassica plant callus; Brassica plant parts (e.g., seed, flower, cotyledon, leaf, stem, bud, root, and root tip); and Brassica plant cells that are intact in a Brassica plant or in a part of a Brassica plant.

As used herein, a plant or Brassica “line” refers to a group of plants that display little genetic variation (e.g., no genetic variation) between individuals for at least one trait. Inbred lines may be created by several generations of self-pollination and selection or, alternatively, by vegetative propagation from a single parent using tissue or cell culture techniques. As used herein, the terms “cultivar,” “variety,” and “type” are synonymous, and these terms refer to a line that is used for commercial production.

Trait or phenotype: The terms “trait” and “phenotype” are used interchangeably herein. For the purposes of the present disclosure, the traits of particular interest are the pod shatter tolerance trait disclosed herein.

A “variety” or “cultivar” is a plant line that is used for commercial production which is distinct, stable and uniform in its characteristics when propagated. In the case of a hybrid variety or cultivar, the parental lines are distinct, stable, and uniform in their characteristics.

The term “POLYGALACTURONASE gene”, “POLYGALACTURONASE allele”, “PGAZ gene” or “PGAZ allele” refers herein to “polygalacturonase expressed in abscission zone” gene. PGAZ is involved in pectin degradation and subsequent loss of cell cohesion (Hadfield and Bennet 1998, Plant physiology, 117 (2), 337-343.). PGAZ expression increases during a number of developmental processes thought to involve cell wall breakdown, including silique shattering (Jenkins et al., 1996, Journal of Exp. Botany, 47 (1), 111-115; Jenkins et al., 1999, Plant, Cell & Environment, 22 (2), 159-167; Ferrándiz, 2002, Journal of Exp. Botany, 53 (377), 2031-2038). Examples of PGAZ genes include BnPGAZ-A (SEQ ID No:7, 15, or 28) and BnPGAZ-C(SEQ ID NO: 9, 18, or 30).

### Detection of Native Deletion in BIND-A.

The methods and assays of the disclosure are based, at least in part, on the discovery of an unexpected deletion of genomic sequence that affects the INDEHISCENT gene on chromosome N03 (BnIND-A) of B. napus. The deletion was discovered by whole genome sequencing B. napus line G00010BC and comparing its BnIND-A sequence to that of a number of other reference genomes, which revealed a large deleted segment. As compared to reference genomes for lines NS1822BC, DH12075_v1.1, Darmor_v4.1.1, and G0055MC, the BnIND-A deletion corresponds to a deleted segment (loss of genomic sequence) ranging from about 200 kb to about 310 kb in length. See Example 1 herein.

The BnIND-A deletion disclosed herein can be detected by nucleotide sequencing and/or amplification of the genomic DNA, which will reveal the absence of the 200 kb to about 310 kb deleted genomic segment disclosed herein. For example, the BnIND-A deletion can be detected by nucleotide sequencing and/or amplification of genomic sequencing flanking and including the deletion breakpoint locus at positions 10,002-10,003 of SEQ ID NO:2. Such sequencing or amplification of a BnIND-A deletion allele will produce a sequencing product or amplicon comprising the following deletion breakpoint locus (start breakpoint and end breakpoint positions shown in bold and underlined): ATTTCTCTATTTGTTTT (SEQ ID NO:2, positions 9995-1011).

In particular examples, detecting the BnIND-A deletion can include DNA sequencing, amplification, or the combined amplification and sequencing of the breakpoint locus and 5 bp or more, 10 bp or more, 15 bp or more, 20 bp or more, 30 bp or more, 40 bp or more, 50 bp or more, 60 bp or more, 70 bp or more, 80 bp or more, 90 bp or more, 100 bp or more, 110 bp or more, 120 bp or more, 130 bp or more, 140 bp or more, 150 bp or more, 175 bp or more, 200 bp or more, 250 bp or more, 300 bp or more, 350 bp or more, 400 bp or more, 450 bp or more, 500 bp or more, 550 bp or more, or 600 bp or more of flanking sequence that is (i) upstream of (i.e., located 5′ to) the deletion start breakpoint at position 10,002 of SEQ ID NO:2 and/or (ii) downstream of (i.e., located 3′ to) the deletion end breakpoint at position 10,003 of SEQ ID NO:2. Thus, in particular examples, the BnIND-A deletion disclosed herein can be detected by amplifying genomic sequence to produce an amplicon comprising one or more of the BnIND-A deletion allele sequences identified in Table 1 above. Additionally, the BnIND-A deletion disclosed herein can be detected by nucleotide sequencing to detect the presence of the genomic sequence (including, e.g., by first amplifying genomic sequence and sequencing the amplicon or amplified genomic sequence) comprising a BnIND-A deletion allele sequence identified in Table 1 above.

By contrast, wildtype BnIND-A sequence does not include the deletion breakpoint locus sequence corresponding to positions 10,002-10,003 of SEQ ID NO:2 because in wild type genomic DNA, the deletion start breakpoint (position 10,002 of SEQ ID NO:2) and end breakpoint (position 10,003 of SEQ ID NO:2) are separated by an intervening genomic segment (the deletion segment) that can range from about 200 kb to about 310 kb in length. Due to the presence of this intervening segment, sequencing or amplification of wildtype BnIND-A sequence will not produce a sequencing product or amplicon comprising any of the sequences disclosed in Table 1.

Detection of the BnIND-A deletion allele disclosed herein can be done using any method for detecting polymorphisms. Additionally, such methods can be used to detect a polymorphic marker that is genetically linked to the BnIND-A deletion allele. These methods include allele-specific amplification and PCR based amplification assays such as TaqMan, rhAmp-SNP, KASPar, and molecular beacons. Such an assay can include the use of one or more probes that detect the breakpoint locus of the BnIND-A deletion allele, a marker associated with the deletion, or an amplicon that is selectively amplified by amplification of genomic sequence comprising the BnIND-A deletion. Optionally, such an assay can further include an additional set of primers and/or one or more probes that detect the presence of a BnIND-A (e.g., wildtype allele) that includes the intervening ˜200 kb to ˜310 kb genomic segment between deletion breakpoints, as disclosed herein.

Additional methods for genotyping and detecting the BnIND-A deletion allele disclosed herein (or a linked marker) include but are not limited to, hybridization, primer extension, oligonucleotide ligation, nuclease cleavage, minisequencing and coded spheres. Such methods are reviewed in publications including Gut, 2001, Hum. Mutat. 17:475; Shi, 2001, Clin. Chem. 47:164; Kwok, 2000, Pharmacogenomics 1:95; Bhattramakki and Rafalski, “Discovery and application of single nucleotide polymorphism markers in plants”, in PLANT GENOTYPING: THE DNA FINGERPRINTING OF PLANTS (CABI Publishing, Wallingford 2001). A wide range of commercially available technologies utilize these and other methods to interrogate the BnIND-A deletion allele disclosed herein (or a linked marker), including Masscode™ (Qiagen, Germantown, Md.), Invader® (Hologic, Madison, Wis.), SnapShot® (Applied Biosystems, Foster City, Calif.), Taqman® (Applied Biosystems, Foster City, Calif.) and Infinium Bead Chip™ and GoldenGate™ allele-specific extension PCR-based assay (Illumina, San Diego, Calif.).

In one method, the BnIND-A deletion allele can be detected by confirming the absence of genomic sequence comprising one or more N03 genomic markers, e.g., SNPs, located within the deleted genomic segment disclosed herein. This absence can be confirmed using a commercially available substrate (e.g., Infinium Bead Chip™) having an array of probes for markers on Brassica chromosome N03. These are suitable for individual or high-throughput screening of N03 markers in genomic samples. For example, Table 4 below provides SNP markers and probes that bind to the markers, which are located within the deleted N03 genomic segment disclosed herein. Because genomic DNA comprising the BnIND-A deletion allele disclosed herein does not include the marker sequences that bind to the probes in Table 4 below, a sample containing genomic DNA or amplified genomic DNA sequence comprising the BnIND-A deletion will not bind to and will not generate a signal from these probes. By contrast, wildtype genomic DNA sequence retains the deleted segment and, therefore, can bind to and generate a signal from these probes. In view of the foregoing, one or more SNP marker shown in Table 4 can be used to distinguish the BnIND-A deletion genomic sequence disclosed herein from wildtype BnIND-A genomic sequence. Table 4 identifies probe sequence, commercial marker name (from Illumina), N03 SNP maker name, assay chemistry type, and genomic position (using DH12075 reference genome) of probes that detect wildtype sequence located within the deleted genomic segment disclosed herein.

TABLE 4

| Probe | | | | Genomic |
|---|---|---|---|---|
| Sequence | Marker Name | SNP Name | Chemistry | Position |
| SEQ ID NO: 59 | N0014XD-001-I002 | N0014XD-001 | Illumina | 14,453,580 |
| SEQ ID NO: 60 | N0014XE-001-I002 | N0014XE-001 | Illumina | 14,454,264 |
| SEQ ID NO: 61 | N0014XF-001-I002 | N0014XF-001 | Illumina | 14,479,202 |
| SEQ ID NO: 62 | N0014XG-001-I002 | N0014XG-001 | Illumina | 14,479,225 |
| SEQ ID NO: 63 | N0014XH-001-I002 | N0014XH-001 | Illumina | 14,479,295 |
| SEQ ID NO: 64 | N0014XJ-001-I002 | N0014XJ-001 | Illumina | 14,483,678 |
| SEQ ID NO: 65 | N0014XK-001-I002 | N0014XK-001 | Illumina | 14,484,035 |
| SEQ ID NO: 66 | N0014XM-001-I002 | N0014XM-001 | Illumina | 14,484,391 |
| SEQ ID NO: 67 | N002C2M-001-I002 | N002C2M-001 | Illumina | 14,484,901 |
| SEQ ID NO: 68 | N0014XP-001-I003 | N0014XP-001 | IlluminaXT | 14,508,983 |
| SEQ ID NO: 69 | N0014XP-001-Q001 | N0014XP-001 | TaqMan | 14,508,983 |
| SEQ ID NO: 70 | N0014XP-001-I002 | N0014XP-001 | Illumina | 14,508,983 |
| SEQ ID NO: 71 | N0014XR-001-I002 | N0014XR-001 | Illumina | 14,509,547 |
| SEQ ID NO: 72 | N0014XT-001-I002 | N0014XT-001 | Illumina | 14,531,140 |
| SEQ ID NO: 73 | N0014XU-001-I002 | N0014XU-001 | Illumina | 14,531,428 |
| SEQ ID NO: 74 | N0014XV-001-I002 | N0014XV-001 | Illumina | 14,535,159 |
| SEQ ID NO: 75 | N0014MV-001-I003 | N0014MV-001 | IlluminaXT | 14,543,573 |
| SEQ ID NO: 76 | N0014MV-001-Q001 | N0014MV-001 | TaqMan | 14,543,573 |
| SEQ ID NO: 77 | N0014MV-001-I002 | N0014MV-001 | Illumina | 14,543,573 |
| SEQ ID NO: 78 | N0014XX-001-I002 | N0014XX-001 | Illumina | 14,544,098 |
| SEQ ID NO: 79 | N0014XY-001-I002 | N0014XY-001 | Illumina | 14,584,738 |
| SEQ ID NO: 80 | N0014Y0-001-I002 | N0014Y0-001 | Illumina | 14,599,964 |
| SEQ ID NO: 81 | N0014Y1-001-I002 | N0014Y1-001 | Illumina | 14,600,153 |
| SEQ ID NO: 82 | N0014Y2-001-I002 | N0014Y2-001 | Illumina | 14,609,027 |
| SEQ ID NO: 83 | N0014Y3-001-I002 | N0014Y3-001 | Illumina | 14,611,514 |
| SEQ ID NO: 84 | N0014Y4-001-I002 | N0014Y4-001 | Illumina | 14,617,650 |
| SEQ ID NO: 85 | N0014Y5-001-I002 | N0014Y5-001 | Illumina | 14,622,130 |
| SEQ ID NO: 86 | N0014Y6-001-I003 | N0014Y6-001 | IlluminaXT | 14,626,122 |
| SEQ ID NO: 87 | N0014Y6-001-Q001 | N0014Y6-001 | TaqMan | 14,626,122 |
| SEQ ID NO: 88 | N0014Y6-001-I002 | N0014Y6-001 | Illumina | 14,626,122 |
| SEQ ID NO: 89 | N0014Y7-001-I002 | N0014Y7-001 | Illumina | 14,627,320 |
| SEQ ID NO: 90 | N0014Y8-001-I002 | N0014Y8-001 | Illumina | 14,630,481 |
| SEQ ID NO: 91 | N0014Y9-001-I002 | N0014Y9-001 | Illumina | 14,630,797 |
| SEQ ID NO: 92 | N0014YA-001-I002 | N0014YA-001 | Illumina | 14,641,501 |

In another method, the BnIND-A deletion allele can be detected by confirming the presence of genomic sequence comprising one or more N03 genomic marker (e.g., SNP) alleles which are located on sequencing flanking the deletion breakpoint disclosed herein and which are genetically linked to the BIND-A deletion allele (but are not genetically linked to the presence of the deleted genomic segment disclosed herein). For example, Table 5 identifies probe sequence, commercial marker name (from Illumina), N03 SNP maker name, assay chemistry type, and genomic position (using DH12075 reference genome) of probes for markers that are flanking the deletion breakpoint of the disclosed BnIND-A deletion.

TABLE 5

| | | | | Genomic |
|---|---|---|---|---|
| SEQ ID NO | Marker Name | SNP Name | Chemistry | Position |
| SEQ ID NO: 93 | N0014ME-001-I002 | N0014ME-001 | Illumina | 14,236,228 |
| SEQ ID NO: 94 | N0014MF-001-I002 | N0014MF-001 | Illumina | 14,247,234 |
| SEQ ID NO: 95 | N0014MG-001-I002 | N0014MG-001 | Illumina | 14,247,416 |
| SEQ ID NO: 96 | N0014MH-001-I002 | N0014MH-001 | Illumina | 14,253,184 |
| SEQ ID NO: 97 | N0014MJ-001-I002 | N0014MJ-001 | Illumina | 14,273,849 |
| SEQ ID NO: 98 | N002W9W-001-I002 | N002W9W-001 | Illumina | 14,294,755 |
| SEQ ID NO: 99 | N0014MK-001-I002 | N0014MK-001 | Illumina | 14,303,293 |
| SEQ ID NO: 100 | N0014MM-001-I002 | N0014MM-001 | Illumina | 14,304,481 |
| SEQ ID NO: 101 | N0014MN-001-I002 | N0014MN-001 | Illumina | 14,304,515 |
| SEQ ID NO: 102 | N0014MP-001-I003 | N0014MP-001 | IlluminaXT | 14,309,064 |
| SEQ ID NO: 103 | N0014MP-001-Q001 | N0014MP-001 | TaqMan | 14,309,064 |
| SEQ ID NO: 104 | N0014MP-001-I002 | N0014MP-001 | Illumina | 14,309,064 |
| SEQ ID NO: 105 | N0014MR-001-I002 | N0014MR-001 | Illumina | 14,310,286 |
| SEQ ID NO: 106 | N0014WT-001-I002 | N0014WT-001 | Illumina | 14,311,970 |
| SEQ ID NO: 107 | N0014WV-001-I002 | N0014WV-001 | Illumina | 14,323,705 |
| SEQ ID NO: 108 | N0014WW-001-I002 | N0014WW-001 | Illumina | 14,331,357 |
| SEQ ID NO: 109 | N0014WX-001-I002 | N0014WX-001 | Illumina | 14,338,096 |
| SEQ ID NO: 110 | N0014WY-001-I002 | N0014WY-001 | Illumina | 14,341,517 |
| SEQ ID NO: 111 | N0014X0-001-I002 | N0014X0-001 | Illumina | 14,348,717 |
| SEQ ID NO: 112 | N0014X1-001-I002 | N0014X1-001 | Illumina | 14,350,104 |
| SEQ ID NO: 113 | N0014X2-001-I002 | N0014X2-001 | Illumina | 14,352,324 |
| SEQ ID NO: 114 | N0014X3-001-I002 | N0014X3-001 | Illumina | 14,356,474 |
| SEQ ID NO: 115 | N0014X4-001-I002 | N0014X4-001 | Illumina | 14,362,485 |
| SEQ ID NO: 116 | N0014X5-001-I002 | N0014X5-001 | Illumina | 14,366,149 |
| SEQ ID NO: 117 | N0014X6-001-I002 | N0014X6-001 | Illumina | 14,370,593 |
| SEQ ID NO: 118 | N0014X7-001-I002 | N0014X7-001 | Illumina | 14,371,157 |
| SEQ ID NO: 119 | N0014X8-001-I002 | N0014X8-001 | Illumina | 14,418,412 |
| SEQ ID NO: 120 | N0014X9-001-I002 | N0014X9-001 | Illumina | 14,422,444 |
| SEQ ID NO: 121 | N0014XA-001-I002 | N0014XA-001 | Illumina | 14,438,774 |
| SEQ ID NO: 122 | N0014XB-001-I002 | N0014XB-001 | Illumina | 14,439,353 |
| SEQ ID NO: 123 | N0014XC-001-I002 | N0014XC-001 | Illumina | 14,447,394 |
| — | — | — | — | — |
| SEQ ID NO: 124 | N0014YK-001-I002 | N0014YK-001 | Illumina | 14,693,565 |
| SEQ ID NO: 125 | N0014YM-001-I002 | N0014YM-001 | Illumina | 14,711,636 |
| SEQ ID NO: 126 | N0014YN-001-I002 | N0014YN-001 | Illumina | 14,713,821 |
| SEQ ID NO: 127 | N0014YP-001-I002 | N0014YP-001 | Illumina | 14,728,197 |
| SEQ ID NO: 128 | N0014YR-001-I002 | N0014YR-001 | Illumina | 14,729,444 |
| SEQ ID NO: 129 | N0014YT-001-I002 | N0014YT-001 | Illumina | 14,729,492 |
| SEQ ID NO: 130 | N0014YU-001-I002 | N0014YU-001 | Illumina | 14,732,311 |
| SEQ ID NO: 131 | N0014YV-001-I002 | N0014YV-001 | Illumina | 14,738,917 |
| SEQ ID NO: 132 | N002C1P-001-I002 | N002C1P-001 | Illumina | 14,747,648 |
| SEQ ID NO: 133 | N0014YY-001-I003 | N0014YY-001 | IlluminaXT | 14,752,111 |
| SEQ ID NO: 134 | N0014YY-001-Q001 | N0014YY-001 | TaqMan | 14,752,111 |
| SEQ ID NO: 135 | N0014YY-001-I002 | N0014YY-001 | Illumina | 14,752,111 |
| SEQ ID NO: 136 | N001500-001-I002 | N001500-001 | Illumina | 14,764,950 |
| SEQ ID NO: 137 | N001501-001-I003 | N001501-001 | IlluminaXT | 14,777,622 |
| SEQ ID NO: 138 | N001501-001-Q001 | N001501-001 | TaqMan | 14,777,622 |
| SEQ ID NO: 139 | N001501-001-I002 | N001501-001 | Illumina | 14,777,622 |
| SEQ ID NO: 140 | N001502-001-I002 | N001502-001 | Illumina | 14,791,402 |
| SEQ ID NO: 141 | N001573-001-I002 | N001573-001 | Illumina | 14,792,796 |
| SEQ ID NO: 142 | N001574-001-Q001 | N001574-001 | TaqMan | 14,793,905 |
| SEQ ID NO: 143 | N001574-001-I002 | N001574-001 | Illumina | 14,793,905 |
| SEQ ID NO: 144 | N001574-001-I003 | N001574-001 | IlluminaXT | 14,793,905 |
| SEQ ID NO: 145 | N001575-001-I002 | N001575-001 | Illumina | 14,794,327 |
| SEQ ID NO: 146 | N001576-001-I002 | N001576-001 | Illumina | 14,804,413 |
| SEQ ID NO: 147 | N001577-001-I002 | N001577-001 | Illumina | 14,805,191 |
| SEQ ID NO: 148 | N001578-001-I002 | N001578-001 | Illumina | 14,805,303 |
| SEQ ID NO: 149 | N001579-001-I003 | N001579-001 | IlluminaXT | 14,806,463 |
| SEQ ID NO: 150 | N001579-001-Q003 | N001579-001 | TaqMan | 14,806,463 |
| SEQ ID NO: 151 | N001579-001-I002 | N001579-001 | Illumina | 14,806,463 |
| SEQ ID NO: 152 | N00157A-001-I002 | N00157A-001 | Illumina | 14,845,698 |
| SEQ ID NO: 153 | N00157B-001-I002 | N00157B-001 | Illumina | 14,890,862 |
| SEQ ID NO: 154 | N00157C-001-I002 | N00157C-001 | Illumina | 14,896,577 |
| SEQ ID NO: 155 | N00157D-001-I002 | N00157D-001 | Illumina | 14,922,691 |
| SEQ ID NO: 156 | N00157E-001-I002 | N00157E-001 | Illumina | 14,927,519 |
| SEQ ID NO: 157 | N00157F-001-I002 | N00157F-001 | Illumina | 14,930,339 |
| SEQ ID NO: 158 | N00157G-001-I002 | N00157G-001 | Illumina | 14,931,181 |
| SEQ ID NO: 159 | N00157H-001-I002 | N00157H-001 | Illumina | 14,940,380 |
| SEQ ID NO: 160 | N00157J-001-I002 | N00157J-001 | Illumina | 14,954,238 |

Other methods of detecting the BnIND-A deletion allele disclosed herein, or a linked marker, includes single base extension (SBE) methods, which involve the extension of a nucleotide primer that is adjacent to a polymorphism to incorporate a detectable nucleotide residue upon extension of the primer through the polymorphism, e.g., extension through the BnIND-A deletion breakpoint disclosed herein (or a linked marker).

Methods of detecting the BnIND-A deletion allele disclosed herein (or a linked marker) also include LCR; and transcription-based amplification methods (e.g., SNP detection, SSR detection, RFLP analysis, and others). Useful techniques include hybridization of a probe nucleic acid to a nucleic acid corresponding to the BnIND-A deletion allele disclosed herein, or a linked marker (e.g., an amplified nucleic acid produced using a genomic canola DNA molecule as a template). Hybridization formats including, for example and without limitation, solution phase; solid phase; mixed phase; and in situ hybridization assays may be useful for allele detection in particular embodiments. An extensive guide to hybridization of nucleic acids is discussed in Tijssen, Laboratory Techniques in Biochemistry and Molecular Biology-Hybridization with Nucleic Acid Probes (Elsevier, NY. 1993).

Markers corresponding to genetic polymorphisms between members of a population may be detected by any of numerous methods including, for example and without limitation, nucleic acid amplification-based methods; and nucleotide sequencing of a polymorphic marker region. Many detection methods (including amplification-based and sequencing-based methods) may be readily adapted to high throughput analysis in some examples, for example, by using available high throughput sequencing methods, such as sequencing by hybridization.

The detecting of a BnIND-A deletion or a SNP allele associated with that BnIND-A deletion can be performed by any of a number or techniques, including, but not limited to, the use of nucleotide sequencing products, amplicons, or probes comprising detectable labels. Detectable labels suitable for use include any composition detectable by spectroscopic, radioisotopic, photochemical, biochemical, immunochemical, electrical, optical, or chemical means. Thus, a particular allele of a SNP may be detected using, for example, autoradiography, fluorography, or other similar detection techniques, depending on the particular label to be detected. Useful labels include biotin (for staining with labeled streptavidin conjugate), magnetic beads, fluorescent dyes, radiolabels, enzymes, and colorimetric labels. Other labels include ligands that bind to antibodies or specific binding targets labeled with fluorophores, chemiluminescent agents, and enzymes. In some embodiments of the present invention, detection techniques include the use of fluorescent dyes.

The BIND-A deletion allele disclosed herein is associated with a pod shatter tolerance trait. Therefore, any of the methods of detecting the BnIND-A deletion can be used to detect the presence of a pod shatter tolerance trait which is heritable and therefore useful in a breeding program, for example to create progeny Brassica plants comprising the BnIND-A deletion and one or more other desirable agronomic or end use qualities. Accordingly, in some aspects, the invention provides a method of selecting, detecting and/or identifying a Brassica plant, cell, or germplasm thereof (e.g., a seed) having the pod shatter tolerance trait. The method comprises detecting in said Brassica plant, cell, or germplasm thereof, the presence of the BnIND-A deletion or a marker associated with the BIND-A deletion and thereby identifying a Brassica plant having the pod shatter tolerance trait.

### Introgression of the Native BnIND-A Deletion in Brassica

As set forth herein, identification of Brassica, e.g., B. napus, B. juncea, B. carinata, B. rapa or B. oleracea plants or germplasm comprising the BnIND-A deletion allele responsible for the pod shatter tolerance trait disclosed herein, provides a basis for performing marker assisted selection of Brassica. For example, at least one Brassica plant that comprises the BnIND-A deletion allele is selected for and plants that do not include the deletion allele may be selected against.

This disclosure thus provides methods for selecting a canola plant exhibiting pod shatter tolerant trait comprising detecting in the plant the BnIND-A deletion allele (or one or more genetic markers associated with the BnIND-A deletion allele). This can be used in a method for selecting such a plant, the method comprises providing a sample of genomic DNA from a Brassica plant; and (b) using any method disclosed herein for detecting in the sample of genomic DNA the BnIND-A deletion allele or at least one genetic marker associated with the with the deletion allele.

This disclosure also provides a method comprising the transfer by introgression of the BnIND-A deletion allele from one plant into a recipient plant by crossing the plants. This transfer can be accomplished using, e.g., traditional breeding techniques to improve the pod shatter tolerance of the recipient plant and/or the progeny of the recipient plant. In one aspect, the BnIND-A deletion is introgressed into one or more commercial or elite Brassica varieties using marker-assisted selection (MAS) or marker-assisted breeding (MAB). MAS and MAB involve the use of one or more molecular markers that indicate the presence or co-segregation with BnIND-A deletion, and used for the identification and selection of those offspring plants that contain BnIND-A deletion. As disclosed herein, the molecular markers for the BnIND-A deletion include the deletion breakpoint sequence disclosed herein and any genomic sequence or amplicon disclosed herein which distinguish the BnIND-A deletion allele from a BIND-A (e.g., wildtype) allele that includes the intervening from about 200 kb to about 310 kb genomic segment between deletion breakpoints.

When a population is segregating for multiple loci affecting one or multiple traits, e.g., multiple loci involved in resistance to a single disease, or multiple loci each involved in resistance to different diseases, the efficiency of MAS compared to phenotypic screening becomes even greater because all the loci can be processed in the lab together from a single sample of DNA. Thus, MAS is particularly suitable for introgressing BnIND-A deletion allele into a plant line that includes one or more additional desirable traits. Additional desirable traits can include another pod shatter tolerance trait, disease resistance trait, or an end use trait such as oil quality or meal quality.

Another use of MAS in plant breeding is to assist the recovery of the recurrent parent genotype by backcross breeding. Backcross breeding is the process of crossing a progeny back to one of its parents. Backcrossing is usually done for the purpose of introgressing one or a few loci from a donor parent, i.e., the BnIND-A deletion, into an otherwise desirable genetic background from the recurrent parent. The more cycles of backcrossing that are done, the greater the genetic contribution of the recurrent parent to the resulting variety. This is desirable when the recurrent parent is an elite variety and/or has more desirable qualities than the donor plant, even though the recurrent parent may need improved pod shatter tolerance. For example, backcrossing can be desirable when a recurrent plant provides better yield, fecundity, oil and/or meal qualities and the like, as compared to the donor BnIND-A deletion plant.

MAB can also be used to develop near-isogenic lines (NIL) harboring the BnIND-A deletion disclosed herein, allowing a more detailed study of an effect of such allele. MAB is also an effective method for development of backcross inbred line (BIL) populations. Brassica plants developed according to these embodiments can derive a majority of their traits from the recipient plant and derive the pod shatter tolerance from the donor BnIND-A deletion plant. MAB/MAS techniques increase the efficiency of backcrossing and introgressing genes using marker-assisted selection (MAS) or marker-assisted breeding (MAB).

Thus, traditional breeding techniques can be used to introgress a nucleic acid sequence associated with native BIND-A deletion into a recipient Brassica plant. For example, inbred BnIND-A deletion Brassica plant lines can be developed using the techniques of recurrent selection and backcrossing, selfing, and/or dihaploids, or any other technique used to make parental lines. In a method of recurrent selection and backcrossing, the BnIND-A deletion can be introgressed into a target recipient plant (the recurrent parent) by crossing the recurrent parent with a first donor plant, which differs from the recurrent parent and is referred to herein as the “non-recurrent parent.” The recurrent parent is a plant, in some cases, comprises commercially desirable characteristics, such as, but not limited to disease and/or insect resistance, valuable nutritional characteristics, valuable abiotic stress tolerance (including, but not limited to, drought tolerance, salt tolerance), and the like. The non-recurrent parent can be any plant variety or inbred line that is cross-fertile with the recurrent parent.

The resulting progeny plant population is then screened for the desired characteristics, including the BnIND-A deletion, which screening can occur in a number of different ways. For instance, the progeny population can be screened using phenotypic pathology screens or quantitative bioassays as are known in the art. Alternatively, instead of using bioassays, MAS or MAB can be performed using one or more of molecular markers described herein to identify progeny plants or germplasm that comprise a BnIND-A deletion allele. Also, MAS or MAB can be used to confirm the results obtained from the quantitative bioassays. The markers, primers, and probes described herein can be used to select progeny plants by genotypic screening.

Following screening, the F1 progeny (e.g., hybrid) plants having the BnIND-A deletion allele can be selected and backcrossed to the recurrent parent for one or more generations in order to allow for the canola plant to become increasingly inbred. This process can be repeated for one, two, three, four, five, six, seven, eight, or more generations. In some examples, the recurrent parent plant or germplasm used in this method is of an elite variety of the Brassica species. Thus, this crossing and introgression method can be used to produce a progeny Brassica plant or germplasm having the BnIND-A deletion allele introgressed into a genome that is at least about 50%, 55%, 60%, 65%, 70%, 75%, 80%, 85%, 90%, 95%, 97%, 99% identical to that of the elite variety of the Brassica species.

Also provided is a method of producing a plant, cell, or germplasm (e.g., seed thereof) that comprises crossing a first Brassica plant or germplasm with a second Brassica plant or germplasm, wherein said first Brassica plant or germplasm comprises within its genome a the BnIND-A deletion allele disclosed herein, collecting seed from the cross and growing a progeny Brassica plant from the seed, wherein said progeny Brassica plant comprises in its genome said BnIND-A deletion allele, thereby producing a progeny plant that carries the deletion associated with the pod shatter tolerance trait disclosed herein.

In addition to the methods described above, a Brassica plant, cell, or germplasm having the BnIND-A deletion may be produced by any method whereby the BnIND-A deletion is introduced into the canola plant or germplasm by such methods that include, but are not limited to, transformation (including, but not limited to, bacterial-mediated nucleic acid delivery (e.g., via Agrobacteria)), viral-mediated nucleic acid delivery, silicon carbide or nucleic acid whisker-mediated nucleic acid delivery, liposome mediated nucleic acid delivery, microinjection, microparticle bombardment, electroporation, sonication, infiltration, PEG-mediated nucleic acid uptake, as well as any other electrical, chemical, physical (mechanical) and/or biological mechanism that results in the introduction of nucleic acid into the plant cell, or any combination thereof, protoplast transformation or fusion, a double haploid technique, embryo rescue, or by any other nucleic acid transfer system.

“Introducing” in the context of a plant cell, plant and/or plant part means contacting a nucleic acid molecule with the plant, plant part, and/or plant cell in such a manner that the nucleic acid molecule gains access to the interior of the plant cell and/or a cell of the plant and/or plant part. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell.

The following are examples of specific embodiments of some aspects of the invention. The examples are offered for illustrative purposes only and are not intended to limit the scope of the invention in any way.

### EXAMPLES

### Example 1: Discovery of a Native Deletion in BnIND-A

A native deletion located in B. napus INDEHISCENT gene on chromosome 3A (IndA) was unexpectedly discovered in a project that used CRISPR-Cas9 gene editing for the targeted genomic deletion (“dropout”) of the IndA gene. Although, the project successfully generated the expected IndA dropout in various B. napus lines, the same IndA dropout could not be generated in B. napus line G00010BC.

In the process of investigating why no BnIND-A dropouts had been identified in G00010BC, whole genome sequence was performed. The two main homoeologous copies of INDEHISCENT genes from chromosomes N03 (BnIND-A; BnaA03g27180D) and N13 (BnIND-C; BnaC03g32180D) were compared to five references genomes using the Basic Local Alignment Search Tool (BLAST™) algorithm available from the National Center for Biotechnology Information (NCBI). See Altschul et al., 1990, J. Mol. Biol. 215:403-10. The five reference genomes were DH12075 (public spring canola), Darmor (public winter canola), and three high quality third generation proprietary spring canola lines (NS1822BC, G00010BC, and G00055MC). Orthologous matches for both genes were found in all genomes, except for BnIND-A in G00010BC. Comparative global sequence alignment of an extended genomic region surrounding BnIND-A revealed a large segmental deletion in the G00010BC genome. The deletion is from 229 kb to 307 kb in length, depending on reference genome used for alignment. The physical starting and ending position of the BnIND-A deletion segment, as determined by alignment to each reference genome, is shown in Table 6.

TABLE 6

| Genome | Start Position | End Position | Length (bp) |
|---|---|---|---|
| NS1822BC | 14,710,075 | 14,958,191 | 248,116 |
| DH12075_v1.1 | 14,449,859 | 14,690,232 | 240,373 |
| Darmor_v4.1.1 | 13,306,888 | 13,535,922 | 229,034 |
| G00555MC | 14,915,907 | 15,223,431 | 307,524 |
| G00010BC | 14,989,780 | 14,989,781 | (deletion breakpoint) |

### Example 2: Molecular Assays to Detect the Native BIND-A Deletion

The G00010BC and wildtype reference N3 sequences were used to develop molecular assays to detect the presence or absence of native BnIND-A deleted segment as well as sequences flanking the breakpoint site of the deletion.

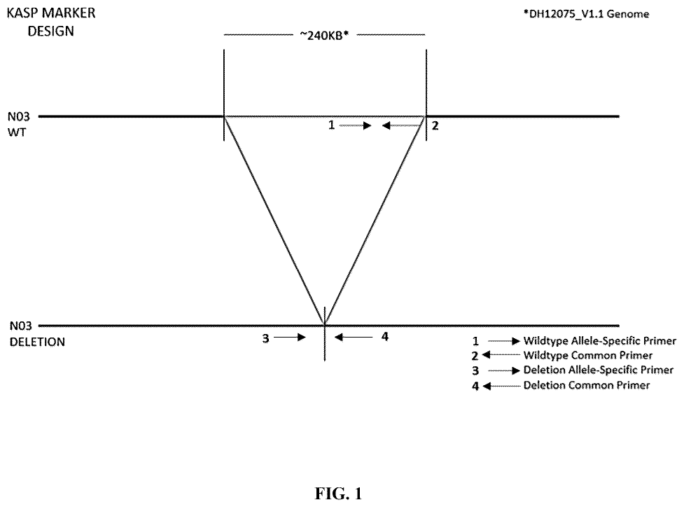

KASPar™ assay comprised of four primers was developed using an assay design algorithm available as Kraken™ (LGC Genomics, Hoddesdon, Hertfordshire, UK). Initially, BnIND-A gene sequence on N03 was compared to the homoeologous BnIND-C gene on N13 to identify unique polymorphisms. Potential primer sequence targets were then identified to detect the wildtype and native deletion states of BnIND-A. To detect the native deletion, allele-specific, fluorescently tagged (FAM) forward primer and a reverse primer flanking the breakpoint were designed as shown in FIG. 1 (N03 Deletion). To detect the wildtype state, a different fluorescently tagged (VIC) forward primer and reverse primer were designed to hybridize within the native deletion segment sequence as shown in FIG. 1 (N03 WT). The primer sequences shown in Table 7 were used in a four primer, co-dominant composite marker KASPar™ assay to detect the presence or absence of BnIND-A deletion segment.

TABLE 7

| SEQ ID NO | Oligo Name | Description |
|---|---|---|
| SEQ ID NO: 32 | IND_A_001_F001 | WT Forward Primer |
| SEQ ID NO: 33 | IND_A_001_R001 | WT Reverse Primer |
| SEQ ID NO: 34 | BP_F003 | Mutant Forward Primer |
| SEQ ID NO: 35 | BP_R003 | Mutant Reverse Primer |

The KASPar™ assay mixture was composed of 12 μl of 100 mM of each forward primer and 30 μl of 100 mM of each reverse primer. 13.6 μl of this mixture was combined with 1000 μl of KASP™ Master Mix™ (LGC Genomics, Hoddesdon, Hertfordshire, UK). A Meridian™ (LGC Genomics) liquid handler dispensed 1.3 μl of the mix onto a 1536 plate containing ˜6 ng of dried DNA. The plate was sealed with a Phusion™ laser sealer (LGC Genomics) and thermocycled using a Hydrocycler (LGC Genomics) under the following conditions: 95° C. for 15 minutes (min), 10 cycles of 95° C. for 20 seconds (sec), 61° C. stepped down to 55° C. for 1 min, 29 cycles of 95° C. for 20 sec, and 55° C. for 1 min. The excitation at wavelengths 485 (FAM) and 520 (VIC) was measured with a Pherastar™ plate reader (BMG Labtech, Offenburg, Germany). Values were normalized against the passive reference dye ROX (5-(and -6)-Carboxy-X-rhodamine, succinimidyl ester), plotted and scored on scatterplots utilizing the Kraken™ software (LGC Genomics).

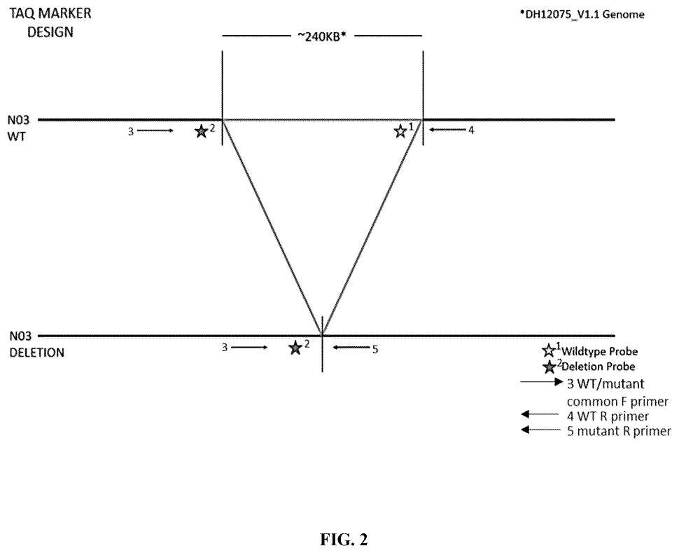

A variation on conventional TAQMAN™ end-point genotyping system was developed. Conventional TAQMAN™ assays use a forward and reverse primer and two fluorescent labeled probes. The TAQMAN™ variation developed to detect the presence or absence of the BnIND-A deletion segment is a compound assay that comprises two independent amplification reactions. The first reaction amplifies and detects wildtype gene sequence using forward and reverse primers capable of hybridizing to sequences that flank the 3′ breakpoint of the BnIND-A deletion segment in wildtype N03 chromosome as shown in upper portion of FIG. 2 (N03 WT). Because the deletion segment is present in wild type N03 chromosome, the wildtype primers amplify sequence upstream the 3′ breakpoint and the amplified sequence is detected using a wild type probe. The second assay reaction detects the presence of BnIND-A deletion using a deletion forward primer that hybridizes upstream of the 5′ breakpoint and a deletion reverse primer that hybridizes downstream of the 3′ breakpoint of the deletion as shown in lower portion of FIG. 2 (N03 Deletion). When the deletion segment is missing, the second assay amplifies sequence containing both the joined 5′ breakpoint and 3′ breakpoint. By contrast the second assay will not amplify N03 genomic sequence that includes the deletion segment because the 5′ and 3′ breakpoints are too far apart to be amplified effectively under assay conditions. The primers and probes for both wildtype and deletion reactions (4 primers and two probes shown in Table 8) were combined and the reactions were run simultaneously.

TABLE 8

| SEQ ID NO | Name | Description |
|---|---|---|
| SEQ ID NO: 36 | N101T11-F001 | WT F Primer/ |
| | | Common F primer |
| SEQ ID NO: 37 | N101T11-R001 | WT R Primer |
| SEQ ID NO: 38 | N101T11-001-X001 | WT Probe |
| SEQ ID NO: 39 | N101T10-F001 | Deletion F Primer |
| SEQ ID NO: 40 | N101T10-R001 | Deletion R Primer |
| SEQ ID NO: 41 | N101T10-001-X001 | Deletion Probe |

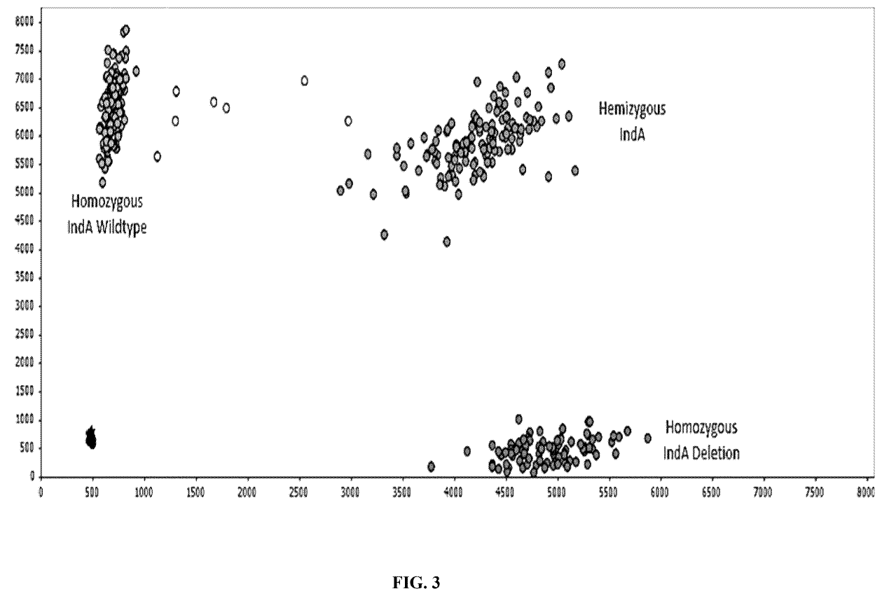

The combination TAQMAN™ assay included 13.6 μl of a primer probe mixture (18 μM of each probe, 4 μM of each primer) and 1000 μl of master mix from ToughMix™ kit (Quanta Beverly, MA). A liquid handler dispensed 1.3 μl of the mix onto a 1536 plate containing ˜6 ng of dried DNA. The plate was sealed with a laser sealer and thermocycled in a Hydrocycler device (LGC Genomic Limited, Middlesex, United Kingdom) under the following conditions: 94° C. for 15 min, 40 cycles of 94° C. for 30 secs, 60° C. for 1 min. PCR products are measured using at wavelengths 485 (FAM) and 520 (VIC) by a Pherastar™ plate reader (BMG Labtech, Offenburg, Germany). The values are normalized against ROX and plotted and scored on scatterplots utilizing the Kraken™ software. FIG. 3 shows results of using this TAQMAN™ assay to evaluate a mapping population segregating for the native BnIND-A deletion disclosed herein. FIG. 3 demonstrates that the disclosed assay can identity and distinguish between clusters of plants that are homozygous wildtype for BnIND-A, homozygous BnIND-A deletions, and hemizygous deletions (wildtype/BnIND-A deletion).

This example describes the development of diagnostic assays useful in methods of detection of and introgression of the BnIND-A native deletion disclosed herein.

### Example 3: Survey of Elite Germplasm

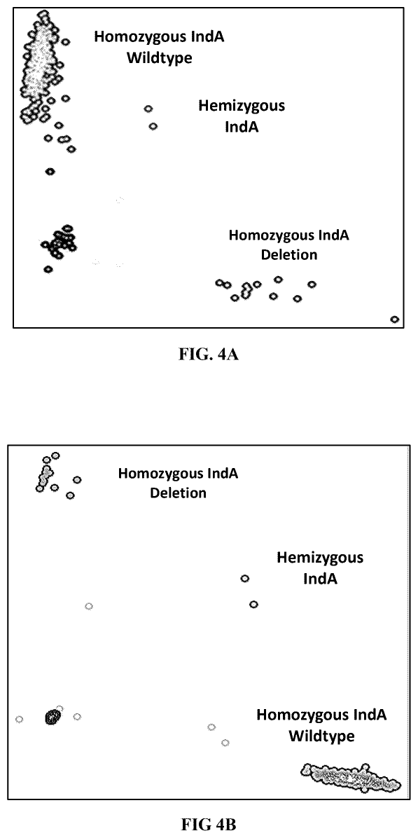

Publicly released and proprietary collections of elite germplasm lines from North America, Australia, and Europe were analyzed using molecular assays described in foregoing Example 2 herein. Results of the KASPar™ assay and combined TAQMAN™ assays for BnIND-A deletion are shown in FIG. 4A and FIG. 4B respectively, which indicated that IndA native deletion can be found in public and proprietary elite global germplasm, although its prevalence is rare: 22 of 947 tested lines were found to contain the BnIND-A native deletion disclosed herein.

The foregoing demonstrates the usefulness of molecular assays disclosed herein for the detection of native BnIND-A deletion. It also confirms that the KASPar™ and combined TAQMAN™ assay produced the results of a co-dominant assay and were able to distinguish and display sample clusters that are homozygous wildtype for BnIND-A, homozygous BnIND-A deletions, and hemizygous deletions (wildtype/BnIND-A deletion). Sec FIG. 4A and FIG. 4B.

### Example 4: Pod Shatter Phenotype of B. napus Line with Native BnIND-A Deletion

A

laboratory assay was developed to evaluate the shatter resistance of pods subjected to mechanical agitation at a specific speeds and times. GENO/GRINDER device (SPEX® SamplePrep, Metuchen, NJ) was used to mechanically break canola pods and thereby assess shattering tolerance or susceptibility. Different speeds (rpm) and times were tested with intact pods from inbred NS1822BC grown in controlled environment growth chamber (Conviron, Winnipeg, Canada). Fully shattered pods (both valves detached from the replum and seeds dispersed), half shattered (one valve detached from the replum and about half seeds dispersed) or unshattered pods (both valves attached and containing seeds) were measured. Fifteen pods were used for each data point. Results showed a linear relationship between the mechanical speed at which pods are agitated and the number of shattered (full+half shattered) pods (r2=0.944).

In another experiment, a group of 15 pods (ranging from 3 cm to 6 cm in length) for three different inbred lines, NS1822BC, G00010BC, and G00055MC grown in a controlled environment were tested. Results indicated a direct relationship between speed of mechanical agitation and pod shatter for the inbreds that were tested. Pods of all three inbreds were almost fully shattered at speeds equal to or greater than 1200 rpm.

At 750 rpm, pod shattering of G00010BC was found to be significantly lower than the other two inbreds. Thus, compared to the other two inbreds evaluated under the foregoing conditions, the native BnIND-A deletion line (G00010BC) provided an improved shatter tolerance phenotype.

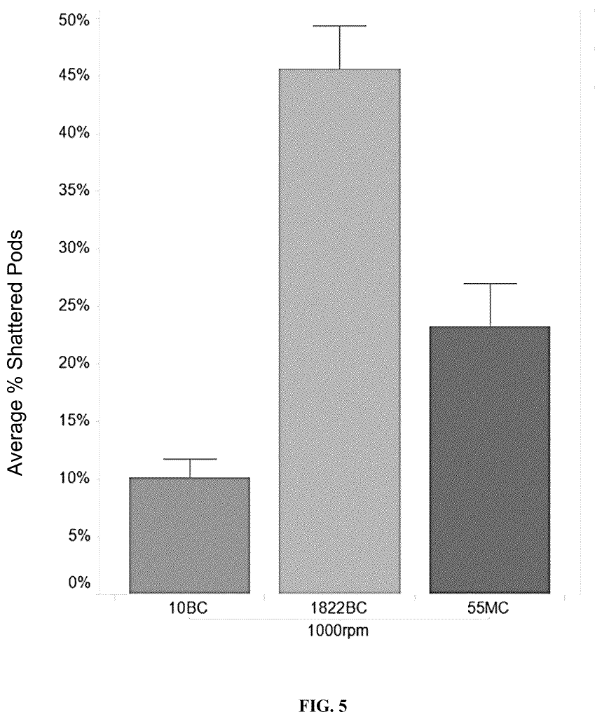

A second laboratory assay phenotyping experiment was conducted using 5 to 6 plants of each the foregoing three genotypes. As described above, plants were grown in a growth chamber, and pods collected at maturity were phenotyped using the Geno/Grinder assay (15 pods at 1000 rpm for 30 sec). Percentage shattered pods (SHTPC) was recorded for each assay repetition. The results shown in FIG. 5 demonstrated statistically significant differences (t-test: two-sample assuming unequal variances, p<0.01) between indicated genotypes. Pods collected from G00010BC were found to be on average significantly more shatter tolerant than pods collected from the other two phenotypes. These results provide additional evidence that the presence of the IND-A native deletion in G00010BC contributes to higher mechanical resistance and an improved shatter tolerance phenotype as compared to the other two genotypes tested.

This example describes a laboratory assay to induce pod shattering and evaluate pod shatter tolerance. Results of two studies using the assay showed increased shatter tolerance for G00010BC relative to lines NS1822BC and G00055MC and provides evidence that the native BnIND-A deletion in G00010BC contributes to increased shatter tolerance.

### Example 5: Pod Shatter Phenotype of BnIndC Dropout Combined with the Native BnIND-A Deletion

CRISPR-Cas9 gene editing was used to generate a targeted genomic deletion (“dropout”) of the INDEHISCENCE gene in the C genome (BnIND-C) in lines G00010BC having the native BnIND-A deletion disclosed herein. Agrobacterium transformation was done according to Moloney et al. (1989) Plant Cell Reports 8:238-242. Second generation (T2) G00010BC BnIND-C homozygous dropout variant and wildtype control plants were grown in controlled environment growth chambers (Conviron, Winnipeg, Canada) under standard conditions. BnIND-C genomic dropout sequences are disclosed herein (SEQ ID NOs: 4, 14, and 25).

Pods were harvested at maturity after 2 weeks without water. Pods were left to acclimate in the laboratory at 23° C. for 5 days. Fifteen pods of similar sizes were harvested for 5 individual G00010BC homozygous dropout plants and 5 segregating wild-type plants. Pods from individual plants were placed in plastic boxes of 12×8.5×6.5 cm and mechanically agitated at 1700 rpm for 30 seconds using GENO/GRINDER device. After disruption, individual pods were scored according to half shattered, fully shattered, and unshattered phenotype. Total number of shattered pods was calculated as the sum of the half shattered and fully shattered pods. The average percentage of shattered pods for G00010BC homozygous BnIND-C dropout variants was near 0.00%, as compared to an average of 92.00% shattered pods for G00010BC plants.

This example shows that non-functional gene deletion of the IND gene in the C genome, combined with the native BIND-A deletion disclosed herein produced a fully controlled genetic trait imparting shatter tolerance in B. napus.

### Example 6: Pod Shatter Phenotype of Heterozygous and Homozygous T3 BnInd-C Combined with Native BnIND-A Deletion

Sixty-four T3 seeds from two T2 G00010BC plants heterozygous for the BnIND-C dropouts (generated as described in Example 5 herein) were planted and genotyped using a dropout specific PCR assay followed by NextGen sequencing. Plants that were homozygous (9), heterozygous (10), and wildtype (8) for the BnIND-C dropout were identified and grown to maturity in a growth chamber in 16 hour light (23° C.) (˜360 μE light intensity) and 8 hour dark (20° C.) regimen at ˜55% humidity. At maturity, plants were allowed to dry. Pods were harvested and phenotyped using the GENO/GRINDER laboratory assay at 1100 rpm for 15 seconds. For each replication using 15 intact pods, pods were visually inspected after the assay and classified according to fully shattered, half shattered or unshattered pod phenotype.

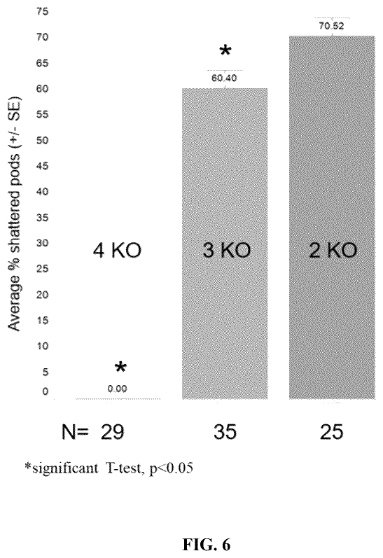

FIG. 6 shows shatter tolerance of G00010BC plants segregating for a BnIND-C dropout calculated as the average percentage of shattered pods from replicated assays. The number of knocked out alleles (KO) are indicated for each zygosity category, where 4 KO are homozygous for the BnIND-C dropout, 3 KO are heterozygous for the BnIND-C dropout, and 2 KO do not include any BnIND-C dropout alleles. Since G00010BC background has a native ˜200 kb-300 kb deletion on chromosome 3 that affects BnIND-A, G00010BC has 2 deleted alleles (2 knockouts or 2 KO). Plants heterozygous for the BnIND-C dropout have three deleted alleles (or 3 knockouts and thus 3 KO) and homozygous plants have four missing alleles (or 4 knockouts and thus 4 KO). B. napus plants with 4 KO showed a strong shatter tolerant phenotype, and plants with 3 KO showed a significant reduction in the percentage of shattered pods compared to unmodified G00010BC plants (t-test, p<0.05).

This example shows that the number of knocked out IND alleles can correlate with pod shatter tolerance, including in backgrounds having the native BnIND-A deletion disclosed herein. Double knockout plants (all A and C alleles knocked out) showed higher shatter tolerance than plants with 3 knocked out alleles (2 KO for A and 1 KO for C).

### Example 6: Validation of Field Phenotyping Study for Pod Shatter Tolerance

Plants were grown in a replicated trial in Rockwood, Ontario, Canada. Plants in the field received a shatter-inducing treatment in the form of 135 km/h wind generated by a blower mounted in front of a tractor. The treatment was applied 12 times at a tractor speed of ˜5 km/h four months after planting. Wind angle compared to planted rows was varied from perpendicular to oblique for maximum effect. The trial saw additional shatter pressure for another two weeks after this shatter inducing treatment due to weather related events such as moisture, rain, dryness, temperature and natural wind. Percent shattered pods (SHTPC) was determined using visual evaluation of plants from 5 replications. Intensity of shatter pressure was evaluated using the following reference lines: 45H33 is a moderately susceptible check line; 45CM39 and 45M35 were used as shatter resistant checks. Results showed statistically significant separation between SHTPC of the most shatter susceptible hybrid 45H33 (60% shattered pods), and shatter tolerant HarvestMax hybrids 45M35 and 45CM39 (˜30% shattered pods). A statistically significant difference was also found between susceptible (wildtype G00555MC and G00182MC) and tolerant (N00644BC and NS7627MC) inbreds, both indicative of significant shatter pressure on the trial.

The foregoing provides a validated field method for phenotyping pod shatter under controlled conditions that induce pod shatter. The method successfully distinguished between shatter susceptible and shatter tolerant lines of B. napus plants.

### Example 7: Laboratory Phenotyping Native BnIND-A Deletion in Combination with BnIND-C Dropout

Pods were collected at maturity from multiple plants grown in one of the six field replications described in Example 6 prior to the field shatter inducing treatment and phenotyped in the laboratory using the GENO/GRINDER assay.

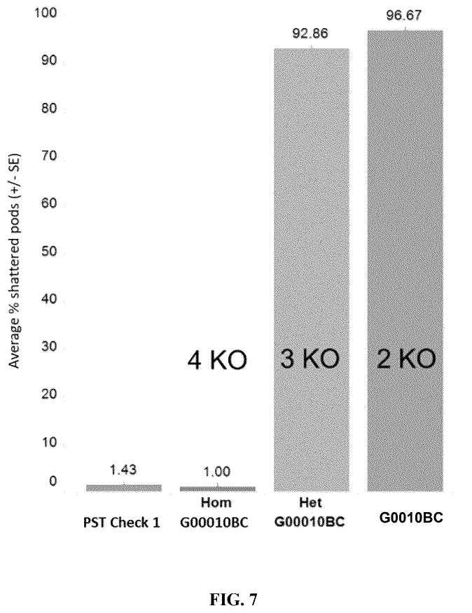

Intact pods of similar sizes were collected from G00010BC plants having the native BnIND-A deletion disclosed herein and gene edited G00010BC plants that were homozygous or heterozygous for an IND-C dropout. The mechanical resistance of these pods was compared to that of a commercially released pod shatter tolerant (PST) line check, which is referenced herein as PST Check 1. Pods were shaken in the GENO/GRINDER at 1500 rpm for 15 sec and results are presented in FIG. 7. At this speed, only about 1% of PST Check 1 and G00010BC BnIND-C homozygous (4 KO) dropout pods shattered. Pods of G00010BC BnIND-C heterozygous plants (3KO) produced approximately 4% fewer shattered pods than G00010BC (2 KO) controls, though this difference was not statistically significant (t-test, p<0.01) because the GENO/GRINDER assay applies significantly higher forces than the forces used in the field phenotyping experiment of Example 6. Because higher forces overcome the mechanical resistance in more pods of both G00010BC plants (2KO) and G00010BC plants with a heterozygous BnIND-C dropout (3KO), the GENO/GRINDER assay did not detect the significant difference in pod shatter tolerance that was observed with this material in the field phenotyping study. Compare the results shown in FIG. 6 with those in FIG. 7.

This example demonstrates that combining native BnIND-A deletion with a second homozygous loss-of-function IND allele improved pod shatter tolerance even under high force shatter-inducing conditions.

### Example 8: Field Phenotyping of Native BnIND-A Deletion Plants

Plants in five of the six field replications described in Example 6 were subjected to pod shatter inducing treatment. Plant pods were scored in the field.

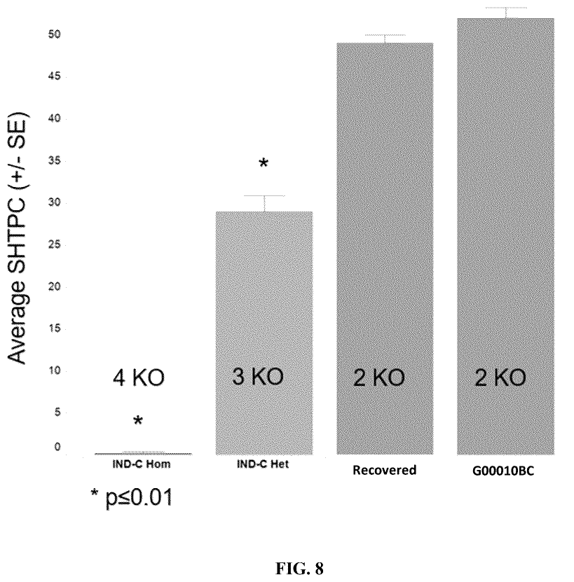

Wildtype and inbred G00010BC plants with different BnIND-C gene-edited dropouts were grown in the same field and characterized. G00010BC plants homozygous for BnIND-A native deletion and heterozygous BnIND-C dropout have three loss of function IND alleles (3 KO). Following shatter inducing treatment in the field, these 3KO plants had significantly reduced SHTPC scores as compared to G00010BC control (2 KO) and segregating plants “Recovered” (2 KO) with homozygous native BnIND-A deletion. SHTPC scores of heterozygous plants were reduced by 40% to 45% compared to the (2 KO) controls. See FIG. 8 and Table 9. Plants homozygous for the BnIND-C dropout did not shatter confirming the previously observed strong shatter tolerance of BnIND-A and BnIND-C double homozygous knockout.

TABLE

| | SHTPC | SHTPC Reduced |
|---|---|---|
| INDEHISCENCE Genotype | (approx.) | vs 2KO (approx.) |
| G00010BC (2 KO) | 50% | — |
| Recovered Segregants with Homozygous | 48% | — |
| BnIND-A deletion (2 KO) | | |
| G00010BC/BnIND-C Dropout | 28% | 40-45% |
| Heterozygous (3 KO) | | |
| G00010BC/BnIND-C Dropout | ~0% | ~99% |
| Homozygous (4 KO) | | |

This example demonstrates that combining native BnIND-A deletion with a second loss-of-function IND allele improved shatter tolerance in the field.

### Example 9: Phenotyping Hybrids with Native BnIND-A Deletion

G00010BC plants and G00010BC plants homozygous for an BnIND-C dropout were crossed with wildtype plants and plants containing homozygous BnIND-A dropouts to create hybrid plants with different allelic combinations of dropout and wildtype alleles. A small amount of hybrid seed was generated to grow plants for laboratory assay pod phenotyping. Specific allelic combinations of BnIND dropout variants are shown in Table 10 (for hybrid genotypes: first letter designates male parent allele, second letter G00010BC female parent allele; upper case designates wild-type allele, and lower case indicates a gene-edited dropout allele, except for the bold and underlined “a” which designates G00010BC BnIND-A native deletion allele).

TABLE 10

| | G00010BC | Hybrid |
|---|---|---|
| Male parent | female parent | genotype |
| IND-A HO Dropout | IND-C HO DO | aa/Cc |
| IND-A HO Dropout | IND-C WT | aa/CC |
| IND-A WT | IND-C HO DO | Aa/Cc |
| IND-A WT | IND-C WT | Aa/CC |

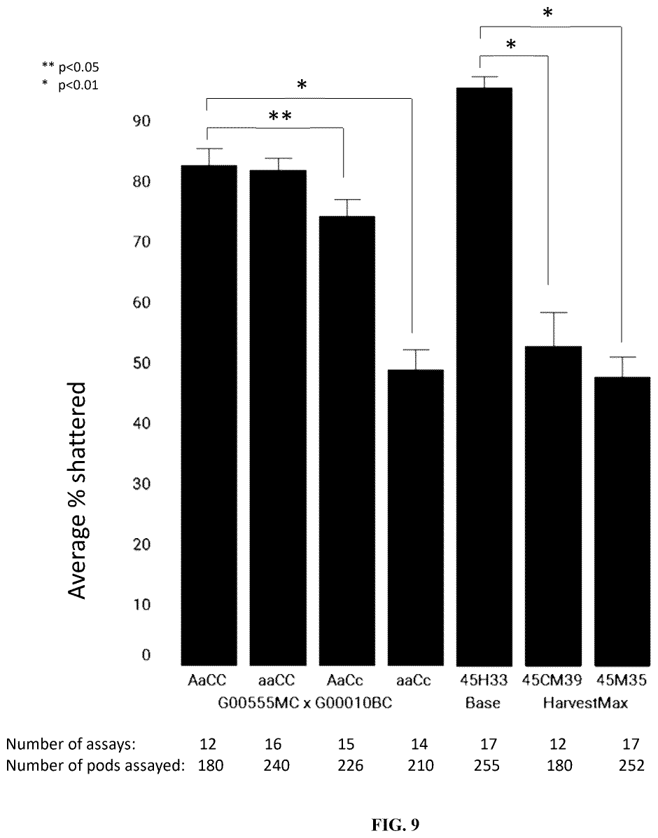

The resulting hybrid plants and checks were grown in a growth chamber for comparison with hybrid checks. The moderately susceptible to pod shatter check used was 45H33. Shatter resistant checks were HARVESTMAX Hybrids 45CM39 and 45M35. Four plants were grown for each entry except for 45CM39 for which only 3 plants were grown. Intact pods were collected from individual plants and Geno/Grinder assays were conducted using 10-15 pods per assays as described in Example 4. Percentages of shattered pods were calculated. Pods from each hybrid ranged from 3 cm to 7 cm in size and hybrids showed comparable pod size distributions except for 45H33, which had a higher number of smaller pods on average. Results of the phenotyping experiment are shown in FIG. 9.

HARVESTMAX hybrids (45CM39 and 45M35) showed a ˜50% reduction in percent shattered pods compared to 45H33 in this assay. This is similar to the difference in SHPTPC observed in the field for these hybrids. Accordingly, these results indicate that the laboratory assay results are sufficiently predictive to identify and distinguish shatter tolerant plants (HARVESTMAX hybrids) from moderately shatter susceptible plants (45H33) in a manner that is consistent with their shatter tolerance performance in the field.

The results in FIG. 9 for the hybrid with 3 dropout alleles (aa/Cc allelic combination) also show that native INDA deletion in G0010BC can be used to breed hybrids having a shatter resistance phenotype comparable to commercial shatter tolerant HarvestMax hybrids and that introgression of the native BnIND-A deletion of G00010BC in combination with other loss of function alleles contributed to shatter tolerance that is comparable to that of commercial shatter-tolerant hybrids.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A Brassica plant, cell, or germplasm thereof comprising a BnIND-A genomic deletion on chromosome N03 and a BnIND-C genomic deletion, wherein the BnIND-A gene deleted segment results in a breakpoint locus corresponding to positions 10,002-10,003 of SEQ ID NO:2.

2. The Brassica plant, cell, or germplasm thereof of claim 1, which is heterozygous for the BnIND-A genomic deletion.

3. The Brassica plant, cell, or germplasm thereof of claim 1, which is homozygous for the BnIND-A genomic deletion.

4. The Brassica plant, cell, or germplasm thereof of claim 1, which is heterozygous for the BnIND-C genomic deletion.

5. The Brassica plant, cell, or germplasm thereof of claim 1, which is homozygous for the BnIND-C genomic deletion.

6. The Brassica plant, cell, or germplasm thereof of claim 2, which is heterozygous for the BnIND-C genomic deletion.

7. The Brassica plant, cell, or germplasm thereof of claim 2, which is homozygous for the BnIND-C genomic deletion.

8. The Brassica plant, cell, or germplasm thereof of claim 3, which is heterozygous for the BnIND-C genomic deletion.

9. The Brassica plant, cell, or germplasm thereof of claim 3, which is homozygous for the BnIND-C genomic deletion.

10. A method of generating Brassica napus plants comprising a BnIND-A genomic deletion and a BnIND-C genomic deletion, the method comprising
a. selecting a first parent Brassica plant comprising a BnIND-A genomic deletion on chromosome N03, wherein the BnIND-A gene deleted segment results in a breakpoint locus corresponding to positions 10,002-10,003 of SEQ ID NO:2; and
b. crossing the first parent Brassica plant with a second parent Brassica plant comprising a BnIND-C genomic deletion; thereby generating one or more progeny plants comprising the BnIND-A genomic deletion and the BnIND-C genomic deletion.

11. The method of claim 10, wherein the first parent plant is heterozygous or homozygous for the BnIND-A genomic deletion.

12. The method of claim 10, wherein the second parent plant is heterozygous or homozygous for the BnIND-C genomic deletion.

13. The method of claim 10, wherein the method generates one or more progeny plants which is heterozygous for the BnIND-A genomic deletion and heterozygous for the BnIND-C genomic deletion.

14. The method of claim 10, wherein the method generates one or more progeny plants which is homozygous for the BnIND-A genomic deletion and heterozygous for the BnIND-C genomic deletion.

15. The method of claim 10, wherein the method generates one or more progeny plants which is homozygous for the BnIND-A genomic deletion and homozyous for the BnIND-C genomic deletion.


SEQUENCE LISTING (159 sequences)

SEQ ID NO: 1 - protein, 182 aa, Brassica napus
MSGSKADAAIAPIVMMEHHHLLMNWNKPIDLITEENSFNHNPHFIVDPPSETLSHFQPPPTIFSDHGGGEEAEEEEGEEEMDPMKKMQYAIAAMQPVDLDPATVPKPNRRNVRVSDDPQTVVARRRRERISEKIRILKRMVPGGAKMDTASMLDEAIRYTKFLKRQVRLASSASHSAWSSYV

SEQ ID NO: 2 - DNA, 20001 bp, Brassica napus
  source 1..20001: mol_type = genomic DNA
tgcggaggtttttgaaattcgcaaagtcgttccagaacatcaatgttcaatagacgaacgtgggggttacgaaacacaagctacagctgcggtaattggtgagcttctgaaaaacaagtttggggcgacaggatcaggtcccaaacctcgggagatccgtcacatgatgcgtggtgacctaaacgtcaatatatcatactggaaggcatggcggtcgagggacgtcgcaatggagaacacgaaagactcagatgtgactgcttacgcaaaactcccagattaccttaacaagctagtgaagtctaatccaggttctctcgtcgaactctgtacagaaccacagcaaaatggtggccaccgtttcaaatatttatttgttgcattggaggcttctatcaagggatacgagtacatgaggaaagttgtggttgtagatggaacccacttgaaaggcaagtatggggggtgtttaataacagcatccgcccaagatggaaattatcagatttttccactggcttttgccattgttgatggtgagaatgatctgtcatgggaatggttcttcactcagctttccaaattagtaccaaaccatgaagatttggtgtttgtatctgataggcatgcttctattttgaaaggaattagcaaggttagtaacattatccaatttattttagaaacatttttaacgtaatcggaaaacaaggaagatgtaggttatgaatctttctggttctcgttgttacaattatttttcgtataatatataacgtttgcttaaaacataatctcaggtatatccctcggccggtcattgcatttgtattgtccacctgaagagaaacatccgctcaaactttaaaggacgacatctcgagtacttggtggccaaagccgcaagaagcttcagactagaagatttttacacatcgttcaacgagattaagagtattgatcctagctgtgcggagtatctgctcgagattgggcttgatcattgggctagagcccaatttcctggaaaacgatataatgtgatgacaagcaatcttgcggagtcttggaatggagttttgcgtgaggcgagagaactaccggtcatacgattggtcgagttcattcgtagtaagctgatgacatggtttgcgagtcgtcgtgaggcggcaaataaaaataaaggtaacctatccaaaaaggtgttaggtattgtaacgaaaaactttgacaactctggtgggtatgaggtattggagattgcagagaaggagtttgaggtccgacagaaagaaggtggaggtttccatgtcaaccttgccaatatgacttgtacgtgctttgaatttcaaatgctggcaatgccctgttctcatgccatagctgcagccttaaaaggaaagatagcggtagaatcgttggtggcaccagtgtatggggttactcaacttcaagcagcatacggggggagtatatatcctgtcgaaggatacagcggaattgcagatttggtcgccaatgttggtggcgtccaaatctttcccccaaatacccgacgtcctccgggaaggccaaaaaagcagagatacttttcaaagggagaaaaagtggtaatctacaatacgtatcattcaaacgtgttcattcttggtaactaatatacaacttctgcacttatgtataacagcttaaacgagggcgtcgcaggacagcttgtagcaggtgcaaaaagttgggacacaacaaagcaacctgcaaggaacctatataagaagaagaaggttggtgggtaaggggaatttagggtcagagggaagatgtaagtgagactggttgtagtatgggaaagctgtatggaacggtgaaggatttaagatggtagaataatgatgtgtgtgtatgtttgtgaaatagtcatgtaaacaaaatccatcaacggaagtggtggcggtggacgttatatatttcttataagaattcccctctctgtatactgtgatttctatattttcagcaataaactaatattgacagtaccaaatatgtagcgaatgaatttctactaaaattctttatgggaaaggataaccggctaacacttaaaataagacttgtaacataattgcctaaccagatttgtaagaaatctggaaaccaatcccaaatactctgatcgtcacaaactctaaattacgggaaaaggctgtagagaaggttacaccgccaacttgatcttagctaataccgtaaagacaacattacgaaaaattaatataaatcaaatgtttttgacatatattttttgtaacgagctactagagttaatctctaaactctaaacctttaacccgaaaacaataaacccaaaacctaaacaatataccgtataaccgttgcagtatcacctataccctaaaccatagacaatacaccctagactgtgatcataaatctgtaggcggctacatgtttgcttatgcgtagacaaagatatgttaacctgctaaatcatgtaacgctaaacaataaactctataccgttaaccattaaccctaaaaacataaagcctataccataaaccctaaaccctaaaccagatacggaaaaacttaaaccctataccctaaacaataaactctttacggtattccataaacccttaccctaaatcatgtaacgctaaaccataaacccttacgctgtagccgtctacattatttcttatccgtttaaaaatatatgttaacctgctaaatcatgtaacgctaaaccataaactctataccgtcaaccatttaccctaaaaccataaagcctataccataaaccgttaaccgtaaacctaaaacccataattcgaaaacactaaaccctaaacaataaaccctaaaccgaataccgtaaaccctagccaatttaaccattaaccctaaacccttaacactaaaccctaaaccctaaaacataataagaaaacactataccctaaaccctaacccagaattggaaaacactaaaccctaaaccctaaaccatataccccaaaccctaaacccaaaaccttaaccctaaaccatataccccaaaccctaaacccaaaacctaaaccctaaacactcaaccataaacactaaaccctaaaccctaaaccctaaaccctaatccgtaaaacataatacgtaaaccctaaaccctaaacctaacccataatttgaaaacactaaaccctaaacccaaaccctaaaccctaaacccgaacccacaaagctctgccttcgtagtaactatgtagccggctaggacctaaaccgtaaacctaaaacccataattcgaaaacactaaaccctaaaccctaaacccttacccaaaccctaaaccgaataccgtaaatcctagccaatttaaccattaaccctaaacccttaacagtaaaccctaaaccctaaaacataattagaaaacactaaaccctaaacctaacccagaattggaaaacactaaaccctaaaccctaaaccccaaaccctaaaccccaaaacctaaaccctaaacaataaaccctaaaccctaaaccctaaaccctaatccatataccctcaaccataatccgtaaaccctaaaccctatccactataccctaaaacgtttacaattatacgtaaaccctaaaccctatccagcggaccctaaaacgtatacgagtaacctaaaaagctaaaacctccgccggcatagtaactctgtagccgggtccatattaacctatccgtaaatttactccgtagccgggaacatataaactgatccgtaaacaaactctatagccgtcatcgtaactatgtagcccgctacatatttacttaaccgtaagtaaatatatgtaaaccagctaattcatgtaacgctaaaccatacaccctaaacccaaatatctaaaccctaaacactaaacactaaacgctaaacactaaaccctaaaccctaaacctaaaccctaaaccctaaacctaaaccctaaaccctaaaccctaaacactaaaccctaaaccctaaaccctaaacactaaaccctaaacactaaacactaaaccctaaaccctaaaccctaaaccctaaaccctaaaccatacaccctaaacccaaatatctaaaccctaaacactaaacactaaacactaaaccctaaaccctaaaccatataccgtaaaccgtaaccaataccctaaaccctaaaactgtaaaacctagccaaattaaaccaaatcttaaagcctaaaccataaaccgcaaaccttaaaccctatccagtcaacacaaaacataaagcataaacctataacccaaaaaccgccgccgtcatagtaactatgtagcccgctacttattttcttattcgtgaaaatatatatgttaacctgctaaagcctgtaacgctaaaccggctaattcatgtaacgcaaaaccccaaaacaattactctttagccgtctttataattgcttatccgtaaaaaaaatatgttaacctggtaaattatgtaacagtaatcaataaaccctataccgttaaccattaaccctataccctataccctaaaatctaaaccctaaaccctaaaccctaaaccctcaaccctaaaccctataccgttaaccattaaccctataccgtataccctaaaccctatacgctaaaccctaaactctaaaccctaaacccaaaaaccctataccctaaacgctaaacccaaatccctaaatcgtaaaccctaaacccaaacctaaaccctaaaccattaaaccgtattccttatccagttaaccgtaatccttataccttcgaccatagtaactctgtaaccggctacatattaacttatccgtaaatttactccgtagccggctacatattaactgttccgtaaattaactccgtagccgtcaaagtaactctgtagcccgctacatattaacttaaccgtaaaccctaaaccctgaacgctaaaccctaaaccatataccatataccatataccatataccatataccatataccctaaaccataaaaactaaaccctaaaccataaaccctgagtgaaccccataatagtaactccatacgcggctatgcattttcatagggggatttttaaatatgttcacctgctaaccatgacattgttatacccgactatataatttttaaccgtatgcatgtatatattaagctggtaaccatctatttaacaaaattaaacatgtccaaatattcatacaaacatatcgaacatccatactagcctttaaaaggcgtcataaaagtcttaaagggcaacaataattacgaaaattatgcaaattttacagcacaacacagcagcccctcatgttcatctgggattcaatcagcatcttcccccgttcaatatctttctgcaatccaccaatttcccctcttaagtcagctatatctcccttcaaatgggtcaccatctcctttagatcgtaaatctcctccaccatacattcatcagtccaggaaaagaggtgagatttgtcctgcacaaattagccaaatccggatcgccttgtttcggaaagtacttccattgaaatagaaaaacataaacgatcttaagtacctcttcagatccatacggacaacaatggaaaagccttccagggttctttgctgttccggaggttttaatgacagcctcttccccacaccgacaaaatgtagggaaccctctctctcttgcccgccgcccccgcctcgagcttactccccctgcaaaaatgtgggttatggtaatggatagtcagaacaaagaaactttaaatccaggttaattacataccagacgtagaggacgagcttgccttccctgattgcgacattcttcttcctctatctttctcctccttaacgattttgtaacttaacctcttccggatcgtcgctttgtcggccaataagagaggaaccaaatacgactagaaaaaggggaacattgaatttaggttacagtattagacgttggcccgaatattatgagcccaagcccaattaatgaaaataggtgatattaaataaattacttgaccggctactcgattttgtaacctccacacagtttccacttaattaggtcgatcataacatatgttttcaaacaaacaacaacactggctgagttaaaacatacgggaatgtgaaaaaaatacattacaaactaggaatgagttcatatgatgccgtggtatctctccacaatggtgacagcaacacgttgaagctctgactcgacaacgttgggggatatgcacttgcagatttctaatccgccaaatgcgtgtgcttgtataaggagaaccgcattaacagccgaatacaaatgattctctaattgcggcaccagacgtggtctatcaatcggtaaagccttaagatctttactggaaatttgtttcccggcttgtctaagtaaataaggaaacatggctgcaattggggcaatctctttgcacatcataacatcagtgcggatagagatgttgcagtccagaacatggagctgccaactggtggcgtcaatgcacaaaccaacccaatgtgtcttatccaagttaaaagggaagtagaacctggaagccgcacctacgctcctgcaacttgtagaaaaactctccaacaaagttgttggaaatttgaaaccatccattttcgccttgttgaaccttccataactcttggataactgagatacgaacttggtgtcgaagaaaactgcgtgatttgttgttatctgaggagaggcactgttatataccgaacgtgtatgatgaataagcagatcaacaacctagcaataaccatacgttcaatatagtggaccctcattaggcacgatataggtataacaatattataaacaaataacggaagacagaccttcgccggtacttgtgcagacttttccaccagggccattatatccttgctttcaattttaactccgtgaacgatgaacgaactaagataaaatgtgtgaaccactaagttaccataatccgacatttcacgtgtgggaaaatagaagtttgttggagatacttacaacggtgttttaagaatgtctctcagtttttcgaatttgtactcagcttccatgtcatcaccggtacgattagccgcaacatgtgcttcccatgctcttgttagaattttcttgtcacattcgtagtcacccactaacgcttttggaacaaccttctggcgcttgcttttcctgaaagggtgacaaggcgcatccccttcttctgctgtaagaacatcatccttgttcaattgtatcactagagtttcctcatttacggcatcacacccttcgatttgataatttcgctcttcctgactcagtccgagcgagaatgtaggtttagggaacaggagggcagggtcgagaatctgcggataaaacatatgaaataggttctgatcagataataaaatatgttaaaattaacaactcatacggtttcatctgcggcagcagaaatattggaatcaggttcagaaccattcaagttagcccttccgagggtgtgcatctccgtgttaactcccacggtatctacctggagaacgacaacaaattcacttgaagactatccgtacggctgtaagtattctgcaaaatatcctcactaccccgctaacattgttattagctacctaacttttgactatcacatatttacttccgatgtcaactacataaccttgttaggtggcattatttacacactgctgaaagaaacacaataaaaaacatacattgggaagttgaaacccttcctcctgcgcgaaccgcaatgcgctgtcgatgatttttgaagcatcaagttcaccattagttgaagctgggggttgattgaggacctcatttaagcctacatgtgaataattggactgatcctctccagtcctccgaacctgcggagatgttaggggtaaatataaattattactttcacgtacaattatgcatatatacttacagctatagacggtcgaacatgttgtatggccggagcaggggaatgattcatctgattacttatactctggaatgtctcagcgtgaaatgactttaacatatcttctactttggaaacaatggtggtttcgagacttgtaaaggcagcaccaaaactcttaaattctgtggcgaggaaggaaagttctgcggaaacacggttgagctcttctgatacacgactcagatctcctgaaatgcgacctttcacagaactagctatggattctgcgtccacatgatcgccacaactcgggtttgatttctgctttgttgttttcttcttctctgcttcagttcttgtttcttcacgcattctcacaacgtccgattttgaaacacctcccgtaaaaaatgaatggctgaacgaaaatccctcgtcaattaacttcagcaaattttgtacacccacatcttcttcatcatcagtaaacccaacctcagaatccccattcacttcctcgttcccgaccgaaataattgaaaacacctccgcctgccgttacatttgttgtcagtcacgattacacaaacattttataccagttgttattttcctaccatattacctttcctgagcagtcagtctcccgggcatgccctggacttatacttcgcttccctttcctttcttcttctgaaaggtcgtcgtcgtccgcaaagtcgccttcagaacccgacgacccggcttcatgaacggcttcggtaagagcaggtacagcttcaatcattacaagctgtaaagctagcacaaatccctttaaagctattgtgttttgcgaaagagaaagttcgttcctctctttgatactgcttatcagcaactcgaatgaaacgcgtccccaaggatatgcaagaaacgcatccaaatctttaattttctccgcatgctcgtgtgaaatccgaggtgtatgagtggttgggagaacaacagaagcaaggagtgcaaggaacgcatactttatccttgtttcttttgcagttacacttctctttttcagcatccttacaacacagctgacagaaacttcttccatggttccgaatagctcaccccagtatggcttctcgttgatatccctctttgccttttttctggaacgagctggatacttacaacaattcagacctgttactttagcaaattccttcacagaaaaccggattggtttgccagcaaacaagaaccaggcttcatgtttcttcttcaccttcaattgcctcgatataatgtatctcccaaaacgtccagagaatgttggtttctccgcgatttctacaagcctaccgaacggtgatcggcgcagagcttcaacttcatctttttccagtgccccgagtatgttacttattgcgaagggtttgtggtaaggggttactctcaccccaacgggttcctcgcccaatgcgaacatcctctccggtatcctttgcgaagactccacttctcgtcggttttccatctccatcgtcggtaccgaaatcttccgtctcagatgattgccaaaaaacttaaggagagagtttccagatttgaggcttgttatacaaaacagattgatttaagagatgaggatttcgattttcgaatttcaaaattacacaagatgctctataatcttttaagatttgagttttctgacacaagatactcaagatacggaaatgatacagaatatcaaggatacgcaaaatataaaggatacgcaaatcttgaaatatttcactttatacgcaaatcttgaaatatctcaccctaaagattacgtaatctttttctgcctcgatatcgataagggtagggaggtctttaacatctgctcaacagtactaaatagtactaataggtatatgcctaaatacgtaatatgtcgagtatagggagcgcaatttctctatttgtttttggatgaatgagcgagagacagaaagcgcaacaacaatcaatacatttttttaattaaattattaataaatgattgggccttttctcttatatctgagtgcaagctttttttttgaaagatggcccattataaatgggctatcgtttgaattaaaacgtgggccattactcattttagtagtttacttacagttaaattggatagacgtgtgagtgatcagatcccctctccgattgattgattctctcctcctccaaacaagccaagccaaggtatgccttctctctctctgcaatgagatcggtttttcagccggaagtcaaaacgtatcttcgtattgttgactacttcttagatctattttgctttttttttcacttgttatggattgctgctagatttaacacatgatctgctttcatttaacggatcaagttatgattctccgaccgaacctctaaaccaacactttgaaacattaatagtcctttttagtactttctgattaatattttacgccaaatagtgatcagttagttggatcacctattagcttgcgtcaaaaaaatgttcagaagttctatttttgggttgatccacaaacttcatttatctaacaagacttatagtttgtgtaggagaaaagagagatggaatcgagcagaggaggaggtcaaggtgggatccagcagttgcttgctgctgaacaggaagctcaacacattgtcaatgctgccagaaccggtaatttctcttatttttctactttcttagtttcccccacatgcttgcattctgaatctaaacataaatagttaaatacagatgatcttgtggccgaaaattatccaaacttgtatatttataaatgataagcacttgttgaggataatgcatagtgtccaatttgtcttgctggagctttgattataaatgtttggagtttgatttgtgatcttgtgttgcagcaaagatggcaaggctgaagcaagccaaagaagaggctgagaaggagattgccgaatacaaggctaaaactgagcaagactttcagagaaaacttgaggaggtaatatatattctacaaagtgatataagacaatctagtgttcttttttcttcttctttgtaataataatccatgtatatacatacatctgggggaaaacaaacagacaagtggagactcaggtgcgaatgtgaagaggctggagcaagagactgatgccaaaatcgaacagctcaagaacgaagcttccaggatttccaatgatgttgtggagatgctcctcaaacacgtcaccactgtcaagaactgagtctcacactcttatttatttctctgctttttgtaatttctctcttgttgttgtattcatatcttgtttgttgttgttggatgcgaggcttgagatgttatgtaaccaaatacacgaaacaataatgttttgagtcttttgcaagaaatatttcttgatgaataaatctcttttgagaaaacatgtctttctcctaacggtttgctacttaagtttagtaaagttggatcaatcgtgaaatcttttggcaaacagttagtttaagtctgtctgtgtcaaagtcatgggctcggttcggttgttcagcctaaaagccatagttaatgagccatagaggtgttatatattgggagttatgaaaaagcccatattccaccactatttgctagggtttctcctcgtcaccgccggtgacacctactttaaatatgaatcatctctctcctccaacttagacttctctctatctctctttcagtttttttggttcatcttcattatttttcctatctagatgttttatttatgtgtgagaaagtacaatgagaatagatcagatgatgagataataactgcaacaatatgatcttcttgatgttcagtatttttttttttaatacaatgttatcttcgttttcaaaatttgtattggttttgatttttgaggataagagaaagaagagatatgatgagcaactaaattggtccgtgtttcagattaaccgtgaccgaaaactttcttaaccaaactctgatctctagctaacatctatcttccttttaaacaataatttgattttatttatttatcttgcttggttcctgcggtgatgattttatgaaatattcacatgtcagacttcagagattcttgacgaatcagtgagaatgtattcatatttctgagcagaagactagtgtttttccaagcctcatagaaagttttcaaccgttgaagaaattggacaagaacaaatacgacttaatcactttaagaacatgattattgccgcgcagaaagaagaaacaaaacttaatattgctgagttgtatcatcgtggaagttatacattatctaatacataagtgcttgaagtacgagctaatcactttgagaatgtgacaattgcatttgtttgccttttatgttaaattataatgagtcaacaaagatctacaatagtctccaaagatgatcaagagcccgtttgttaagagtctaagaccgtagcggaacaactaaagaataccctatggactgttgtggtcgtttatggatccaaaatgttgtcctgtcttcaaaatatgatggtataattaggggttccaaatatttcttgaataacaaggcacctccgtgtattacattttaacagggtttaattgttacgaaactcaaactacatttaatttgtttgatatcaagtattctttctttttgggaaaattggaaaaatgcaaaatgtaatcaggtccttcaaaacattattaggtttcttgcttaacaagctatgtgttattcatgatatcatgtgataaacaaggtacttcaaaccttacccgaaaagtgtaagtcgaatgcatcggccgagtggagcctataattaagtaacacagaatgtgaacaaaatttactgaacaggttttataaaacatttctttaagtctagttaaacctgctttcagacactttcatattacaaatctaaaagtgtataactaaaagaccaaaataaaatcaaactttagtaaattttctgtaaatcttcagactgaatatatcagattagatcatacctaacaaatcagttcttagcagaattttataataaacaaatgtagtatgaaagcctgatatgtcagctactgtgttttgattataacctttttaactctttgcacaaatggtttagaataagaaaaagctcgaaagcaaccaaaaagctaaattacatttttctcttctgcatattctggaatccttggattttattctttcacagtttttattttttattttcttttcttgaccaaaaatcacaccagaacgctaataacgacagatagatttttatgtaaacattgataagtgacacagtggaccatgtgatacgttagtttgttagtaatcacatttaataaaaagtttcccatcactatcagctcacggacgatattacgctatagcgtcaagtcgtggctccattttgactagaaattaaaaaacaataaagcatatttttatttattttaggtattagtcttaacggcacggccagtgacgattgattgtcccttttatgtcctcgtgcttaatcagcgttcgttagatattcttaatatatttactttccaaagtctatctatgttttgccttcttcacaagcattatatattgtcgtcgctattgctctgccttgtgatcatcattgttttactggtaactctctctttgtctccttcatcttgttctctgcgagagaggagtagatatggaagtagaagaaggagcgagtgttgatgttggtggtgggaagataaaggttggagtatgcgtcatggaaaagaaggtgaaatgcggctccgatgttatctttcatttttttaaaaaaaaattgttactgctcgatgttttcttctactgaccaacagactcggatggttggtttgtagtttattttggagtcctcttgtttttactatgtgttcttctcgatcgctcaaagatagctcgttgtggatttataggtctcctcaagtcccatgggtcaaattttggacagactggagtcgtttggtgaatttgaggtaactttgtactcacgggtttcttctttgttccaagatttttatatgctataattttctggtttgtagtactctatatatagtttactatttaacctggagagaggttgcttttgattagatggcttgttatttagcttgggagatgctctttttgtgttcattttccaagttgttgaattagtttgcgactagaaatgttgttgactaatttgtctttgtatattctctggtgtattctttatatttttcatgtcatggacaactttatccgcagatcttacattttcgggataaagtcatacttgaagatccaatagaaaggtttgttgtactaaagagtttactttttcccttttccagttatgaatgacgtcccattattagtgtactaggagaaatagtagttaatctctatatatatcaagtactttctccaatatatatatatatatatatatatatatatatatatatatatatatcaagtacaaaaacctgacatgagaatttgctgtttcagctggcccatttgtgattgcttgattgctttccattccactggatatcctcttgaaaaggctcaggcttatgctgctttaagaaagtgagtttttttcaccttttttatttcttgattttggctcccatataaaagctttctcatgactccggcttctgaggttgacaacttttcaaacttgtcttgttcaggccatttttagtgaatgaactagatccacagtatcttcttcatgatcgccgcaaggtgtacgaggtattgcatattaggatgcaataagttggttctgtgtacttttgataatttttcaaagtttccagggctaatgagacaaagaagagctactacatatattttatttttccttgctactaacaaatagcttttgtgtgcatttttcagcatcttgagatgtatggaatcccagttcctaggtatgcttgtgtcaacagaaaggtaccaaatgaagaagtagattattttgtcgaggaagaagattttgttgaggttaatggtgaacgcttctggaagccatttgtggaaaagcctgtcaatggtgatcttctcttttttgcctcttgctctctctttttcatgccagcttattatctatttatccataagtctaaagtagaaaaataaccataggagtgtgttttgtattttcctctgatatttcgtaaccaatttcacttgaatctgttatctcatatgagttaaatattgaggtacaatgaatataaacagaggtgatttttcaaggctatattctcatttaatgttcttagttgttatatgatgatggctatcctcgttatatttatcttccgacagttaacattctttgtgcgtttgtaggagatgaccatagtatcatgatatactaccctagctcggcaggtggaggcatgaaagaattgttccgcaaggtatctctccagaagaatttggctgctatggattcaattgttgatattctaatatatttttcataataattagtgtcatattcctctgggactcgttttcagctaatgtatctcgttcgtctccattttactttaagattgggaaccgatcaagtgagtttcatccagacgtcagaagagtaaggagagagggttcctttatatatgaagagtttatgtccaccggaggaactgatgtcaaggtatgtgtttattttccttctgcatgtttctctattagctcatttcatcttttagaccacattaatctttctgagaatatcgtactattcttggtgtcaagtgtcctacatttataaggttatcatgttaatggcctatatgtttatatgacttagctcctatttgctggttgtttttttgtctggtgaacattttttcgtgaagttttggctttcacgagctctgtactgacaatctggatgctggcttaaatacaggtatacacagtgggtcctgaatacgcacatgctgaagcaagaaagtcgcctgttgttgatggtgttgttatgaggaatactgatggcaaggaagtacgtcgtctaccgatcatttaccttaaatagaatgaaatttgacactatctcattttcttatggagagagggctatattttaggtgcacaaaaaaaaagaagaggggctatattttgaatagaatatcaaagtctattgattatgatttggtaaatgtttcatgatggtgtaacactattattttatttttttattgaacaggtgagatatccagtgttgcttacccctgcagaaaagcaaatggctagacaactttgcattgcatttaggcaaactgtatgttgtgaatccaatccctctggctctcgcagagttttattttctttataatggtactaaattatttttattttttttacaaatttcatcaggtgtgtgggtttgatcttctacgatctgagggatgttcatatgtttgtgatgtaaatggatggagttttgtcaagaactcttacaagtaagctaatagcattcttcactttattttaactctctaaaatgtgcaccgttaaagtattattcacaaacctctcatactatactatgtaaggtattacgacgatgctgcttgtgtgctaagaaaaatgtgtttggacgtaaaggctcctcatctgtcatcgactctccctcacactttgccttggaagttcagtaagcctgtacagcctaataaaagactaacccgtcaagggaatggacagtcagaagagttgcgttgtgtcattgctgttattcgacagtatgttttttttagcatataaatcacctatatctcatctatacttctctctgttaaaattttgttattacatgacagtggtgatcgaactccaaaacagaaggtgaaactaaatgttacagaggcgaaactgttaaacctgatgttgaagtacaatggtggaaagccaagagctgaggtgaggagatcgactttttctgtcatccatcttttatctgccttgttcattttatatattttgtcaaattttgcagacaaaactaaaaagtgcagtccagttgcaagacctattagatgccacaagaatgttagttccccgtacaaggtacaaccctgaactctatcttctctccaaaaagcatgtctggtctaccttttccatgtgttgaatactcgtaccgacaatatggtctttctttttttatatgtagaccaggtcgtgagagtgatagtgatccagaagaccttgaacatgctgagaagcttcgccaagttaaagcagttcttgaagaggttctttagtactttctcttttttttttttgaatatagtccgtggaaagtattacatagtatcatttagggagttaaaactcttataagtaagctagcattatgcattttttatttaattcatgaggtcatattcttatagcagaaaaccttttggtatggcgcagggaggaaatttttctgggatatacaggaaggttcagctgaagccgctgaagtgggatggtgaaggcgaagaagaaagacctgtagaggcccttatgattcttaaatacggcggtgttctaactcacgctggtagaaaacaggtgcttacatactctaccatttaatttgtcattgctatatatatatatatatgtttcccaaatatggaaaattgtttaacttattgatgtttgcttcatctgcatgcaggcagaagagcttggtagattcttccgatacaatatgtatccaggtaaggccgcctaatctgtttttttttatttgttactttttttttggagtatttgacgatgaaatggctttactatatgttttttcaggtgaagggactggtttgcttcgtctccatagtacataccgtcatgaccttaaaatttacagctctgacgagggacgtgttcaggtacgtacaccttttacattgatactactcaagataattaaacatatgtgttggaaaaaataattattgaattggtttgcatatgttgaccagatgtctgcagctgcttttgctaaaggtctgcttgacctagaaggacagctgacaccaatcctggtttttctttctttttttaaatcttgttggtgcttaatttgcaatgtcatcatgtgtattactaataatatttttatctgtcttgcttctctaggtttctttggttagcaaggactcttccatgttggatggtcttgatactgccagcattgaaatggaagcggccaaggtttgtcccatgatgaatcattttctgaatagtccaccttaaatatgttaacagttttgatttcactacttggactatgcaggctagattgaatgagattgtaacgtgtggcaaaaaaatgaccaatgagcatggctcctctgaagaattcccttggatgactgatggagctggagttcctcccaatgctaatggactcctccgtgaattggtactcacacttttccatctagtcttagtttactactggtgtgaagtacatcaccaaccttttttttttagagttttgtttgcacaacaacgtactattgcataggcactgatgagcacattggtaacctctgtgggtattgctctcatttttgtggctccctcattaggtgacattgaccaagaatgtgactgaacaagtaagaatacttgcaatggatgagaaggagaacctcactgagccatatgatatgtatgatcaagcaaaggctcttggaaagacaaacattgacagtgataggattgcttctggattaccatgcggtagtgaagggtttcttctcatgtttgctcggtggataaaactcgcaagggatctctataatgaaagaaaagagtaagtttcttatcaaattttttaaacattatttagaggcttgtgagtgagtgagagtagcactaatagttcagttttaacttgcagccgatttgacatcacgcagattccggatatttatgattcatgcaagtaagttatatatgttatggtggttttttggtgtatctcgtttatgaatttactttaatatgtctgttaacgtcaccaggtacgacctgttgcataattcccatctaggtctacaaggattagatgaactcttcaaagttgcacaggtacaaactgtattttctcttttccagttttcacttacaataatactttgttatcttccaaggcatactaatgaacatgacctttcttggtagatctctgtattgcgttgactgtgttagcaattatgactaaataatgtgttttgtggttgcagttgcttgcagatggtgtgatcccaaacgagtatggaatcaatccacaacagaagcttaaaatcggttcaaaggttgacattttataggctagctacgagagtagtttatatatggtgtttccagtggatctctcacatctttatcaaaatctatttttccgtgaaaaaaggttgctaggcgcttaatggggaaaatcttgatagacttgaggaatactcgagaagaagcaatgagtgttgctgaactcaaagaacaacatgtcacattgtcattatctgcctcgaaaaaacaacaagacaaaaatattcaaccaaagagagatgatttgagacgacctggcactggtgataaggatgaagatgatgacgataaagaaaccaaataccgattagatcctaagtaatagcttactctctgcttgtatagtttctttcttcttctccttttcttggtttttgcatattcttaagtgttttatactaattgcttttcattttcccattttaggtatgcaaatgtcaagacacctgaacgtcatgtgaggactcgactctacttcacatctg

SEQ ID NO: 3 - DNA, 6454 bp, Brassica napus
  source 1..6454: mol_type = genomic DNA
aagccagaaaaaaatgtggaagtgtaataaaacaattttctccaggaagaagatatttcagccattcgcctatttatgtgtatgagatcccattcaaggacatactgttttccgtgagctactgaatgaatacgatgcttttgttgtaaacgcaacctattggttttttttttccgccaagttttgtttttattaatcaaacgggccaagcccaaacggtcgaagcccaaatacactaagtaccaaacaaaaggcccacagacacaacgggctacaacattaatggctgaagcccaagtaaactaaacggcccaaaggcccaaacattgaaaccggacagcttgacctaggcgcgcgataaggagactggggtttgaacacgtgtaagaatctgggccattagtatgacacgcgtcgcccgcctcgacttgatcgaaacctcttcgcacagccgcatgaacttgccaccgacgacctcgtttcgccggagctcagagactgaagagcctccaacgaaaccatagctattctacgccgctctgtcttcacgccggagagttccatcgatctaacgagatctcctccgactatgtatcaccacaaaccctagacaggcgagccgagaaccactgatatcctcctcttgtacgtcctcgccgttgtcggagagtttcatcatcatccgagatctcatccgcgatggcccacgaccccatacctgaccacaagaaccacaccgcacttatctcacctcgatctaatcaccacaacccaaaggacatcgggttccaagacggcagcgcggagctttgcaagcctccactctccgtgaccaaaagccggcgaagacggagctgaatgagcctccccttcccggaagctaaagcggcgacggtggatctgagaaagcctccacctcccgcataaagaccggcgacgatggatctagggtagcctccatctcccggaaaaaacaccacctgaacatgcaagccgctccatctccaccgggagcctccaattgatcgcgtcgttactccaaaggccgagccaccgttgatggatggttgcggtcccagagctccgacaaggcgatcgatgacgggactgcgaagaaggagcggagagtgacaactacaaaggaagggaaagagctctggcgacagcacggacgctcacgcgccagccgtacgccgaacccaaaagtagatctggtttctctcttttctctctctcgcaaaagaaaacacgcaacctattgttctccattttgaatttgaagtgttacattattaataaaaaggaagaaagtacaaagcttttcatttagtggttgttgcatatatagtatacttatttgatgggcttatgataagtatgaacatttcacgtaacgggtttattttgtcgtcaacacattaccggcaatgtgaaaagaaggggcgaggtatctccatttcaattcttctctttatagattaatcgaattatttacatatgaaatgagcgtttatatagaattttcgtgtggaaaacgacatgtacacggcatcgaaagaccaattagtaatatactttagtggtgattacatgtttacttatccaattgagaatttaaagcatcgacaataccttaatgtcgattaagccgtccccacttcatgtaatgagttatggggggagagagaggtcccgaaattcgtcaaataaaacaacttagaactaaaaaccgacaccaagtatcataaaggaaacgttgaagaagtcatttatcgtatccagctcacaattcctaagattaaatcatgaccgttggaagagcttataagattaaattgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctatatggcaatgatatcatcaccaaagtgcaatgcccctttttgccctagtttcgtaaagtctctttccttcttcatccttaggaaaaaccctaaattaaatcctgtgttcttgatcttgctttttgagtaaccatgattttgaccacacactagttcttctatattttgtggtctataggattttgctttatatgtgtttcttgtattgcaccgtacgtgcgtatataaatttaaatggttacaacaaggtttattataaacaggcacaaattagtccatgaggttatttagcttgcacaagaggtatataaatgtgtgtgtgtgtgttaagtatttaaatatataaatttgctacaaaacttaattaactttatctgattatattttctttagtgttcttcatttgccaacgttgaggtagctattattattattattattattattattattattattattattattattattattattattattattattattattattttgaacattatgtactatgtacgtagttattttggctagttatgattcaaattcttaatttggataacacttaacagtatttaaaaaaaaaatactatttaaaatattcttagtactaaaataattaagatttaacttgaaattgaagtattcgtgttaaacagaaactacaataaacaaatgattgcatgttaatttttttttcgattatcctatatatcagaataaacacatgattgcatgcaaattttgtttttgattatgtcatcttttgtttattttagttttgatgctaattaatattttttttattaacaacttccatacattctacctgattctaggtcagataatgacacagcgcaacaaaattatacaaaactttcggaaagtagattaccgcggaagtaacatttttgggtagacatacgaagtagtttgaatcttcaaaaatcatgccatacataaccacggatcatagtcgacacctcaacgtgaagcaaatttgacaatctacatacataaccaacaaaaagtagaataccgtgaaaacctaaacccaaaatatgatgtaaaactcaagcttggtccagagcataaaaaaattaaagccatcgctttggtatcacatatttaaacgtcagtttttttttggggaagtaatataaaaatataattaacaagaaaatttatgaaataattagcatgtaaaacactagtcttttggttgtgacaaaacgttttcacaaatgttctataaataaattcaagcacattttatctgcaaaatatatactttcactcataaaataagagcgtttaaaacattcatatacgcactacattgacatgacaaaagaaatccgcaaatacaaacatatttagttcggatatatctaggaaataagactatattatatatataaagaaattagaaaaaaagaaaattggtatgtataaaagaaaggtctatgcgtctctagtccaaaaactctatatgtctggttcaaaagcagatgcagcagccatagccccaatagtcatgatggagcctcatcatctccttatgaactggaacaaacctattgatctcattacacaagaaaactcttttaaccacaatcctcatttcatggtagatccaccttccgaaaccctaagccacttccagcccccgccgacagtcttctccgatcccggaggaggagaggaagcagaagacgaagaaggagaggaagagatagatgagatgaaggagatgcaatacgcgattgctgccatgcagcccgtagacatcgatccagccaccgttcctaagccgaaccgccgtaacgtaagggtaagcgaggacccccagacggtggtggctcgtcggcgtagagaaaggataagcgagaagatccggatattgaagaggatggtgccaggcggtgcaaagatggacactgcctccatgcttgacgaagccatccgctacaccaagttcttgaaacggcaggtgaggcttcttcagcctcacactcagcttggggctcctatgtctgacccttctcgcctttgttattaccacaactcggatacctaattataattctatcacgcgtttcatgttgatatatatagataaatggttgaataaggatttcgatcgaagattgtatggctattgattacattatatattgtacaataaatgatgtgtgtatttctattaatgtatatatgatatatatctgtttgcagtatgcatttatattctattctttatagggaggcaacatgccggattagggctttgatcgtatgcaagttttccgaccaaaaatatgaaatacttgtttggatataacatatgaatcggataagtgttactagttatataactggaaaaaaattgtttggtataagaattcccgggagaaccaagcctttctctaatccctaagatcatagctactgaaataatgaaaaaaaacaaaaaaaaaacaatgaagaatcagttgggcattagtccaaaaaaaaaaaagaatcagttggattgcttataaaattttgttataaaatttatgtcgtatgtgtgttagccgtagctgtaaatttatttcctttgtatgttaattgtaaaatttaatttattattcccgatgtttttaatgatgtaagtttacatcaagaaaacatattaagttctgttaataaatgtattatttttgtttactaatttccaataattttttcaagaagtacaattttttgtttgaaatttctaatttatcaacatacctaaaaaccataaacatgtattttttgaaacatttttttaatataaaaattattttttaggagcaggtgaagtatttcttttaataaaattgttagtgtatttacgttggttatagttgtagatattttgcataataattaataatatataagatgtgtaatgtatatatgtgagagaaataccatatttactaagagtttttcgcaatattgactcaaaattcaaagtcaatcacaaaactaacatatgtttctttttagacatttcctttgccttattcactccacgggttcagattattcacgaaaatgtcattaaattttttttcttttcgaaaatggcatttttactctctcaatttcatcctcttcaagtatttacaagatttccattgccatcaatacaccaaccacaatgaacaaccaatttgaagctcttaatgcacctcaaatcgatttacacttcttctttttcaattcttatgaactaaaaacaacatctctttcaatttctccccatattcatccaaaaaacccaagatttttattctaaaattttaatggttcatagagccattgaagcttacgattcttggtgggtcacttttgttcgagattttaggtacttggagaagacttttgtatgctaaacaagttatctcattggctgaaactatgaaatcagttttttttccagatctgttcgcccggacgacttccggataagtcttctggctgtagacgacttacatcgaagtattctggttaatgcagatgttagttttgcaattgacttttaagatgcttactcgaagattagtactcttctgttcttgtaccttatgagcattccaatagtattatatttttcattctaaaatatatttcaatgatattttacttttcactctataatagagtgaaaatgagtttactttataatcccctatatattatttgtgaaacactacaacttctttttgtagccacatgtcatcactaggatgatttttagaattattagagaaataggttggtccatctaattatataataagttttttattaaattaaccataaattcattattaatgtcatttattatttccttaaataaagattacggaattgcctaatgtgggtaaagtatatatgacaattaatgatcttgaataataaagatatgataaaaaaaatgtatcttctatcaaatttgtttaatttaaaactattaaaataatttttaaaaaaacacaataaccatattataaaaatttagatttttctgtatatttttatattttgaactttaaaaaatgacgataaattactaaaactgttaaaagtctcacattcaaattttgctatccatgttttaaaatttttgttatgacaaaatacaaatgattacaaaatcatataagtaaaagtctaatttaattaatcattaagatttaaaatatatatgtatatatatcattctaaattaaactataaaccatattgaataaataaatattttagtttcaaaattttactttgaataattttttttgataaaagttttgaactaacattgataaatgtttttaaactatcaattaccaaaattattgatcccacaatgaaaattttgttatcagtaatttaaa

SEQ ID NO: 4 - DNA, 4772 bp, Brassica napus
  source 1..4772: mol_type = genomic DNA
gaattacgaaaacaaaatttgaaaaaaaaataataaaaaatttcaaaaaaaaattataaaaagttcgaatttgaaaacatataatctgaaactataaaaattaattattattattattatttgtatttatatatctagggtattaaaatctttttacttattttatgaaacattttggttatctttctccttacaatctatttttgtgataaaaacttgaaaatagtctacttaggagaattaccctttacattatctaataaactgatcttctagaagagagaaacgttaatggtggacgaagcccccacctcaattgacaataaaagaaagtggaaaagagagaagatagcagagtgaaaggtccgaagagaggagatcatgaagagaagaagacaatgtaacctacttatttgccttgatgaacaattacgagaagaaataaacataaaatgagaagagagaggtgggatatacagaagagagaaagatttggcaggaaacccgattgaccatatatcagatctagagtggattggattagataactcgagcttatttggttaatctttttagtttagggaaggttatgaaaaattaaccccgatctgatttttaaattttttatcttatattgtgcccccggctaacaatatatctagatccgtccctgatcaggtccatgagatatatcatatttttatttttcttatttaattaattatatatataatatttgaggatatatttaaaattttacatatcgttgaaagccagaaaaaaatgtggaagtgtaataaaacaattttctccaggaagaagatatttcagccattcgcctatttatgtgtatgagatcccattcaaggacatactgttttccgtgagctactgaatgaatacgatgcttttgttgtaaacgcaacctattggttttttttttccgccaagttttgtttttattaatcaaacgggccaagcccaaacggtcgaagcccaaatacactaagtaccaaacaaaaggcccacagacacaacgggctacaacattaatggctgaagcccaagtaaactaaacggcccaaaggcccaaacattgaaaccggacagcttgacctaggcgcgcgataaggagactggggtttgaacacgtgtaagaatctgggccattagtatgacacgcgtcgcccgcctcgacttgatcgaaacctcttcgcacagccgcatgaacttgccaccgacgacctcgtttcgccggagctcagagactgaagagcctccaacgaaaccatagctattctacgccgctctgtcttcacgccggagagttccatcgatctaacgagatctcctccgactatgtatcaccacaaaccctagacaggcgagccgagaaccactgatatcctcctcttgtacgtcctcgccgttgtcggagagtttcatcatcatccgagatctcatccgcgatggcccacgaccccatacctgaccacaagaaccacaccgcacttatctcacctcgatctaatcaccacaacccaaaggacatcgggttccaagacggcagcgcggagctttgcaagcctccactctccgtgaccaaaagccggcgaagacggagctgaatgagcctccccttcccggaagctaaagcggcgacggtggatctgagaaagcctccacctcccgcataaagaccggcgacgatggatctagggtagcctccatctcccggaaaaaacaccacctgaacatgcaagccgctccatctccaccgggagcctccaattgatcgcgtcgttactccaaaggccgagccaccgttgatggatggttgcggtcccagagctccgacaaggcgatcgatgacgggactgcgaagaaggagcggagagtgacaactacaaaggaagggaaagagctctggcgacagcacggacgctcacgcgccagccgtacgccgaacccaaaagtagatctggtttctctcttttctctctctcgcaaaagaaaacacgcaacctattgttctccattttgaatttgaagtgttacattattaataaaaaggaagaaagtacaaagcttttcatttagtggttgttgcatatatagtatacttatttgatgggcttatgataagtatgaacatttcacgtaacgggtttattttgtcgtcaacacattaccggcaatgtgaaaagaaggggcgaggtatctccatttcaattcttctctttatagattaatcgaattatttacatatgaaatgagcgtttatatagaattttcgtgtggaaaacgacatgtacacggcatcgaaagaccaattagtaatatactttagtggtgattacatgtttacttatccaattgagaatttaaagcatcgacaataccttaatgtcgattaagccgtccccacttcatgtaatgagttatggggggagagagaggtcccgaaattcgtcaaataaaacaacttagaactaaaaaccgacaccaagtatcataaaggaaacgttgaagaagtcatttatcgtatccagctcacaattcctaagattaaatcatgaccgttggaagagcttataagattaaattgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctaccaagcctttctctaatccctaagatcatagctactgaaataatgaaaaaaaacaaaaaaaaaacaatgaagaatcagttgggcattagtccaaaaaaaaaaaagaatcagttggattgcttataaaattttgttataaaatttatgtcgtatgtgtgttagccgtagctgtaaatttatttcctttgtatgttaattgtaaaatttaatttattattcccgatgtttttaatgatgtaagtttacatcaagaaaacatattaagttctgttaataaatgtattatttttgtttactaatttccaataattttttcaagaagtacaattttttgtttgaaatttctaatttatcaacatacctaaaaaccataaacatgtattttttgaaacatttttttaatataaaaattattttttaggagcaggtgaagtatttcttttaataaaattgttagtgtatttacgttggttatagttgtagatattttgcataataattaataatatataagatgtgtaatgtatatatgtgagagaaataccatatttactaagagtttttcgcaatattgactcaaaattcaaagtcaatcacaaaactaacatatgtttctttttagacatttcctttgccttattcactccacgggttcagattattcacgaaaatgtcattaaattttttttcttttcgaaaatggcatttttactctctcaatttcatcctcttcaagtatttacaagatttccattgccatcaatacaccaaccacaatgaacaaccaatttgaagctcttaatgcacctcaaatcgatttacacttcttctttttcaattcttatgaactaaaaacaacatctctttcaatttctccccatattcatccaaaaaacccaagatttttattctaaaattttaatggttcatagagccattgaagcttacgattcttggtgggtcacttttgttcgagattttaggtacttggagaagacttttgtatgctaaacaagttatctcattggctgaaactatgaaatcagttttttttccagatctgttcgcccggacgacttccggataagtcttctggctgtagacgacttacatcgaagtattctggttaatgcagatgttagttttgcaattgacttttaagatgcttactcgaagattagtactcttctgttcttgtaccttatgagcattccaatagtattatatttttcattctaaaatatatttcaatgatattttacttttcactctataatagagtgaaaatgagtttactttataatcccctatatattatttgtgaaacactacaacttctttttgtagccacatgtcatcactaggatgatttttagaattattagagaaataggttggtccatctaattatataataagttttttattaaattaaccataaattcattattaatgtcatttattatttccttaaataaagattacggaattgcctaatgtgggtaaagtatatatgacaattaatgatcttgaataataaagatatgataaaaaaaatgtatcttctatcaaatttgtttaatttaaaactattaaaataatttttaaaaaaacacaataaccatattataaaaatttagatttttctgtatatttttatattttgaactttaaaaaatgacgataaattactaaaactgttaaaagtctcacattcaaattttgctatccatgttttaaaatttttgttatgacaaaatacaaatgattacaaaatcatataagtaaaagtctaatttaattaatcattaagatttaaaatatatatgtatatatatcattctaaattaaactataaaccatattgaataaataaatattttagtttcaaaattttactttgaataattttttttgataaaagttttgaactaacattgataaatgtttttaaactatcaattaccaaaattattgatcccac

SEQ ID NO: 5 - DNA, 3100 bp, Brassica napus
  source 1..3100: mol_type = genomic DNA
attatatatggaccaaaacatgaatgatttttcggaatcagatactccattcagcttcgaacagttattatatggagatgattggcagaggtgggacacttatccgagcgttgatgttaatgccaactcaatagagcctaatcattcgatggtcgagatgaaccatgatggtatgacaatagatgctactccaatttcaatgatatatccaatggtaataatctactttctgcatgttttttttagagatattaaagtatggataaataatataagacttattgaaaatgtataggaagaaacattcgagcagtggttacagacactggccaatggatcatttgaaataccatcatatgaagaactcaacttcacgtatgaggtatggtgaaagataatatatataaaggtatattttatgtaacatataatcaatttattggtaacttatttggttatattgcatttgtacatttttttctcagtggatatgcatttgaatcatagttgaagaaaacaaaaaggaatatatatgctgcaattggattgttgatttttaaaatgatatgacgtccaatgacttatttcattatttccataaatttttacagtatttaacatgaatagaatgaaatactcattccatcaattccacaaacattccaaaaaatatacaaaaaatacattcatatgcaattttaattatgagtgaatagaatgaaaatatttcttttttaactaattccattcatcttattccatctattttgattccactaatttcatttcattgttaccattgacaaccataatgtgcatggttgtaaatttagaactctgtaaccatatatatatatattcaaaagtttattagtgtatttttttctaggaattattgctcatgagtgagcagataggagatgtctgcattggtgtagacgttgatatcattgaggggaatctgaaacgaagaaagtatgaagatcgttctggtcaagcggagaagtgtgtgatatgtctcgatgaacttaagtgcaatgatgaggcttctacactggcatgtgggcatgattttcactacgagtgtatcaaaaactggctaatggtcaagaacaagtgtcctctttgcaagcaacaagctctttaattgtttcatgatgctgaatatttgaatgtgttagtattgctaacattggtttcttttgttcaagtatcggtattcggtagtttaataaaaacaaatatggaagtttcataacaaaaaaaaaagtctaccaaaatttatgcaagttaattatcattcaagacaacacccactgcaatttgtcaaaaatggtgaaactcttgcaagaccttacctgtgaagtgattgtcctttttcatgtttaataataataattaattacagtattatttatgactttcagaaaactctatctatcaaccgttctttactgatttttccacccatcactgctctattttttatttatggcatcgggaaaaaaagagaagaaaaagaaatattatatgcaatctttttttttttttatgcaatcttaccgggtgttaatacttataactacatttagtgataaatctgctttgttaaatccagtacaaaatcaattattgaatttatagactataaagttgtgaacaaaatacaaattatcactaatgggaatattcatatacagtataataacgtaagccaccagattatggctagagtgatttgccacgcgcctgcctatttattatgaaaagcctcagtaactttgtgatgagaagaattcacagagagagagaggagagagatgggtaattccgacgaaggtgatcgtcttcctgctccatcttcttcggacgaactctcgagcattctccggcaggtactgtcccgtgctcccacagctcaaccttctttctcaccgaagaaaatcgtttcctccgctgagatgttcaaccgaaccttccccctcgttcccggcggagcggtttcttacgccgcttgtgcagccgctgaaactggggaaggcaaatgtggtttcgaaaacaaggtaaacttaacgatgttagtttccgtgaaactaccctcgcttttgattaaagaaaaaataacttagttgttgtttttgcttggtaatggttaacacaacactaaagagaaatggagctagacagcgaaattcattgaagagaaacattgatgcacagttccacaacttgtctgaaaaggtttcgtctttatccttttaagattcttgatttggtttaaaaaaaaactagagataataatagaaactggatatatgcagaggaggaggagcaagatcaatgagaaaatgaaagctttgcagaaactgatacccaattccaacaaggtggtaaataccaagttcgcctttttatccttcagaactcagttgtgagaaccatttgttttgttttttatgttttgtagacagataaggcctcaatgcttgatgaagctatagaatatctgaaacagcttcaacttcagtttcaggttctttttctatatgttccttacgctatgatcatgaacaactaaatttgtaaaaccaaacatctgattaacttaatgaatgcagacgttagccgctatgaatggtttaggcctaaatcctctgcgattaccaccaattctaccgcctacgcagacaaggatcactggaacctcagaacaagggctgaaccttgagactctgcttggtggttctcactcgatggctaaccgtgaaccacccgaaactcaggaaatgtgctttcccacaaccactctgctttgaagacaacgttcagacagtgaagaggatccgaagtcagatttcctctccacagaaacatgagtcgaaaatgatttgtagagtctagtatttggttatataagtgttatgctaataagctttaggactgaacaaaactgagtagcttcattataatgatgtcagaataaatttaactacatgattaagatcaaatggctatgtctgtgagttgcagccatccgtacgcacaccggtctcggatacttcgagccatggatgctgctgactgaaccgcaggtcttatc

SEQ ID NO: 6 - DNA, 7672 bp, Brassica napus
  source 1..7672: mol_type = genomic DNA
aacatgaatgatttttcggaatcagatactccattcagcttccaacagttattatatggagataattggcagaggtgggacacttatccgagcgttgatgttaatgccaactcaatagagcctaatcattcgatggttgagatgaaccatgatggtctgacaatagagcctaatccaatttcaatgatatatccaatggtaataatctactttctgcatgtttttttgagagatattaaagtatggatgaacaatataagacttattgaaaatgtataggaagaaacattcgagcagtggttacagacactggccaatggatcatttgaaataccatcatatgaagaactcaacttcatatatgaggtatggtgaaagataatatatatacatagaaaacaatatatatatatatatatataaaaaaataaaagggtatattttatgtaacatataatcaatttattggtaacttaattggttatattgcatttgtacattttttcgagtggatatgcatttgaatcatagttgaagaaaactaaaaggaatatatatgctgcaattggattgttgatttttggaatgatatgacgtggaatgacttatttcattatttccataaaaaattttcagtatttaacatgaatataataaaatattcattccatcaattccacaaacattccaaaaaaaaacattcatatgcaattttagttatgaatgaataaaatgaaaatatttcttttttaactaattccattcatcttattccatctatttttattccattaatttcatttcattgttaccattgacaaccataatgtgcatggttgtaaatttagaactctgtaactatatatattcaaaaatttattagtgcatttttttctaggaattattgctcattagtgagcagataggagatgtctgcattggtttagacgttgatatcattgaggggaatctgaaaggaagaaagtatgaagatcgttctggtgaagcggagaagtgtgtgatatgtctcgatgaacttaagtgcaatgatgaggcttctacactggcatgtggacatgattttcactacgagtgtatcaagaaatggctaatggtcaagaacaaatgtcctctttgcaagcaacaagctctttaagtgtttcattatgctgaatatttgaatgtgttagtattgccaacattggtttcttttgttcaagtatcggtattcggtagtttaatgaatacaaatatggaagtttcataacaaaaaaaaaagtctaccgaaatttatgcaagttaattatcattcaagacaacacccactgcaatttgtcaaaaatggtgaaactcttgcaagaccttacctgtgaagtgattgtcctttttcatgtttaataataataattaattacagtattatttgaaactctttgtaaacttaaagtacttttcttattgaatgaaggcaacataagagttgcttaaataacaaagcgtacagatactcccgacagttttggtaaagtcttaaccacttcctatagtagaggaagacttattcaaataacttaaagcagaaaataattcaaaactaccaactaaagactctaccaactaaagactcgatcttttttattcattaatccctcccttaaacttgaactcctttataaagtttaaacttcctttaagctgactcccatgtgtgctctcagcttctcaaacgcttcaagtttcaccggctttgtcatgatatccgacaattgcttatgagttgaacaatactccattgagatgatgccttcattcacaagctctcttaagaaatgaaacctcacatgaatatgcttgcttcttccatgtagaacaggattccttgacaacttgatagctgaactattatcacagaaaagaacagttccatcaccttgatcaaacccaatttcagccaacacgttcctcatccatacagcttgacaagctccaattgctgcagcaacatactctgcttccgtggtagagagtgtgacaattggttgtttctttgagctccaggacacagctcctcctccaaacatgaaagtgtagcctgatgtactctttctgtcatcctcatcgcctgcataatcactgtccacataccctacaagcttttcttctccaccacgcaagtactgaataccatagcttgcagttccttgcacatacctcaaaatacgcttaacagccatcatgtgcccttcatttggattctccatgtatctactaaccacattcacagagtatatcagatcaggtcgagtagctgttaagtacctcaagcttcccacgagttgcttgtaagcggttggatcaacttcttctccagctcctgtcttggtcagtttgttccctggaaccattggattcttcacagagttgcagtctttcattccatacttctttaaagtctccagtgcatacttcttttggccaataaagatcccaacctcgtcttgcacaacctccacgcctagaaagtacttcatctttcctagatcagtcattgagaactcttcttgcattgaagttttgaagctctccagcatgctttccgagttactagtgtaaatgaggtcgtctacgtacaagctcaggatcaacgagttcccttcttctgtttttacgaacaatgtgtgttcgcaataacatttctcaaagccttcctttataaagtaactctcgatcctgctataccaggccctaggagcttgtcttaatccatacaaagcctttttcaacttgtagaccttctcactgtcaacctgagactcgaaccccttaggctgctcaacgtaaacgtcctccttaagctcaccatgtaggaacgcgctcttcacatctagttgatatacactccatcttctttgagccgctaagccgagaatcagcctgatcgtgtcccatctggccacaggtgcaaaaacttcatggaagtctactccataggtttggtgaaagccctttgccaccagtctcgctttgtacttatccacctctcctttttcattcaacttggtcttgaagacccatttcactcctatcaccttcacattggcaggtagactaaccagctcccaagtgttgttatcctctattgattcaatctcagtttccattgcctttctccatttgtcatgttggatagcatcttcaaacttctctggatcctcatctgacaagaacagcgcaactagttcttctccatcatcttccatgataagacccctcccatcaaccacgtaatctttcatccagactggtcttagtttgcttcttcctgctctgcttgtgtctgatctttcttctggtgtagagacagttctttctccatcagactcagtgttatctcctccttctgtctcttcgttagttgctggtgctgtcaccaggttctcaccctgattcctctcttcctcttcttcagcttctccacattcgacaaatttgtcttcatcaatgttatcttcccagttccaccgcgtttcttcgtcaaacctcacgtctttgcttattatgatcctcttcccaataggatcataaagtctgtaagccttcgactccttgcttacaccaaacatgacacatctcacacttttctcatccagcttcacacgcttctcataaggtataagtgcataagccacacagccaaacacacggagatgctctacagacggcttgtgcttactccacttctcacttggcgtcacttctcccaatatctttgttggacttctgttaagtatatggacagcatactgtgttgcttctgtccaatacttgatcggcaccttcataccaaacagcatgcacctgaccatattcagaatgcttctattcttcctctctgccacgccgttctgctgaggcgtgtatgcggctgtcagctgtcgtttgatgccgtgctccacacaatagtcctgaaacgcctttgaattgtattctccacctctgtcggttcttagacagacgagcagctcaccactctccctctcagcggcatttttgaactccttgaaagttctaagtgcctctgatttctctgctagaaagaagctccaacactttcggctaaactcatcaatgaaattgattatgtacctcttgccgctttcagatgtgggtgatattggcccgcagatgtcactgtgaactagctccagtcgttgtgttgctctccatagactcttctttggtatcttttctctgatctgcttccccttcatacacacctcacatactgtcccgttggtgttgatctttggtagaccttccaccatcgccttttctgctagtgttagcaaactgttgtggttcaaatgtccgaagcgtttgtgccaagtttcctccattgtctccatcgtattctgaacttggtttgcctccaacgcttccttaggctccctgacaatagcagttactacaaacatcctgtttgtggtcatcagagaatgcatgatcagtctccgttgttgtttgtgccagatctcacacgtatctccttcaattataatcctcaaccccttctgttgcaactgaccgacagacagtagattgttcttcaagccaggcacaaagtatactgcagtgatcacttgagtagtaccattgatctccaatcttaggttacctcttccctcaacttgcattcgcctatcatctccaagctttacttgttgtctgaactggctatcaaactctgaaaaccactccttggttccacacatgtggttgctacaccctgagtcaaggtaccacacgtgtctttcttcagtctcaacttgagtcatcagcaaaatatcctcctctaactctgcatagttagctgccttctcccagctaggacattcgtttttgaaatgccccatcttgtggcacttaaagcactcaatgttgctcttgtcaaagcttgtatttcctctgtcttgtcctccacgacctcctctgtaactgcctcttcctctggtttgatatcctcctcgtccattggagtaacctgagtagccacctcgtccccttcctccttccatcttcaggacttgttcctctcctgtgtgtttactcaggttctgctcatgtacgagtaatgagctttgtaactcatccaccgacagctccttgatatcctttgattcctctatcgcacataccacatatgtgaacttctccacaagggttcttaggatcttctcaaccaccttttcatctggcatgtcttctccacggttccgcatgtcattagcaaccaccatgactctagagaaatagtctgcgattgagtcaccttccttcatctccaggacttcaaagtttcttctcagcctctgtaattgcgcactttgaaccctcttgttgccttggtactttgccttcagcgactcccatatgtcttttgcggtgtccttcttcacaatcgtcttcagaacagtcttgtcgatggaggcaaagagatagttcttcaccttgaagtctttgagcttttgctccgcaagctcggttctctgagcttcagacagtgtcacactactttcatgttgaatgattccggtattgatcagttcccaccactccttggatctcaacaaattctccataagcattgcccaatgctcatagtctccatcaagctttggtatgctcaacgaggactccttttcactcatctctcttactttcggattacctagaactcacaacagtttttgttagagctctgataccacttgaaactctttgtaaacttaaagtatttttcttattgaatgaaggcaacataagagttgcttaaataacaaagcgtacagatactcccgacagttttggtaaagtcttaaccacttcctatagtagaggaagacttattcaaataacttaaagcagaaaataattcaaaactaccaactaaagactctaccaactaaagactcgatcttttttattcattattatttatgactttcagaaaactctatctatcaaccgttctttactgatttttccacccatcactgctctattttttatttatttatggcatcgggaaaaaaagagaagaaaaagaaaaattatatgcaattttaccgggtgttaaaacttataattacatttggtgataaatctgctttgttaaatccagtacaaaatcaattattgaattcatagactataaagttgtgaaccaaatacaaattatcactaatgggaatattcatatacagtatagtaacgtaagccaccagattatgtctagagtgatttgccacgcgcctgcctatttattatgaaaagcctcagtaacttgtgatgagaagaattcacagagagagaggagagagatgggtaattccgatgaaggtgatcgtcttcctgctccatcttcttcggacgaactctcgagcattctccggcaggtactgtcccgtactcccacagctcaaccttctttctcaccgaagaaaatcgtttcctccgctgagatgttcaaccgaaccttccccctcgttcccggcggagcggtttcttacgccgcttgtgcagtcgctgaaactggggaaggcaaatgtggtttcgaaaacaaggtaaacttaacgatgttagttgccgtgaaactaccctcgcttgttgattaaagaaaaaaataactttattgtgtttttggttggtaatggttaaaaaacactaaagagaaatggagctagacagcgaaattcattgaagagaaacattgatgcacagttccacaccttgtctgaaaaggttttgtctttatccttttaagattcttgatttggtttaaaaaaaaaactagagataataatagaaactggatatattgcagaggaggaggagcaagatcaacgagaaaatgaaagctttgcagaaactgatacccaattccaacaaggtggtaaataccaagttcgactttttatctttcagaactcagttgtgagatacatttgttttggtttttttttatgttttgtagacagataaggcctcaatgcttgatgaagctatagaatatctgaaacagcttcaacttcagtttcaggttctttttctatatgttccttacgctatgatcataaacaactaaatttgtaaacccaaacatctgattaacttgatgaatgcagacgttagccgctatgaatggtttaggcctaaatcctctgcgattaccaccaattctaccgcctacgcagacagggatcactggaacctcagaacaagggctgaaccttgagactctgcttggtggttctcactcgatggctaaccttgaaccacccgaaccaactcaggaaatgtgctttcccacaaccactctgctttgaagacaacgttcagacagtgaagaggattcgaagtcagatttcctctccacagaaacatgagccgaaaatgatttggttatatttcaaagtgttatgctaataagctttaggactgaacaaaactgctactggttatatttcaaagtgttatgctaataagctttaggactgaacaaaactgagtagcttcattataatgatgtaagaaaaaaaatgtataaatttaactacatgattacgatcaaatggctatgtctgtgagttgcagccatccgtacgcacaccgg

SEQ ID NO: 7 - DNA, 7235 bp, Brassica napus
  source 1..7235: mol_type = genomic DNA
atataagcatgttgagtcctattcatcagaaaattttctccaaaggatgaacaataacctacaattaaggaaacttattataacaacgaccaagaagttttcccataagtcatgctttcgtccataccttcgggtctttcaagcaagcttcacacatgccttatcgtatatttgtatacgttttcattttgtgtacgtctattaattagtatatgcctagattatatatttatatagtcgacaattacctatcctaaaggtttattttaatttttattcagaaagatatgcacgtagggccactagtgtttcccgttgttattggaaattccatgtgttgaactgtttgattaagtttttgaacccgtaagtcaaatttatttaggtttaacagggtttcattactgttataagcttgtcaagaaagaatttgtcacaaatttcgagactcatgcatgcatgcatgtttcttaactttcgagaaacatgaagtgtggttttaccagctgaagtttgtgttaaaacacatttttaatttgaccacgtgtgatattcaaatatcaacaattctgttcaacgggagattcatacatatgttcagatgttctttaatcttgagcacctaactttgcttcccatacgcaaatgtgtgtgccgcgaactatggtgaatgtacggcatcgtttttctttttggagcatttttaaaagaaaatataatttagttatttccgttttgattgatatagatgcattaataaaccgacttacgtgggtgatgggatgagcaaaattaaaatatgcattttagtaaccaattacggtccatcttgacaaactaacaaacaaaaagtctattatgacgttgatttgtgaccttgccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaatgacaactcctgttcttgtatcgtgtatctagcacgtaaatttgaattatggcggcattatcgataaaatatgtaaagaatatattttcatcatacaccacaaacaaatgttctggaaatatcaccttttatcacatatacaaagctgaccctgaatttttggaaactacaagccaaaaaaaaatggacaaaagaaaaattatgcacaaaagtggactcgtcctaactctttttataatgataattaacctttgaacaactaattaatacatacaatatgttttttttttctaaaaattggtcgattaaatattcatatataatcctcatcatcatttattagccttttgtagcttacttaaaaaatcacttcccaccaaattgattattttttcacttcatacgttgatcaatttaaaaaaaaaataataataatatatgcacgtccggcgtcccatgacttcagacgcttttctgtttccttgattttataggttagaaatttccaaaatgctcatatttttcactgatatgtattatacgcacaaaatagtaaagtcataaaaatttatgatggttcttttatatgtatagagtagtgaaaatttatatatattgtagttccgcttaaaataaattaatatttttcttttatcgcccatatttaagttactaaacgtatataattaaaaactgaaatgcaatatgtagaaatccgggcattgattgttgattaacattgatatacccatgcgactggcttatgatgaaccatacctgctaatactcattgttacaaactcgcagcaacatcataaatccatcactcgccacaataattttcttcagtgtatgttcaattgatcacattttgacccttcactttcttgaatgagaaattatacaagaaagtgatttgagtgtgttaaaaaatgatttgtcattctgaccaattatctggcagagagtataataaaatacttctaattaccttaaatcattaggcgttgtttcttgccgaatatatgatggagcatggtctcgctaagagacaacaaaatctttaaataaattaaagaaattgtttagcaaaaaaattaaataaattaaacaagagagagtacaaatataaaacactctaacggaagataaatatataaaatacaaaatatagaatagaggcctcaagtagtgtttacgtgacaatctcaatttcactccatacctcgaactcattcaagcttagaaacctaacttctactgttccttttgatttgcctataaatacacgttggtccatacttctctaatttcaaatatacaaagcaaaacacagagacaagaatcataaataacaaacattttcaacataatcccaaaaaacgacacgcgttccttttccaaaaattcacttctctacgtaccaaccacaatggctcattgttgcacagttcttaccggtttactagtatggtctctttttatgctctcatggtgtgaagcttcaagaaacaacattaatagatatgatcattcttatggaacgttcaaatccaatagcttaatcaagcgaagagacgatataactcgtctgaaaagtgttgcaagagcctctttgcggcttccaaccaccgttagtgtttctgattttggagctaaaggagatggaaaaaccgacgacacgcaggtatatacgcatatacacatatctacatgacttataaatattttgagacggtcacgcatataaatttacgttgtttgtactaatgatccatgcatgcgcatgcatatgatcaggcgttcgtgaatgcatggaagaaagcttgttcttcaagtggagctgtaaatctcttagttcctgaaggaaaaacttattttctcaagtctcttcgattaaatggcccatgcaaatcagttcttaccgttcaggtaaattacaaagtttttttttcttaaatttgggtaaattacaaagttgactatttgttaattaaatacgtatactacatgtgatcacgtatatggttttgaatatttaaatgagcggttgatgaatggaacgacagattttaggaacgttatcggcttctcaacaacgatcggattatgaagatctcagcaaatggattacgtttgacggcgttaacagtctaacggttgatggcggagccaccggaactgtcaacggaaacggcgaaacgtggtgggaaaactcatgcaaacggaacgaagctaaggtgtgtgtttgtacatgtttcatttacttaatatatatctactgtctacatgtcatcttgatatattaacactcttttattgttattgttttgaagaaatgcacaaaagcccctacggtacgtagaaacatatttgacattttcatgtaatactgacgatggcggtgatcatgattggacaaatgttttacatgcatgaatatatgtattttatatgcaggctcttactttctacaactcgaagaatttaagagtgaataatctgagggtgaaagatgcgcaacagattcagatttcgattgagaaatgctccaacgttcaggtctcaaatgttgaggtcactgcacctgctgatagtcctaacactgatggtatccacatcactaacactcaaaacattcaagtctccaattccatcattggaacaggttagtagttatatcctcgttatttaaaagagttgcgtgcatatccagaaaactaaaatttaagagtatcgttataatttataattttatagagattggtcttaaaagagtttttaatggttgcttttctttgcaggtgatgattgtatatctattgaaagtggatcacaaaacgttaaaatcaacgatttaacttgcggcccaggccacggtatcaggtatcaatgaattgttttcagtatatgtacctaagaaattcaaccgtaatcttttaaattaacatagaaatttgatatttccatataaaacatgagcagcattgggagcttaggagatgacaattcaaaggcatttgtatcaggcgtgactgtggatggtgctaaactttccggtacagacaatggagttaggatcaaaacttaccaggtgaaaatcacaaactagaccttcaattttaactagcctattgacaagttcatctcaatttaacttttaaacccaattcaatatagggagggtctggaactgctagcaacattatatttcagaacattcagatggagaatgtgaagaatccaatcataattgaccaagattattgcgacaagagcaaatgcactgaacaagttcgtttgaatctgatcaaatctcccattactaatcatcatgataataaacactattgtctctaatcatttactacatatattttcagaaatcagcggtccaaataaagaacgtggtgtaccggaacataagcggcacaagcgcatcggacatagcaataactttcaactgcagcaagaactatccatgccaaggaattgtactcgacaaagtgaacattaagggagggaaagccagttgcagcaatgctaatgtgatggataaagacggtgttttacctcagtgcaaatccacttgattaataagacctataaaaaaaactcacatagatgtataaaaatacatatataccgatacaaagtatacaaaattatgatatacacttgtggatatatacatggacaatgaatttaaacatattgtctatatgtcaaaaatagtttatgaatatacatatagacaacatggtcaagagatttattgttttaatgtatatatatactactaaactacttgtctaaaatttcatgaataaaggattacgtttttaagtggttatgtatcatgatgtggttacagtatgatgtgtattgtagattttcaattaaatacataaaaatgaaaaagatgacatggctggtcaatgatgttacttgaccaactaattcaagctaattgatacacgatgaacatctaacgaaacagaagcgactttcatattgtttagttgtgctcaaaccaggccgataaactcattgggcctataagcccattaatatttatccgtgagagcgaagtctgttacttgggttactaaacaaagaggcctttagaaaaccctaactcttaccgtagcacctgagctatatattgccgcagctagggtttccactccctcacgctaacacacatcaccgcagccatttccatctctcaaccaacttcaaacaatggctgaacgaggcggagaacgaggtgccgagcgaggaggagaccgcggcggtttcggacgcggattcggaggaggtcgcggcgacagaggaggccggggaggtccgagaggccgaggcggcagacgcggaggacgtccgacagaggaggagaagtggaccccagtgaccaagctcggccgtctcgtgcaagccggaaagatccagaagctcgagcagatctacctccactccctccccgtcaaggagtaccagatcatcgacctcctcgtcggccccaccttgaaagacgaggtgatgaagatcatgccggttcagaaacagaccagggccggtcagaggacgaggttcaaggcctttgtcgtcgtcggagacacgaacgggcacgttggtttgggtgtgaagtgctccaaggaggtggcgacggcgatcagaggtgggattatattggcgaagctgtcggttattcccgtgaggagagggtactgggggaacaagattgggaagccgcatacggttccttgtaaggttacggggaagtgtgggtctgttacggtgaggatggtgcctgctccgaggggtgcggggattgtggcggctagggttcctaagaaggtgcttcagttcgctgggattgatgatgtgtttacttcgtcgagggggtctactaagactcttggaaacttcgtaaaggtttgaactttcttcatttatttagagtttggagttgtgtatggattgaaagttgattcctttttgatggttgcgtgtggagttgttttgggtaatttagcttgttgtagttgatgtaaagtgagaattgctaagcttgattagtttgaataagctgtctgtgttaaatgatgatttgtttgtgttgtattgagctgtctgaatttcattatttgtttgtgttattgctaaattacaagcttaaatttgataacttttgattctttcgtgagagttttttttgggtaatatagcttgatgtagttgatggaagtaaattgttaacctttagagctgtctgttaatggtgatttgtttgtgttgtattgagctgtctgaattataatttgtttgtgtttattgctaaatcacaaagctttgaagtcgtttgtacatattcatcatttttgtgtttattgacttgtttgtgtttattgctaaatcacaagtttctatgattaatcttcatgatttggttgtgtttattactaaatcacaagtttctatgattaatcttaatgatttggttgtgtttattgctaaatttgagattacattataatcttagtttatacatgtataacatcatttcacttgaattttcacagacgagctggtgatatgtttttttagtattttccatgtattgctttgtaagaacttttaaatgtttgttttcttcctttgaagaacaattaaatgtttgttcagtatttttgaatcattcattggttaatcatgtgtaattttatttttgtaggctacattcgactgtctccagaagacttatggattcctgacaccagagttctggaaagagacgagctttaaaaagtcaccgtaccaggagtacactgatctgctggccgagaagggaattgcaaccaaagccatcaccgaggttgaagaccaagccgcctcctaaataaacaaacaaacaatgtcccccttgttttgtttttggatgatcctttcttgagttttattttgttcgaacctttagtatatgttgtttgctggattgctgctttcttatgttttcatcatctcccccaaacaaagttatttttgctctagtttttttttataatcgatggttatgccctctagtattttttttacttgaatttttcactaattttctatcaacttccctatgttacactttctaaggttgattacataggtggattgttgtg

SEQ ID NO: 8 - DNA, 4453 bp, Brassica napus
  source 1..4453: mol_type = other DNA
ttaaacgtaaacgtctccgctgtatttatataaacttttcaaaatcacggtgtcgctcgtgttaagatttggatttctaatccaactttcagaaagtttatgtgaaagataatactcaaacacaatagttcgtttagaacgctcttaagattaatgtcacacacacaaaattgatgtcactagatttatatgttttgtcgtttaaggggatccatcaactacacatggaagaatagaaaacactggtcacacacaaaaaataatataagcatgttgagtcctattcatcagaaaattttctccaaaggatgaacaataacctacaattaaggaaacttattataacaacgaccaagaagttttcccataagtcatgctttcgtccataccttcgggtctttcaagcaagcttcacacatgccttatcgtatatttgtatacgttttcattttgtgtacgtctattaattagtatatgcctagattatatatttatatagtcgacaattacctatcctaaaggtttattttaatttttattcagaaagatatgcacgtagggccactagtgtttcccgttgttattggaaattccatgtgttgaactgtttgattaagtttttgaacccgtaagtcaaatttatttaggtttaacagggtttcattactgttataagcttgtcaagaaagaatttgtcacaaatttcgagactcatgcatgcatgcatgtttcttaactttcgagaaacatgaagtgtggttttaccagctgaagtttgtgttaaaacacatttttaatttgaccacgtgtgatattcaaatatcaacaattctgttcaacgggagattcatacatatgttcagatgttctttaatcttgagcacctaactttgcttcccatacgcaaatgtgtgtgccgcgaactatggtgaatgtacggcatcgtttttctttttggagcatttttaaaagaaaatataatttagttatttccgttttgattgatatagatgcattaataaaccgacttacgtgggtgatgggatgagcaaaattaaaatatgcattttagtaaccaattacggtccatcttgacaaactaacaaacaaaaagtctattatgacgttgatttgtgaccttgccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaatgacaactcctgttcttgtatcgtgtatctagcacgtaaatttgaattatggcggcattatcgataaaatatgtaaagaatatattttcatcatacaccacaaacaaatgttctggaaatatcaccttttatcacatatacaaagctgaccctgaatttttggaaactacaagccaaaaaaaaatggacaaaagaaaaattatgcacaaaagtggactcgtcctaactctttttataatgataattaacctttgaacaactaattaatacatacaatatgttttttttttctaaaaattggtcgattaaatattcatatataatcctcatcatcatttattagccttttgtagcttacttaaaaaatcacttcccaccaaattgattattttttcacttcatacgttgatcaatttaaaaaaaaaataataataatatatgcacgtccggcgtcccatgacttcagacgcttttctgtttccttgattttataggttagaaatttccaaaatgctcatatttttcactgatatgtattatacgcacaaaatagtaaagtcataaaaatttatgatggttcttttatatgtatagagtagtgaaaatttatatatattgtagttccgcttaaaataaattaatatttttcttttatcgcccatatttaagttactaaacgtatataattaaaaactgaaatgcaatatgtagaaatccgggcattgattgttgattaacattgatatacccatgcgactggcttatgatgaaccatacctgctaatactcattgttacaaactcgcagcaacatcataaatccatcactcgccacaataattttcttcagtgtatgttcaattgatcacattttgacccttcactttcttgaatgagaaattatacaagaaagtgatttgagtgtgttaaaaaatgatttgtcattctgaccaattagggtttccactccctcacgctaacacacatcaccgcagccatttccatctctcaaccaacttcaaacaatggctgaacgaggcggagaacgaggtgccgagcgaggaggagaccgcggcggtttcggacgcggattcggaggaggtcgcggcgacagaggaggccggggaggtccgagaggccgaggcggcagacgcggaggacgtccgacagaggaggagaagtggaccccagtgaccaagctcggccgtctcgtgcaagccggaaagatccagaagctcgagcagatctacctccactccctccccgtcaaggagtaccagatcatcgacctcctcgtcggccccaccttgaaagacgaggtgatgaagatcatgccggttcagaaacagaccagggccggtcagaggacgaggttcaaggcctttgtcgtcgtcggagacacgaacgggcacgttggtttgggtgtgaagtgctccaaggaggtggcgacggcgatcagaggtgggattatattggcgaagctgtcggttattcccgtgaggagagggtactgggggaacaagattgggaagccgcatacggttccttgtaaggttacggggaagtgtgggtctgttacggtgaggatggtgcctgctccgaggggtgcggggattgtggcggctagggttcctaagaaggtgcttcagttcgctgggattgatgatgtgtttacttcgtcgagggggtctactaagactcttggaaacttcgtaaaggtttgaactttcttcatttatttagagtttggagttgtgtatggattgaaagttgattcctttttgatggttgcgtgtggagttgttttgggtaatttagcttgttgtagttgatgtaaagtgagaattgctaagcttgattagtttgaataagctgtctgtgttaaatgatgatttgtttgtgttgtattgagctgtctgaatttcattatttgtttgtgttattgctaaattacaagcttaaatttgataacttttgattctttcgtgagagttttttttgggtaatatagcttgatgtagttgatggaagtaaattgttaacctttagagctgtctgttaatggtgatttgtttgtgttgtattgagctgtctgaattataatttgtttgtgtttattgctaaatcacaaagctttgaagtcgtttgtacatattcatcatttttgtgtttattgacttgtttgtgtttattgctaaatcacaagtttctatgattaatcttcatgatttggttgtgtttattactaaatcacaagtttctatgattaatcttaatgatttggttgtgtttattgctaaatttgagattacattataatcttagtttatacatgtataacatcatttcacttgaattttcacagacgagctggtgatatgtttttttagtattttccatgtattgctttgtaagaacttttaaatgtttgttttcttcctttgaagaacaattaaatgtttgttcagtatttttgaatcattcattggttaatcatgtgtaattttatttttgtaggctacattcgactgtctccagaagacttatggattcctgacaccagagttctggaaagagacgagctttaaaaagtcaccgtaccaggagtacactgatctgctggccgagaagggaattgcaaccaaagccatcaccgaggttgaagaccaagccgcctcctaaataaacaaacaaacaatgtcccccttgttttgtttttggatgatcctttcttgagttttattttgttcgaacctttagtatatgttgtttgctggattgctgctttcttatgttttcatcatctcccccaaacaaagttatttttgctctagtttttttttataatcgatggttatgccctctagtattttttttacttgaatttttcactaattttctatcaacttccctatgttacactttctaaggttgattacataggtggattgttgtgaaacaaatactaataataaactaatacttttagaataatataattagtgggattaaattataaattataaattaaaaagacaagtctgaaatggattgtttaactgtaaaaggtaaagagactttgaatagaagagaataaatcaaaagttgacgtatcttctccgaaatttcagct

SEQ ID NO: 9 - DNA, 7172 bp, Brassica napus
  source 1..7172: mol_type = genomic DNA
gtgacctggccatataaatgccaaattttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaaagacaactcctgtgtcttgtatcgtgtatctcgcacataaaatttgaattatgggggcattatcgataaaatatataaagaatatattttcatcatacaccacaaacaaatgttctggaaatgtcacattttatcacagatacaaagctgaccctgaatttttggaaactacaagccaaaaaaaaatggtcaaaagaaaaattatgcacaaaggtggactcgccctaactctttttataatgataattaacctttgaacaactaattaaagatacatacaatatgttttttttctctaaaaattggtcgattaaatattcatatataatccttatcatcatttattagcctgttgtagcttacttaaaaaatcacttcctaccaatgtttttaaactcgacgcgaaccggacgatttaccgggtcgctgggtcactgtatcgaccgcgagcgaaccgcgggttaataaataaactaaaatttaaaataatatatcagctatgtaaataaaaatatagaaactaaaatttaatattttctaaatgttcttataaacataaaataatagtctggatgtgtatatttttatgtttaataacatttagaaaatacttaactttaatttttatatttttattttcatttgatatacaaaatatcaaaaaatagtttagattatttataattttgtctaacaaggtaagagttatggcatctaagaaaatcaagacataaaatttataaatatttaaatgtttaatgtgaataataaattaaatttcaaatacaaaataaaccaaaagtgaaaaatcattataaaaaattatcaataacagaaataaaccaactccaaatcacatcaactagttttggttctgtctaccattgttcacttttattttcttcaagtggcatagtgaaatcttcattaaaatctaaaaatcacaaatataaaactgaaaaggaaaaacttaacttttttttgtcggtatatcacttcttaattagaaatttagaatataaaacataatctaaaaacataattaaaaattaaaaatgaagtaaacgttatttattggttcaaccagtggttcaaccggtatcgggttccgggttctattgggtttttgcgggtttctaaatattgtgtttttcacaaaactcaaaccggatttttttagggttgccgggtttaccggttcaaccgcaggttcgggtcgggtttcaaaacactgcttcccaccaaattgattaatgtagattgattattttttcacttcatacgttgatcaatttttaaaaagaaataatatatgcacgtccggcgtcccatgacttcagacgcttttctgtttccttgattttataggttagaaatttccaaaatgctcatatttttcactcatgtattatacgcacaaaatagtaaattcataaaaatttatgatggttctttatatgtatagagtagtgaaatttatatatattgtagttccgcttaaaataaattattatttttcttttatcgcccatttttaagttactaaacgtatataattaaaaactgaaatgcaatatgtagaaatccgggcattgattgttgattaacattgatgatgatactcatgcgactggcttatgatgaaccatactaatactcattgttacaaactcacagcaacatcataaatccatcactcgccacaataattttctttagtgtatgttcaatttcacattttgacccttcactttcttgaatgaaaaattatacaagaaagtgatttgagtgtgttagaaaaaaaaaagatttgagtcattctgaccaattatctgggagagactataataaaatacttccaattaccttaaatcatcaggcgttgtttcttgcctaatatatgatggagcatggtctcgctaagagacaacaaaatctttaaataaattaaacaagttgtttagcaaaaataaaaaaaataaattaaacaagagaaagtacaaatataaaactctaaaggaagataaatgtataaaatacaagatatagaatagaagcgtttacgtgacaatctcaatttcactccatacctcgaactcattcaagcttagaaacctaacttttactgttccttttgatttgcctataaatacacgttcgtccatacttctctaatttcaaatatacaaagcaaaacacagagacaagaatcataaacaacaaacattttcaacataatcccaaaaagacgacacgcattccttcttccaaaaatccacttcctacgtaccaaccacaatggctcattgttgcacagttcttaccggtttactagtatggtctcttttgatgctctcatggtgcgaagcttcaagaaacaacattaatagatatgatcattcttatggaacgttcaaatccaatagcttaatcaagcgaagagacgatatcactcgtctgaaaagtgttgcaagagcctctttgcagcttccaaccaccgttagtgtttctgattttggagctaaaggagatggaaaaaccgacgacacgcaggtatatacgcatacacacatatctacatgacttataaatattttgagacggtcatgtatataaatttacgttatttgtactaatgatccatgcatgcatgcatatgatcaggcgttcgtgaatgcatggaagaaagcttgttcttcaagtggagctgtaaatctcttagttcctgaaggaaaaacttatttccttaagtctattcgattaaatggcccatgcaaatcaattcttaccgttcaggtaaattacaaagttgacaaaaaaacaattacaaagttgactatttgttaattatatacgtatactacatgtgatcacgtatatggttttgaatatataaatgagcggttgacgaatggaatgacagattttaggaacgctatcggcatctcaacaacgatcggattataaagatctcagcaaatggattacgtttgacggcgttaacagtctaacggttgatggcggggccaccggaactgtcaacggaaacggcgaaacgtggtgggaaaactcatgcaaacggaacgaagctaaggtgtgtgtctgtacatgtttcatttacttaatatatatctactatttacatgtcatcttgatatattgacactttttattgttattgttattgttttcaagaaatgcacaaaagccccaacggtacgtagaaacatatttgacattttcatgtaatactgacgatggctgtgatcatgattggacaaatgttttacatgcatgaatatatgtattttatatgcaggctcttactttctacaactcaaagaatttgagagttaataatctgagggtgaaagatgcgcaacagatccagatttcgattgagaaatgctccaacgttcaggtctcaaatgttgaggtcactgcgcctgctgatagtcctaacactgatggtatccacatcactaacactcaaaacattcaagtctccaaatccatcattggaacaggttagtaattacatcctcgttatttaaaagagttgcgtgcatatccagaaaactaatatttaagggtttatcgttataatttatagagattggtcttaaaagagttttttaatggttgcttttctatgcaggtgatgattgtatatctattgaaagtggatcacaaaacgttaaaatcaacgatttaacttgcggccccggtcacggtatcaggtatcaatgaattgttttcagtatatttacctaagaaattcaaccgtaatcttttacatataaatttgaaatttccttataaaacatgagcagcattgggagcttaggagatgacaattcaaaggcatttgtatcaggcgtgactgtggatggtgctaaactttccggtacagacaatggagttaggatcaaaacttaccaggtgaaaatcacaaactaaaccttcaattttaactagcctatcaagttcatctcaatttaacttttaaacccaattcaatatagggagggtctggaactgctagcaacattatatttcagaacattcagatggaaaatgtgaagaatccaattataatcgaccaagattattgcgacaagagcaaatgcactgaacaagttcgtttgaatctgatcaaatcttccattactaagcgtcataataatataaacttttctctaatcatttactacatattttcagaaatcagcggtccaaataaagaacgtggtgtaccggaacataagcggcacaagcgcatcggacatagcaataactttcaactgcagcaagaactatccatgccaaggaattgtagtcgacaaagtgaacattaagggagggaaagcaagttgcagcaacgctaatgtgatggataaagacggtgttttgcctcagtgcaaatccacttgattaactagacctatcaaaaactcacatagatgtataaatacatatagaccgatacaaagtatacaaaattatgatatatacttgtggatatatacatggacaatgaatttaaacatattgtttatatgtcaaaatagtttatgaatagacatataaacaacatgttcaagagatttattgtcttaatgtatatactgcatactaaattgtttgtctaaaacttcatataataaaaactgttaatcttcatgatggtgtgatttgttgtgataacagtatgatgtctatatttgtaaaaaaaaaaacagtatgatgtctattgtagatttttatctaaatacataaaaattgaaaaaaaaaaaatgacatggctacttgaccgattgattcaagttaattgttactaaccaggccgataaacaaattgggcctttaaagcccgttaatatttatcagtgaaagcgaagtctgttacttgggttactaaacaaaggcctttagaaaaccctaactcttatcgcagcacctgagctatatattgccgcagctagggttttcgctagctcacgctaacacacaaagccgcagccatttccatctctctcgcaactcaacttcgaacaatggctgaacgaggcggagaacgaggcgccgagcgaggaggagaccgcggcggtttcggacggggattcggaggaggtcgcggcgacagaggaggccgcggaggtccgagaggccgaggcggcagacgcggaggacgtcccacagaggaggagaagtggaccccagtgaccaagctcggccgtctcgtgcaagccggaaagatccagaagctcgagcagatatacctccactccctccccgtcaaggagtaccagatcatcgacctcctcgtcggtccaactttaaaagacgaggtgatgaagatcatgccggttcagaaacagaccagggccggtcagaggacgaggttcaaggcttttgtcgttgtaggagacacgaatggtcatgttggtttgggtgtgaagtgctccaaggaggtggcgacggcgatcaggggtgggattatattggcgaagctgtctgtgattcccgtgaggagagggtactgggggaacaagattgggaagccgcatacggttccttgtaaggttacggggaagtgtgggtctgttacggtgaggatggtgcctgctccgaggggggctgggattgtggcggctagggttcctaagaaggtgcttcagttcgctgggattgatgatgtgtttacttcctcgagggggtctactaagactcttggaaacttcgtaaaggtttgaactttattattttacttggtgtttggagttgtgtatggattgaaagttgattcctttttgatcgttgcttgtggaggtgttttgggtaatattgctaaatgtaaaaattgccaagcttgattagtttgaataagctgtctgtgttaaatgatgatttgtttgtgttgtaatgagctgtctgaatttcattatttgtttgtgttattgctaatttacaagcttaaatttgattccttttgatcgtttcgtgtgagttgttttgtgtaatttagcttgatgtagttgatggaagtaagaattgttaagctttagagctgtctgttaatgttgaattgtttgtgttgtattgagctgtctgaattataatttgtttgcgatttattgctaaatcacaaagctctaaagtcgtttgaacatatgcatcatttggttagtgtttattgctgaattttcatgatttggttgtgtttattgctaaatttgagattacattataatcttaatttgtacatatattcatcatttttgtgtttattgatttgtttgtgtttattgatgaatcacaagtttctatgattaatcttcatgatttggttgtgattattgctaaatttgagattacattataagcttagtttgtacatgtatacatcatttcacttacatttcacttggtggtgatatatattttttttttagtattttccatgtattgctttgtaagaacgtttaaatgttagttttcttgaatgtttttttctttgtaagaacaattaaatgtttgttcagtatttttgaatcattcaatggttaatcatgttcaacatgtattttttgtaggctacatttgactgtctccagaagacttatggattcctgacaccagagttctggaaagagacgagctttaaaaagtcaccgtaccaggagtacactgatttgctggccgagaagggaactgcaaccaaagccatcaccgaggttgaagaccaagccgcctcctaaataaacaaacaagcaatgtccaccttgttttgtttttggatgatcctttcatgagttttattttgttcgaaccttttagtatatgttgtttgctggattgctgctatcttaagttttcatcatcttcccaaacaaagttatttttgctctagtttttttttttttaataatcgatggttatactctagtatttttttacttgaatttctctaattttctatca

SEQ ID NO: 10 - DNA, 188 bp, Brassica napus
  source 1..188: mol_type = other DNA
cattttgacccttcactttcttgaatgaaaaattatacaagaaagtgatttgagtgtgttagaaaaaaaaaagatttgagtcattctgaccaattagctagggttttcgctagctcacgctaacacacaaagccgcagccatttccatctctctcgcaactcaacttcgaacaatggctgaacgaggc

SEQ ID NO: 11 - DNA, 15000 bp, Brassica napus
  source 1..15000: mol_type = genomic DNA
gtagttccaatactaaatcctcaactctaatcagtgaaccctaaactttatcccaaccccaccccatcccctaaaatactaaaccctaaacactaatcagcgaacctaaacccaaatataaaccataaatccaaatataaaccctaaacccaaatagaatggaacaactatgaacccaaatatcaaaatctaaaccttaaccagtgtttaaccccaattttccataaactaaattcaaattttaaaatgccagtaaaatacattaccattctacatgagtcatatagaatagaaatagagaaaatgtagttccaatactaaatcctcaactctaatcagtgaaccctaaactttatcccaaccctaccccatcccctaaaatactaaaccctaaactctaatcagcgaacctaaacccaaatataaaccataaatccaaatataaaccctaaacccaaatagaatggaacaaaataaatagaatagtacatactggtgaaattatgaaatatctatgattgcatgaaagaagttaataatgactccaaccataacccctcaccttctaattagtaaaccctaaactataatcactaaaccctaaacgcaaatatcaaaatataaaaatgtatgcaatattatgcaatattaaatcatattatataatattaaactatacaatataaatcatagtacggtttttacagattcaaaacatgtaatgatagttagatattaagaaaattacaaaatatatttttaaacaaaatagaacactctttattaatcatcaaatggaatataacataattaaatagtatagtaacaaaaaggtagaaaacaacaagacacatctgggaagaagaactggccgttgttacttcatgttgccgtgtctcctcttccgtcaaacaagacaccatcgatttcacaatcagagaaacctccccttcagccacccaccgagaatgcaaagatgttatactcttgcaaaaatcaactctgataatcgtgtccgtctcattgctctctcccaaatctgagaatcaaacgatttaatttctcgaaaaagaagaaaggtaaaaattacggagatgatgacggagcaaatacggtggcggcgatgaaacaaacgatgatgatgatggttgtgctggttgtacatgtagagatgattgaagctatgtgacagagatttatgtgttcgtcgtataaaaaaacagaaatttgagagagagagagagagagagaggaacacgaagaaacagggaagaagaagaaaaaattatgtgtggatgtgtgactttctactggacacaaattatttttaaaatataatgattattaatttttttttctgcaggttttaccggttacaacagagggcaatacggcaatattatataaaagacacatgatacagtgaaaaaatagggtttttagttatagattgaattataatttttttggtggttatacagcccaatttcccttaaaattttacatatcgttgaaagccagaaaaatatgtggaagtgtaataaaacaattttctccaggaagaagatatttcagccattcgcccatctatgtgtatgagatcccattcaaggccatattctaagattgactttatctaatttactgtattttccgtgggctactgaatgaatacgatgcttttgttgtaaaacgcaacctattgttctccattttgaatttgaagtgttacattgttgataaaaaggaagaaagtacaaagcttttcatttagtggttgttgcatatatatatacttatttgatgggtttatgataagtatgaacattattagattatggttaagtgatggtttaataactggtttagcttccttttactaaaccggttgtataaatacatcctcttgtacattgtaaactttaacttgagcaatatactttttccttttcatcgtaacaaactttctccattgatcttgagctcaagttctaatatggtatcagagccaatctgatcttcgttcttccgcttcgttctttctggattcaagcttctgtagctgcgattcagtctgatttcatcttctttttcgttttctcacttctttttctcgggaaaatcagctcgatcttcttcgcttaagcgcattcttcatctcaatgagtctctcgttacggcgcctgatcgatctttttgttgagctgtatcttcccatcctttttgcgatttatcagtcggtttctatgataatcgcgattctgtttctttcttttctttctctttgatcttcgatttgcgtattctcttctcgcgcaatcgttttctggttttccaaattgattcttgttttctcatgacgactcctgcatcaacgatccctctctccgacgataatccacctgtttcggcgtcacttcttgatcccaattccaatcctctgtatgttcacaatgccgatcacgctggtatctctcttgtttctgaaaagctagctggtttggggaattttaatacttggcgtagatcgatgatcatggcactgggagctaggaataaggcagtttttgtggatggtacttatctggaattatctgaaacgcatccggattatgcatcttggtcacgttgtaacaatatcgtttgcacatggattgtgaatgctgtcgagaaatccatcgccaagagcataatgtaccttcctacggctcgtcaaatgtggcttgacatccatgatcagtttaagcagagtgatggaccaagaacagccgagataaagcaacaaatttttgcggaggtgcagggctctcaaactgttagtgaatactacactcgtttgaagcaacaatgggaggagttgaagaatcatgagagtccttatacttgctgttgtggacgttttgattgttcgagcatgaagaggattgatgagggagaggaacaggacaagatcttgaagtttctcacggggttgaatgatacattcactgctactcggggtcagatactgatgatggatccaaaaccgaacatttctagagtgtttaatcttgtttcacaagaggagcgacagaggtctatgaagagcacaagtgtggtagcgtttcaagtttctcaaggagcatctaatgatgagatggttgctgcatatcactctggttataacaaacagaagcagcgctcgatatgctctcactgtggcattgctggtcacactgttaaccgctgctacaagcttcatggttatccacaaggatatagaggtgctacgggttcctccaagttctctactcaggcttcttcttcatcttcttctgtgaagagtcaaccagctggtgggattgatcttttggaccatcaaggtgcgtgcttggggtctgtcactacagcacaagtcagtcgatttctaagtgtcttgggggctcatccgtcgagtctcaatgacaatgtttctcaggcttcagctagcactattaatcattctgatggaacagtctctacaccaaaaccacaaccccacctcgtcccccatttaccgttacctcacccttcttcaggtaccactcattacatcaattctcattttgtttctgctggaaccaataatggccttacctttctaagtgatgcgtggatcatagatactggtgctagttgtcacgttagttcgaacttaggtttgtttcattcaactagaaccattagtaacacatctgtcactcttcctgatggaacccaaattccggtcacaacttctggttccattactctctctgaacatttaactcttcttgatgtcttatacattccgcattttcaattcaatcttttgagcattagtgctctcactcgcaacagtactattgctgttttgttttcttctcattcatgtttcatttttcctaacaatccatttctcctccaggagcatactctggattgcacgattgggaagggtaacctcctgcgcaatctgtatgtcctggagtctcctacactttcttcacattccgcgcattctttgtcggtttctacaccttcttctatggatcttcctcttttacaagtctctgctgatgtgtggcatcaacgccttggtcatccttctcaaaacaagattcaagctctttcaaaacttttgaagatttcttcggctaagattgatcatagtagtttgtgttctgtgtgtccattagctaagcaaaagcgtctgtcgtttcctttgtctcctcatttgtccaaaactcctttcgatcttattcatatagatatatggggtcctttctcgactactacacatgatggttatagatattttttaactttggtcgatgattgtactcgtgctacttggttatatcttttaaaggataagagttcggtttcaacaatttttcctgagtttcttgcttttgttcatactcaatataagactcatgttaaatccattcgttctgacaatgccccagagcttgcctttactgatctgttgcgtgaaaaaggaattatgcattactttgcatgtccatacacacctcaacagaattctgtagttgaacgaaaacaccaacatattctaaatgtcgctagagctttgctgtttcaatcaaaagtaccattgatatattggggagattgtgttcaaactgctgtgtatttgataaataggactccctcaccgcttttgaatgataagtctccatttgaacttctctcaaaacagattccttcatatgatcatctaaaagtgtttggttgcttatgttactcttctacactagctaaagatagaaataaatttactcctcgtgctgacgcatgtatttttcttggttatccgaatggctataaagggtacaaagttttaaatctcacgtctcattcgataaatgtcactagaaatgttgtttttcatgaaacagttttcccatttcataaatctactgattcttctactcatgatgatttctttgctcaggatgtcttaccattacctgttccggattctatgtttcctgctttctttgacttgggttctgatattgattcttcaatgtttcctgatccaactactattagttcttccaacatttctaatcattcgtttgatgcgtctcttcatccttctgtttctacttctgttccttcttcttcttcttcattaccatcaggaactagaaatgaaactaggactcgtcgtcaggttaaggcaccagcttatctagatcaataccattgctatcttttacaaaaagtgcctatggaaccagttcatcctactcatacgacttcataccctatatctgcatttctttcttatgataaatttgatgaaacacaccgaaacttcattctttccataaccaccaccaaaccaccccaaacttaccaagaggctgttctatctgaagaatttaagaatgctatgaagtctgaaatgacttctctagaagagacagggacttggactgtttgtgagttgcctgcaggtaaacacccggttggttgcaaatgggttaatacttacaagtataaagctgatggtactgtggaaagaccgaagtctcgtcttgttgccaagggctatactcagttggagggtcttgattatcttgacactttttctccggttgcgaaacttggtacacttcgtcttctcttagctgttgcagcagctaaaggatggtctattcaacagctggatataagcaacgcatttctcaatggtgacttggatgaagagatatatatgcggcttcctcagggatatgaagaactcactggaaagaagtgtcctccaaactctgtttgtcggctccataagtctctttatggccttaaacaggcttcaagacaatggaatcacaaactctctagtgtgatcttgggtgatggatttgttcaaacacactctgaccattctctcttcatcaaacacaatgagaaggcttttctagcagttttggtctatgtcgatgacatattgatcgtctccaacgatgactctgcttctgctacattcatgaatgtcctcaagtcggcattcaagttacgtgatcttggtcaagctaggtacttcttaggctttgagattgcacgtaactcctctggaatttctttgaatcaacggaagtattgcttggagctattggaagaagctggaatgctgggttgcaagcctgtatctgttcctatggaacctaatctgaagctttcggctactcctggtgacgttttaccggatgcttctgtttacaggaggctagtgggtcgcttattatacttgactcacacaagacctgacatcacttatgctgttcataagctaagtcagttcatgtctgcgcctacgactgatcatctacaagcagcatacagagttcttcgctatctcaagaatgatccagctcagggtcttttctattctgcaacttctgcgatcaaactcacagcttattctgatgctgattgggcttcatgtccagattctcgtcaatctgtcacaggttactgcatgttcttaggtgactcgttgatatcttggcgtgcaaagaaacaacctactgtttcccggagtagctcagaggcagaatacagagccatggctgatgctacttgtgaacttatctggttgactgcgttgttgattgagatgcattgtgctcctgttgctcctgcaaccttgttttgtgacaatcagtcagcgttatacattgcgtctaacccggtctatcatgaacgcaccaagcatgttgagatcgactgtcacattgttcgggagcggttgcagagcggcttcatgaaggctctacacgtccggtctgagcttcagttagctgacatattcactaaggttgttcaaccaggcttgttcaagagtttagtaagcaagattggaattcatagtctatgcattccatcttgaggggagggtattagattatggttaagtgatggtttaataactggtttagcttccttttactaaaccggttatataaatacatcctcttgtacattgtaaactttaacttgagcaatatactttttccttttcatcgtaacatactttccccattgatcttgagctcaagttctaataaacatattacgtaacgggtttattttgtcgtcaacacattatcggcaatgtgaaaagaaggggtgaggtatctccatttcaattcttctctttatatattaatcgaattatttacgtatgaaatgaacgtttatatagaaatttcgtgtggaaaacgacatgtacacggcatctcaagaccaattagtaatatactttagtggtgattacatgtttacttatccaattgagaatttaaagcatcgacaataccttaatgtcgattaagccgtccccacttcatgtaatgagttatggggggagagagagatcccgaaattcgtcaaataaaacaacttagaactaaaaaccgacaccaagtatcataaaggaaatgttgaagaagtcatttatcgtatccagctcacaattcctaagattaaatcatgaccgttggaagagcttataagattaaactgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctatatggcaatgatatcatcaccaaagtgcaatgcccctttttgccctagtttcgtaaagtctctctccttcttcgtccttaggaaaaaccctaaattaaatcctgtgttcttgatctttctttttgagtaaccatgattttgaccacacactagttcttctatattttgtggtctataggattttgctttatatgtgtttcttgtattgctccgtacgtgcgtatataaatttaaatggttacaacaaggtttattataaataggcacaaattagtccatgaagttatttagcttgcacaagtataatttgttaagtatttaaatatataaatttgttacaaaacttaattaaatttatctgattatattttctttagtgttcttcctttgccaacgttgaggtagctattattattattattttgaacattatgtacgtagttatcttggctagttatgattcgaattcttaatttggatcacacttaacagtatttaaaatattcttagaactaaaataattaagagttacctttaaattgaagtattcgtgctaaacagaaactagaataaacaaatgattgcatgttaattttttttttcgattttcctatcagaataaacacatgattgcatgcaaattttgtttttgattacgttatcttttgtttattttagttttgatgctaattaatattttttattaacaactcacatacattctacctgattctaggtcagataatgacacagcgcaacaaaattaatacaaaaccttcggaaagtagaataccgcagaagtaacttttttgggtacatacgaaatacagtgaaatctctataaattaataatgttgggactataccaaaactataattttttattaatttatagagattaatttatcgatatactaattgaatcaaaaacttaatttgagactaaaaaattatattattttatagagatttttagtgtatattaatttatagaatattattttataaaaaattttagtgtgtattaatttatagagtattaatttaaagaggttatactgtaatgtgaatcttcgaaaaacatgccatacataaccacggatcatagtcgacacctcaacgtgaagcaaatttgacaatctacatacataaccaacaaaaagtagaataccttgaaaatctaaaacccaaaatatgatgtaaaactcaagcttggtccagagcataaaaaaattaaagccatcgctttggtatcacatatttaaacgtcagttttttttttttttttggggggggggggggggggggtaatataaaaatataattaacaaaaaaaaattatgaaacaattagcatgtaaaacactaatcttttggttgtgacaaaacgttttcacaaatgttctataaataaattcaagtgcattttatctgcaaaatatatactttcactcataaaataagagcgtttaaaacattcatacacgcactacattgacatgacaaaagaaatccgcaaatacacatgatgtatgtcgaaaaaaacaaaaaatacacatgatgtatatatagagaggatagtatctaggaaataagactatattatatatataaagaaaatagagaaaagataaaaatataaattggtatgtataaaagaaaggtctatgcgtctctagtccaaaaactctatatatgtctggctcaaaagcagatgcagccatagccccaatagtcatgatggagcatcatcatctccttatgaattggaacaaacctattgatctcattacagaagaaaactcttttaaccacaatcctcatttcatagtagatccaccttccgaaaccctaagccacttccagcccccgccgacaatcttctccgatcacggaggaggagaggaagcagaagaagaagaagaagaagaaggagaggaagagatggatccgatgaagaagatgcaatacgcgattgctgccatgcagcccgtagacctcgatccagccaccgttcctaagccgaaccgccgtaacgtaagggtaagcgacgaccctcagacggtggtggctcgtcggcgtagagaaaggataagcgagaagatccggatattgaagaggatggtgccaggcggtgcaaagatggacactgcctccatgctcgacgaagccatccgctacaccaagttcttgaaacggcaggtgaggctagcttcttcagcctcacactcagcttggagctcctatgtctgacccttcttgcctttgttattaccacaactcggatacctaattataattctatcacgcgtttcatgttgatatatatagataaatggtcgaataaggatttcgatcgaagattgtatgtacaataaatgatgtgtgtatttcaattaatgtatgatatatatatatatatgtatgcagtatgcatttatattctattctctataaggaggcaacattgccggattagggctttgatcttatgcaagttttccgaccaaaaatatgaaatacttgtttggatataacatatgaatcggataagtgttactagttatataactggaaaacaaatgtctggaataagaattcccgggagaaccaagcctttctctaatccctaagattatagctactgaaacaatgaaacaatgaagaatcagttgggcattagtaaaaaaaaaagaatcagttgggttgcttataaaattttgttataaaatttatgtcgtatgtgtgttagccgtagctgtaaatttatttcctttgtatgttaattgtaaaagtaaatttattattcccgatgtttttaatgatgtaaatttaaatcaagaaaacatattaagttctgttaataaatgtattatttttgtttactaatttccaataattttttcaagaagtacaattttttgtttgaaatttctaatttatcattattgaataatagaacatacctaaaaaccataaaacatgtattccttgaaacattttttttaatataaaaattatcttttaggagcaggtaaagtacttttttttataaaattgttagtgtatttatgttcgttatagatgtagatattttgcataataattaataatatatgagatgtgtaatgtatatatgtgagagaattgccatatttattaagcggtttttgtaaaattgactcaaaatttaaagtcaaccacaaaactaacatatgtttttttggacatttcatttgccctattcactccacaagttcagattatttacgaaaatgtcattaattttttttctttctgaaatggtatttttactctctcaacctcatcatcttcaagtatttacaagattgtcattgccatcaatacaccaaccaccatgaacaaccaatttgaataatgcaccttaaatcgatttacacttcttccttctcaattcttatgaactaaaaacaacatctctttcactttctctccatattcatccaaaaaatctaagatttttattcaaaaatttttatggttcatagaaccattgaagcttacgattcttggtgtgtcacttttgtttgagattataggtgcttggagaagacttttgtgtgctaaacaagttatctcaatggttgaactatgaaaatcagtgttttttccagatttgttcgcccaaccgactttcgggtaagttttctggctgtagacgactaacatggaagtcttctggtcaatgcacttctctttgattgtcttcttctgatttacattatgtagaatgaacaagagatgggaggaactaggggtaggtaaaaaaaacggaaccgaattatcgaaccgaacagacccgaaaaaaaccgaaccaaatttatgaattatccaattggatactgaaattgtatacccgaagaattagaatcgaactcgtaccgaaccgaaaaccgaataatacccatatagaaccagatgaaaaacctttatacactacttatacgtatatctttatagtttatattttatacacttgttttttttattgatcaacgcttgttttataggagaaccttgtattgtttggatattttatcctcataatttaagttttggtttctgaatgggaaaatcatttgaatgttattttggtgttggttttaattcatttatttgcaagtacatcagattaatttggataatatggttacaaagtaaaaagaaaaaactgaacttcaggaacctaaaaaaccgagaaccgatctgatctgatccgaaatttttatggttctttaatggttaccaatttcaagaaccgaaagaaacgagaaccgaaagaaccaataaccgaagaaccgagcgcccactcctaggaggaaccaaacaatactttgatttgaagcccaagataattggaaccatgttatattacgcagcttatctgatgtgggctttatgtagaactgataatgggctttaaggcagcctatttcggtccgtcaagaagattcctcctaacgtaaggtaggggcaatgagctgctactctgaccaattaattacgtttcaatagtgatctctcatctgttgtcggtgtgaaaggaatcgtcttttgaatttatcttcatgcgattcattgttttataactaccacacatgtgacataactaatagtctcgaatcaggacatcaaaatttatttttttgtttgtggcaatgattcaaatcgacggcatagcatactgattaaaaatatatacaatacactggttattaaaacgttatagttgtctagattttaataacaggtgataatagatcaagtatatagtaaatgttttatctcacccattacatatttcatattaaacatttcagatttatttgtaaatggattcaactgcctgttacccttcactccttttgattaacatcgcgtcgcaagtcacaaccaccatttagttggggcgattcgtatattttgggagtatataataacatacataactatcaaaccaactggtgtcgacctttggtgttaactaatttgatttaactcttttttggaataaataaaataaaatttcagagcagctgttcaatagtaaaatgacaggtgggatctagggtttgtggtccttacgatcgattgattactactacttaacttggcattatttggagtcacttttgcttattaataattagattttgtttacaaacgtgtggtgatagtctgggacaagttgacctgttgacacccaatcatttagaacaccttttacaaatataaatattcgcacactgttacaaacattatcatcaaattattgtggaattttgcagacaaagcatatgccattgtgtttccttcgatttggcttttatgcttattcgtggtgtcataattaattcgtgaaaacgaatgattttacatactgtaagtagggctgaaagcggatcgaatagtaccatattttctactatttgtgatttattccatatttcagttttcttcgtaaaaatacgaatatccagtttttcgaatatccggaatttttttgacatttgcgaatattcacagatatttacagatgattcatccactttgttcaatacaagtctaaaaaaatatcaaattattttttagaaatatcttttacatgataaaaatattaagtaaaaattaatgaaactaaatgttttattaaattttaaaattagtaatttttcttataaaaatgtttttagaaaaatatgtagttttgtataaagatttatatatttttaatttaatattttataagtagtttttaaaataaaatttaataaattatatgttaaataattatataagattatacatttaaaactctatatttaatcgtagatatacatctatttgtacaactccgatccgtctctaattttttttagtatttgtggtttggttcgtttttaacggatattgatttttaatatttgcttttttcagagacttacgaatattcatatttttttggatcgagtcgaaacaaataataaattgaatcaaatttaacggataaaatgttcagccctaactatggttaactatggatactgaaaccaccacttatcaacaaaaaaaagtagttgttcgcttctggtttagagtgaggttacaaattcaaaacataataattattttctcggacgataaccagatcctttttagaggctttaaaaggtaaatcatagtcatgtgtagcctatacatacaaattaaattacaatggaatatgcatttattgtcaatatgtataacacatatgtagacgtaaggtaatatatcattttggatttgaaggaggaagctgttgctgcatttgctgatagaacgcagccatcctgctatatgcttccatcgtcgttggctgcatgtcaatgcttcattagatttaaacaaaaatgaagtataaaagttttatataaaacaaaaagttgtataacctgcgaggagcatgcaagaaaggctgacataggatcggggataagaggagactgaaatcgagggtcgatggaagaggaggcatcccatgatgtagaagtcatgggcacaaatgggttctgcatcatgttggcatggatattaggagccgttgcagtagcagctcggttcatagaactaaggtcgacaagacctagaccgggcatccccatgcgaagccctaagcccatggaactggacatgagatttagttgaagttgttgttgttgctgctgcatggccatgggaagcatcatggagggcatgttcattctgctcatcatgcacacttgtgcttggagttgcttcaagtactctatcacttcatccaacatggacgctttatccgtctagttccaaattgtaacttatcaaaccgttcactccttttgatgtattatttaaccaaaccaactcaagggatgatcgatgaacacatgtctacaataataatagtacactatacattttttttttgtcaaacatagtacactatacatatgtaacgtagattgtgttaattgattgtgttggttgaattttgattgaccgccaaaaggtggcatccctttcttattatttgaggaacattagataaaactaacatttacttcatgtgtttattacatgtgtgtcatttcctacgtggcatcaccacaagctctctcacaccttatattcaaacaccctttcttaactagactaattacaaaatttgccattgctaattacattaaaacttcaatat

SEQ ID NO: 12 - DNA, 4081 bp, Brassica napus
  source 1..4081: mol_type = other DNA
ttagctgttgcagcagctaaaggatggtctattcaacagctggatataagcaacgcatttctcaatggtgacttggatgaagagatatatatgcggcttcctcagggatatgaagaactcactggaaagaagtgtcctccaaactctgtttgtcggctccataagtctctttatggccttaaacaggcttcaagacaatggaatcacaaactctctagtgtgatcttgggtgatggatttgttcaaacacactctgaccattctctcttcatcaaacacaatgagaaggcttttctagcagttttggtctatgtcgatgacatattgatcgtctccaacgatgactctgcttctgctacattcatgaatgtcctcaagtcggcattcaagttacgtgatcttggtcaagctaggtacttcttaggctttgagattgcacgtaactcctctggaatttctttgaatcaacggaagtattgcttggagctattggaagaagctggaatgctgggttgcaagcctgtatctgttcctatggaacctaatctgaagctttcggctactcctggtgacgttttaccggatgcttctgtttacaggaggctagtgggtcgcttattatacttgactcacacaagacctgacatcacttatgctgttcataagctaagtcagttcatgtctgcgcctacgactgatcatctacaagcagcatacagagttcttcgctatctcaagaatgatccagctcagggtcttttctattctgcaacttctgcgatcaaactcacagcttattctgatgctgattgggcttcatgtccagattctcgtcaatctgtcacaggttactgcatgttcttaggtgactcgttgatatcttggcgtgcaaagaaacaacctactgtttcccggagtagctcagaggcagaatacagagccatggctgatgctacttgtgaacttatctggttgactgcgttgttgattgagatgcattgtgctcctgttgctcctgcaaccttgttttgtgacaatcagtcagcgttatacattgcgtctaacccggtctatcatgaacgcaccaagcatgttgagatcgactgtcacattgttcgggagcggttgcagagcggcttcatgaaggctctacacgtccggtctgagcttcagttagctgacatattcactaaggttgttcaaccaggcttgttcaagagtttagtaagcaagattggaattcatagtctatgcattccatcttgaggggagggtattagattatggttaagtgatggtttaataactggtttagcttccttttactaaaccggttatataaatacatcctcttgtacattgtaaactttaacttgagcaatatactttttccttttcatcgtaacatactttccccattgatcttgagctcaagttctaataaacatattacgtaacgggtttattttgtcgtcaacacattatcggcaatgtgaaaagaaggggtgaggtatctccatttcaattcttctctttatatattaatcgaattatttacgtatgaaatgaacgtttatatagaaatttcgtgtggaaaacgacatgtacacggcatctcaagaccaattagtaatatactttagtggtgattacatgtttacttatccaattgagaatttaaagcatcgacaataccttaatgtcgattaagccgtccccacttcatgtaatgagttatggggggagagagagatcccgaaattcgtcaaataaaacaacttagaactaaaaaccgacaccaagtatcataaaggaaatgttgaagaagtcatttatcgtatccagctcacaattcctaagattaaatcatgaccgttggaagagcttataagattaaactgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctgaaccaagcctttctctaatccctaagattatagctactgaaacaatgaaacaatgaagaatcagttgggcattagtaaaaaaaaaagaatcagttgggttgcttataaaattttgttataaaatttatgtcgtatgtgtgttagccgtagctgtaaatttatttcctttgtatgttaattgtaaaagtaaatttattattcccgatgtttttaatgatgtaaatttaaatcaagaaaacatattaagttctgttaataaatgtattatttttgtttactaatttccaataattttttcaagaagtacaattttttgtttgaaatttctaatttatcattattgaataatagaacatacctaaaaaccataaaacatgtattccttgaaacattttttttaatataaaaattatcttttaggagcaggtaaagtacttttttttataaaattgttagtgtatttatgttcgttatagatgtagatattttgcataataattaataatatatgagatgtgtaatgtatatatgtgagagaattgccatatttattaagcggtttttgtaaaattgactcaaaatttaaagtcaaccacaaaactaacatatgtttttttggacatttcatttgccctattcactccacaagttcagattatttacgaaaatgtcattaattttttttctttctgaaatggtatttttactctctcaacctcatcatcttcaagtatttacaagattgtcattgccatcaatacaccaaccaccatgaacaaccaatttgaataatgcaccttaaatcgatttacacttcttccttctcaattcttatgaactaaaaacaacatctctttcactttctctccatattcatccaaaaaatctaagatttttattcaaaaatttttatggttcatagaaccattgaagcttacgattcttggtgtgtcacttttgtttgagattataggtgcttggagaagacttttgtgtgctaaacaagttatctcaatggttgaactatgaaaatcagtgttttttccagatttgttcgcccaaccgactttcgggtaagttttctggctgtagacgactaacatggaagtcttctggtcaatgcacttctctttgattgtcttcttctgatttacattatgtagaatgaacaagagatgggaggaactaggggtaggtaaaaaaaacggaaccgaattatcgaaccgaacagacccgaaaaaaaccgaaccaaatttatgaattatccaattggatactgaaattgtatacccgaagaattagaatcgaactcgtaccgaaccgaaaaccgaataatacccatatagaaccagatgaaaaacctttatacactacttatacgtatatctttatagtttatattttatacacttgttttttttattgatcaacgcttgttttataggagaaccttgtattgtttggatattttatcctcataatttaagttttggtttctgaatgggaaaatcatttgaatgttattttggtgttggttttaattcatttatttgcaagtacatcagattaatttggataatatggttacaaagtaaaaagaaaaaactgaacttcaggaacctaaaaaaccgagaaccgatctgatctgatccgaaatttttatggttctttaatggttaccaatttcaagaaccgaaagaaacgagaaccgaaagaaccaataaccgaagaaccgagcgcccactcctaggaggaaccaaacaatactttgatttgaagcccaagataattggaaccatgttatattacgcagcttatctgatgtgggctttatgtagaactgataatgggctttaaggcagcctatttcggtccgtcaagaagattcctcctaacgtaaggtaggggcaatgagctgctactctgaccaattaattacgtttcaatagtgatctctcatctgttgtcggtgtgaaaggaatcgtcttttgaatttatct

SEQ ID NO: 13 - DNA, 14640 bp, Brassica napus
  source 1..14640: mol_type = genomic DNA
attattatttgtatttatatatctagggtattaaaatctttttacttattttatgaaacattttggttatctttctccttacaatctatttttgtgataaaaacttgaaaatagtctacttaggagaattaccctttacattatctaataaactgatcttctagaagagagaaacgttaatggtggacgaagcccccacctcaattgacaataaaagaaagtggaaaagagagaagatagcagagtgaaaggtccgaagagaggagatcatgaagagaagaagacaatgtaacctacttatttgccttgatgaacaattacgagaagaaataaacataaaatgagaagagagaggtgggatatacagaagagagaaagatttggcaggaaacccgattgaccatatatcagatctagagtggattggattagataactcgagcttatttggttaatctttttagtttagggaaggttatgaaaaattaaccccgatctgatttttaaattttttatcttatattgtgcccccggctaacaatatatctagatccgtccctgatcaggtccatgagatatatcatatttttatttttcttatttaattaattatatatataatatttgaggatatatttaaaattttacatatcgttgaaagccagaaaaaaatgtggaagtgtaataaaacaattttctccaggaagaagatatttcagccattcgcctatttatgtgtatgagatcccattcaaggacatactgttttccgtgagctactgaatgaatacgatgcttttgttgtaaacgcaacctattggttttttttttccgccaagttttgtttttattaatcaaacgggccaagcccaaacggtcgaagcccaaatacactaagtaccaaacaaaaggcccacagacacaacgggctacaacattaatggctgaagcccaagtaaactaaacggcccaaaggcccaaacattgaaaccggacagcttgacctaggcgcgcgataaggagactggggtttgaacacgtgtaagaatctgggccattagtatgacacgcgtcgcccgcctcgacttgatcgaaacctcttcgcacagccgcatgaacttgccaccgacgacctcgtttcgccggagctcagagactgaagagcctccaacgaaaccatagctattctacgccgctctgtcttcacgccggagagttccatcgatctaacgagatctcctccgactatgtatcaccacaaaccctagacaggcgagccgagaaccactgatatcctcctcttgtacgtcctcgccgttgtcggagagtttcatcatcatccgagatctcatccgcgatggcccacgaccccatacctgaccacaagaaccacaccgcacttatctcacctcgatctaatcaccacaacccaaaggacatcgggttccaagacggcagcgcggagctttgcaagcctccactctccgtgaccaaaagccggcgaagacggagctgaatgagcctccccttcccggaagctaaagcggcgacggtggatctgagaaagcctccacctcccgcataaagaccggcgacgatggatctagggtagcctccatctcccggaaaaaacaccacctgaacatgcaagccgctccatctccaccgggagcctccaattgatcgcgtcgttactccaaaggccgagccaccgttgatggatggttgcggtcccagagctccgacaaggcgatcgatgacgggactgcgaagaaggagcggagagtgacaactacaaaggaagggaaagagctctggcgacagcacggacgctcacgcgccagccgtacgccgaacccaaaagtagatctggtttctctcttttctctctctcgcaaaagaaaacacgcaacctattgttctccattttgaatttgaagtgttacattattaataaaaaggaagaaagtacaaagcttttcatttagtggttgttgcatatatagtatacttatttgatgggcttatgataagtatgaacatttcacgtaacgggtttattttgtcgtcaacacattaccggcaatgtgaaaagaaggggcgaggtatctccatttcaattcttctctttatagattaatcgaattatttacatatgaaatgagcgtttatatagaattttcgtgtggaaaacgacatgtacacggcatcgaaagaccaattagtaatatactttagtggtgattacatgtttacttatccaattgagaatttaaagcatcgacaataccttaatgtcgattaagccgtccccacttcatgtaatgagttatggggggagagagaggtcccgaaattcgtcaaataaaacaacttagaactaaaaaccgacaccaagtatcataaaggaaacgttgaagaagtcatttatcgtatccagctcacaattcctaagattaaatcatgaccgttggaagagcttataagattaaattgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctatatggcaatgatatcatcaccaaagtgcaatgcccctttttgccctagtttcgtaaagtctctttccttcttcatccttaggaaaaaccctaaattaaatcctgtgttcttgatcttgctttttgagtaaccatgattttgaccacacactagttcttctatattttgtggtctataggattttgctttatatgtgtttcttgtattgcaccgtacgtgcgtatataaatttaaatggttacaacaaggtttattataaacaggcacaaattagtccatgaggttatttagcttgcacaagaggtatataaatgtgtgtgtgtgtgttaagtatttaaatatataaatttgctacaaaacttaattaactttatctgattatattttctttagtgttcttcatttgccaacgttgaggtagctattattattattattattattattattattattattattattattattattattattattattattattattattattattattttgaacattatgtactatgtacgtagttattttggctagttatgattcaaattcttaatttggataacacttaacagtatttaaaaaaaaaatactatttaaaatattcttagtactaaaataattaagatttaacttgaaattgaagtattcgtgttaaacagaaactacaataaacaaatgattgcatgttaatttttttttcgattatcctatatatcagaataaacacatgattgcatgcaaattttgtttttgattatgtcatcttttgtttattttagttttgatgctaattaatattttttttattaacaacttccatacattctacctgattctaggtcagataatgacacagcgcaacaaaattatacaaaactttcggaaagtagattaccgcggaagtaacatttttgggtagacatacgaagtagtttgaatcttcaaaaatcatgccatacataaccacggatcatagtcgacacctcaacgtgaagcaaatttgacaatctacatacataaccaacaaaaagtagaataccgtgaaaacctaaacccaaaatatgatgtaaaactcaagcttggtccagagcataaaaaaattaaagccatcgctttggtatcacatatttaaacgtcagtttttttttggggaagtaatataaaaatataattaacaagaaaatttatgaaataattagcatgtaaaacactagtcttttggttgtgacaaaacgttttcacaaatgttctataaataaattcaagcacattttatctgcaaaatatatactttcactcataaaataagagcgtttaaaacattcatatacgcactacattgacatgacaaaagaaatccgcaaatacaaacatatttagttcggatatatctaggaaataagactatattatatatataaagaaattagaaaaaaagaaaattggtatgtataaaagaaaggtctatgcgtctctagtccaaaaactctatatgtctggttcaaaagcagatgcagcagccatagccccaatagtcatgatggagcctcatcatctccttatgaactggaacaaacctattgatctcattacacaagaaaactcttttaaccacaatcctcatttcatggtagatccaccttccgaaaccctaagccacttccagcccccgccgacagtcttctccgatcccggaggaggagaggaagcagaagacgaagaaggagaggaagagatagatgagatgaaggagatgcaatacgcgattgctgccatgcagcccgtagacatcgatccagccaccgttcctaagccgaaccgccgtaacgtaagggtaagcgaggacccccagacggtggtggctcgtcggcgtagagaaaggataagcgagaagatccggatattgaagaggatggtgccaggcggtgcaaagatggacactgcctccatgcttgacgaagccatccgctacaccaagttcttgaaacggcaggtgaggcttcttcagcctcacactcagcttggggctcctatgtctgacccttctcgcctttgttattaccacaactcggatacctaattataattctatcacgcgtttcatgttgatatatatagataaatggttgaataaggatttcgatcgaagattgtatggctattgattacattatatattgtacaataaatgatgtgtgtatttctattaatgtatatatgatatatatctgtttgcagtatgcatttatattctattctttatagggaggcaacatgccggattagggctttgatcgtatgcaagttttccgaccaaaaatatgaaatacttgtttggatataacatatgaatcggataagtgttactagttatataactggaaaaaaattgtttggtataagaattcccgggagaaccaagcctttctctaatccctaagatcatagctactgaaataatgaaaaaaaacaaaaaaaaaacaatgaagaatcagttgggcattagtccaaaaaaaaaaaagaatcagttggattgcttataaaattttgttataaaatttatgtcgtatgtgtgttagccgtagctgtaaatttatttcctttgtatgttaattgtaaaatttaatttattattcccgatgtttttaatgatgtaagtttacatcaagaaaacatattaagttctgttaataaatgtattatttttgtttactaatttccaataattttttcaagaagtacaattttttgtttgaaatttctaatttatcaacatacctaaaaaccataaacatgtattttttgaaacatttttttaatataaaaattattttttaggagcaggtgaagtatttcttttaataaaattgttagtgtatttacgttggttatagttgtagatattttgcataataattaataatatataagatgtgtaatgtatatatgtgagagaaataccatatttactaagagtttttcgcaatattgactcaaaattcaaagtcaatcacaaaactaacatatgtttctttttagacatttcctttgccttattcactccacgggttcagattattcacgaaaatgtcattaaattttttttcttttcgaaaatggcatttttactctctcaatttcatcctcttcaagtatttacaagatttccattgccatcaatacaccaaccacaatgaacaaccaatttgaagctcttaatgcacctcaaatcgatttacacttcttctttttcaattcttatgaactaaaaacaacatctctttcaatttctccccatattcatccaaaaaacccaagatttttattctaaaattttaatggttcatagagccattgaagcttacgattcttggtgggtcacttttgttcgagattttaggtacttggagaagacttttgtatgctaaacaagttatctcattggctgaaactatgaaatcagttttttttccagatctgttcgcccggacgacttccggataagtcttctggctgtagacgacttacatcgaagtattctggttaatgcagatgttagttttgcaattgacttttaagatgcttactcgaagattagtactcttctgttcttgtaccttatgagcattccaatagtattatatttttcattctaaaatatatttcaatgatattttacttttcactctataatagagtgaaaatgagtttactttataatcccctatatattatttgtgaaacactacaacttctttttgtagccacatgtcatcactaggatgatttttagaattattagagaaataggttggtccatctaattatataataagttttttattaaattaaccataaattcattattaatgtcatttattatttccttaaataaagattacggaattgcctaatgtgggtaaagtatatatgacaattaatgatcttgaataataaagatatgataaaaaaaatgtatcttctatcaaatttgtttaatttaaaactattaaaataatttttaaaaaaacacaataaccatattataaaaatttagatttttctgtatatttttatattttgaactttaaaaaatgacgataaattactaaaactgttaaaagtctcacattcaaattttgctatccatgttttaaaatttttgttatgacaaaatacaaatgattacaaaatcatataagtaaaagtctaatttaattaatcattaagatttaaaatatatatgtatatatatcattctaaattaaactataaaccatattgaataaataaatattttagtttcaaaattttactttgaataattttttttgataaaagttttgaactaacattgataaatgtttttaaactatcaattaccaaaattattgatcccacaatgaaaattttgttatcagtaatttaaagtttttgctattaaaaatacgcataataaaacaaaacatatgagtagaaagcatcatttaaagaaaaataaaaaaataaaatagacattaatattaaaaatatactgtgtatgttaatatcatttaaatttaattacatatcctatcaaaaaaaatttaaaaaaatgtttggattaataaaattgatttatacgttcgcaccaatataattatatacgtaatagttactgacttttaattattcaatatatatttattatttcataatatgtaagaacatataatacataaaataatttttatatataatgtttatctcgcgcaaggcgcggatcttaatctagtctatattataagagttattcttgggttcatcccctagggtgaacctccagccaataggatttctttatttcaaatacgatatattttaaaaaaataaaccaaatattatcaagttatattatgtttttaaaataaaaaagtaaaaaaaaagtagcagttacagaaaaaagaattaaaaaaaaaaatttaacgtcgtcagcaaaacactaaaccctaaatcataatccctaaaccctaaatcataaattctaaacccttgggtaaacctaaactcttgggtaaacctaaactcttgggtaaaccctaaaccattgggtaaactctaaacccttgggtaaaccctaaacccttggataaatcataaactctaaataaaaactctaaaccctaaaactctaaaccataaatcctaaaccctaaacccttgagtgttttagtgtttagtgattttgatttagagtttaagatttatcctagagtttaagatttatgctagggtttatggtttaccaaagggtttagggtttaagatttagggtttagggattaggatttagagtttaatgttttgctgatgacgttaaaaatatttttttttgtaattactgttttttttttttttaccttttaattttataaagataatataatttgacaatattggtcggtgaataagtcatattataatgccaacttttctctttcctaaaggagcctgcgtgtgcattctataaagcatgggggacccacttaatgtctggtttattttggtcgaataggctatagtttggtttggtcagatttgggttttctacgtttggtttgattattaagggcctggctggttcaaccgcagcggttgcggttgcggctgcgggagtttgcggatgcgggtggttgcggtttctaacggttttaagagatttatacgactggttctgcggttagaaattggtgcgtttgatacttatgactggttaactaccaaacgcaacagcggttaaataataaattaacaaaatttacattttatatcattataaaatatcaaaaataataatattataataaatataaaatttatatttagaaagttatagtttaaactttttttaaattatagaaaatattttcattttaaagttttataatattaattaaaatataatagatatattttagtatttttataattctaatttaaattttttattgaatatttttatttttgtatttaaattgttttagaaaaaaaaaaatttatcctcccgcaaccgcccgcaaccgcaaacgctagttggaaccagtttttgaatttatgaggtttagagcgatttggagcggtttggagcggtttgagcgattgttgcaaaacgccaacaaccgctaccaaccgcaaaagctgcgtttgcgggtggtagcgggaaaaccagtcttacccttgtcattagggcttcattagttgttagcgttcttccacacttcgagtgtgattggtaacatcagagtaaagtggagtaaaaaaaagtggtgaaaaagaggaaaataaatggtggaaaaatgtgagagagaaaagaatctggaataacatgagtgtacctaaagctagaggaaaaataggtgtatatttccctcataatcaatagtgcctagagtaaatatgaaaagatagtatttcattttattggtgactttttggcttaactgagagtaaatatatatctagagtaatagtttactctatagataccatacaatcatacccttcatcctcgccgtgaatctgcaaacctaatgacttctcctcccatctctttaccttcccttccgttcaatatatttcttttaattcagcaatagttgttattcacattaaacccagtaaggatgtcgacctcttcgttttcttcacgtcaacctataaataggagaaggttgagagtaggatggatcttcagaaccactgtctgttaatcttctttgtcactctcactcactctctctctcatctgcaactggtttggccctcccttcgagttatgtggaccttgtggtttgtctttctccagtgagaaacttggtgagcaaacttaatattattcatcttatgcattacttattgaactggaagtatcttatacataatacccgtctcaagaacctatcaaagaggcgggaattttggagggtcgcaatggtataaaggagacatgccgtcaggtatgcttaccgtggaagtcatgttgcaacagttaacaatcatgattatgtttttaaatttaacttttaaatttgctttatttctgtgatcacattggtgcttttatgtttgtttttaggggatagtctaattattttcatatatggacccatctttcttaagtatgctggttgctcatgtcgtgttgcatattagggacagcgccctaagaaccccccatgttagataattctaattaacttaaagagcaagttaactcattaaagtgaagtttgatgggttaaggaaaaaggaaaacatatcgagcttaggaatgtttccatattattattaggaaaagatgtagcttcgcctttaggaaaagatgtagtttcgcctttaggaaaagatgtagcttcttcctatataaagagttcttatggagagatgttctataaaaaaaaacacattgaaaggtttagttttgagagagtttctaaatctaataagaagtgaagttcttataatctttgtgtatatgcaactttaaattggtatcagagctccaggtttcgaaggtcgtttacaagaggagttgtaacttcgatcaaaacatgggagacgattctcaagcacgtgataaaggtcttgggatgaagaaggacatacgatgtccgatgctaacatcgaccaactacaccgtatggtcgatgcgaatgaaagtgatgcttcgtttatacgaagtttgggatacgattgatccagggagtaacgatgcaaagaagaataatatggcgattgctctaatctttcaatcagtcccggaggcgctaatacttcagattggagaacatgatacatcgaagaagatttgggaagctatcaaatcccgtaacctaggtgctgatcgcgtgagagaagcaaggttacaaactttgatgtctgagttcgacaaactgaagatgacagacacagacacggtggatgactacgcagaaaaactttcaggcttagcatcgagagcagccgcattaggagagataatggaagcatcaaggctggtaaagaagtttctgaagggacttccaagaacgaagttcattcacatcgtagcttcgttagaacaagtgctggatctaaattctacgggattcgaagacattgttgggagattgaaggctttcgaagaacgcatcaaagaagaaacccaagatgaaggtcaatcgaagctcatgtttggaaagaatgatatgcaaggtagtggaagccataacaactcgcgaggaagaggcataggtagaggttatggtagaagaggaagaggacaaggaaggtctaatgtttctgatggtcataacaaaggaggagaagcttcagacaagaccaagaaggactactctaaggttagatgttggcgatgcgacaagatggggcacttcgtgtcacattgtcctacacgaccaagagaagaagctaacctaacggaaacacaagaagcggatgcattatatatgcatgaagtagtattcctcaacgaagagagagtgtttcctaagaggtttgatgaatgcgatggaaatacgagtatatggtaccttgacaatggtgcaagtaaccatatgacaggcaagagagagttcttctcaaacctagatgatagcatcaaaggcaaagtcaaattcggtgatggatcaaacatcaagattgttgggaaaggttcgatcacgttcaccggaaaaacaggggagagaagagcactcaaagatatctactacatcccaagtcttaaacacaatatcataagtcttggacaagcaaccgagatgggttgtgaggttaatatgaaagaagacttactgatgctgaaagatccccgtggaaggttattagtacaagtcgcaaggcaaccaaaccgattgtacaagacgccaatggaggttgaatatccgaagtgtcttcaaatacaagaaactgatgttacatggacgtggcatgcacggctaggacatgtaaactttggagtcatgaagaatatggtagacaaagagatggtagtagggatgcctcaggtgatacacgagaaagatgtgtgcaacgcctgcttagttgggaagcaaactcggaagtctttcctgcctaaagcaaagtatcgagcatcacacgcattggatttggtacatggtgacttgtacggtccgatatcaccatcaacaccagcaaacaatagatatgtatacgtcttaattgatgattactcaagatatatgtggacgatgctgctaagagagaagagtgaagcgttcgatcggttcaaaaagttcaaggagtacgtggagaatcaaacgaagctacaactcaagaattttcgcaccgatagaggaggagagttcacatcttctgatttcattcgtttttgtgaagaaaacggtgtaattagacatctcaccgcatcatatacactatcaagaaacgtggacgaagaaccaggatcattcaagcttcctcatattgatgttacagaaggagatggtgatgagcatcaacatcaccaacaccacgaagagcaaaacaataatgaagaagatgtgaacttgtttgtaacggggacttcgctcagtgttatcaagcaattcaaagatgatatgtcaagaatatttgagatgtcagacctcgagatgcttacatactatcttggtatagaagtaacacaaggagctgatggcattcacatcaaacaagaaggatacgcgcaaggtatacttgtcaagacgaagatggagtcatgtaactatactcacgttctgatgcacacgagtttgaaagtatcaaaggcagaggaggagcctgagattgatgcaacatcgtatcgaagcaccataggatgcttaagagagagtataaatgcttcactgcttctctatgactctctcttaagcatcttatggtgcttcgatacgatgttgcatcaatctcaggctcctcctctgcctttgatactttcaaactcgtgtgcatcagaacgtgagtatagttacatgactccatcttcgtcttgacaattataccttgcgcgtatccttcttgtttgatgtgaatgccatcagctccttgtgttacttctataccaagatagtatgtaagcatctcgaggtctgacatctcaaatattcttgacatatcatctttgaattgcttgataacactgagcgaagtccctgttacaaacaagtcatcgatgtatatagctatgatcagaagctcccccttctgtttcttttgatataccgcaggttccgtggtgcacttcgtgaactccatctccttgagaacacggtcgagtttgatgttccaagctcttagagcaactggtgcaaataattcgtcgaagtctatgccttgttgttgcacgtagccttttgcgactagccttgctttgtatttgatcaccgttccatctacattcctcttgatcttatagatacacttcaaacctatcagtttcacacctgccggcttcttgacgagcttccaggtcttgtttttgatgatagatttgatctctgccttcattgcatcgatccaagcttgtattactgcagcttcgatgtaactttctggttcaccatcgatggtgagcaagagtctgccactttcaacttcagcaagtaggatgtaatcatcgaaccgctttggttttctgatgttacgaccatatcttgatgttacatgctggtcttggtttgcatcgttgttctctgctacttgctcttgttcacctgcgtctacagcttcttcttcttcattattgttttactcttcgttctgatgcaacatcgtatcgaagaaatgcgatgcacctagtaacggtctatggaagttcatcggagttcagaagatcaacatacctaagattcaagttggaattaagggggagaatgttggataattccaattaacttaaggagcaagttaactcattaaagtgaagtttgatgggttaaggaaaaaggaaaacatatcgagcttaggaatgtttccatattattattaggaaaagatgtagtttcgcctttaggaaaagatgtagtttcgcctttaggaaaagatgtagcttcttcctatataaagagttcttatggagagatgttccatcaagaaaaacacattgaaaggtttagttttgagagagtttctaaatctaataagaagtgaagttcttataatctttgtgtttatgcaactttaaattatatctcttatttaagagacggttcttaacttttcttagttaaagctaagaaccgtctcttagctgaagctaagaacccagtctcttatctggggttaatcatggtcgtctaatgtctcatttattagtgtttttatttttcacagtcgttaggtactctcatcatgctctctcctatataattcgttaatgcatgcgcagataccaattatcatgcttgttgtggaagtcccttgtgatctggtatgcagtatacagtcaccaaaagatggcccagactgtatcaccgttgaccaagacagcaatagtaacactgatgtggttggatttgaagactctgcagcagcaaactctttaccgacaaaggctggaaacttggcaaatgcaaaatgtgtccgtcttgacatcagtttcaaaacgccatcgcatacgtgtcttcaaaccacacaattggtgtttgtttaattcttctttcacagcaagatatttgtttttcttacattatacctcttcttgtctatgtatctctgtaaccttttttctttctttgggcttatgtttcctaaaatgttcgtttctgcaggtgaaagatctgacagcaatttccagctgccacaccacttgctttggtgttaaagcagtttttggctgatcgtaccctggatcaatcatactccggcggattgagctcttattgcctggtgagccgtgttcctttacattaagta

SEQ ID NO: 14 - DNA, 3781 bp, Brassica napus
  source 1..3781: mol_type = other DNA
agaggagatcatgaagagaagaagacaatgtaacctacttatttgccttgatgaacaattacgagaagaaataaacataaaatgagaagagagaggtgggatatacagaagagagaaagatttggcaggaaacccgattgaccatatatcagatctagagtggattggattagataactcgagcttatttggttaatctttttagtttagggaaggttatgaaaaattaaccccgatctgatttttaaattttttatcttatattgtgcccccggctaacaatatatctagatccgtccctgatcaggtccatgagatatatcatatttttatttttcttatttaattaattatatatataatatttgaggatatatttaaaattttacatatcgttgaaagccagaaaaaaatgtggaagtgtaataaaacaattttctccaggaagaagatatttcagccattcgcctatttatgtgtatgagatcccattcaaggacatactgttttccgtgagctactgaatgaatacgatgcttttgttgtaaacgcaacctattggttttttttttccgccaagttttgtttttattaatcaaacgggccaagcccaaacggtcgaagcccaaatacactaagtaccaaacaaaaggcccacagacacaacgggctacaacattaatggctgaagcccaagtaaactaaacggcccaaaggcccaaacattgaaaccggacagcttgacctaggcgcgcgataaggagactggggtttgaacacgtgtaagaatctgggccattagtatgacacgcgtcgcccgcctcgacttgatcgaaacctcttcgcacagccgcatgaacttgccaccgacgacctcgtttcgccggagctcagagactgaagagcctccaacgaaaccatagctattctacgccgctctgtcttcacgccggagagttccatcgatctaacgagatctcctccgactatgtatcaccacaaaccctagacaggcgagccgagaaccactgatatcctcctcttgtacgtcctcgccgttgtcggagagtttcatcatcatccgagatctcatccgcgatggcccacgaccccatacctgaccacaagaaccacaccgcacttatctcacctcgatctaatcaccacaacccaaaggacatcgggttccaagacggcagcgcggagctttgcaagcctccactctccgtgaccaaaagccggcgaagacggagctgaatgagcctccccttcccggaagctaaagcggcgacggtggatctgagaaagcctccacctcccgcataaagaccggcgacgatggatctagggtagcctccatctcccggaaaaaacaccacctgaacatgcaagccgctccatctccaccgggagcctccaattgatcgcgtcgttactccaaaggccgagccaccgttgatggatggttgcggtcccagagctccgacaaggcgatcgatgacgggactgcgaagaaggagcggagagtgacaactacaaaggaagggaaagagctctggcgacagcacggacgctcacgcgccagccgtacgccgaacccaaaagtagatctggtttctctcttttctctctctcgcaaaagaaaacacgcaacctattgttctccattttgaatttgaagtgttacattattaataaaaaggaagaaagtacaaagcttttcatttagtggttgttgcatatatagtatacttatttgatgggcttatgataagtatgaacatttcacgtaacgggtttattttgtcgtcaacacattaccggcaatgtgaaaagaaggggcgaggtatctccatttcaattcttctctttatagattaatcgaattatttacatatgaaatgagcgtttatatagaattttcgtgtggaaaacgacatgtacacggcatcgaaagaccaattagtaatatactttagtggtgattacatgtttacttatccaattgagaatttaaagcatcgacaataccttaatgtcgattaagccgtccccacttcatgtaatgagttatggggggagagagaggtcccgaaattcgtcaaataaaacaacttagaactaaaaaccgacaccaagtatcataaaggaaacgttgaagaagtcatttatcgtatccagctcacaattcctaagattaaatcatgaccgttggaagagcttataagattaaattgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctagaaccaagcctttctctaatccctaagatcatagctactgaaataatgaaaaaaaacaaaaaaaaaacaatgaagaatcagttgggcattagtccaaaaaaaaaaaagaatcagttggattgcttataaaattttgttataaaatttatgtcgtatgtgtgttagccgtagctgtaaatttatttcctttgtatgttaattgtaaaatttaatttattattcccgatgtttttaatgatgtaagtttacatcaagaaaacatattaagttctgttaataaatgtattatttttgtttactaatttccaataattttttcaagaagtacaattttttgtttgaaatttctaatttatcaacatacctaaaaaccataaacatgtattttttgaaacatttttttaatataaaaattattttttaggagcaggtgaagtatttcttttaataaaattgttagtgtatttacgttggttatagttgtagatattttgcataataattaataatatataagatgtgtaatgtatatatgtgagagaaataccatatttactaagagtttttcgcaatattgactcaaaattcaaagtcaatcacaaaactaacatatgtttctttttagacatttcctttgccttattcactccacgggttcagattattcacgaaaatgtcattaaattttttttcttttcgaaaatggcatttttactctctcaatttcatcctcttcaagtatttacaagatttccattgccatcaatacaccaaccacaatgaacaaccaatttgaagctcttaatgcacctcaaatcgatttacacttcttctttttcaattcttatgaactaaaaacaacatctctttcaatttctccccatattcatccaaaaaacccaagatttttattctaaaattttaatggttcatagagccattgaagcttacgattcttggtgggtcacttttgttcgagattttaggtacttggagaagacttttgtatgctaaacaagttatctcattggctgaaactatgaaatcagttttttttccagatctgttcgcccggacgacttccggataagtcttctggctgtagacgacttacatcgaagtattctggttaatgcagatgttagttttgcaattgacttttaagatgcttactcgaagattagtactcttctgttcttgtaccttatgagcattccaatagtattatatttttcattctaaaatatatttcaatgatattttacttttcactctataatagagtgaaaatgagtttactttataatcccctatatattatttgtgaaacactacaacttctttttgtagccacatgtcatcac

SEQ ID NO: 15 - DNA, 8640 bp, Brassica napus
  source 1..8640: mol_type = genomic DNA
tgtgaaattttgtgtattcactttagttataatatctatgtataccaaagagaagaagataacatatattatagtattggtacatactataatataacatacttaattagtaaatttcacaccacagcaaaaataatataaaaggtcactttttaatatacaacgtgccaaacttcgtccatttataatcttatatatttttgtcttcgcatatgatagatcataagatttggagaaacatataaaattaacctatgaaattaaggggatctatatattatagtatgtatccaaagaacatatatatgaaatatttgaatacatcataagatttggggaatataaattaatataaaataaaatcgtccaattttaaacgtaaacgtctccgctgtatttatataaacttttcaaaatcacggtgtcgctcgtgttaagatttggatttctaatccaactttcagaaagtttatgtgaaagataatactcaaacacaatagttcgtttagaacgctcttaagattaatgtcacacacacaaaattgatgtcactagatttatatgttttgtcgtttaaggggatccatcaactacacatggaagaatagaaaacactggtcacacacaaaaaataatataagcatgttgagtcctattcatcagaaaattttctccaaaggatgaacaataacctacaattaaggaaacttattataacaacgaccaagaagttttcccataagtcatgctttcgtccataccttcgggtctttcaagcaagcttcacacatgccttatcgtatatttgtatacgttttcattttgtgtacgtctattaattagtatatgcctagattatatatttatatagtcgacaattacctatcctaaaggtttattttaatttttattcagaaagatatgcacgtagggccactagtgtttcccgttgttattggaaattccatgtgttgaactgtttgattaagtttttgaacccgtaagtcaaatttatttaggtttaacagggtttcattactgttataagcttgtcaagaaagaatttgtcacaaatttcgagactcatgcatgcatgcatgtttcttaactttcgagaaacatgaagtgtggttttaccagctgaagtttgtgttaaaacacatttttaatttgaccacgtgtgatattcaaatatcaacaattctgttcaacgggagattcatacatatgttcagatgttctttaatcttgagcacctaactttgcttcccatacgcaaatgtgtgtgccgcgaactatggtgaatgtacggcatcgtttttctttttggagcatttttaaaagaaaatataatttagttatttccgttttgattgatatagatgcattaataaaccgacttacgtgggtgatgggatgagcaaaattaaaatatgcattttagtaaccaattacggtccatcttgacaaactaacaaacaaaaagtctattatgacgttgatttgtgaccttgccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaatgacaactcctgttcttgtatcgtgtatctagcacgtaaatttgaattatggcggcattatcgataaaatatgtaaagaatatattttcatcatacaccacaaacaaatgttctggaaatatcaccttttatcacatatacaaagctgaccctgaatttttggaaactacaagccaaaaaaaaatggacaaaagaaaaattatgcacaaaagtggactcgtcctaactctttttataatgataattaacctttgaacaactaattaatacatacaatatgttttttttttctaaaaattggtcgattaaatattcatatataatcctcatcatcatttattagccttttgtagcttacttaaaaaatcacttcccaccaaattgattattttttcacttcatacgttgatcaatttaaaaaaaaaataataataatatatgcacgtccggcgtcccatgacttcagacgcttttctgtttccttgattttataggttagaaatttccaaaatgctcatatttttcactgatatgtattatacgcacaaaatagtaaagtcataaaaatttatgatggttcttttatatgtatagagtagtgaaaatttatatatattgtagttccgcttaaaataaattaatatttttcttttatcgcccatatttaagttactaaacgtatataattaaaaactgaaatgcaatatgtagaaatccgggcattgattgttgattaacattgatatacccatgcgactggcttatgatgaaccatacctgctaatactcattgttacaaactcgcagcaacatcataaatccatcactcgccacaataattttcttcagtgtatgttcaattgatcacattttgacccttcactttcttgaatgagaaattatacaagaaagtgatttgagtgtgttaaaaaatgatttgtcattctgaccaattatctggcagagagtataataaaatacttctaattaccttaaatcattaggcgttgtttcttgccgaatatatgatggagcatggtctcgctaagagacaacaaaatctttaaataaattaaagaaattgtttagcaaaaaaattaaataaattaaacaagagagagtacaaatataaaacactctaacggaagataaatatataaaatacaaaatatagaatagaggcctcaagtagtgtttacgtgacaatctcaatttcactccatacctcgaactcattcaagcttagaaacctaacttctactgttccttttgatttgcctataaatacacgttggtccatacttctctaatttcaaatatacaaagcaaaacacagagacaagaatcataaataacaaacattttcaacataatcccaaaaaacgacacgcgttccttttccaaaaattcacttctctacgtaccaaccacaatggctcattgttgcacagttcttaccggtttactagtatggtctctttttatgctctcatggtgtgaagcttcaagaaacaacattaatagatatgatcattcttatggaacgttcaaatccaatagcttaatcaagcgaagagacgatatcactcgtctgaaaagtgttgcaagagcttctttgcggcttccaaccaccgttagtgtttctgattttggagctaaaggagatggaaaaaccgacgacacgcaggtatatacgcatatacacatatctacatgacttataaatattttgagacggtcacgcatataaatttacgttgtttgtactaatgatccatgcatgcgcatgcatatgatcaggcgttcgtgaatgcatggaagaaagcttgttcttcaagtggagctgtaaatctcttagttcctgaaggaaaaacttattttctcaagtctcttcgattaaatggcccatgcaaatcagttcttaccgttcaggtaaattacaaagtttttttttcttaaatttgggtaaattacaaagttgactatttgttaattaaatacgtatactacatgtgatcacgtatatggttttgaatatttaaatgagcggttgatgaatggaacgacagattttaggaacgttatcggcttctcaacaacgatcggattatgaagatctcagcaaatggattacgtttgacggcgttaacagtctaacggttgatggcggagccaccggaactgtcaacggaaacggcgaaacgtggtgggaaaactcatgcaaacggaacgaagctaaggtgtgtgtttgtacatgtttcatttacttaatatatatctactgtctacatgtcatcttgatatattaacactcttttattgttattgttttgaagaaatgcacaaaagcccctacggtacgtagaaacatatttgacattttcatgtaatactgacgatggcggtgatcatgattggacaaatgttttacatgcatgaatatatgtattttatatgcaggctcttactttctacaactcgaagaatttgagagttaataatctgagggtgaaagatgcgcaacagattcagatttcgattgagaaatgctccaacgttcaggtctcaaatgttgaggtcactgcacctgctgatagtcctaacactgatggtatccacatcactaacactcaaaacattcaagtctccaattccatcattggaacaggttagtagttatatcctcgttatttaaaagagttgcgtgcatatccagaaaactaaaatttaagagtatcgttataatttataattttatagagattggtcttaaaagagtttttaatggttgcttttctttgcaggtgatgattgtatatctattgaaagtggatcacaaaacgttaaaatcaacgatttaacttgcggcccaggccacggtatcaggtatcaatgaattgttttcagtatatgtacctaagaaattcaaccgtaatcttttaaattaacatagaaatttgatatttccatataaaacatgagcagcattgggagcttaggagatgacaattcaaaggcatttgtatcaggcgtgactgtggatggtgctaaactttccggtacagacaatggagttaggatcaaaacttaccaggtgaaaatcacaaactagaccttcaattttaactagcctattgacaagttcatctcaatttaacttttaaacccaattcaatatagggagggtctggaactgctagcaacattatatttcagaacattcagatggagaatgtgaagaatccaatcataattgaccaagattattgcgacaagagcaaatgcactgaacaagttcgtttgaatctgatcaaatctcccattactaatcatcatgataataaacactattgtctctaatcatttactacatatattttcagaaatcagcggtccaaataaagaacgtggtgtaccggaacataagcggcacaagcgcatcggacatagcaataactttcaactgcagcaagaactatccatgccaaggaattgtactcgacaaagtgaacattaagggagggaaagccagttgcagcaatgctaatgtgatggataaagacggtgttttacctcagtgcaaatccacttgattaataagacctataaaaaaaactcacatagatgtataaaaatacatatataccgatacaaagtatacaaaattatgatatacacttgtggatatatacatggacaatgaatttaaacatattgtctatatgtcaaaaatagtttatgaatatacatatagacaacatggtcaagagatttattgttttaatgtatatatatactactaaactacttgtctaaaatttcatgaataaaggattacgtttttaagtggttatgtatcatgatgtggttacagtatgatgtgtattgtagattttcaattaaatacataaaaatgaaaaagatgacatggctggtcaatgatgttacttgaccaactaattcaagctaattgatacacgatgaacatctaacgaaacagaagcgactttcatattgtttagttgtgctcaaaccaggccgataaactcattgggcctataagcccattaatatttatccgtgagagcgaagtctgttacttgggttactaaacaaagaggcctttagaaaaccctaactcttaccgtagcacctgagctatatattgccgcagctagggtttccactccctcacgctaacacacatcaccgcagccatttccatctctcaaccaacttcaaacaatggctgaacgaggcggagaacgaggtgccgagcgaggaggagaccgcggcggtttcggacgcggattcggaggaggtcgcggcgacagaggaggccggggaggtccgagaggccgaggcggcagacgcggaggacgtccgacagaggaggagaagtggaccccagtgaccaagctcggccgtctcgtgcaagccggaaagatccagaagctcgagcagatctacctccactccctccccgtcaaggagtaccagatcatcgacctcctcgtcggccccaccttgaaagacgaggtgatgaagatcatgccggttcagaaacagaccagggccggtcagaggacgaggttcaaggcctttgtcgtcgtcggagacacgaacgggcacgttggtttgggtgtgaagtgctccaaggaggtggcgacggcgatcagaggtgggattatattggcgaagctgtcggttattcccgtgaggagagggtactgggggaacaagattgggaagccgcatacggttccttgtaaggttacggggaagtgtgggtctgttacggtgaggatggtgcctgctccgaggggtgcggggattgtggcggctagggttcctaagaaggtgcttcagttcgctgggattgatgatgtgtttacttcgtcgagggggtctactaagactcttggaaacttcgtaaaggtttgaactttcttcatttatttagagtttggagttgtgtatggattgaaagttgattcctttttgatggttgcgtgtggagttgttttgggtaatttagcttgttgtagttgatgtaaagtgagaattgctaagcttgattagtttgaataagctgtctgtgttaaatgatgatttgtttgtgttgtattgagctgtctgaatttcattatttgtttgtgttattgctaaattacaagcttaaatttgataacttttgattctttcgtgagagttttttttgggtaatatagcttgatgtagttgatggaagtaaattgttaacctttagagctgtctgttaatggtgatttgtttgtgttgtattgagctgtctgaattataatttgtttgtgtttattgctaaatcacaaagctttgaagtcgtttgtacatattcatcatttttgtgtttattgacttgtttgtgtttattgctaaatcacaagtttctatgattaatcttcatgatttggttgtgtttattactaaatcacaagtttctatgattaatcttaatgatttggttgtgtttattgctaaatttgagattacattataatcttagtttatacatgtataacatcatttcacttgaattttcacagacgagctggtgatatgtttttttagtattttccatgtattgctttgtaagaacttttaaatgtttgttttcttcctttgaagaacaattaaatgtttgttcagtatttttgaatcattcattggttaatcatgtgtaattttatttttgtaggctacattcgactgtctccagaagacttatggattcctgacaccagagttctggaaagagacgagctttaaaaagtcaccgtaccaggagtacactgatctgctggccgagaagggaattgcaaccaaagccatcaccgaggttgaagaccaagccgcctcctaaataaacaaacaaacaatgtcccccttgttttgtttttggatgatcctttcttgagttttattttgttcgaacctttagtatatgttgtttgctggattgctgctttcttatgttttcatcatctcccccaaacaaagttatttttgctctagtttttttttataatcgatggttatgccctctagtattttttttacttgaatttttcactaattttctatcaacttccctatgttacactttctaaggttgattacataggtggattgttgtgaaacaaatactaataataaactaatacttttagaataatataattagtgggattaaattataaattataaattaaaaagacaagtctgaaatggattgtttaactgtaaaaggtaaagagactttgaatagaagagaataaatcaaaagttgacgtatcttctccgaaatttcagctatttctaaatacgcggattctgattactctgttctctctcgcctgataaaccttagaattgagttttttttttttttatctttttggttcttttttagttcttgtcggaatcgtgagaagctctgtctctctgtcggaaccaatggggactccggagtttccagatctggggaaacactgctccgtcgatatctgcaagcaaatcgatttcttgcctttcacctgcgaccgttgtctccaggttcctctctctctatctctgttttgaatcacatgcaaaaaagaatcgtctttgttgcgaatagttgtgtcgtatgactgtctgaatcgcctgtcttcgatcctgggaactactatatcatcatccttgttcttttgtcgttcgtgttctgggagaaccaagtgaaaagtctccgtctttctcttaattaccgcgaggaaaattcatcttttctacatgcgaccagtttgatttgatttgttccgacattaagtttatcacttaagttcttgtaattacgaaccaacgtgttgagccatctgtcatttcgagcttaatcatcgacatttcgcaattctccgcacttaaattca

SEQ ID NO: 16 - DNA, 5668 bp, Brassica napus
  source 1..5668: mol_type = other DNA
tggtgttaagatttgatttatggatgcacatagttggatacattttatgtttaaaatgtgtttttaatattattattgctgtggtgtgaaattttgtgtattcactttagttataatatctatgtataccaaagagaagaagataacatatattatagtattggtacatactataatataacatacttaattagtaaatttcacaccacagcaaaaataatataaaaggtcactttttaatatacaacgtgccaaacttcgtccatttataatcttatatatttttgtcttcgcatatgatagatcataagatttggagaaacatataaaattaacctatgaaattaaggggatctatatattatagtatgtatccaaagaacatatatatgaaatatttgaatacatcataagatttggggaatataaattaatataaaataaaatcgtccaattttaaacgtaaacgtctccgctgtatttatataaacttttcaaaatcacggtgtcgctcgtgttaagatttggatttctaatccaactttcagaaagtttatgtgaaagataatactcaaacacaatagttcgtttagaacgctcttaagattaatgtcacacacacaaaattgatgtcactagatttatatgttttgtcgtttaaggggatccatcaactacacatggaagaatagaaaacactggtcacacacaaaaaataatataagcatgttgagtcctattcatcagaaaattttctccaaaggatgaacaataacctacaattaaggaaacttattataacaacgaccaagaagttttcccataagtcatgctttcgtccataccttcgggtctttcaagcaagcttcacacatgccttatcgtatatttgtatacgttttcattttgtgtacgtctattaattagtatatgcctagattatatatttatatagtcgacaattacctatcctaaaggtttattttaatttttattcagaaagatatgcacgtagggccactagtgtttcccgttgttattggaaattccatgtgttgaactgtttgattaagtttttgaacccgtaagtcaaatttatttaggtttaacagggtttcattactgttataagcttgtcaagaaagaatttgtcacaaatttcgagactcatgcatgcatgcatgtttcttaactttcgagaaacatgaagtgtggttttaccagctgaagtttgtgttaaaacacatttttaatttgaccacgtgtgatattcaaatatcaacaattctgttcaacgggagattcatacatatgttcagatgttctttaatcttgagcacctaactttgcttcccatacgcaaatgtgtgtgccgcgaactatggtgaatgtacggcatcgtttttctttttggagcatttttaaaagaaaatataatttagttatttccgttttgattgatatagatgcattaataaaccgacttacgtgggtgatgggatgagcaaaattaaaatatgcattttagtaaccaattacggtccatcttgacaaactaacaaacaaaaagtctattatgacgttgatttgtgaccttgccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaatgacaactcctgttcttgtatcgtgtatctagcacgtaaatttgaattatggcggcattatcgataaaatatgtaaagaatatattttcatcatacaccacaaacaaatgttctggaaatatcaccttttatcacatatacaaagctgaccctgaatttttggaaactacaagccaaaaaaaaatggacaaaagaaaaattatgcacaaaagtggactcgtcctaactctttttataatgataattaacctttgaacaactaattaatacatacaatatgttttttttttctaaaaattggtcgattaaatattcatatataatcctcatcatcatttattagccttttgtagcttacttaaaaaatcacttcccaccaaattgattattttttcacttcatacgttgatcaatttaaaaaaaaaataataataatatatgcacgtccggcgtcccatgacttcagacgcttttctgtttccttgattttataggttagaaatttccaaaatgctcatatttttcactgatatgtattatacgcacaaaatagtaaagtcataaaaatttatgatggttcttttatatgtatagagtagtgaaaatttatatatattgtagttccgcttaaaataaattaatatttttcttttatcgcccatatttaagttactaaacgtatataattaaaaactgaaatgcaatatgtagaaatccgggcattgattgttgattaacattgatatacccatgcgactggcttatgatgaaccatacctgctaatactcattgttacaaactcgcagcaacatcataaatccatcactcgccacaataattttcttcagtgtatgttcaattgatcacattttgacccttcactttcttgaatgagaaattatacaagaaagtgatttgagtgtgttaaaaaatgatttgtcattctgaccaattatctggcagagagtataataaaatacttctaattaccttaaatcattaggcgttgtttcttgccgaatatagctagggtttccactccctcacgctaacacacatcaccgcagccatttccatctctcaaccaacttcaaacaatggctgaacgaggcggagaacgaggtgccgagcgaggaggagaccgcggcggtttcggacgcggattcggaggaggtcgcggcgacagaggaggccggggaggtccgagaggccgaggcggcagacgcggaggacgtccgacagaggaggagaagtggaccccagtgaccaagctcggccgtctcgtgcaagccggaaagatccagaagctcgagcagatctacctccactccctccccgtcaaggagtaccagatcatcgacctcctcgtcggccccaccttgaaagacgaggtgatgaagatcatgccggttcagaaacagaccagggccggtcagaggacgaggttcaaggcctttgtcgtcgtcggagacacgaacgggcacgttggtttgggtgtgaagtgctccaaggaggtggcgacggcgatcagaggtgggattatattggcgaagctgtcggttattcccgtgaggagagggtactgggggaacaagattgggaagccgcatacggttccttgtaaggttacggggaagtgtgggtctgttacggtgaggatggtgcctgctccgaggggtgcggggattgtggcggctagggttcctaagaaggtgcttcagttcgctgggattgatgatgtgtttacttcgtcgagggggtctactaagactcttggaaacttcgtaaaggtttgaactttcttcatttatttagagtttggagttgtgtatggattgaaagttgattcctttttgatggttgcgtgtggagttgttttgggtaatttagcttgttgtagttgatgtaaagtgagaattgctaagcttgattagtttgaataagctgtctgtgttaaatgatgatttgtttgtgttgtattgagctgtctgaatttcattatttgtttgtgttattgctaaattacaagcttaaatttgataacttttgattctttcgtgagagttttttttgggtaatatagcttgatgtagttgatggaagtaaattgttaacctttagagctgtctgttaatggtgatttgtttgtgttgtattgagctgtctgaattataatttgtttgtgtttattgctaaatcacaaagctttgaagtcgtttgtacatattcatcatttttgtgtttattgacttgtttgtgtttattgctaaatcacaagtttctatgattaatcttcatgatttggttgtgtttattactaaatcacaagtttctatgattaatcttaatgatttggttgtgtttattgctaaatttgagattacattataatcttagtttatacatgtataacatcatttcacttgaattttcacagacgagctggtgatatgtttttttagtattttccatgtattgctttgtaagaacttttaaatgtttgttttcttcctttgaagaacaattaaatgtttgttcagtatttttgaatcattcattggttaatcatgtgtaattttatttttgtaggctacattcgactgtctccagaagacttatggattcctgacaccagagttctggaaagagacgagctttaaaaagtcaccgtaccaggagtacactgatctgctggccgagaagggaattgcaaccaaagccatcaccgaggttgaagaccaagccgcctcctaaataaacaaacaaacaatgtcccccttgttttgtttttggatgatcctttcttgagttttattttgttcgaacctttagtatatgttgtttgctggattgctgctttcttatgttttcatcatctcccccaaacaaagttatttttgctctagtttttttttataatcgatggttatgccctctagtattttttttacttgaatttttcactaattttctatcaacttccctatgttacactttctaaggttgattacataggtggattgttgtgaaacaaatactaataataaactaatacttttagaataatataattagtgggattaaattataaattataaattaaaaagacaagtctgaaatggattgtttaactgtaaaaggtaaagagactttgaatagaagagaataaatcaaaagttgacgtatcttctccgaaatttcagctatttctaaatacgcggattctgattactctgttctctctcgcctgataaaccttagaattgagttttttttttttttatctttttggttcttttttagttcttgtcggaatcgtgagaagctctgtctctctgtcggaaccaatggggactccggagtttccagatctggggaaacactgctccgtcgatatctgcaagcaaatcgatttcttgcctttcacctgcgaccgttgtctccaggttcctctctctctatctctgttttgaatcacatgcaaaaaagaatcgtctttgttgcgaatagttgtgtcgtatgactgtctgaatcgcctgtcttcgatcctgggaactactatatcatcatccttgttcttttgtcgttcgtgttctgggagaaccaagtgaaaagtctccgtctttctcttaattaccgcgaggaaaattcatcttttctacatgcgaccagtttgatttgatttgttccgacattaagtttatcacttaagttcttgtaattacgaaccaacgtgttgagccatctgtcatttcgagcttaatcatcgacatttcgcaattctccgcacttaaattcaccagagttgttcaaaattcacttaattcatcatgattgccattaaagattcgatctttttgttggtttacattaacatatttgagtttatg

SEQ ID NO: 17 - DNA, 5717 bp, Brassica napus
  source 1..5717: mol_type = other DNA
catatatggatggacatagttggacatatttaggtccttatccaaatgatgttttaagatttgatttatggatgcacatagttggatacattttatgtttaaaatgtgtttttaatattattattgctgtggtgtgaaattttgtgtattcactttagttataatatctatgtataccaaagagaagaagataacatatattatagtattggtacatactataatataacatacttaattagtaaatttcacaccacagcaaaaataatataaaaggtcactttttaatatacaacgtgccaaacttcgtccatttataatcttatatatttttgtcttcgcatatgatagatcataagatttggagaaacatataaaattaacctatgaaattaaggggatctatatattatagtatgtatccaaagaacatatatatgaaatatttgaatacatcataagatttggggaatataaattaatataaaataaaatcgtccaattttaaacgtaaacgtctccgctgtatttatataaacttttcaaaatcacggtgtcgctcgtgttaagatttggatttctaatccaactttcagaaagtttatgtgaaagataatactcaaacacaatagttcgtttagaacgctcttaagattaatgtcacacacacaaaattgatgtcactagatttatatgttttgtcgtttaaggggatccatcaactacacatggaagaatagaaaacactggtcacacacaaaaaataatataagcatgttgagtcctattcatcagaaaattttctccaaaggatgaacaataacctacaattaaggaaacttattataacaacgaccaagaagttttcccataagtcatgctttcgtccataccttcgggtctttcaagcaagcttcacacatgccttatcgtatatttgtatacgttttcattttgtgtacgtctattaattagtatatgcctagattatatatttatatagtcgacaattacctatcctaaaggtttattttaatttttattcagaaagatatgcacgtagggccactagtgtttcccgttgttattggaaattccatgtgttgaactgtttgattaagtttttgaacccgtaagtcaaatttatttaggtttaacagggtttcattactgttataagcttgtcaagaaagaatttgtcacaaatttcgagactcatgcatgcatgcatgtttcttaactttcgagaaacatgaagtgtggttttaccagctgaagtttgtgttaaaacacatttttaatttgaccacgtgtgatattcaaatatcaacaattctgttcaacgggagattcatacatatgttcagatgttctttaatcttgagcacctaactttgcttcccatacgcaaatgtgtgtgccgcgaactatggtgaatgtacggcatcgtttttctttttggagcatttttaaaagaaaatataatttagttatttccgttttgattgatatagatgcattaataaaccgacttacgtgggtgatgggatgagcaaaattaaaatatgcattttagtaaccaattacggtccatcttgacaaactaacaaacaaaaagtctattatgacgttgatttgtgaccttgccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaatgacaactcctgttcttgtatcgtgtatctagcacgtaaatttgaattatggcggcattatcgataaaatatgtaaagaatatattttcatcatacaccacaaacaaatgttctggaaatatcaccttttatcacatatacaaagctgaccctgaatttttggaaactacaagccaaaaaaaaatggacaaaagaaaaattatgcacaaaagtggactcgtcctaactctttttataatgataattaacctttgaacaactaattaatacatacaatatgttttttttttctaaaaattggtcgattaaatattcatatataatcctcatcatcatttattagccttttgtagcttacttaaaaaatcacttcccaccaaattgattattttttcacttcatacgttgatcaatttaaaaaaaaaataataataatatatgcacgtccggcgtcccatgacttcagacgcttttctgtttccttgattttataggttagaaatttccaaaatgctcatatttttcactgatatgtattatacgcacaaaatagtaaagtcataaaaatttatgatggttcttttatatgtatagagtagtgaaaatttatatatattgtagttccgcttaaaataaattaatatttttcttttatcgcccatatttaagttactaaacgtatataattaaaaactgaaatgcaatatgtagaaatccgggcattgattgttgattaacattgatatacccatgcgactggcttatgatgaaccatacctgctaatactcattgttacaaactcgcagcaacatcataaatccatcactcgccacaataattttcttcagtgtatgttcaattgatcacattttgacccttcactttcttgaatgagaaattatacaagaaagtgatttgagtgtgttaaaaaatgatttgtcattctgaccaattatctggcagagagtataataaaatacttctaattaccttaaatcattaggcgttgtttcttgccgaatataagctagggtttccactccctcacgctaacacacatcaccgcagccatttccatctctcaaccaacttcaaacaatggctgaacgaggcggagaacgaggtgccgagcgaggaggagaccgcggcggtttcggacgcggattcggaggaggtcgcggcgacagaggaggccggggaggtccgagaggccgaggcggcagacgcggaggacgtccgacagaggaggagaagtggaccccagtgaccaagctcggccgtctcgtgcaagccggaaagatccagaagctcgagcagatctacctccactccctccccgtcaaggagtaccagatcatcgacctcctcgtcggccccaccttgaaagacgaggtgatgaagatcatgccggttcagaaacagaccagggccggtcagaggacgaggttcaaggcctttgtcgtcgtcggagacacgaacgggcacgttggtttgggtgtgaagtgctccaaggaggtggcgacggcgatcagaggtgggattatattggcgaagctgtcggttattcccgtgaggagagggtactgggggaacaagattgggaagccgcatacggttccttgtaaggttacggggaagtgtgggtctgttacggtgaggatggtgcctgctccgaggggtgcggggattgtggcggctagggttcctaagaaggtgcttcagttcgctgggattgatgatgtgtttacttcgtcgagggggtctactaagactcttggaaacttcgtaaaggtttgaactttcttcatttatttagagtttggagttgtgtatggattgaaagttgattcctttttgatggttgcgtgtggagttgttttgggtaatttagcttgttgtagttgatgtaaagtgagaattgctaagcttgattagtttgaataagctgtctgtgttaaatgatgatttgtttgtgttgtattgagctgtctgaatttcattatttgtttgtgttattgctaaattacaagcttaaatttgataacttttgattctttcgtgagagttttttttgggtaatatagcttgatgtagttgatggaagtaaattgttaacctttagagctgtctgttaatggtgatttgtttgtgttgtattgagctgtctgaattataatttgtttgtgtttattgctaaatcacaaagctttgaagtcgtttgtacatattcatcatttttgtgtttattgacttgtttgtgtttattgctaaatcacaagtttctatgattaatcttcatgatttggttgtgtttattactaaatcacaagtttctatgattaatcttaatgatttggttgtgtttattgctaaatttgagattacattataatcttagtttatacatgtataacatcatttcacttgaattttcacagacgagctggtgatatgtttttttagtattttccatgtattgctttgtaagaacttttaaatgtttgttttcttcctttgaagaacaattaaatgtttgttcagtatttttgaatcattcattggttaatcatgtgtaattttatttttgtaggctacattcgactgtctccagaagacttatggattcctgacaccagagttctggaaagagacgagctttaaaaagtcaccgtaccaggagtacactgatctgctggccgagaagggaattgcaaccaaagccatcaccgaggttgaagaccaagccgcctcctaaataaacaaacaaacaatgtcccccttgttttgtttttggatgatcctttcttgagttttattttgttcgaacctttagtatatgttgtttgctggattgctgctttcttatgttttcatcatctcccccaaacaaagttatttttgctctagtttttttttataatcgatggttatgccctctagtattttttttacttgaatttttcactaattttctatcaacttccctatgttacactttctaaggttgattacataggtggattgttgtgaaacaaatactaataataaactaatacttttagaataatataattagtgggattaaattataaattataaattaaaaagacaagtctgaaatggattgtttaactgtaaaaggtaaagagactttgaatagaagagaataaatcaaaagttgacgtatcttctccgaaatttcagctatttctaaatacgcggattctgattactctgttctctctcgcctgataaaccttagaattgagttttttttttttttatctttttggttcttttttagttcttgtcggaatcgtgagaagctctgtctctctgtcggaaccaatggggactccggagtttccagatctggggaaacactgctccgtcgatatctgcaagcaaatcgatttcttgcctttcacctgcgaccgttgtctccaggttcctctctctctatctctgttttgaatcacatgcaaaaaagaatcgtctttgttgcgaatagttgtgtcgtatgactgtctgaatcgcctgtcttcgatcctgggaactactatatcatcatccttgttcttttgtcgttcgtgttctgggagaaccaagtgaaaagtctccgtctttctcttaattaccgcgaggaaaattcatcttttctacatgcgaccagtttgatttgatttgttccgacattaagtttatcacttaagttcttgtaattacgaaccaacgtgttgagccatctgtcatttcgagcttaatcatcgacatttcgcaattctccgcacttaaattcaccagagttgttcaaaattcacttaattcatcatgattgccattaaagattcgatctttttgttggtttacattaacatatttgagtttatg

SEQ ID NO: 18 - DNA, 8882 bp, Brassica napus
  source 1..8882: mol_type = genomic DNA
gtgaaattttgtgttattcacttaaattataatatctatgtataccaaagagaagaagataacatatattatagtatttggtacatactatactataacatacttagtaaatttcacaccacaacaaaaataatataaaaggtcacattttaatatacaacgtgccaaacttcgtccatttgtaatcttatacattttcgtcttcgcatatgatagatcataagatttggagaaacatataaaattaacctatgaaattaagggggtctatatcttatagtatgtatccaaagaacatatataggaaatatttgaatacatcataagatttggggaatataaattaatataaaataaaatcttccaattttaaacgtaaacgtctcctctgtatttatataaacttttcaaaatcacggtgtcgctcgtgttaagatttggatttctaatccaactttcagaaagtatatgtgaaagataaaactcaaacacaatagttcgtttagaacgctcttaagattaatgtcacacacacaaaattgatgtcactagatttatttgttttgtcgtttaaggggatccatcaactacacatggaagaatagaaaacactggtcacacacaaaaaataatataagcatgttgaattctattcatcaaagaattttcttcaaaggatgaacaataacctataattaggaaacattataacaacgagcaggaagttttcccagaagtcatgccttcgtccataccttcgggtctttcaagcaagcttcacacatgccttatcatatgtttgtatacgttttcattttgtgtacgtctattaactagctagtatatgccaagattatatattttatatagtcgacaattacctatcctaaaagttcattttaatttttattcagaaagatatgcacgtagggccactagtgtttcccgttgttattggaaattcatttccatgtgttgaattgtttgtttaagtttttgaacccgtaagtcaaatttattttggtttaacagggtttcattactgttataagcttgtcaagaaagaatttgttacaaatttcgaggctcatgcatgcatgattcttaactttctagaaacatgaaatgtggttttaccagctcaagcttgtgttaaaacacatttttaatttgaccacgtgtgatattcaaatatcaacaattctgttcaacgggagattcatacatatgttcagatgttctttaatcttaagcacctaactttgcttcccatacgcaaatgtgtgtgccgcgaactatggtgaaatgttttctttttggagcatttttaaaagaaaatataatttacttatttccgttttgattgatatacatgcattaataaaccgacttacgtgggtgatgggatgagcaaaattaaaatatgcattttagtaaccaattacggtctatcttgacaaactaacaaacaaaaagtctattatgacgttgatttgtgacctggccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaaatacaactcctgttcttgtatcgtgtatctctcgcacgtaaatttgaattatgggggcattatcgataaaatatgtaaagaatatattttcatcatacaccacaaacaaatgttctggaaatgtcaccttttatcacatatacaaagctgaccctgaaattttggaaactacaagccaaaaaaaatggtcaaaagaaatattatgcacaaaggttggactcgtcctaactctttttacaatgataattaacctttgaacaactaattaaagatacatacaatatgttttttttctaaaaattggtcgattaaatattcatatataatccttatcatcatttattagccttttgtagcttacttaaaaaaccccttcccaccaaattgattagtgtagattgattatttttcacttcatacgttgatcaattttaaaatatatatatatatatatatatatatatatatgcacgtccggcgtcccatgacttcagacgcttttctgtttccttgattttataggttaaaattttccaaaatgctcatatttttcactcatatgtataatgtattattatacgcacaaaatagtaaagtcataaacatttatgatggctcttttatatgtatagagtagtgaaatttatatatatcggagttccgcttaaaataaattaatattgttcttttatcgcccattttttagttactaaacgtatataattaaaaactgaaatgcaatatgtagaaatccggcttatgatgaaccatactaatactcattgttacaaactcacagcaacatcataattccatcactcgccacaataattttctttagtgtatgttctatttcacattttgacccttcactttcttgaatgaaaattatacaagaaaatgatttgagtgtgttaaaaaaaaaatgttttgagtcattctgaccaattatctggcagagactataataaaatacttccaattaccttaaatcattaggcgttgtttcttgccgaatatatgatggagcatggtctcgctaagagacaacaaaatctttaaataaattaaacaagttgtttagcaaaaaataaaaaataaattaaacaagagagagtacaaatataaaactctaaaggaagataaatgtataaaatacaaaatatagaatagaggccttaagtagtgtttacgtgacaatctcaatttcactccatacctcgaactcattcaagcttagaaacctaacttttactgttccttttgatttgcctataaatacacgttcatccatacttctgtaatttcaaataacaaagcaaaacacaaagataagaatcacaaacaacaaacattctcaacctaatcccaaaaagacgacacgcattcctttttccaaaaatccacttcctacgtaccaaccacaatggctcattgttggacagttcttaccggtttactagtatggtctcttttgatgctctcatggtgcgaagcttcaagaaacaacattaatagatatgatcattcttatggaacgttcaaatccaatagcttaatcaagcgaagagacgatatcactcgtctgaaaagtgttgcaagagcttctttgcggcttccaaccaccgttagtgtttctgattttggagctaaaggagatggaaaaaccgacgacacgcaggtatatacgcatacacacatatctacatgacttataagttataaatattgtgagacggtcacgtatataaatttacgttgtttgtactaatgatccatgcatgcgcatgcatatgatcaggcgttcgtgaatgcatggaagaaagcttgttcttcaagtggacctgtaaatctcttagttcctgaaggaaaaacttattttctcaagtctcttcgattaaatggcccatgcaaatcagttcttaccgttcaggtaaattacaaagtttcttttgttttgcttaaatttgggtaaattacaaagttgacaaaaaaattacaaagttgactatttgttaattatatacgtatactacatgtgatcacgtatatggttttgaatatattaaagagcggttggtgaatggaacgacagattttaggaacgttatcggcatctcaacaacgatcggattataaagatctcagcaaatggattacgtttgacggcgttaacagtctaacggttgatggcggggccaccggaactgtcaacggaaacggcgaaacgtggtgggaaaactcatgcaaacggaacgaagctaaggtgtgtgtctgtacatgtttcatttacttaatatatatctactatctacatgtcatcttgatatattgacacttttttattgttattgttttgaagaaatgcacaaaagccccaacggtacgtagaaacatatttgacattttcatgtaatactgacgatggctgtgatcatgattggacaaatgttttacatgcatgaatatatgtattttatatgcaggctcttactttctacaactcgaagaatttgagagttaataatctgagggtgaaagatgcgcaacagatccagatttcgattgagaaatgctccaacgttcaggtctcaaatgttgaggtcactgcgcctgctgatagtcctaacactgatggtatccacatcactaacactcaaaacattcaagtctccaaatccatcattggaacaggttagtaattacatcctcgttatttaaaagagttgcgtgcatatccagaaaactaatatttaagggtttatcgttataatttatagagattggtcttaaaagagttttttaatgtttgcttttctatgcaggtgatgattgtatatctattgaaagtggatcacaaaacgttaaaatcaacgatttaacttgcggccccggtcacggtatcaggtatcaatgaattgttttcagtaaatatacctaagaaatacaaccgtaatcttttacatataaatttgaaatttccttataaaacatgagcagcattgggagcttaggagatgacaattcaaaggcatttgtatcaggcgtgactgtggatggtgctaaactttccggtacagacaatggagttaggatcaaaacttaccaggtgaaaatcacaaactaaaccttcaattttaactagcctatcaagttcatcccaatttaacttttaaacccaattcaatatagggagggtctggaactgctagcaacataatatttcagaacattcagatggagaatgtgaagaatccaatcataatcgaccaagattattgcgacaagagcaaatgcaccgaacaagttcgtttgaacctaatcaaatcttccattactaatcctcatgataataaacacttttatctctaatcatttactacatatattttcagaaatcagcggtccaaatcaagaacgtggtgtaccggaacataagcggcacaagcgcatcagacatagcaataactttcaactgtagcaagaactatccatgccaaggaattgtactcgacaaagtgaacattaagggagggaaagcgagttgcagcaacgctaatgtgatggataaagacggtgttttgcctcagtgcaaatccacttgattaactagacctatcaaaaactcacacagatgtataaatacatatagaccgatacaaagtatacaaaattatgatatatacttgtggatatatacatggacaatgaatttaaacatattatttatatgtcaaaatagtttatgaatagacatataaacaacatgttcaagagatttattgtcttaatgtatatactccatactaaattgtttgtctaaaacttcatataataaaaaactgttaatcttcatgatggtgtgatttgttgtgataacagtatgatgtctattgtagatttttatttaaatacataaaaattgaaagaaaaaaaatgacatggctacttgaccaattgattcaagttaattgttactaaccaggccgataaactaattgggcctttaaagcccattaatatttgtcagtggaagcgaagtctgttacttgggttacaaaacaaaggcctttagaaaaccctaactcttatcgcagcacctgagctatatattgccgcagctagggttttccctagctcacgctaacacacaaagccgcagccatttccatctctctcgcaactcaacttcgaacaatggctgaacgaggcggagaacgaggcgccgagcgaggaggagaccgcggcggtttcggacggggattcggaggaggtcgcggcgaccgaggaggccgcggaggtccgagaggccgaggcggcagacgcggaggacgtcccacagaggaggagaagtggaccccagtgaccaagctcggccgtctcgtgcaagccggaaagatccagaagctcgagcagatctacctccactcgctccccgtcaaggagtaccagatcatcgacctcctcgtcggtccaactttaaaagacgaggtgatgaagatcatgccggttcagaaacagaccagggccggtcagaggacgaggttcaaggcttttgtcgttgtcggagacacgaatggtcatgttggtttgggtgtgaagtgctccaaggaggtggcgacggcgatcagaggtgggattatattggcgaagctgtcggtgattcccgtgaggagagggtactgggggaacaagattgggaagccgcatacggttccttgtaaggttacggggaagtgtgggtctgttacggtgaggatggtgcctgctccgaggggggcggggattgtggcggctagggttcctaagaaggtgcttcagttcgctgggattgatgatgtgtttacttcttccaggggatctactaagactcttggaaacttcgtaaaggtttgaactttattcatttacttggtgtttggagctgtgtatagattgaaagttgattcctttttgatcgttgcgtgtggaggtgttttgggtaatattgctaaatgtaaaaattgctaagcttgattagtttgaataagctgtctgtgttaaatgatgatttgtttgtgttgtaatgagctgtctgaatttcattatttgtttgtgttattgctaatttacaagcttaaatttgattccttttgatcgtttcgtgtgagttgttttgtgtaatttagcttgatgtagttgatggaagtaagaattgttaagctttagagctgtctgttaatgttgaattgtttgtgttgtattgagctgtctgaattattatttgtttgcgatttatttctaaatcacaaagctctaaagtcgtttgaacatatgcatcatttggttagtgtttattgctgaattttcatgatttggttgtgtttattgctaaatttgagattacattataatcttaatttgtacatatattcatcatttttgtgtttattgatttgtttgtgtttattgatgaatcacaagtttctatgattaatcttcatgatttggttgtgattattgctaaatttgagattacattataagcttagtttgtacatgtatacatcatttcacttgaattttcacagacgagctggtgatatatttttttttttagtattttccatgtattgctttgtaagaacgtttaaatgttagttttcttgaatgtttttttctttgtaagaacaattaaatgtttgttcagtatttttgaatcattcattggttaatcatgttcaacatgtattttttgtaggctacatttgactgtctccagaagacttatggattcctgacaccagagttctggaaagagacgagctttaaaaagtcaccgtaccaggagtacactgatttgctggccgagaagggaactgcaaccaaagccatcaccgaggttgaagaccaagccgcctcctaaataaacaaacaagcaatgtccaccttgttttgtttttggatgatcctttcatgagttttattttgttcgaaccttttagtatatgttgtttgctggattgctgctatcttaagttttcatcatcttcccaaacaaagttatttttgctctagtttttttttttttaataatcgatggttatactctagtatttttttacttgaatttctctaattttctatcagttgcgctatgttacacattctaaggttgattacatagttgatccgaaatgattaaaaaggctggatgttgtaataaattgttggatatacttttagaataatataattagggggattaaattataaattaaaattaaaaaagacaagtcttaaatggattgttgaactgtgagaaggtgaaaaggtggagactttgaatagaagagaataaatcaaaagttgacgtatcttctccgaaatttcagctgtttctaaatacgcggattctgattactctgttctctcttgcctgataaaccttcgaattgagttttttttttttttttcatttctttttttggttcttgtcggaatcgtgagaagctctgtctctttgtcggaaccaatggggactccggaatttccagatctggggaaacactgctccgtcgatatctgcaagcagatcgatttcttgcctttcacctgcgaccgttgtctccaggttcctctttccttctctctctgttttgaatcacatgcaaaatgaatcgtctttgttgcgaataattgtgttgtatcactgtctgaatcgcatgtcttcgatcctgggaactactatatcatcttccttgttcttttgtcgttcgtgttcttggagaaccaagagaaaagtccccgtctttctcttattacccaagaggaaaatcatcttttctacatgcgaccagtttgatataggatcgagtttgatttgatttgttccgacattaagtttgtcacttaagttctttgtaattacgaccttgttctttggcatttcgcaattctccgcacttaattcaccagagttcttcaaaattcacttaactcatcatgattgccattaaagattcgatctttttgttggtttacattaacatatttgagtttatggctggttcttatgttttcgctagaaatttgcatttaaagatttgttctttttatgttaatgacttgtgtgttcgctcttgtttacattgatttgatctttttcaggtcttttgtttggatcatcggagctatatgaaacacacttgccccaaagg

SEQ ID NO: 19 - DNA, 4463 bp, Brassica napus
  source 1..4463: mol_type = other DNA
catatatggatggacatagttggacatatttaggtccttatccaaatgctggtgttaagaattgatttatggatgtacatagttggatacattttatgtttaaaatgtgatttttatattattattgctgtggtgtgaaattttgtgttattcacttaaattataatatctatgtataccaaagagaagaagataacatatattatagtatttggtacatactatactataacatacttagtaaatttcacaccacaacaaaaataatataaaaggtcacattttaatatacaacgtgccaaacttcgtccatttgtaatcttatacattttcgtcttcgcatatgatagatcataagatttggagaaacatataaaattaacctatgaaattaagggggtctatatcttatagtatgtatccaaagaacatatataggaaatatttgaatacatcataagatttggggaatataaattaatataaaataaaatcttccaattttaaacgtaaacgtctcctctgtatttatataaacttttcaaaatcacggtgtcgctcgtgttaagatttggatttctaatccaactttcagaaagtatatgtgaaagataaaactcaaacacaatagttcgtttagaacgctcttaagattaatgtcacacacacaaaattgatgtcactagatttatttgttttgtcgtttaaggggatccatcaactacacatggaagaatagaaaacactggtcacacacaaaaaataatataagcatgttgaattctattcatcaaagaattttcttcaaaggatgaacaataacctataattaggaaacattataacaacgagcaggaagttttcccagaagtcatgccttcgtccataccttcgggtctttcaagcaagcttcacacatgccttatcatatgtttgtatacgttttcattttgtgtacgtctattaactagctagtatatgccaagattatatattttatatagtcgacaattacctatcctaaaagttcattttaatttttattcagaaagatatgcacgtagggccactagtgtttcccgttgttattggaaattcatttccatgtgttgaattgtttgtttaagtttttgaacccgtaagtcaaatttattttggtttaacagggtttcattactgttataagcttgtcaagaaagaatttgttacaaatttcgaggctcatgcatgcatgattcttaactttctagaaacatgaaatgtggttttaccagctcaagcttgtgttaaaacacatttttaatttgaccacgtgtgatattcaaatatcaacaattctgttcaacgggagattcatacatatgttcagatgttctttaatcttaagcacctaactttgcttcccatacgcaaatgtgtgtgccgcgaactatggtgaaatgttttctttttggagcatttttaaaagaaaatataatttacttatttccgttttgattgatatacatgcattaataaaccgacttacgtgggtgatgggatgagcaaaattaaaatatgcattttagtaaccaattacggtctatcttgacaaactaacaaacaaaaagtctattatgacgttgatttgtgacctggccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaaatacaactcctgttcttgtatcgtgtatctctcgcacgtaaatttgaattatgggggcattatcgataaaatatgtaaagaatatattttcatcatacaccacaaacaaatgttctggaaatgtcaccttttatcacatatacaaagctgaccctgaaattttggaaactacaagccaaaaaaaatggtcaaaagaaatattatgcacaaaggttggactcgtcctaactctttttacaatgataattaacctttgaacaactaattaaagatacatacaatatgttttttttctaaaaattggtcgattaaatattcatatataatccttatcatcatttattagccttttgtagcttacttaaaaaaccccttcccaccaaattgattagtgtagattgattatttttcacttcatacgttgatcaattttaaaatatatatatatatatatatatatatatatatgcacgtccggcgtcccatgacttcagacgcttttctgtttccttgattttataggttaaaattttccaaaatgctcatatttttcactcatatgtataatgtattattatacgcacaaaatagtaaagtcataaacatttatgatggctcttttatatgtatagagtagtgaaatttatatatatcggagttccgcttaaaataaattaatattgttcttttatcgcccattttttagttactaaacgtatataattaaaaactgaaatgcaatatgtagaaatccggcttatgatgaaccatactaatactcattgttacaaactcacagcaacatcataattccatcactcgccacaataattttctttagtgtatgttctatttcacattttgacccttcactttcttgaatgaaaattatacaagaaaatgatttgagtgtgttaaaaaaaaaatgttttgagtcattctgaccaattatctggcagagactataataaaatacttccaattaccttaaatcattaggcgttgtttcttgccgaatatactagggttttccctagctcacgctaacacacaaagccgcagccatttccatctctctcgcaactcaacttcgaacaatggctgaacgaggcggagaacgaggcgccgagcgaggaggagaccgcggcggtttcggacggggattcggaggaggtcgcggcgaccgaggaggccgcggaggtccgagaggccgaggcggcagacgcggaggacgtcccacagaggaggagaagtggaccccagtgaccaagctcggccgtctcgtgcaagccggaaagatccagaagctcgagcagatctacctccactcgctccccgtcaaggagtaccagatcatcgacctcctcgtcggtccaactttaaaagacgaggtgatgaagatcatgccggttcagaaacagaccagggccggtcagaggacgaggttcaaggcttttgtcgttgtcggagacacgaatggtcatgttggtttgggtgtgaagtgctccaaggaggtggcgacggcgatcagaggtgggattatattggcgaagctgtcggtgattcccgtgaggagagggtactgggggaacaagattgggaagccgcatacggttccttgtaaggttacggggaagtgtgggtctgttacggtgaggatggtgcctgctccgaggggggcggggattgtggcggctagggttcctaagaaggtgcttcagttcgctgggattgatgatgtgtttacttcttccaggggatctactaagactcttggaaacttcgtaaaggtttgaactttattcatttacttggtgtttggagctgtgtatagattgaaagttgattcctttttgatcgttgcgtgtggaggtgttttgggtaatattgctaaatgtaaaaattgctaagcttgattagtttgaataagctgtctgtgttaaatgatgatttgtttgtgttgtaatgagctgtctgaatttcattatttgtttgtgttattgctaatttacaagcttaaatttgattccttttgatcgtttcgtgtgagttgttttgtgtaatttagcttgatgtagttgatggaagtaagaattgttaagctttagagctgtctgttaatgttgaattgtttgtgttgtattgagctgtctgaattattatttgtttgcgatttatttctaaatcacaaagctctaaagtcgtttgaacatatgcatcatttggttagtgtttattgctgaattttcatgatttggttgtgtttattgctaaatttgagattacattataatcttaatttgtacatatattcatcatttttgtgtttattgatttgtttgtgtttattgatgaatcacaagtttctatgattaatcttcatgatttggttgtgattattgctaaatttgagattacattataagcttagtttgtacatgtatacatcatttcacttgaattttcacagacgagctggtgatatatttttttttttagtattttccatgtattgctttgtaagaacgtttaaatgttagttttcttgaatgtttttttctttgtaagaacaattaaatgtttgttcagtatttttgaatcattcaatggttaatcatgttcaacatgtattttttgtaggctaca

SEQ ID NO: 20 - DNA, 4789 bp, Brassica napus
  source 1..4789: mol_type = other DNA
tgccttatgaagatctacgtctaactgcatcatctgagccatcctatgaaaatttatttttgaccagatttacttgcaccatgttgaaacttctttataaacctttcttttctagtttgcattaataaatcgctcttttctctttctctgatacatattattcatgttggttatacctgtacacgcacggacaatattttccaactttaaaacgaatcaaatcagttaatttaataactttggactagtaactgtatttgaatttaaatttctcttttcagaatattctacaaaacagttttaattccgtttgtcagtagaatcacatatatggatggacatagttggacatatttaggtccttatccaaatgctggtgttaagaattgatttatggatgtacatagttggatacattttatgtttaaaatgtgatttttatattattattgctgtggtgtgaaattttgtgttattcacttaaattataatatctatgtataccaaagagaagaagataacatatattatagtatttggtacatactatactataacatacttagtaaatttcacaccacaacaaaaataatataaaaggtcacattttaatatacaacgtgccaaacttcgtccatttgtaatcttatacattttcgtcttcgcatatgatagatcataagatttggagaaacatataaaattaacctatgaaattaagggggtctatatcttatagtatgtatccaaagaacatatataggaaatatttgaatacatcataagatttggggaatataaattaatataaaataaaatcttccaattttaaacgtaaacgtctcctctgtatttatataaacttttcaaaatcacggtgtcgctcgtgttaagatttggatttctaatccaactttcagaaagtatatgtgaaagataaaactcaaacacaatagttcgtttagaacgctcttaagattaatgtcacacacacaaaattgatgtcactagatttatttgttttgtcgtttaaggggatccatcaactacacatggaagaatagaaaacactggtcacacacaaaaaataatataagcatgttgaattctattcatcaaagaattttcttcaaaggatgaacaataacctataattaggaaacattataacaacgagcaggaagttttcccagaagtcatgccttcgtccataccttcgggtctttcaagcaagcttcacacatgccttatcatatgtttgtatacgttttcattttgtgtacgtctattaactagctagtatatgccaagattatatattttatatagtcgacaattacctatcctaaaagttcattttaatttttattcagaaagatatgcacgtagggccactagtgtttcccgttgttattggaaattcatttccatgtgttgaattgtttgtttaagtttttgaacccgtaagtcaaatttattttggtttaacagggtttcattactgttataagcttgtcaagaaagaatttgttacaaatttcgaggctcatgcatgcatgattcttaactttctagaaacatgaaatgtggttttaccagctcaagcttgtgttaaaacacatttttaatttgaccacgtgtgatattcaaatatcaacaattctgttcaacgggagattcatacatatgttcagatgttctttaatcttaagcacctaactttgcttcccatacgcaaatgtgtgtgccgcgaactatggtgaaatgttttctttttggagcatttttaaaagaaaatataatttacttatttccgttttgattgatatacatgcattaataaaccgacttacgtgggtgatgggatgagcaaaattaaaatatgcattttagtaaccaattacggtctatcttgacaaactaacaaacaaaaagtctattatgacgttgatttgtgacctggccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaaatacaactcctgttcttgtatcgtgtatctctcgcacgtaaatttgaattatgggggcattatcgataaaatatgtaaagaatatattttcatcatacaccacaaacaaatgttctggaaatgtcaccttttatcacatatacaaagctgaccctgaaattttggaaactacaagccaaaaaaaatggtcaaaagaaatattatgcacaaaggttggactcgtcctaactctttttacaatgataattaacctttgaacaactaattaaagatacatacaatatgttttttttctaaaaattggtcgattaaatattcatatataatccttatcatcatttattagccttttgtagcttacttaaaaaaccccttcccaccaaattgattagtgtagattgattatttttcacttcatacgttgatcaattttaaaatatatatatatatatatatatatatatatatgcacgtccggcgtcccatgacttcagacgcttttctgtttccttgattttataggttaaaattttccaaaatgctcatatttttcactcatatgtataatgtattattatacgcacaaaatagtaaagtcataaacatttatgatggctcttttatatgtatagagtagtgaaatttatatatatcggagttccgcttaaaataaattaatattgttcttttatcgcccattttttagttactaaacgtatataattaaaaactgaaatgcaatatgtagaaatccggcttatgatgaaccatactaatactcattgttacaaactcacagcaacatcataattccatcactcgccacaataattttctttagtgtatgttctatttcacattttgacccttcactttcttgaatgaaaattatacaagaaaatgatttgagtgtgttaaaaaaaaaatgttttgagtcattctgaccaattatctggcagagactataataaaatacttccaattaccttaaatcattaggcgttgtttcttgccgaatatagctagggttttccctagctcacgctaacacacaaagccgcagccatttccatctctctcgcaactcaacttcgaacaatggctgaacgaggcggagaacgaggcgccgagcgaggaggagaccgcggcggtttcggacggggattcggaggaggtcgcggcgaccgaggaggccgcggaggtccgagaggccgaggcggcagacgcggaggacgtcccacagaggaggagaagtggaccccagtgaccaagctcggccgtctcgtgcaagccggaaagatccagaagctcgagcagatctacctccactcgctccccgtcaaggagtaccagatcatcgacctcctcgtcggtccaactttaaaagacgaggtgatgaagatcatgccggttcagaaacagaccagggccggtcagaggacgaggttcaaggcttttgtcgttgtcggagacacgaatggtcatgttggtttgggtgtgaagtgctccaaggaggtggcgacggcgatcagaggtgggattatattggcgaagctgtcggtgattcccgtgaggagagggtactgggggaacaagattgggaagccgcatacggttccttgtaaggttacggggaagtgtgggtctgttacggtgaggatggtgcctgctccgaggggggcggggattgtggcggctagggttcctaagaaggtgcttcagttcgctgggattgatgatgtgtttacttcttccaggggatctactaagactcttggaaacttcgtaaaggtttgaactttattcatttacttggtgtttggagctgtgtatagattgaaagttgattcctttttgatcgttgcgtgtggaggtgttttgggtaatattgctaaatgtaaaaattgctaagcttgattagtttgaataagctgtctgtgttaaatgatgatttgtttgtgttgtaatgagctgtctgaatttcattatttgtttgtgttattgctaatttacaagcttaaatttgattccttttgatcgtttcgtgtgagttgttttgtgtaatttagcttgatgtagttgatggaagtaagaattgttaagctttagagctgtctgttaatgttgaattgtttgtgttgtattgagctgtctgaattattatttgtttgcgatttatttctaaatcacaaagctctaaagtcgtttgaacatatgcatcatttggttagtgtttattgctgaattttcatgatttggttgtgtttattgctaaatttgagattacattataatcttaatttgtacatatattcatcatttttgtgtttattgatttgtttgtgtttattgatgaatcacaagtttctatgattaatcttcatgatttggttgtgattattgctaaatttgagattacattataagcttagtttgtacatgtatacatcatttcacttgaattttcacagacgagctggtgatatatttttttttttagtattttccatgtattgctttgtaagaacgtttaaatgttagttttcttgaatgtttttttctttgtaagaacaattaaatgtttgttcagtatttttgaatcattcaatggttaatcatgttcaacatgtattttttgtaggctaca

SEQ ID NO: 21 - DNA, 4465 bp, Brassica napus
  source 1..4465: mol_type = other DNA
catatatggatggacatagttggacatatttaggtccttatccaaatgctggtgttaagaattgatttatggatgtacatagttggatacattttatgtttaaaatgtgatttttatattattattgctgtggtgtgaaattttgtgttattcacttaaattataatatctatgtataccaaagagaagaagataacatatattatagtatttggtacatactatactataacatacttagtaaatttcacaccacaacaaaaataatataaaaggtcacattttaatatacaacgtgccaaacttcgtccatttgtaatcttatacattttcgtcttcgcatatgatagatcataagatttggagaaacatataaaattaacctatgaaattaagggggtctatatcttatagtatgtatccaaagaacatatataggaaatatttgaatacatcataagatttggggaatataaattaatataaaataaaatcttccaattttaaacgtaaacgtctcctctgtatttatataaacttttcaaaatcacggtgtcgctcgtgttaagatttggatttctaatccaactttcagaaagtatatgtgaaagataaaactcaaacacaatagttcgtttagaacgctcttaagattaatgtcacacacacaaaattgatgtcactagatttatttgttttgtcgtttaaggggatccatcaactacacatggaagaatagaaaacactggtcacacacaaaaaataatataagcatgttgaattctattcatcaaagaattttcttcaaaggatgaacaataacctataattaggaaacattataacaacgagcaggaagttttcccagaagtcatgccttcgtccataccttcgggtctttcaagcaagcttcacacatgccttatcatatgtttgtatacgttttcattttgtgtacgtctattaactagctagtatatgccaagattatatattttatatagtcgacaattacctatcctaaaagttcattttaatttttattcagaaagatatgcacgtagggccactagtgtttcccgttgttattggaaattcatttccatgtgttgaattgtttgtttaagtttttgaacccgtaagtcaaatttattttggtttaacagggtttcattactgttataagcttgtcaagaaagaatttgttacaaatttcgaggctcatgcatgcatgattcttaactttctagaaacatgaaatgtggttttaccagctcaagcttgtgttaaaacacatttttaatttgaccacgtgtgatattcaaatatcaacaattctgttcaacgggagattcatacatatgttcagatgttctttaatcttaagcacctaactttgcttcccatacgcaaatgtgtgtgccgcgaactatggtgaaatgttttctttttggagcatttttaaaagaaaatataatttacttatttccgttttgattgatatacatgcattaataaaccgacttacgtgggtgatgggatgagcaaaattaaaatatgcattttagtaaccaattacggtctatcttgacaaactaacaaacaaaaagtctattatgacgttgatttgtgacctggccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaaatacaactcctgttcttgtatcgtgtatctctcgcacgtaaatttgaattatgggggcattatcgataaaatatgtaaagaatatattttcatcatacaccacaaacaaatgttctggaaatgtcaccttttatcacatatacaaagctgaccctgaaattttggaaactacaagccaaaaaaaatggtcaaaagaaatattatgcacaaaggttggactcgtcctaactctttttacaatgataattaacctttgaacaactaattaaagatacatacaatatgttttttttctaaaaattggtcgattaaatattcatatataatccttatcatcatttattagccttttgtagcttacttaaaaaaccccttcccaccaaattgattagtgtagattgattatttttcacttcatacgttgatcaattttaaaatatatatatatatatatatatatatatatatgcacgtccggcgtcccatgacttcagacgcttttctgtttccttgattttataggttaaaattttccaaaatgctcatatttttcactcatatgtataatgtattattatacgcacaaaatagtaaagtcataaacatttatgatggctcttttatatgtatagagtagtgaaatttatatatatcggagttccgcttaaaataaattaatattgttcttttatcgcccattttttagttactaaacgtatataattaaaaactgaaatgcaatatgtagaaatccggcttatgatgaaccatactaatactcattgttacaaactcacagcaacatcataattccatcactcgccacaataattttctttagtgtatgttctatttcacattttgacccttcactttcttgaatgaaaattatacaagaaaatgatttgagtgtgttaaaaaaaaaatgttttgagtcattctgaccaattatctggcagagactataataaaatacttccaattaccttaaatcattaggcgttgtttcttgccgaatataagctagggttttccctagctcacgctaacacacaaagccgcagccatttccatctctctcgcaactcaacttcgaacaatggctgaacgaggcggagaacgaggcgccgagcgaggaggagaccgcggcggtttcggacggggattcggaggaggtcgcggcgaccgaggaggccgcggaggtccgagaggccgaggcggcagacgcggaggacgtcccacagaggaggagaagtggaccccagtgaccaagctcggccgtctcgtgcaagccggaaagatccagaagctcgagcagatctacctccactcgctccccgtcaaggagtaccagatcatcgacctcctcgtcggtccaactttaaaagacgaggtgatgaagatcatgccggttcagaaacagaccagggccggtcagaggacgaggttcaaggcttttgtcgttgtcggagacacgaatggtcatgttggtttgggtgtgaagtgctccaaggaggtggcgacggcgatcagaggtgggattatattggcgaagctgtcggtgattcccgtgaggagagggtactgggggaacaagattgggaagccgcatacggttccttgtaaggttacggggaagtgtgggtctgttacggtgaggatggtgcctgctccgaggggggcggggattgtggcggctagggttcctaagaaggtgcttcagttcgctgggattgatgatgtgtttacttcttccaggggatctactaagactcttggaaacttcgtaaaggtttgaactttattcatttacttggtgtttggagctgtgtatagattgaaagttgattcctttttgatcgttgcgtgtggaggtgttttgggtaatattgctaaatgtaaaaattgctaagcttgattagtttgaataagctgtctgtgttaaatgatgatttgtttgtgttgtaatgagctgtctgaatttcattatttgtttgtgttattgctaatttacaagcttaaatttgattccttttgatcgtttcgtgtgagttgttttgtgtaatttagcttgatgtagttgatggaagtaagaattgttaagctttagagctgtctgttaatgttgaattgtttgtgttgtattgagctgtctgaattattatttgtttgcgatttatttctaaatcacaaagctctaaagtcgtttgaacatatgcatcatttggttagtgtttattgctgaattttcatgatttggttgtgtttattgctaaatttgagattacattataatcttaatttgtacatatattcatcatttttgtgtttattgatttgtttgtgtttattgatgaatcacaagtttctatgattaatcttcatgatttggttgtgattattgctaaatttgagattacattataagcttagtttgtacatgtatacatcatttcacttgaattttcacagacgagctggtgatatatttttttttttagtattttccatgtattgctttgtaagaacgtttaaatgttagttttcttgaatgtttttttctttgtaagaacaattaaatgtttgttcagtatttttgaatcattcaatggttaatcatgttcaacatgtattttttgtaggctaca

SEQ ID NO: 22 - DNA, 6656 bp, Brassica napus
  source 1..6656: mol_type = genomic DNA
aaaaatatttgatttatatattcttattaaaaatattaatttactgtttattattttatataattttataaatcattttatttagagtttaatttatttacgatttattgttttaatttgttaggcaaattagtctaacaaattaaacaaaaaaaaatataaataaattaaattttaaacaaaatgttttataaaaatatttatatccttataaggatataaactataattttgtgttatttttatatttaaactattattatatttaattatttttaaataaatttatttatatttttgtatatttttattaaaactaatattttatttaattatgaaaattgaaaacgtttttttggttatttttgaaaattttgacaaggttgaaaaatcgctggaaatatttttgaggaattttagacaaaaaaatatcgatgataaacaattggttggaatccgtcaagaatttttgacaaaataaactcgaaatttgttttcactgggaattagttattttcttgtatttgcattatctaataaacaatgcaaccataaacaacaaatgaaataaagttttaattgacaaatcttttagaagagtgaaacgttaatggtggacgaagcccccacctcaattgacaataaaaagagtggaaaagagggaagatggcagagagaaaagtccaaagagaggaaatcatgaagagaagaagacaatgtaacctacttatttgcctcgatgaagatagaggagaagatagaggtggaatatatatagaagagagaaagatttgggaggaaacccgattgagcatatatcagatctagaatggattggattagataactcgaacttatttggttaatctttttagtttagggaaggttatgaaaaattacctctgatctaatttttaaattttttatcttatatacattgtgccccccgactaacaatatatctagatccatccctgattaggtccatgagatatatcatattttttatttttcttatttaattaattatctataatatttgttgatatatttaaaattttacatatcgttgaaagccagaaaaatatgtggaagtgtaataaaacaattttctccaggaagaagatatttcagccattcgcccatctatgtgtatgagatcccattcaaggccatattctaagattgactttatctaatttactgtattttccgtgggctactgaatgaatacgatgcttttgttgtaaaacgcaacctattgttctccattttgaatttgaagtgttacattgttgataaaaaggaagaaagtacaaagcttttcatttagtggttgttgcatatatatacttatttgatgggtttatgataagtatgaacatttcacgtaacgggtttattttgtcgtcaacacattatcggcaatgtgaaaagaaggggtgaggtatctccatttcaattcttctctttatatattaatcgaattatttacgtatgaaatgaacgtttatatagaaatttcgtgtggaaaacgacatgtacacggcatctcaagaccaattagtaatatactttagtggtgattacatgtttacttatccaattgagaatttaaagcatcgacaataccttaatgtcgattaagccgtccccacttcatgtaatgagttatggggggagagagagatcccgaaattcgtcaaataaaacaacttagaactaaaaaccgacaccaagtatcataaaggaaatgttgaagaagtcatttatcgtatccagctcacaattcctaagattaaatcatgaccgttggaagagcttataagattaaactgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctatatggcaatgatatcatcaccaaagtgcaatgcccctttttgccctagtttcgtaaagtctctctccttcttcgtccttaggaaaaaccctaaattaaatcctgtgttcttgatctttctttttgagtaaccatgattttgaccacacactagttcttctatattttgtggtctataggattttgctttatatgtgtttcttgtattgctccgtacgtgcgtatataaatttaaatggttacaacaaggtttattataaataggcacaaattagtccatgaagttatttagcttgcacaagtataatttgttaagtatttaaatatataaatttgttacaaaacttaattaaatttatctgattatattttctttagtgttcttcctttgccaacgttgaggtagctattattattattattttgaacattatgtacgtagttatcttggctagttatgattcgaattcttaatttggatcacacttaacagtatttaaaatattcttagaactaaaataattaagagttacctttaaattgaagtattcgtgctaaacagaaactagaataaacaaatgattgcatgttaattttttttttcgattttcctatcagaataaacacatgattgcatgcaaatattgtttttgattacgttatcttttgtttattttagttttgatgctaattaatattttttattaacaactcacatacattctacctgattctaggtcagataatgacacagcgcaacaaaattaatacaaaaccttcggaaagtagaataccgcagaagtaacttttttgggtacatacgaaatacagtgaaatctctataaattaataatgttgagactataccaaaactataaaatttttattaatttatagagattaatttatcgatatactaattgaatcaaaaacttaatttgagattaaaaaattatattattttatagagatttttagtgtatattaatttatagaatattattttataaaaaattttagtgtgtattaatttatagagtattaatttaaagaagttatactgtaatgtgaatcttcgaaaaacatgccatacataaccacggatcatagtcgacacctcaacgtgaagcaaatttgacaatctacatacataaccaacaaaaagtagaataccttgaaaatctaaaacccaaaatatgatgtaaaactcaagcttggtccagagcataaaaaaattaaagccatcgctttggtatcacatatttaaacgtcagttttttttttttttttggggggggggggggggggtaatataaaaatataattaacaaaaacaatttatgaaacaattagcatgtaaaacactaatcttttggttgtgacaaaacgttttcacaaatgttctataaataaattcaagtgcattttatctgcaaaatatatactttcactcataaaataagagcgtttaaaacattcatacacgcactacattgacatgacaaaagaaatccgcaaatacacatgatgtatgtcgaaaaaaacaaaaaaatacacatgatgtatatatagagaggatagtatctaggaaataagactatattatatatataaagaaaatagagaaaagataaaaatataaattggtatgtataaaataaaggtctatgcgtctctagtccaaaaactctatatatgtctggctcaaaagcagatgcagccatagccccaatagtcatgatggagcatcatcatctccttatgaattggaacaaacctattgatctcattacagaagaaaactcttttaaccacaatcctcatttcatagtagatccaccttccgaaaccctaagccacttccagcccccgccgacaatcttctccgatcacggaggaggagaggaagcagaagaagaagaaggagaggaagagatggatccgatgaagaagatgcaatacgcgattgctgccatgcagcccgtagacctcgatccagccaccgttcctaagccgaaccgccgtaacgtaagggtaagcgacgaccctcagacggtggtggctcgtcggcgtagagaaaggataagcgagaagatccggatattgaagaggatggtgccaggcggtgcaaagatggacactgcctccatgctcgacgaagccatccgctacaccaagttcttgaaacggcaggtgaggctagcttcttcagcctcacactcagcttggagctcctatgtctgacccttcttgcctttgttattaattaccacaactcggataactaattataattctatcacgcgtttcatgttgatatatatagataaatggtcgaataaggatttcgatcgaagattgtatggctattgattacattatatattgtacaataaatgatgtgtgtatttcaattaatgtatgatatatatatatatgtatgcagtatgcatttatattctattctctataaggaggcaacattgccggattagggctttgatcttatgcaagttttccgaccaaaaatatgaaatacttgtttggatataacatatgaatcggataagtgttactagttatataactggaaaacaaatgtctggaataagaattcccgggagaaccaagcctttctctaatccctaagattatagctactgaaacaatgaaacaatgaagaatcagttgggcattagtaaaaaaaaaagaatcagttgggttgcttataaaattttgttataaaatttatgtcgtatgtgtgttagccgtagctgtaaatttatttcctttgtatgttaattgtaaaagtaaatttattattcccgatgtttttaatgatgtaaatttaaatcaagaaaacatattaagttctgttaataaatgtattatttttgtttactaatttccaataattttttcaagaagtacaattttttgtttgaaatttctaatttatcattattgaataatagaacatacctaaaaaccataaaacatgtattccttgaaacattttttttaatataaaaattatcttttaggagcaggtaaagtacttttttttataaaattgttagtgtatttatgttcgttatagatgtagatattttgcataataattaataatatatgagatgtgtaatgtatatatgtgagagaattgccatatttattaagcggtttttgtaaaattgactcaaaatttaaagtcaaccacaaaactaacatatgtttttttggacatttcatttgccatattcactccacaagttcagattatttacgaaaatgtcattaattttttttctttctgaaatggtatttttactctctcaacctcatcatcttcaagtatttacaagattgtcattgccatcaatacaccaaccaccatgaacaaccaatttgaataatgcaccttaaatcgatttacacttcttccttctcaattcttatgaactaaaaacaacatctctttcactttctctccatattcatccaaaaaatctaagatttttattcaaaaaaatttatggttcatagaaccattgaagcttacgattcttggtgtgtcacttttgtttgagattataggtgcttggagaagacttttgtgtgctaaacaagttatctcaatggttgaactatgaaaatcagtgtttttttccagatttgttcgcccaaccgactttcgggtaagttttctggctgtagacgactaacatggaagtcttctggtcaatgcacttctctttgattgtcttcttctgatttacattatgtagaatgaacaagagatgggaggaactaggggtaggtaaaaaaaccggaatcgaattatcaaaccgaacaaacccgaaaaaaccgaaccaaatttatgaattatccaattggatactgaaattgtatacccgaagaattagaaccgaactcgtaccgaaccgaaaaccgaataatacccatatagaaccagatgaaaaacctttatacactacttatacgtatatctttatagtttatattttatacacttgttttttttattgatcaacgcttgttttataggagaaccttgtattgtttggatattttatcctcataatttaagttttggtttctgaatgggaaaatcatttgaatgttattttggtgttggttttaattcatttatttgcaagtacatcagattaatttggataatatggttacaaagtaaaaagaaaaaactgaacttcaggaacctaaaaaaccgagaaccgatctgatctgatccgaaatttttatggttctttaatggttaccaatttcaagaaccgaaataaacgagaaccgaaagaaccaataaccgaagaaccgagcgcccactcctaggaggaaccaaacaatactttgatttgaagcccaagataattggaaccatgttatattacgcagcttatctgatgtgggctttatgtagaactgataatgggctttaaggcagcctatttcggtccgtcaagaagattcctcctaacgtaaggtaggggcaatgagctgctactctgaccaattaattacgtttcaatagtgatctctcatctgttgtcggtgtgaaaggaatcgtcttttgaatttatcttcatgcgattcattg

SEQ ID NO: 23 - DNA, 7512 bp, Brassica napus
  source 1..7512: mol_type = other DNA
aacaaaaatgatagatggtgcactaataattaatttatagtcagtttaataaaaagcttattatatatttagatgaaccaacatatttttctaatgattataagaatcatcatggtgatgacctaaaaagttgtaatgctcttcttttaacatataggggatttcacatctgattctttattataagctctattcttgtttggaaaatataatatacatattaaggaaattcgaggggtttaattttgttgtgatttattagaattaatttttatggaactttttaactaatttattatgtgagaatacatatggtgttgatacgtcaacttttttttttgtgagaaatattattgtattgacacttcaactttttttgtgagaaacattttggtgacgacacatcaacaaggctctctcaatgattctcaaataatatatagaagattttaaggagaaaataggaatataaatagtaatttcataaatttgagagcatactattcacaatcttattttatttttgaacattgttatttataaactgatccttaaattttatgtgttttgttattaatacaattataagttcatataaatcattgtatattccgatggaattaaagtttagggtttaaaaacacgtgcaaaataggttacacacgattcgttaaagatccacgagcactacaataagacataaaaactttatatggtataacatatgctaactaatcgaatattacaatatttgtatacggtaaataaaaaatcaacttacaaagacgatttcgggaattctcatagtattttcaaatctattttaccagtgattttgaagcagttcaaacatcataaacctcgttaaatcattgtataatgcatttatcgttgatatgcttttctttggatttggtaaccttaactccaaatcgttagaaatccgtcaagaatttgtcaagaacctgttgagaatttatatctgattctgacaaatcggggtttagggtcttgtttaacgaaaacaaattcttgatggcttcttgatggatgtgtatttcatcagaaaactaaatttttacccataaaacaaagcaccaatattttggcaacctcctaaagtgaatatttctttacagattcccgacaacatatcatttgctcgataatccgttacgaatctgaaaatttacccataaaaagaaaactaaatttttctatacataatttagccgatttttttcttcaatatttccatttgatttattctctcaaaatacattaaactcctctttatcctcaaaatcataatcgaaatcacatgttaagttggttgctttaggaattagaatattttgttagattttttcaatgttaagttaaataagttgttggttttaagattgttaattttagaaatttagatgtttgttttaccgatttagatgttagttttatttttgaatgttaatttagggtttgcatgttagttttacgatgtagttgttaattttaggatttgtagtttaaaattttttttttacgttcgcgtaacattaaaaatatttttcatactgaaaacatattttttaattttaaagttattatattatttaaaagtaattctaaacatttattatatatatttactaatttatgaatgtttttacaaaaaaaaattaatttacaattgattttttattattttatattttatatgtatttttattataaataatctttacaatttgtttttgtattttacatatcttcaaacactttattatttaaagatgattttatttaattttattaaaaatatttgatttatatattcttattaaaaatattaatttactgtttattattttatataattttataaatcattttatttagagtttaatttatttacgatttattgttttaatttgttaggcaaattagtctaacaaattaaacaaaaaaaaatataaataaattaaattttaaacaaaatgttttataaaaatatttatatccttataaggatataaactataattttgtgttatttttatatttaaactattattatatttaattatttttaaataaatttatttatatttttgtatatttttattaaaactaatattttatttaattatgaaaattgaaaacgtttttttggttatttttgaaaattttgacaaggttgaaaaatcgctggaaatatttttgaggaattttagacaaaaaaatatcgatgataaacaattggttggaatccgtcaagaatttttgacaaaataaactcgaaatttgttttcactgggaattagttattttcttgtatttgcattatctaataaacaatgcaaccataaacaacaaatgaaataaagttttaattgacaaatcttttagaagagtgaaacgttaatggtggacgaagcccccacctcaattgacaataaaaagagtggaaaagagggaagatggcagagagaaaagtccaaagagaggaaatcatgaagagaagaagacaatgtaacctacttatttgcctcgatgaagatagaggagaagatagaggtggaatatatatagaagagagaaagatttgggaggaaacccgattgagcatatatcagatctagaatggattggattagataactcgaacttatttggttaatctttttagtttagggaaggttatgaaaaattacctctgatctaatttttaaattttttatcttatatacattgtgccccccgactaacaatatatctagatccatccctgattaggtccatgagatatatcatattttttatttttcttatttaattaattatctataatatttgttgatatatttaaaattttacatatcgttgaaagccagaaaaatatgtggaagtgtaataaaacaattttctccaggaagaagatatttcagccattcgcccatctatgtgtatgagatcccattcaaggccatattctaagattgactttatctaatttactgtattttccgtgggctactgaatgaatacgatgcttttgttgtaaaacgcaacctattgttctccattttgaatttgaagtgttacattgttgataaaaaggaagaaagtacaaagcttttcatttagtggttgttgcatatatatacttatttgatgggtttatgataagtatgaacatttcacgtaacgggtttattttgtcgtcaacacattatcggcaatgtgaaaagaaggggtgaggtatctccatttcaattcttctctttatatattaatcgaattatttacgtatgaaatgaacgtttatatagaaatttcgtgtggaaaacgacatgtacacggcatctcaagaccaattagtaatatactttagtggtgattacatgtttacttatccaattgagaatttaaagcatcgacaataccttaatgtcgattaagccgtccccacttcatgtaatgagttatggggggagagagagatcccgaaattcgtcaaataaaacaacttagaactaaaaaccgacaccaagtatcataaaggaaatgttgaagaagtcatttatcgtatccagctcacaattcctaagattaaatcatgaccgttggaagagcttataagattaaactgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctagaaccaagcctttctctaatccctaagattatagctactgaaacaatgaaacaatgaagaatcagttgggcattagtaaaaaaaaaagaatcagttgggttgcttataaaattttgttataaaatttatgtcgtatgtgtgttagccgtagctgtaaatttatttcctttgtatgttaattgtaaaagtaaatttattattcccgatgtttttaatgatgtaaatttaaatcaagaaaacatattaagttctgttaataaatgtattatttttgtttactaatttccaataattttttcaagaagtacaattttttgtttgaaatttctaatttatcattattgaataatagaacatacctaaaaaccataaaacatgtattccttgaaacattttttttaatataaaaattatcttttaggagcaggtaaagtacttttttttataaaattgttagtgtatttatgttcgttatagatgtagatattttgcataataattaataatatatgagatgtgtaatgtatatatgtgagagaattgccatatttattaagcggtttttgtaaaattgactcaaaatttaaagtcaaccacaaaactaacatatgtttttttggacatttcatttgccatattcactccacaagttcagattatttacgaaaatgtcattaattttttttctttctgaaatggtatttttactctctcaacctcatcatcttcaagtatttacaagattgtcattgccatcaatacaccaaccaccatgaacaaccaatttgaataatgcaccttaaatcgatttacacttcttccttctcaattcttatgaactaaaaacaacatctctttcactttctctccatattcatccaaaaaatctaagatttttattcaaaaaaatttatggttcatagaaccattgaagcttacgattcttggtgtgtcacttttgtttgagattataggtgcttggagaagacttttgtgtgctaaacaagttatctcaatggttgaactatgaaaatcagtgtttttttccagatttgttcgcccaaccgactttcgggtaagttttctggctgtagacgactaacatggaagtcttctggtcaatgcacttctctttgattgtcttcttctgatttacattatgtagaatgaacaagagatgggaggaactaggggtaggtaaaaaaaccggaatcgaattatcaaaccgaacaaacccgaaaaaaccgaaccaaatttatgaattatccaattggatactgaaattgtatacccgaagaattagaaccgaactcgtaccgaaccgaaaaccgaataatacccatatagaaccagatgaaaaacctttatacactacttatacgtatatctttatagtttatattttatacacttgttttttttattgatcaacgcttgttttataggagaaccttgtattgtttggatattttatcctcataatttaagttttggtttctgaatgggaaaatcatttgaatgttattttggtgttggttttaattcatttatttgcaagtacatcagattaatttggataatatggttacaaagtaaaaagaaaaaactgaacttcaggaacctaaaaaaccgagaaccgatctgatctgatccgaaatttttatggttctttaatggttaccaatttcaagaaccgaaataaacgagaaccgaaagaaccaataaccgaagaaccgagcgcccactcctaggaggaaccaaacaatactttgatttgaagcccaagataattggaaccatgttatattacgcagcttatctgatgtgggctttatgtagaactgataatgggctttaaggcagcctatttcggtccgtcaagaagattcctcctaacgtaaggtaggggcaatgagctgctactctgaccaattaattacgtttcaatagtgatctctcatctgttgtcggtgtgaaaggaatcgtcttttgaatttatcttcatgcgattcattgttttataactaccacacatgtgacataactaatagtctcgaatcaggacatcaaaatttatttttttgtttgtggcaatgattcaaatcgacggcatagcatactgattaaaaatatatacaatacactggttattaaaacgttatagttgtctagattttaataacaggtgataatagatcaagtatatagtaaatgttttatctcacccattacatatttcatattaaacatttcagatttatttgtaaatggattcaactgcctgttacccttcactccttttgattaacatcgcgtcgcaagtcacaaccaccatttagttggggcgattcgtatattttgggagtatataataacatacataactatcaaaccaactggtgtcgacctttggtgttaactaatttgatttaactcttttttggaataaataaaataaaatttcagagcagctgttcaatagtaaaatgacaggtgggatctagggtttgtggtccttacgatcgattgattactactacttaacttggcattatttggagtcacttttgcttattaataattagattttgtttacaaacgtgtggtgatagtctgggacaagttgacctgttgacacccaatcatttagaacaccttttacaaatataaatattcgcacactgttacaaacattatcatcaaattattgtggaattttgcagacaaagcatatgccattgtgtttccttcgatttggcttttatgcttattcgtggtgtcataattaattcgtgaaaacgaatgattttacatactgtaagtagggctgaaagcggatcgaatagtaccatattttctactatttgtgatttattccatatttcagttttcttcgtaaaaatacgaatatccagtttttcgaatatccggaatttttttgacatttgcgaatattcacagatatttacagatgattcatccactttgttcaatacaaatctaaaaaaatatcaaattattttttagaaatatcttttacatgataaaaatattaagtaaaaattaatgaaactaaatgttttattaaattttaaaattagtaatttttcttataaaaatgtttttagaaaaatatgcagttttgtataaagaattatatatttttaatttaatattttataagtagtttttaaaataaaatttaataaattatatgttaaataattatataagattatacatttaaaactctatatttaatcgtagatatacatctatttgtacaactccgatccgtctctaattttttttagtatttgtggtttggttcgtttttaacggatattgatttttaatatttgcttttttttagagacttacgaatattcatatttttttggatcgagtcgaaacaaataataaattgaatcaatttaacggataaaatgttcagccctaactatggttaactatggatactgaaaccaccacttatcaacaaaaaaaagtagttgttcgcttctggtttagagtgaggttacaaattcaaaacataataattattttctcggacgataaccagatcctttttagaggctttaaaatgtaaatcatagtcatgtgtagcctatacatacaaattaaattacaatggaattttgaccaaaaaaaaaaaaaaa

SEQ ID NO: 24 - DNA, 6463 bp, Brassica napus
  source 1..6463: mol_type = genomic DNA
aagccagaaaaaaatgtggaagtgtaataaaacaattttctccaggaagaagatatttcagccattcgcctatttatgtgtatgagatcccattcaaggacatactgttttccgtgagctactgaatgaatacgatgcttttgttgtaaacgcaacctattggttttttttttccgccaagttttgtttttattaatcaaacgggccaagcccaaacggtcgaagcccaaatacactaagtaccaaacaaaaggcccacagacacaacgggctacaacattaatggctgaagcccaagtaaactaaacggcccaaaggcccaaacattgaaaccggacagcttgacctaggcgcgcgataaggagactggggtttgaacacgtgtaagaatctgggccattagtatgacacgcgtcgcccgcctcgacttgatcgaaacctcttcgcacagccgcatgaacttgccaccgacgacctcgtttcgccggagctcagagactgaagagcctccaacgaaaccatagctattctacgccgctctgtcttcacgccggagagttccatcgatctaacgagatctcctccgactatgtatcaccacaaaccctagacaggcgagccgagaaccactgatatcctcctcttgtacgtcctcgccgttgtcggagagtttcatcatcatccgagatctcatccgcgatggcccacgaccccatacctgaccacaagaaccacaccgcacttatctcacctcgatctaatcaccacaacccaaaggacatcgggttccaagacggcagcgcggagctttgcaagcctccactctccgtgaccaaaagccggcgaagacggagctgaatgagcctccccttcccggaagctaaagcggcgacggtggatctgagaaagcctccacctcccgcataaagaccggcgacgatggatctagggtagcctccatctcccggaaaaaacaccacctgaacatgcaagccgctccatctccaccgggagcctccaattgatcgcgtcgttactccaaaggccgagccaccgttgatggatggttgcggtcccagagctccgacaaggcgatcgatgacgggactgcgaagaaggagcggagagtgacaactacaaaggaagggaaagagctctggcgacagcacggacgctcacgcgccagccgtacgccgaacccaaaagtagatctggtttctctcttttctctctctcgcaaaagaaaacacgcaacctattgttctccattttgaatttgaagtgttacattattaataaaaaggaagaaagtacaaagcttttcatttagtggttgttgcatatatagtatacttatttgatgggcttatgataagtatgaacatttcacgtaacgggtttattttgtcgtcaacacattaccggcaatgtgaaaagaaggggcgaggtatctccatttcaattcttctctttatagattaatcgaattatttacatatgaaatgagcgtttatatagaattttcgtgtggaaaacgacatgtacacggcatcgaaagaccaattagtaatatactttagtggtgattacatgtttacttatccaattgagaatttaaagcatcgacaataccttaatgtcgattaagccgtccccacttcatgtaatgagttatggggggagagagaggtcccgaaattcgtcaaataaaacaacttagaactaaaaaccgacaccaagtatcataaaggaaacgttgaagaagtcatttatcgtatccagctcacaattcctaagattaaatcatgaccgttggaagagcttataagattaaattgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctatatggcaatgatatcatcaccaaagtgcaatgcccctttttgccctagtttcgtaaagtctctttccttcttcatccttaggaaaaaccctaaattaaatcctgtgttcttgatcttgctttttgagtaaccatgattttgaccacacactagttcttctatattttgtggtctataggattttgctttatatgtgtttcttgtattgcaccgtacgtgcgtatataaatttaaatggttacaacaaggtttattataaacaggcacaaattagtccatgaggttatttagcttgcacaagaggtatataaatgtgtgtgtgtgtgttaagtatttaaatatataaatttgctacaaaacttaattaactttatctgattatattttctttagtgttcttcatttgccaacgttgaggtagctattattattattattattattattattattattattattattattattattattattattattattattattattattattattattttgaacattatgtactatgtacgtagttattttggctagttatgattcaaattcttaatttggataacacttaacagtatttaaaaaaaaaatactatttaaaatattcttagtactaaaataattaagatttaacttgaaattgaagtattcgtgttaaacagaaactacaataaacaaatgattgcatgttaatttttttttcgattatcctatatatcagaataaacacatgattgcatgcaaattttgtttttgattatgtcatcttttgtttattttagttttgatgctaattaatattttttttattaacaacttccatacattctacctgattctaggtcagataatgacacagcgcaacaaaattatacaaaactttcggaaagtagattaccgcggaagtaacatttttgggtagacatacgaagtagtttgaatcttcaaaaatcatgccatacataaccacggatcatagtcgacacctcaacgtgaagcaaatttgacaatctacatacataaccaacaaaaagtagaataccgtgaaaacctaaacccaaaatatgatgtaaaactcaagcttggtccagagcataaaaaaattaaagccatcgctttggtatcacatatttaaacgtcagtttttttttggggaagtaatataaaaatataattaacaagaaaatttatgaaataattagcatgtaaaacactagtcttttggttgtgacaaaacgttttcacaaatgttctataaataaattcaagcacattttatctgcaaaatatatactttcactcataaaataagagcgtttaaaacattcatatacgcactacattgacatgacaaaagaaatccgcaaatacaaacatatttagttcggatatatctaggaaataagactatattatatatataaagaaattagaaaaaaagaaaattggtatgtataaaagaaaggtctatgcgtctctagtccaaaaactctatatgtctggttcaaaagcagatgcagcagccatagccccaatagtcatgatggagcctcatcatctccttatgaactggaacaaacctattgatctcattacacaagaaaactcttttaaccacaatcctcatttcatggtagatccaccttccgaaaccctaagccacttccagcccccgccgacagtcttctccgatcccggaggaggagaggaagcagaagacgaagaaggagaggaagagatagatgagatgaaggagatgcaatacgcgattgctgccatgcagcccgtagacatcgatccagccaccgttcctaagccgaaccgccgtaacgtaagggtaagcgaggacccccagacggtggtggctcgtcggcgtagagaaaggataagcgagaagatccggatattgaagaggatggtgccaggcggtgcaaagatggacactgcctccatgcttgacgaagccatccgctacaccaagttcttgaaacggcaggtgaggcttcttcagcctcacactcagcttggggctcctatgtctgacccttctcgcctttgttattaccacaactcggatacctaattataattctatcacgcgtttcatgttgatatatatagataaatggttgaataaggatttcgatcgaagattgtatggctattgattacattatatattgtacaataaatgatgtgtgtatttctattaatgtatatatgatatatatctgtttgcagtatgcatttatattctattctttatagggaggcaacatgccggattagggctttgatcgtatgcaagttttccgaccaaaaatatgaaatacttgtttggatataacatatgaatcggataagtgttactagttatataactggaaaaaaattgtttggtataagaattcccgggagaaccaagcctttctctaatccctaagatcatagctactgaaataatgaaaaaaaacaaaaaaaaaacaatgaagaatcagttgggcattagtccaaaaaaaaaaaagaatcagttggattgcttataaaattttgttataaaatttatgtcgtatgtgtgttagccgtagctgtaaatttatttcctttgtatgttaattgtaaaatttaatttattattcccgatgtttttaatgatgtaagtttacatcaagaaaacatattaagttctgttaataaatgtattatttttgtttactaatttccaataattttttcaagaagtacaattttttgtttgaaatttctaatttatcaacatacctaaaaaccataaacatgtattttttgaaacatttttttaatataaaaattattttttaggagcaggtgaagtatttcttttaataaaattgttagtgtatttacgttggttatagttgtagatattttgcataataattaataatatataagatgtgtaatgtatatatgtgagagaaataccatatttactaagagtttttcgcaatattgactcaaaattcaaagtcaatcacaaaactaacatatgtttctttttagacatttcctttgccttattcactccacgggttcagattattcacgaaaatgtcattaaattttttttcttttcgaaaatggcatttttactctctcaatttcatcctcttcaagtatttacaagatttccattgccatcaatacaccaaccacaatgaacaaccaatttgaagctcttaatgcacctcaaatcgatttacacttcttctttttcaattcttatgaactaaaaacaacatctctttcaatttctccccatattcatccaaaaaacccaagatttttattctaaaattttaatggttcatagagccattgaagcttacgattcttggtgggtcacttttgttcgagattttaggtacttggagaagacttttgtatgctaaacaagttatctcattggctgaaactatgaaatcagttttttttccagatctgttcgcccggacgacttccggataagtcttctggctgtagacgacttacatcgaagtattctggttaatgcagatgttagttttgcaattgacttttaagatgcttactcgaagattagtactcttctgttcttgtaccttatgagcattccaatagtattatatttttcattctaaaatatatttcaatgatattttacttttcactctataatagagtgaaaatgagtttactttataatcccctatatattatttgtgaaacactacaacttctttttgtagccacatgtcatcactaggatgatttttagaattattagagaaataggttggtccatctaattatataataagttttttattaaattaaccataaattcattattaatgtcatttattatttccttaaataaagattacggaattgcctaatgtgggtaaagtatatatgacaattaatgatcttgaataataaagatatgataaaaaaaatgtatcttctatcaaatttgtttaatttaaaactattaaaataatttttaaaaaaacacaataaccatattataaaaatttagatttttctgtatatttttatattttgaactttaaaaaatgacgataaattactaaaactgttaaaagtctcacattcaaattttgctatccatgttttaaaatttttgttatgacaaaatacaaatgattacaaaatcatataagtaaaagtctaatttaattaatcattaagatttaaaatatatatgtatatatatcattctaaattaaactataaaccatattgaataaataaatattttagtttcaaaattttactttgaataattttttttgataaaagttttgaactaacattgataaatgtttttaaactatcaattaccaaaattattgatcccacaatgaaaattttgttatcagtaatttaaa

SEQ ID NO: 25 - DNA, 4034 bp, Brassica napus
  source 1..4034: mol_type = other DNA
aagccagaaaaaaatgtggaagtgtaataaaacaattttctccaggaagaagatatttcagccattcgcctatttatgtgtatgagatcccattcaaggacatactgttttccgtgagctactgaatgaatacgatgcttttgttgtaaacgcaacctattggttttttttttccgccaagttttgtttttattaatcaaacgggccaagcccaaacggtcgaagcccaaatacactaagtaccaaacaaaaggcccacagacacaacgggctacaacattaatggctgaagcccaagtaaactaaacggcccaaaggcccaaacattgaaaccggacagcttgacctaggcgcgcgataaggagactggggtttgaacacgtgtaagaatctgggccattagtatgacacgcgtcgcccgcctcgacttgatcgaaacctcttcgcacagccgcatgaacttgccaccgacgacctcgtttcgccggagctcagagactgaagagcctccaacgaaaccatagctattctacgccgctctgtcttcacgccggagagttccatcgatctaacgagatctcctccgactatgtatcaccacaaaccctagacaggcgagccgagaaccactgatatcctcctcttgtacgtcctcgccgttgtcggagagtttcatcatcatccgagatctcatccgcgatggcccacgaccccatacctgaccacaagaaccacaccgcacttatctcacctcgatctaatcaccacaacccaaaggacatcgggttccaagacggcagcgcggagctttgcaagcctccactctccgtgaccaaaagccggcgaagacggagctgaatgagcctccccttcccggaagctaaagcggcgacggtggatctgagaaagcctccacctcccgcataaagaccggcgacgatggatctagggtagcctccatctcccggaaaaaacaccacctgaacatgcaagccgctccatctccaccgggagcctccaattgatcgcgtcgttactccaaaggccgagccaccgttgatggatggttgcggtcccagagctccgacaaggcgatcgatgacgggactgcgaagaaggagcggagagtgacaactacaaaggaagggaaagagctctggcgacagcacggacgctcacgcgccagccgtacgccgaacccaaaagtagatctggtttctctcttttctctctctcgcaaaagaaaacacgcaacctattgttctccattttgaatttgaagtgttacattattaataaaaaggaagaaagtacaaagcttttcatttagtggttgttgcatatatagtatacttatttgatgggcttatgataagtatgaacatttcacgtaacgggtttattttgtcgtcaacacattaccggcaatgtgaaaagaaggggcgaggtatctccatttcaattcttctctttatagattaatcgaattatttacatatgaaatgagcgtttatatagaattttcgtgtggaaaacgacatgtacacggcatcgaaagaccaattagtaatatactttagtggtgattacatgtttacttatccaattgagaatttaaagcatcgacaataccttaatgtcgattaagccgtccccacttcatgtaatgagttatggggggagagagaggtcccgaaattcgtcaaataaaacaacttagaactaaaaaccgacaccaagtatcataaaggaaacgttgaagaagtcatttatcgtatccagctcacaattcctaagattaaatcatgaccgttggaagagcttataagattaaattgaagaaattgtgggttttagaagaaagacaagaaagagaagaacatgatcttacattgcctattttggtgtataggagttgtcaaaaagaggagagagaggagacaattaggtcaaataaatgagcactaaaaatggagacatgtgttgagtaactattacaagagcgacttatgcttctagaaccaagcctttctctaatccctaagatcatagctactgaaataatgaaaaaaaacaaaaaaaaaacaatgaagaatcagttgggcattagtccaaaaaaaaaaaagaatcagttggattgcttataaaattttgttataaaatttatgtcgtatgtgtgttagccgtagctgtaaatttatttcctttgtatgttaattgtaaaatttaatttattattcccgatgtttttaatgatgtaagtttacatcaagaaaacatattaagttctgttaataaatgtattatttttgtttactaatttccaataattttttcaagaagtacaattttttgtttgaaatttctaatttatcaacatacctaaaaaccataaacatgtattttttgaaacatttttttaatataaaaattattttttaggagcaggtgaagtatttcttttaataaaattgttagtgtatttacgttggttatagttgtagatattttgcataataattaataatatataagatgtgtaatgtatatatgtgagagaaataccatatttactaagagtttttcgcaatattgactcaaaattcaaagtcaatcacaaaactaacatatgtttctttttagacatttcctttgccttattcactccacgggttcagattattcacgaaaatgtcattaaattttttttcttttcgaaaatggcatttttactctctcaatttcatcctcttcaagtatttacaagatttccattgccatcaatacaccaaccacaatgaacaaccaatttgaagctcttaatgcacctcaaatcgatttacacttcttctttttcaattcttatgaactaaaaacaacatctctttcaatttctccccatattcatccaaaaaacccaagatttttattctaaaattttaatggttcatagagccattgaagcttacgattcttggtgggtcacttttgttcgagattttaggtacttggagaagacttttgtatgctaaacaagttatctcattggctgaaactatgaaatcagttttttttccagatctgttcgcccggacgacttccggataagtcttctggctgtagacgacttacatcgaagtattctggttaatgcagatgttagttttgcaattgacttttaagatgcttactcgaagattagtactcttctgttcttgtaccttatgagcattccaatagtattatatttttcattctaaaatatatttcaatgatattttacttttcactctataatagagtgaaaatgagtttactttataatcccctatatattatttgtgaaacactacaacttctttttgtagccacatgtcatcactaggatgatttttagaattattagagaaataggttggtccatctaattatataataagttttttattaaattaaccataaattcattattaatgtcatttattatttccttaaataaagattacggaattgcctaatgtgggtaaagtatatatgacaattaatgatcttgaataataaagatatgataaaaaaaatgtatcttctatcaaatttgtttaatttaaaactattaaaataatttttaaaaaaacacaataaccatattataaaaatttagatttttctgtatatttttatattttgaactttaaaaaatgacgataaattactaaaactgttaaaagtctcacattcaaattttgctatccatgttttaaaatttttgttatgacaaaatacaaatgattacaaaatcatataagtaaaagtctaatttaattaatcattaagatttaaaatatatatgtatatatatcattctaaattaaactataaaccatattgaataaataaatattttagtttcaaaattttactttgaataattttttttgataaaagttttgaactaacattgataaatgtttttaaactatcaattaccaaaattattgatcccacaatgaaaattttgttatcagtaatttaaa

SEQ ID NO: 26 - DNA, 3168 bp, Brassica napus
  source 1..3168: mol_type = genomic DNA
atatggaccaaaacatgaatgatttttcggaatcagatactccattcagcttcgaacagttattatatggagatgattggcagaggtgggacacttatccgagcgttgatgttaatgccaactcaatagagcctaatcattcgatggtcgagatgaaccatgatggtatgacaatagatgctactccaatttcaatgatatatccaatggtaataatctactttctgcatgttttttttagagatattaaagtatggataaataatataagacttattgaaaatgtataggaagaaacattcgagcagtggttacagacactggccaatggatcatttgaaataccatcatatgaagaactcaacttcacgtatgaggtatggtgaaagataatatatataaaggtatattttatgtaacatataatcaatttattggtaacttatttggttatattgcatttgtacatttttttctcagtggatatgcatttgaatcatagttgaagaaaactaaaaggaatatatatgctgcaattggattgttgatttttaaaatgatatgacgtccaatgacttatttcattatttccataaatttttacagtatttaacatgaatagaatgaaatactcattccatcaattccacaaacattccaaaaaatatacaaaaaatacattcatatgcaattttaattatgagtgaatagaatgaaaatatttcttttttaactaattccattcatcttattccatctattttgattccactaatttcatttcattgttaccattgacaaccataatgtgcatggttgtaaatttagaactctgtaaccatatatatatatattcaaaagtttattagtgtatttttttctaggaattattgctcattagtgagcagataggagatgtctgcattggtgtagacgttgatatcattgaggggaatctgaaacgaagaaagtatgaagatcgttctggtcaagcggagaagtgtgtgatatgtctcgatgaacttaagtgcaatgatgaggcttctacactggcatgtgggcatgattttcactacgagtgtatcaaaaactggctaatggtcaagaacaagtgtcctctttgcaagcaacaagctctttaattgtttcatgatgctgaatatttgaatgtgttagtattgctaacattggtttcttttgttcaagtatcggtattcggtagtttaataaaaacaaatatggaagtttcataacaaaaaaaaaagtctaccaaaatttatgcaagttaattatcattcaagacaacacccactgcaatttgtcaaaaatggtgaaactcttgcaagaccttacctgtgaagtgattgtcctttttcatgtttaataataataattaattacagtattatttatgactttcagaaaactctatctatcaaccgttctttactgatttttccacccatcactgctctattttttatttatggcatcgggaaaaaaagagaagaaaaagaaatattatatgcaatctttttttttttttttttatgcaatcttaccgggtgttaatacttataactacatttagtgataaatctgctttgttaaatccagtacaaaatcaattattgaatttatagactataaagttgtgaacaaaatacaaattatcactaatgggaatattcatatacagtataataacgtaagccaccagattatggctagagtgatttgccacgcgcctgcctatttattatgaaaagcctcagtaactctgtgacgagaagaattcacagagagagagaggagagagatgggtaattccgacgaaggtggtcgtcttcctgctccatcttcttcagacgaactctcgagcattctgcggcaggtactgtcccgtactcccacagctcaaccttctttctcaccgaagaaaatcgtttcctccgctgagatgttcaaccgaacattccccctcgttcccggcggagcggtttcttacgccgcttgtgcagccgctgaaactggggaaggcaaatgtggtttcgaaaacaaggtaaacttaacgatgttagttgccgtgaaactaccctccttttgattaaagaaaaaaaataacttagttgttgtttttgcttggtaatggttaacacaacactaaagagaaatggagctaaacagcgaaattcattgaagagaaacattgatgcacagtttcacaacttgtctgaaaaggtttttgtctttatcctttttaagattcttgatttggtaaaaaaaaaactagagataataatagaaactggatatattgcagaggaggaggagcaagatcaatgagaaaatgaaagctttgcagaaactgatacccaattccaacaaggtggtaaataccaagttcgcctttttatccttcagaactcagttgtgagaaccatttgttttgttttttatgttttgtagacagataaggcctcaatgcttgatgaagctatagaatatctgaaacagcttcaacttcagtttcaggttctttttctatatgttccttacgctatgatcatgaacaactaaatttgtaaaaccaaacatctgattaacttaatgaatgcagacgttagccgctatgaatggtttaggcctaaatcctctgcgattaccaccaattctaccgcctacgcagacaaggatcactggaacctcagaacaagggctgaaccttgagactctgcttggtggttctcactcgatggctaaccgtgaaccacccgaaactcaggaaatgtgctttcccacaaccactctgctttgaagacaacgttcagacagtgaagaggatccgaagtcagatttcctctccacagaaacatgagtcgaaaatgatttgtagagtctagtatttggttatataagtgttatgctaataagctttaggactgaacaaaactgagtagcttcattataatgatgtcagaataaatttaactacatgattaagatcaaatggctatgtctgtgagttgcagccatccgtacgcacaccggtctcggatacttcgagccatggatgctgctgactgaaccgcaggtcttatcttcgtcaatgctttctctacttgaattctgacaccaacatccatctgcactgattccggcgccata

SEQ ID NO: 27 - DNA, 7697 bp, Brassica napus
  source 1..7697: mol_type = genomic DNA
acatgaatgatttttcggaatcagatactccattcagcttccaacagttattatatggagataattggcagaggtgggacacttatccgagcgttgatgttaatgccaactcaatagagcctaatcattcgatggttgagatgaaccatgatggtctgacaatagagcctaatccaatttcaatgatatatccaatggtaataatctactttctgcatgtttttttgagagatattaaagtatggatgaacaatataagacttattgaaaatgtataggaagaaacattcgagcagtggttacagacactggccaatggatcatttgaaataccatcatatgaagaactcaacttcatatatgaggtatggtgaaagataatatatatacatagaaaacaatatatatatatatatatataaaaaaataaaagggtatattttatgtaacatataatcaatttattggtaacttaattggttatattgcatttgtacattttttcgagtggatatgcatttgaatcatagttgaagaaaactaaaaggaatatatatgctgcaattggattgttgatttttggaatgatatgacgtggaatgacttatttcattatttccataaaaaattttcagtatttaacatgaatataataaaatattcattccatcaattccacaaacattccaaaaaaaaacattcatatgcaattttagttatgaatgaataaaatgaaaatatttcttttttaactaattccattcatcttattccatctatttttattccattaatttcatttcattgttaccattgacaaccataatgtgcatggttgtaaatttagaactctgtaactatatatattcaaaaatttattagtgcatttttttctaggaattattgctcattagtgagcagataggagatgtctgcattggtttagacgttgatatcattgaggggaatctgaaaggaagaaagtatgaagatcgttctggtgaagcggagaagtgtgtgatatgtctcgatgaacttaagtgcaatgatgaggcttctacactggcatgtggacatgattttcactacgagtgtatcaagaaatggctaatggtcaagaacaaatgtcctctttgcaagcaacaagctctttaagtgtttcattatgctgaatatttgaatgtgttagtattgccaacattggtttcttttgttcaagtatcggtattcggtagtttaatgaatacaaatatggaagtttcataacaaaaaaaaaagtctaccgaaatttatgcaagttaattatcattcaagacaacacccactgcaatttgtcaaaaatggtgaaactcttgcaagaccttacctgtgaagtgattgtcctttttcatgtttaataataataattaattacagtattatttgaaactctttgtaaacttaaagtacttttcttattgaatgaaggcaacataagagttgcttaaataacaaagcgtacagatactcccgacagttttggtaaagtcttaaccacttcctatagtagaggaagacttattcaaataacttaaagcagaaaataattcaaaactaccaactaaagactctaccaactaaagactcgatcttttttattcattaatccctcccttaaacttgaactcctttataaagtttaaacttcctttaagctgactcccatgtgtgctctcagcttctcaaacgcttcaagtttcaccggctttgtcatgatatccgacaattgcttatgagttgaacaatactccattgagatgatgccttcattcacaagctctcttaagaaatgaaacctcacatgaatatgcttgcttcttccatgtagaacaggattccttgacaacttgatagctgaactattatcacagaaaagaacagttccatcaccttgatcaaacccaatttcagccaacacgttcctcatccatacagcttgacaagctccaattgctgcagcaacatactctgcttccgtggtagagagtgtgacaattggttgtttctttgagctccaggacacagctcctcctccaaacatgaaagtgtagcctgatgtactctttctgtcatcctcatcgcctgcataatcactgtccacataccctacaagcttttcttctccaccacgcaagtactgaataccatagcttgcagttccttgcacatacctcaaaatacgcttaacagccatcatgtgcccttcatttggattctccatgtatctactaaccacattcacagagtatatcagatcaggtcgagtagctgttaagtacctcaagcttcccacgagttgcttgtaagcggttggatcaacttcttctccagctcctgtcttggtcagtttgttccctggaaccattggattcttcacagagttgcagtctttcattccatacttctttaaagtctccagtgcatacttcttttggccaataaagatcccaacctcgtcttgcacaacctccacgcctagaaagtacttcatctttcctagatcagtcattgagaactcttcttgcattgaagttttgaagctctccagcatgctttccgagttactagtgtaaatgaggtcgtctacgtacaagctcaggatcaacgagttcccttcttctgtttttacgaacaatgtgtgttcgcaataacatttctcaaagccttcctttataaagtaactctcgatcctgctataccaggccctaggagcttgtcttaatccatacaaagcctttttcaacttgtagaccttctcactgtcaacctgagactcgaaccccttaggctgctcaacgtaaacgtcctccttaagctcaccatgtaggaacgcgctcttcacatctagttgatatacactccatcttctttgagccgctaagccgagaatcagcctgatcgtgtcccatctggccacaggtgcaaaaacttcatggaagtctactccataggtttggtgaaagccctttgccaccagtctcgctttgtacttatccacctctcctttttcattcaacttggtcttgaagacccatttcactcctatcaccttcacattggcaggtagactaaccagctcccaagtgttgttatcctctattgattcaatctcagtttccattgcctttctccatttgtcatgttggatagcatcttcaaacttctctggatcctcatctgacaagaacagcgcaactagttcttctccatcatcttccatgataagacccctcccatcaaccacgtaatctttcatccagactggtcttagtttgcttcttcctgctctgcttgtgtctgatctttcttctggtgtagagacagttctttctccatcagactcagtgttatctcctccttctgtctcttcgttagttgctggtgctgtcaccaggttctcaccctgattcctctcttcctcttcttcagcttctccacattcgacaaatttgtcttcatcaatgttatcttcccagttccaccgcgtttcttcgtcaaacctcacgtctttgcttattatgatcctcttcccaataggatcataaagtctgtaagccttcgactccttgcttacaccaaacatgacacatctcacacttttctcatccagcttcacacgcttctcataaggtataagtgcataagccacacagccaaacacacggagatgctctacagacggcttgtgcttactccacttctcacttggcgtcacttctcccaatatctttgttggacttctgttaagtatatggacagcatactgtgttgcttctgtccaatacttgatcggcaccttcataccaaacagcatgcacctgaccatattcagaatgcttctattcttcctctctgccacgccgttctgctgaggcgtgtatgcggctgtcagctgtcgtttgatgccgtgctccacacaatagtcctgaaacgcctttgaattgtattctccacctctgtcggttcttagacagacgagcagctcaccactctccctctcagcggcatttttgaactccttgaaagttctaagtgcctctgatttctctgctagaaagaagctccaacactttcggctaaactcatcaatgaaattgattatgtacctcttgccgctttcagatgtgggtgatattggcccgcagatgtcactgtgaactagctccagtcgttgtgttgctctccatagactcttctttggtatcttttctctgatctgcttccccttcatacacacctcacatactgtcccgttggtgttgatctttggtagaccttccaccatcgccttttctgctagtgttagcaaactgttgtggttcaaatgtccgaagcgtttgtgccaagtttcctccattgtctccatcgtattctgaacttggtttgcctccaacgcttccttaggctccctgacaatagcagttactacaaacatcctgtttgtggtcatcagagaatgcatgatcagtctccgttgttgtttgtgccagatctcacacgtatctccttcaattataatcctcaaccccttctgttgcaactgaccgacagacagtagattgttcttcaagccaggcacaaagtatactgcagtgatcacttgagtagtaccattgatctccaatcttaggttacctcttccctcaacttgcattcgcctatcatctccaagctttacttgttgtctgaactggctatcaaactctgaaaaccactccttggttccacacatgtggttgctacaccctgagtcaaggtaccacacgtgtctttcttcagtctcaacttgagtcatcagcaaaatatcctcctctaactctgcatagttagctgccttctcccagctaggacattcgtttttgaaatgccccatcttgtggcacttaaagcactcaatgttgctcttgtcaaagcttgtatttcctctgtcttgtcctccacgacctcctctgtaactgcctcttcctctggtttgatatcctcctcgtccattggagtaacctgagtagccacctcgtccccttcctccttccatcttcaggacttgttcctctcctgtgtgtttactcaggttctgctcatgtacgagtaatgagctttgtaactcatccaccgacagctccttgatatcctttgattcctctatcgcacataccacatatgtgaacttctccacaagggttcttaggatcttctcaaccaccttttcatctggcatgtcttctccacggttccgcatgtcattagcaaccaccatgactctagagaaatagtctgcgattgagtcaccttccttcatctccaggacttcaaagtttcttctcagcctctgtaattgcgcactttgaaccctcttgttgccttggtactttgccttcagcgactcccatatgtcttttgcggtgtccttcttcacaatcgtcttcagaacagtcttgtcgatggaggcaaagagatagttcttcaccttgaagtctttgagcttttgctccgcaagctcggttctctgagcttcagacagtgtcacactactttcatgttgaatgattccggtattgatcagttcccaccactccttggatctcaacaaattctccataagcattgcccaatgctcatagtctccatcaagctttggtatgctcaacgaggactccttttcactcatctctcttactttcggattacctagaactcacaacagtttttgttagagctctgataccacttgaaactctttgtaaacttaaagtatttttcttattgaatgaaggcaacataagagttgcttaaataacaaagcgtacagatactcccgacagttttggtaaagtcttaaccacttcctatagtagaggaagacttattcaaataacttaaagcagaaaataattcaaaactaccaactaaagactctaccaactaaagactcgatcttttttattcattattatttatgactttcagaaaactctatctatcaaccgttctttactgatttttccacccatcactgctctattttttatttatttatggcatcgggaaaaaaagagaagaaaaagaaaaattatatgcaattttaccgggtgttaaaacttataattacatttggtgataaatctgctttgttaaatccagtacaaaatcaattattgaattcatagactataaagttgtgaaccaaatacaaattatcactaatgggaatattcatatacagtatagtaacgtaagccaccagattatgtctagagtgatttgccacgcgcctgcctatttattatgaaaagcctcagtaacttgtgatgagaagaattcacagagagagaggagagagatgggtaattccgatgaaggtgatcgtcttcctgctccatcttcttcggacgaactctcgagcattctccggcaggtactgtcccgtactcccacagctcaaccttctttctcaccgaagaaaatcgtttcctccgctgagatgttcaaccgaaccttccccctcgttcccggcggagcggtttcttacgccgcttgtgcagtcgctgaaactggggaaggcaaatgtggtttcgaaaacaaggtaaacttaacgatgttagttgccgtgaaactaccctcgcttgttgattaaagaaaaaaataactttattgtgtttttggttggtaatggttaaaaaacactaaagagaaatggagctagacagcgaaattcattgaagagaaacattgatgcacagttccacaccttgtctgaaaaggttttgtctttatccttttaagattcttgatttggtttaaaaaaaaaactagagataataatagaaactggatatattgcagaggaggaggagcaagatcaacgagaaaatgaaagctttgcagaaactgatacccaattccaacaaggtggtaaataccaagttcgactttttatctttcagaactcagttgtgagatacatttgttttggtttttttttatgttttgtagacagataaggcctcaatgcttgatgaagctatagaatatctgaaacagcttcaacttcagtttcaggttctttttctatatgttccttacgctatgatcataaacaactaaatttgtaaacccaaacatctgattaacttgatgaatgcagacgttagccgctatgaatggtttaggcctaaatcctctgcgattaccaccaattctaccgcctacgcagacagggatcactggaacctcagaacaagggctgaaccttgagactctgcttggtggttctcactcgatggctaaccttgaaccacccgaaccaactcaggaaatgtgctttcccacaaccactctgctttgaagacaacgttcagacagtgaagaggattcgaagtcagatttcctctccacagaaacatgagccgaaaatgatttggttatatttcaaagtgttatgctaataagctttaggactgaacaaaactgctactggttatatttcaaagtgttatgctaataagctttaggactgaacaaaactgagtagcttcattataatgatgtaagaaaaaaaatgtataaatttaactacatgattacgatcaaatggctatgtctgtgagttgcagccatccgtacgcacaccggtctcggatacttcgagccatggatgc

SEQ ID NO: 28 - DNA, 7239 bp, Brassica napus
  source 1..7239: mol_type = genomic DNA
ataatataagcatgttgagtcctattcatcagaaaattttctccaaaggatgaacaataacctacaattaaggaaacttattataacaacgaccaagaagttttcccataagtcatgctttcgtccataccttcgggtctttcaagcaagcttcacacatgccttatcgtatatttgtatacgttttcattttgtgtacgtctattaattagtatatgcctagattatatatttatatagtcgacaattacctatcctaaaggtttattttaatttttattcagaaagatatgcacgtagggccactagtgtttcccgttgttattggaaattccatgtgttgaactgtttgattaagtttttgaacccgtaagtcaaatttatttaggtttaacagggtttcattactgttataagcttgtcaagaaagaatttgtcacaaatttcgagactcatgcatgcatgcatgtttcttaactttcgagaaacatgaagtgtggttttaccagctgaagtttgtgttaaaacacatttttaatttgaccacgtgtgatattcaaatatcaacaattctgttcaacgggagattcatacatatgttcagatgttctttaatcttgagcacctaactttgcttcccatacgcaaatgtgtgtgccgcgaactatggtgaatgtacggcatcgtttttctttttggagcatttttaaaagaaaatataatttagttatttccgttttgattgatatagatgcattaataaaccgacttacgtgggtgatgggatgagcaaaattaaaatatgcattttagtaaccaattacggtccatcttgacaaactaacaaacaaaaagtctattatgacgttgatttgtgaccttgccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaatgacaactcctgttcttgtatcgtgtatctagcacgtaaatttgaattatggcggcattatcgataaaatatgtaaagaatatattttcatcatacaccacaaacaaatgttctggaaatatcaccttttatcacatatacaaagctgaccctgaatttttggaaactacaagccaaaaaaaaatggacaaaagaaaaattatgcacaaaagtggactcgtcctaactctttttataatgataattaacctttgaacaactaattaatacatacaatatgttttttttttctaaaaattggtcgattaaatattcatatataatcctcatcatcatttattagccttttgtagcttacttaaaaaatcacttcccaccaaattgattattttttcacttcatacgttgatcaatttaaaaaaaaaataataataatatatgcacgtccggcgtcccatgacttcagacgcttttctgtttccttgattttataggttagaaatttccaaaatgctcatatttttcactgatatgtattatacgcacaaaatagtaaagtcataaaaatttatgatggttcttttatatgtatagagtagtgaaaatttatatatattgtagttccgcttaaaataaattaatatttttcttttatcgcccatatttaagttactaaacgtatataattaaaaactgaaatgcaatatgtagaaatccgggcattgattgttgattaacattgatatacccatgcgactggcttatgatgaaccatacctgctaatactcattgttacaaactcgcagcaacatcataaatccatcactcgccacaataattttcttcagtgtatgttcaattgatcacattttgacccttcactttcttgaatgagaaattatacaagaaagtgatttgagtgtgttaaaaaatgatttgtcattctgaccaattatctggcagagagtataataaaatacttctaattaccttaaatcattaggcgttgtttcttgccgaatatatgatggagcatggtctcgctaagagacaacaaaatctttaaataaattaaagaaattgtttagcaaaaaaattaaataaattaaacaagagagagtacaaatataaaacactctaacggaagataaatatataaaatacaaaatatagaatagaggcctcaagtagtgtttacgtgacaatctcaatttcactccatacctcgaactcattcaagcttagaaacctaacttctactgttccttttgatttgcctataaatacacgttggtccatacttctctaatttcaaatatacaaagcaaaacacagagacaagaatcataaataacaaacattttcaacataatcccaaaaaacgacacgcgttccttttccaaaaattcacttctctacgtaccaaccacaatggctcattgttgcacagttcttaccggtttactagtatggtctctttttatgctctcatggtgtgaagcttcaagaaacaacattaatagatatgatcattcttatggaacgttcaaatccaatagcttaatcaagcgaagagacgatataactcgtctgaaaagtgttgcaagagcctctttgcggcttccaaccaccgttagtgtttctgattttggagctaaaggagatggaaaaaccgacgacacgcaggtatatacgcatatacacatatctacatgacttataaatattttgagacggtcacgcatataaatttacgttgtttgtactaatgatccatgcatgcgcatgcatatgatcaggcgttcgtgaatgcatggaagaaagcttgttcttcaagtggagctgtaaatctcttagttcctgaaggaaaaacttattttctcaagtctcttcgattaaatggcccatgcaaatcagttcttaccgttcaggtaaattacaaagtttttttttcttaaatttgggtaaattacaaagttgactatttgttaattaaatacgtatactacatgtgatcacgtatatggttttgaatatttaaatgagcggttgatgaatggaacgacagattttaggaacgttatcggcttctcaacaacgatcggattatgaagatctcagcaaatggattacgtttgacggcgttaacagtctaacggttgatggcggagccaccggaactgtcaacggaaacggcgaaacgtggtgggaaaactcatgcaaacggaacgaagctaaggtgtgtgtttgtacatgtttcatttacttaatatatatctactgtctacatgtcatcttgatatattaacactcttttattgttattgttttgaagaaatgcacaaaagcccctacggtacgtagaaacatatttgacattttcatgtaatactgacgatggcggtgatcatgattggacaaatgttttacatgcatgaatatatgtattttatatgcaggctcttactttctacaactcgaagaatttaagagtgaataatctgagggtgaaagatgcgcaacagattcagatttcgattgagaaatgctccaacgttcaggtctcaaatgttgaggtcactgcacctgctgatagtcctaacactgatggtatccacatcactaacactcaaaacattcaagtctccaattccatcattggaacaggttagtagttatatcctcgttatttaaaagagttgcgtgcatatccagaaaactaaaatttaagagtatcgttataatttataattttatagagattggtcttaaaagagtttttaatggttgcttttctttgcaggtgatgattgtatatctattgaaagtggatcacaaaacgttaaaatcaacgatttaacttgcggcccaggccacggtatcaggtatcaatgaattgttttcagtatatgtacctaagaaattcaaccgtaatcttttaaattaacatagaaatttgatatttccatataaaacatgagcagcattgggagcttaggagatgacaattcaaaggcatttgtatcaggcgtgactgtggatggtgctaaactttccggtacagacaatggagttaggatcaaaacttaccaggtgaaaatcacaaactagaccttcaattttaactagcctattgacaagttcatctcaatttaacttttaaacccaattcaatatagggagggtctggaactgctagcaacattatatttcagaacattcagatggagaatgtgaagaatccaatcataattgaccaagattattgcgacaagagcaaatgcactgaacaagttcgtttgaatctgatcaaatctcccattactaatcatcatgataataaacactattgtctctaatcatttactacatatattttcagaaatcagcggtccaaataaagaacgtggtgtaccggaacataagcggcacaagcgcatcggacatagcaataactttcaactgcagcaagaactatccatgccaaggaattgtactcgacaaagtgaacattaagggagggaaagccagttgcagcaatgctaatgtgatggataaagacggtgttttacctcagtgcaaatccacttgattaataagacctataaaaaaaactcacatagatgtataaaaatacatatataccgatacaaagtatacaaaattatgatatacacttgtggatatatacatggacaatgaatttaaacatattgtctatatgtcaaaaatagtttatgaatatacatatagacaacatggtcaagagatttattgttttaatgtatatatatactactaaactacttgtctaaaatttcatgaataaaggattacgtttttaagtggttatgtatcatgatgtggttacagtatgatgtgtattgtagattttcaattaaatacataaaaatgaaaaagatgacatggctggtcaatgatgttacttgaccaactaattcaagctaattgatacacgatgaacatctaacgaaacagaagcgactttcatattgtttagttgtgctcaaaccaggccgataaactcattgggcctataagcccattaatatttatccgtgagagcgaagtctgttacttgggttactaaacaaagaggcctttagaaaaccctaactcttaccgtagcacctgagctatatattgccgcagctagggtttccactccctcacgctaacacacatcaccgcagccatttccatctctcaaccaacttcaaacaatggctgaacgaggcggagaacgaggtgccgagcgaggaggagaccgcggcggtttcggacgcggattcggaggaggtcgcggcgacagaggaggccggggaggtccgagaggccgaggcggcagacgcggaggacgtccgacagaggaggagaagtggaccccagtgaccaagctcggccgtctcgtgcaagccggaaagatccagaagctcgagcagatctacctccactccctccccgtcaaggagtaccagatcatcgacctcctcgtcggccccaccttgaaagacgaggtgatgaagatcatgccggttcagaaacagaccagggccggtcagaggacgaggttcaaggcctttgtcgtcgtcggagacacgaacgggcacgttggtttgggtgtgaagtgctccaaggaggtggcgacggcgatcagaggtgggattatattggcgaagctgtcggttattcccgtgaggagagggtactgggggaacaagattgggaagccgcatacggttccttgtaaggttacggggaagtgtgggtctgttacggtgaggatggtgcctgctccgaggggtgcggggattgtggcggctagggttcctaagaaggtgcttcagttcgctgggattgatgatgtgtttacttcgtcgagggggtctactaagactcttggaaacttcgtaaaggtttgaactttcttcatttatttagagtttggagttgtgtatggattgaaagttgattcctttttgatggttgcgtgtggagttgttttgggtaatttagcttgttgtagttgatgtaaagtgagaattgctaagcttgattagtttgaataagctgtctgtgttaaatgatgatttgtttgtgttgtattgagctgtctgaatttcattatttgtttgtgttattgctaaattacaagcttaaatttgataacttttgattctttcgtgagagttttttttgggtaatatagcttgatgtagttgatggaagtaaattgttaacctttagagctgtctgttaatggtgatttgtttgtgttgtattgagctgtctgaattataatttgtttgtgtttattgctaaatcacaaagctttgaagtcgtttgtacatattcatcatttttgtgtttattgacttgtttgtgtttattgctaaatcacaagtttctatgattaatcttcatgatttggttgtgtttattactaaatcacaagtttctatgattaatcttaatgatttggttgtgtttattgctaaatttgagattacattataatcttagtttatacatgtataacatcatttcacttgaattttcacagacgagctggtgatatgtttttttagtattttccatgtattgctttgtaagaacttttaaatgtttgttttcttcctttgaagaacaattaaatgtttgttcagtatttttgaatcattcattggttaatcatgtgtaattttatttttgtaggctacattcgactgtctccagaagacttatggattcctgacaccagagttctggaaagagacgagctttaaaaagtcaccgtaccaggagtacactgatctgctggccgagaagggaattgcaaccaaagccatcaccgaggttgaagaccaagccgcctcctaaataaacaaacaaacaatgtcccccttgttttgtttttggatgatcctttcttgagttttattttgttcgaacctttagtatatgttgtttgctggattgctgctttcttatgttttcatcatctcccccaaacaaagttatttttgctctagtttttttttataatcgatggttatgccctctagtattttttttacttgaatttttcactaattttctatcaacttccctatgttacactttctaaggttgattacataggtggattgttgtga

SEQ ID NO: 29 - DNA, 4666 bp, Brassica napus
  source 1..4666: mol_type = other DNA
gtcttcgcatatgatagatcataagatttggagaaacatataaaattaacctatgaaattaaggggatctatatattatagtatgtatccaaagaacatatatatgaaatatttgaatacatcataagatttggggaatataaattaatataaaataaaatcgtccaattttaaacgtaaacgtctccgctgtatttatataaacttttcaaaatcacggtgtcgctcgtgttaagatttggatttctaatccaactttcagaaagtttatgtgaaagataatactcaaacacaatagttcgtttagaacgctcttaagattaatgtcacacacacaaaattgatgtcactagatttatatgttttgtcgtttaaggggatccatcaactacacatggaagaatagaaaacactggtcacacacaaaaaataatataagcatgttgagtcctattcatcagaaaattttctccaaaggatgaacaataacctacaattaaggaaacttattataacaacgaccaagaagttttcccataagtcatgctttcgtccataccttcgggtctttcaagcaagcttcacacatgccttatcgtatatttgtatacgttttcattttgtgtacgtctattaattagtatatgcctagattatatatttatatagtcgacaattacctatcctaaaggtttattttaatttttattcagaaagatatgcacgtagggccactagtgtttcccgttgttattggaaattccatgtgttgaactgtttgattaagtttttgaacccgtaagtcaaatttatttaggtttaacagggtttcattactgttataagcttgtcaagaaagaatttgtcacaaatttcgagactcatgcatgcatgcatgtttcttaactttcgagaaacatgaagtgtggttttaccagctgaagtttgtgttaaaacacatttttaatttgaccacgtgtgatattcaaatatcaacaattctgttcaacgggagattcatacatatgttcagatgttctttaatcttgagcacctaactttgcttcccatacgcaaatgtgtgtgccgcgaactatggtgaatgtacggcatcgtttttctttttggagcatttttaaaagaaaatataatttagttatttccgttttgattgatatagatgcattaataaaccgacttacgtgggtgatgggatgagcaaaattaaaatatgcattttagtaaccaattacggtccatcttgacaaactaacaaacaaaaagtctattatgacgttgatttgtgaccttgccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaatgacaactcctgttcttgtatcgtgtatctagcacgtaaatttgaattatggcggcattatcgataaaatatgtaaagaatatattttcatcatacaccacaaacaaatgttctggaaatatcaccttttatcacatatacaaagctgaccctgaatttttggaaactacaagccaaaaaaaaatggacaaaagaaaaattatgcacaaaagtggactcgtcctaactctttttataatgataattaacctttgaacaactaattaatacatacaatatgttttttttttctaaaaattggtcgattaaatattcatatataatcctcatcatcatttattagccttttgtagcttacttaaaaaatcacttcccaccaaattgattattttttcacttcatacgttgatcaatttaaaaaaaaaataataataatatatgcacgtccggcgtcccatgacttcagacgcttttctgtttccttgattttataggttagaaatttccaaaatgctcatatttttcactgatatgtattatacgcacaaaatagtaaagtcataaaaatttatgatggttcttttatatgtatagagtagtgaaaatttatatatattgtagttccgcttaaaataaattaatatttttcttttatcgcccatatttaagttactaaacgtatataattaaaaactgaaatgcaatatgtagaaatccgggcattgattgttgattaacattgatatacccatgcgactggcttatgatgaaccatacctgctaatactcattgttacaaactcgcagcaacatcataaatccatcactcgccacaataattttcttcagtgtatgttcaattgatcacattttgacccttcactttcttgaatgagaaattatacaagaaagtgatttgagtgtgttaaaaaatgatttgtcattctgaccaattagctagggtttccactccctcacgctaacacacatcaccgcagccatttccatctctcaaccaacttcaaacaatggctgaacgaggcggagaacgaggtgccgagcgaggaggagaccgcggcggtttcggacgcggattcggaggaggtcgcggcgacagaggaggccggggaggtccgagaggccgaggcggcagacgcggaggacgtccgacagaggaggagaagtggaccccagtgaccaagctcggccgtctcgtgcaagccggaaagatccagaagctcgagcagatctacctccactccctccccgtcaaggagtaccagatcatcgacctcctcgtcggccccaccttgaaagacgaggtgatgaagatcatgccggttcagaaacagaccagggccggtcagaggacgaggttcaaggcctttgtcgtcgtcggagacacgaacgggcacgttggtttgggtgtgaagtgctccaaggaggtggcgacggcgatcagaggtgggattatattggcgaagctgtcggttattcccgtgaggagagggtactgggggaacaagattgggaagccgcatacggttccttgtaaggttacggggaagtgtgggtctgttacggtgaggatggtgcctgctccgaggggtgcggggattgtggcggctagggttcctaagaaggtgcttcagttcgctgggattgatgatgtgtttacttcgtcgagggggtctactaagactcttggaaacttcgtaaaggtttgaactttcttcatttatttagagtttggagttgtgtatggattgaaagttgattcctttttgatggttgcgtgtggagttgttttgggtaatttagcttgttgtagttgatgtaaagtgagaattgctaagcttgattagtttgaataagctgtctgtgttaaatgatgatttgtttgtgttgtattgagctgtctgaatttcattatttgtttgtgttattgctaaattacaagcttaaatttgataacttttgattctttcgtgagagttttttttgggtaatatagcttgatgtagttgatggaagtaaattgttaacctttagagctgtctgttaatggtgatttgtttgtgttgtattgagctgtctgaattataatttgtttgtgtttattgctaaatcacaaagctttgaagtcgtttgtacatattcatcatttttgtgtttattgacttgtttgtgtttattgctaaatcacaagtttctatgattaatcttcatgatttggttgtgtttattactaaatcacaagtttctatgattaatcttaatgatttggttgtgtttattgctaaatttgagattacattataatcttagtttatacatgtataacatcatttcacttgaattttcacagacgagctggtgatatgtttttttagtattttccatgtattgctttgtaagaacttttaaatgtttgttttcttcctttgaagaacaattaaatgtttgttcagtatttttgaatcattcattggttaatcatgtgtaattttatttttgtaggctacattcgactgtctccagaagacttatggattcctgacaccagagttctggaaagagacgagctttaaaaagtcaccgtaccaggagtacactgatctgctggccgagaagggaattgcaaccaaagccatcaccgaggttgaagaccaagccgcctcctaaataaacaaacaaacaatgtcccccttgttttgtttttggatgatcctttcttgagttttattttgttcgaacctttagtatatgttgtttgctggattgctgctttcttatgttttcatcatctcccccaaacaaagttatttttgctctagtttttttttataatcgatggttatgccctctagtattttttttacttgaatttttcactaattttctatcaacttccctatgttacactttctaaggttgattacataggtggattgttgtgaaacaaatactaataataaactaatacttttagaataatataattagtgggattaaattataaattataaattaaaaagacaagtctgaaatggattgtttaactgtaaaaggtaaagagactttgaatagaagagaataaatcaaaagttgacgtatcttctccgaaatttcagctatttctaaatacgcggattctgattactctgttctctct

SEQ ID NO: 30 - DNA, 7200 bp, Brassica napus
  source 1..7200: mol_type = genomic DNA
ctggtcacacacaaaaaataatataagcatgttgaattctattcatcaaagaattttcttcaaaggatgaacaataacctataattaggaaacattataacaacgagcaggaagttttcccagaagtcatgccttcgtccataccttcgggtctttcaagcaagcttcacacatgccttatcatatgtttgtatacgttttcattttgtgtacgtctattaactagctagtatatgccaagattatatattttatatagtcgacaattacctatcctaaaagttcattttaatttttattcagaaagatatgcacgtagggccactagtgtttcccgttgttattggaaattcatttccatgtgttgaattgtttgtttaagtttttgaacccgtaagtcaaatttattttggtttaacagggtttcattactgttataagcttgtcaagaaagaatttgttacaaatttcgaggctcatgcatgcatgattcttaactttctagaaacatgaaatgtggttttaccagctcaagcttgtgttaaaacacatttttaatttgaccacgtgtgatattcaaatatcaacaattctgttcaacgggagattcatacatatgttcagatgttctttaatcttaagcacctaactttgcttcccatacgcaaatgtgtgtgccgcgaactatggtgaaatgttttctttttggagcatttttaaaagaaaatataatttacttatttccgttttgattgatatacatgcattaataaaccgacttacgtgggtgatgggatgagcaaaattaaaatatgcattttagtaaccaattacggtctatcttgacaaactaacaaacaaaaagtctattatgacgttgatttgtgacctggccatataaatgccaaaatttcattctttcaataagcaacgagatcaataagatgtggcagtcacgtgaacgttagttatcgccatttcaactgaaccttcacgaacatataaagttcatttgttctttgctaaaattacgcaaatctctgttgtgcacgaaattacgactattttttaaatacaactcctgttcttgtatcgtgtatctctcgcacgtaaatttgaattatgggggcattatcgataaaatatgtaaagaatatattttcatcatacaccacaaacaaatgttctggaaatgtcaccttttatcacatatacaaagctgaccctgaaattttggaaactacaagccaaaaaaaatggtcaaaagaaatattatgcacaaaggttggactcgtcctaactctttttacaatgataattaacctttgaacaactaattaaagatacatacaatatgttttttttctaaaaattggtcgattaaatattcatatataatccttatcatcatttattagccttttgtagcttacttaaaaaaccccttcccaccaaattgattagtgtagattgattatttttcacttcatacgttgatcaattttaaaatatatatatatatatatatatatatatatatgcacgtccggcgtcccatgacttcagacgcttttctgtttccttgattttataggttaaaattttccaaaatgctcatatttttcactcatatgtataatgtattattatacgcacaaaatagtaaagtcataaacatttatgatggctcttttatatgtatagagtagtgaaatttatatatatcggagttccgcttaaaataaattaatattgttcttttatcgcccattttttagttactaaacgtatataattaaaaactgaaatgcaatatgtagaaatccggcttatgatgaaccatactaatactcattgttacaaactcacagcaacatcataattccatcactcgccacaataattttctttagtgtatgttctatttcacattttgacccttcactttcttgaatgaaaattatacaagaaaatgatttgagtgtgttaaaaaaaaaaatgttttgagtcattctgaccaattatctggcagagactataataaaatacttccaattaccttaaatcattaggcgttgtttcttgccgaatatatgatggagcatggtctcgctaagagacaacaaaatctttaaataaattaaacaagttgtttagcaaaaaataaaaaataaattaaacaagagagagtacaaatataaaactctaaaggaagataaatgtataaaatacaaaatatagaatagaggccttaagtagtgtttacgtgacaatctcaatttcactccatacctcgaactcattcaagcttagaaacctaacttttactgttccttttgatttgcctataaatacacgttcatccatacttctgtaatttcaaataacaaagcaaaacacaaagataagaatcacaaacaacaaacattctcaacctaatcccaaaaagacgacacgcattcctttttccaaaaatccacttcctacgtaccaaccacaatggctcattgttggacagttcttaccggtttactagtatggtctcttttgatgctctcatggtgcgaagcttcaagaaacaacattaatagatatgatcattcttatggaacgttcaaatccaatagcttaatcaagcgaagagacgatatcactcgtctgaaaagtgttgcaagagcttctttgcggcttccaaccaccgttagtgtttctgattttggagctaaaggagatggaaaaaccgacgacacgcaggtatatacgcatacacacatatctacatgacttataagttataaatattgtgagacggtcacgtatataaatttacgttgtttgtactaatgatccatgcatgcgcatgcatatgatcaggcgttcgtgaatgcatggaagaaagcttgttcttcaagtggacctgtaaatctcttagttcctgaaggaaaaacttattttctcaagtctcttcgattaaatggcccatgcaaatcagttcttaccgttcaggtaaattacaaagtttcttttgttttgcttaaatttgggtaaattacaaagttgacaaaaaaattacaaagttgactatttgttaattatatacgtatactacatgtgatcacgtatatggttttgaatatattaaagagcggttggtgaatggaacgacagattttaggaacgttatcggcatctcaacaacgatcggattataaagatctcagcaaatggattacgtttgacggcgttaacagtctaacggttgatggcggggccaccggaactgtcaacggaaacggcgaaacgtggtgggaaaactcatgcaaacggaacgaagctaaggtgtgtgtctgtacatgtttcatttacttaatatatatctactatctacatgtcatcttgatatattgacacttttttattgttattgttttgaagaaatgcacaaaagccccaacggtacgtagaaacatatttgacattttcatgtaatactgacgatggctgtgatcatgattggacaaatgttttacatgcatgaatatatgtattttatatgcaggctcttactttctacaactcgaagaatttgagagttaataatctgagggtgaaagatgcgcaacagatccagatttcgattgagaaatgctccaacgttcaggtctcaaatgttgaggtcactgcgcctgctgatagtcctaacactgatggtatccacatcactaacactcaaaacattcaagtctccaaatccatcattggaacaggttagtaattacatcctcgttatttaaaagagttgcgtgcatatccagaaaactaatatttaagggtttatcgttataatttatagagattggtcttaaaagagttttttaatgtttgcttttctatgcaggtgatgattgtatatctattgaaagtggatcacaaaacgttaaaatcaacgatttaacttgcggccccggtcacggtatcaggtatcaatgaattgttttcagtaaatatacctaagaaatacaaccgtaatcttttacatataaatttgaaatttccttataaaacatgagcagcattgggagcttaggagatgacaattcaaaggcatttgtatcaggcgtgactgtggatggtgctaaactttccggtacagacaatggagttaggatcaaaacttaccaggtgaaaatcacaaactaaaccttcaattttaactagcctatcaagttcatcccaatttaacttttaaacccaattcaatatagggagggtctggaactgctagcaacataatatttcagaacattcagatggagaatgtgaagaatccaatcataatcgaccaagattattgcgacaagagcaaatgcaccgaacaagttcgtttgaacctaatcaaatcttccattactaatcctcatgataataaacacttttatctctaatcatttactacatatattttcagaaatcagcggtccaaatcaagaacgtggtgtaccggaacataagcggcacaagcgcatcagacatagcaataactttcaactgtagcaagaactatccatgccaaggaattgtactcgacaaagtgaacattaagggagggaaagcgagttgcagcaacgctaatgtgatggataaagacggtgttttgcctcagtgcaaatccacttgattaactagacctatcaaaaactcacacagatgtataaatacatatagaccgatacaaagtatacaaaattatgatatatacttgtggatatatacatggacaatgaatttaaacatattatttatatgtcaaaatagtttatgaatagacatataaacaacatgttcaagagatttattgtcttaatgtatatactccatactaaattgtttgtctaaaacttcatataataaaaaactgttaatcttcatgatggtgtgatttgttgtgataacagtatgatgtctattgtagatttttatttaaatacataaaaattgaaagaaaaaaaatgacatggctacttgaccaattgattcaagttaattgttactaaccaggccgataaactaattgggcctttaaagcccattaatatttgtcagtggaagcgaagtctgttacttgggttacaaaacaaaggcctttagaaaaccctaactcttatcgcagcacctgagctatatattgccgcagctagggttttccctagctcacgctaacacacaaagccgcagccatttccatctctctcgcaactcaacttcgaacaatggctgaacgaggcggagaacgaggcgccgagcgaggaggagaccgcggcggtttcggacggggattcggaggaggtcgcggcgaccgaggaggccgcggaggtccgagaggccgaggcggcagacgcggaggacgtcccacagaggaggagaagtggaccccagtgaccaagctcggccgtctcgtgcaagccggaaagatccagaagctcgagcagatctacctccactcgctccccgtcaaggagtaccagatcatcgacctcctcgtcggtccaactttaaaagacgaggtgatgaagatcatgccggttcagaaacagaccagggccggtcagaggacgaggttcaaggcttttgtcgttgtcggagacacgaatggtcatgttggtttgggtgtgaagtgctccaaggaggtggcgacggcgatcagaggtgggattatattggcgaagctgtcggtgattcccgtgaggagagggtactgggggaacaagattgggaagccgcatacggttccttgtaaggttacggggaagtgtgggtctgttacggtgaggatggtgcctgctccgaggggggcggggattgtggcggctagggttcctaagaaggtgcttcagttcgctgggattgatgatgtgtttacttcttccaggggatctactaagactcttggaaacttcgtaaaggtttgaactttattcatttacttggtgtttggagctgtgtatagattgaaagttgattcctttttgatcgttgcgtgtggaggtgttttgggtaatattgctaaatgtaaaaattgctaagcttgattagtttgaataagctgtctgtgttaaatgatgatttgtttgtgttgtaatgagctgtctgaatttcattatttgtttgtgttattgctaatttacaagcttaaatttgattccttttgatcgtttcgtgtgagttgttttgtgtaatttagcttgatgtagttgatggaagtaagaattgttaagctttagagctgtctgttaatgttgaattgtttgtgttgtattgagctgtctgaattattatttgtttgcgatttatttctaaatcacaaagctctaaagtcgtttgaacatatgcatcatttggttagtgtttattgctgaattttcatgatttggttgtgtttattgctaaatttgagattacattataatcttaatttgtacatatattcatcatttttgtgtttattgatttgtttgtgtttattgatgaatcacaagtttctatgattaatcttcatgatttggttgtgattattgctaaatttgagattacattataagcttagtttgtacatgtatacatcatttcacttgaattttcacagacgagctggtgatatatttttttttttagtattttccatgtattgctttgtaagaacgtttaaatgttagttttcttgaatgtttttttctttgtaagaacaattaaatgtttgttcagtatttttgaatcattcattggttaatcatgttcaacatgtattttttgtaggctacatttgactgtctccagaagacttatggattcctgacaccagagttctggaaagagacgagctttaaaaagtcaccgtaccaggagtacactgatctgctggccgagaagggaactgcaaccaaagccatcaccgaggttgaagaccaagccgcctcctaaataaacaaacaagcaatgcccaccttgttttgtttttggatgatcctttcatgagttttattttgttcgaaccttttagtatatgttgtttgctggattgctgctatcttaagttttcatcatcttcccaaacaaagttatttttgctctagttttttttttttataatcgatggttatactctagtatttttttacttgaatttctctaattttccatcag

SEQ ID NO: 31 - DNA, 185 bp, Brassica napus
  source 1..185: mol_type = other DNA
cattttgacccttcactttcttgaatgaaaattatacaagaaaatgatttgagtgtgttaaaaaaaaaatgttttgagtcattctgaccaatagctagggttttccctagctcacgctaacacacaaagccgcagccatttccatctctctcgcaactcaacttcgaacaatggctgaacgaggc

SEQ ID NO: 32 - DNA, 41 bp, synthetic construct
  misc_feature 1..41: note = synthesized oligonucleotide
  source 1..41: mol_type = other DNA
gaaggtcggagtcaacggatttgaaacggcaggtgaggcta

SEQ ID NO: 33 - DNA, 23 bp, synthetic construct
  misc_feature 1..23: note = synthesized oligonucleotide
  source 1..23: mol_type = other DNA
yccaagctgagtgtgaggctgaa

SEQ ID NO: 34 - DNA, 43 bp, synthetic construct
  misc_feature 1..43: note = synthesized oligonucleotide
  source 1..43: mol_type = other DNA
gaaggtgaccaagttcatgctgggaggtctttaacatctgctc

SEQ ID NO: 35 - DNA, 24 bp, synthetic construct
  misc_feature 1..24: note = synthesized oligonucleotide
  source 1..24: mol_type = other DNA
atgtattgattgttgttgcgcttt

SEQ ID NO: 36 - DNA, 20 bp, synthetic construct
  misc_feature 1..20: note = synthesized oligonucleotide
  source 1..20: mol_type = other DNA
tctctcgctcattcatccaa

SEQ ID NO: 37 - DNA, 23 bp, synthetic construct
  misc_feature 1..23: note = synthesized oligonucleotide
  source 1..23: mol_type = other DNA
tgattcgtattgaaagcaacaaa

SEQ ID NO: 38 - DNA, 17 bp, synthetic construct
  misc_feature 1..17: note = synthesized oligonucleotide
  source 1..17: mol_type = other DNA
ctctaacccaaagtaaa

SEQ ID NO: 39 - DNA, 20 bp, synthetic construct
  misc_feature 1..20: note = synthesized oligonucleotide
  source 1..20: mol_type = other DNA
tctctcgctcattcatccaa

SEQ ID NO: 40 - DNA, 21 bp, synthetic construct
  misc_feature 1..21: note = synthesized oligonucleotide
  source 1..21: mol_type = other DNA
tcgataagggtagggaggtct

SEQ ID NO: 41 - DNA, 19 bp, synthetic construct
  misc_feature 1..19: note = synthesized oligonucleotide
  source 1..19: mol_type = other DNA
ggcatatacctattagtac

SEQ ID NO: 42 - DNA, 20 bp, synthetic construct
  misc_feature 1..20: note = synthesized oligonucleotide
  source 1..20: mol_type = other DNA
tcgagtatagggagcgcaat

SEQ ID NO: 43 - DNA, 22 bp, synthetic construct
  misc_feature 1..22: note = synthesized oligonucleotide
  source 1..22: mol_type = other DNA
gggaggtctttaacatctgctc

SEQ ID NO: 44 - DNA, 14 bp, synthetic construct
  misc_feature 1..14: note = synthesized oligonucleotide
  source 1..14: mol_type = other DNA
gctccctatactcg

SEQ ID NO: 45 - DNA, 121 bp, synthetic construct
  misc_feature 1..121: note = synthesized oligonucleotide
  source 1..121: mol_type = other DNA
aaaaatagagmcagacgtctttagcgtccaaccatcttggacaactaggcctctcckgtcmcattagagatctcttggaccctagatataaccattctctccttggtttgtacgtaacagg

SEQ ID NO: 46 - DNA, 21 bp, synthetic construct
  misc_feature 1..21: note = synthesized oligonucleotide
  source 1..21: mol_type = other DNA
agagaaaaggcccaatcattt

SEQ ID NO: 47 - DNA, 16 bp, synthetic construct
  misc_feature 1..16: note = synthesized oligonucleotide
  source 1..16: mol_type = other DNA
gcaacaacaatcaata

SEQ ID NO: 48 - DNA, 25 bp, synthetic construct
  misc_feature 1..25: note = synthesized oligonucleotide
  source 1..25: mol_type = other DNA
aaacgtggatggttactttactttg

SEQ ID NO: 49 - DNA, 18 bp, synthetic construct
  misc_feature 1..18: note = synthesized oligonucleotide
  source 1..18: mol_type = other DNA
ctaacccaaagtaaagta

SEQ ID NO: 50 - DNA, 17 bp, synthetic construct
  misc_feature 1..17: note = synthesized oligonucleotide
  source 1..17: mol_type = other DNA
atattcctgacactctc

SEQ ID NO: 51 - DNA, 42 bp, synthetic construct
  misc_feature 1..42: note = synthesized oligonucleotide
  source 1..42: mol_type = other DNA
gaaggtgaccaagttcatgcttcgataagggtagggaggtct

SEQ ID NO: 52 - DNA, 20 bp, synthetic construct
  misc_feature 1..20: note = synthesized oligonucleotide
  source 1..20: mol_type = other DNA
ttgattgttgttgcgctttc

SEQ ID NO: 53 - DNA, 43 bp, synthetic construct; identical to SEQ ID NO: 34
  misc_feature 1..43: note = synthesized oligonucleotide
  source 1..43: mol_type = other DNA

SEQ ID NO: 54 - DNA, 24 bp, synthetic construct
  misc_feature 1..24: note = synthesized oligonucleotide
  source 1..24: mol_type = other DNA
atgtattgattgttgttgcgcttt

SEQ ID NO: 55 - DNA, 21 bp, synthetic construct
  misc_feature 1..21: note = synthesized oligonucleotide
  source 1..21: mol_type = other DNA
agagaaaaggcccaatcattt

SEQ ID NO: 56 - DNA, 20 bp, synthetic construct
  misc_feature 1..20: note = synthesized oligonucleotide
  source 1..20: mol_type = other DNA
tctctcgctcattcatccaa

SEQ ID NO: 57 - DNA, 20 bp, synthetic construct
  misc_feature 1..20: note = synthesized oligonucleotide
  source 1..20: mol_type = other DNA
tgttgcgctttctgtctctc

SEQ ID NO: 58 - DNA, 24 bp, synthetic construct
  misc_feature 1..24: note = synthesized oligonucleotide
  source 1..24: mol_type = other DNA
acataggagcyccaagctgagtgt

SEQ ID NO: 59 - DNA, 301 bp, synthetic construct
  misc_feature 1..301: note = synthesized oligonucleotide
  source 1..301: mol_type = other DNA
ataaaacgctagatggaacctgcatcaataccaaaamaaaaaaaaaaggttagaaaaatatgcgaggctcatttaattcaggatccggtgaaagctataatactatacattgtgacgagtcccctcttcaaaatccttctttgaagcctcratggcagctctgatcatttcttcttccgcatcgttcccatagacaggtgattcagcatccctatgtgttgttccaccatagacttgggatccgtagctaggtgaatgtctagcataaccgtctcctccgacatctccaagtccaggagga

SEQ ID NO: 60 - DNA, 301 bp, synthetic construct
  misc_feature 1..301: note = synthesized oligonucleotide
  source 1..301: mol_type = other DNA
acattccaagtttctttttggtgtaatgtgttgaaactctttaagctaactaaagactttgtactttgaagcccgggagctctacattacaagaaaagcgcgaagatattaaacaaaagcagccaattgatgtaagctctcagcacggacrtgaagcttcttatcctgaagaggtaattattcaaaaacagtttgatttctcttatcagtgatagtagcaaaagcatctttttagtgactagcttctagttgatcatgtgttcttcttaccctgttttcacctcctgttgctggacttcta

SEQ ID NO: 61 - DNA, 121 bp, synthetic construct
  misc_feature 1..121: note = synthesized oligonucleotide
  source 1..121: mol_type = other DNA
ttataaatgttttacttattctcagtactgatccggaytgatatggcacctaaaacagacmaaaaaatactgccatgtaactataactttttgcttattctaatcatcttaagagccaaaa

SEQ ID NO: 62 - DNA, 121 bp, synthetic construct
  misc_feature 1..121: note = synthesized oligonucleotide
  source 1..121: mol_type = other DNA
ataagcaaaaagttatagttacatggcagtattttttkgtctgttttaggtgccatatcartccggatcagtactgagaataagtaaaacatttataatgctttagcatatatcgaacaaa

SEQ ID NO: 63 - DNA, 121 bp, synthetic construct
  misc_feature 1..121: note = synthesized oligonucleotide
  source 1..121: mol_type = other DNA
gtactgagaataagtaaaacatttataatgctttagcatatatcgaacaaactagcataaraagtttctaaaaatccacmgaaattagagcataagttcaatgcaattagcatgkgaaaat

SEQ ID NO: 64 - DNA, 121 bp, synthetic construct
  misc_feature 1..121: note = synthesized oligonucleotide
  source 1..121: mol_type = other DNA
atgttaattttgtaattggtcraaawwttgcaaaatttaatttyttcatataaaatttayrgagaagtttgatattagtaaacttatgaaaaatactttactatcatatgaagttttcggg

SEQ ID NO: 65 - DNA, 121 bp, synthetic construct
  misc_feature 1..121: note = synthesized oligonucleotide
  source 1..121: mol_type = other DNA
cttgtagaagactttatgagagaaaattcatagaatagatattttataaagtgaaacatgrtcaattataaaattaatttgcctatattttttkgcaatatttmgaagwrtgtgattggta

SEQ ID NO: 66 - DNA, 121 bp, synthetic construct
  misc_feature 1..121: note = synthesized oligonucleotide
  source 1..121: mol_type = other DNA
gccawattaatcaaatattcgmatatttcgcaagctgtttcctatcgaataaatttcaatraaacaaatgaaaagacaattagcgacaacaatacaataatgattgaaaggcaccaatcca

SEQ ID NO: 67 - DNA, 301 bp, synthetic construct
  misc_feature 1..301: note = synthesized oligonucleotide
  source 1..301: mol_type = other DNA
aacaacaacacaaagatccttgagatttttcaatcgcagtttctatgcgactgctgtgacactcgtctaatttgcaggactgagaagaagctcctagagatttcagcatgggaaaaaaagcaaacaaccaagatcgaatctcaactcgctratacccaggtgattccaacaaaaagacctctccacttttaatgcattaatactgttctattgctaatggacaaaagggttgttttgttttgcgcagcggaagatggacagtaagaaaaaggagaaagcggagaaactgaggcgcaagaaa

SEQ ID NO: 68 - DNA, 121 bp, synthetic construct
  misc_feature 1..121: note = synthesized oligonucleotide
  source 1..121: mol_type = other DNA
cgattaaaaccgcaaacatccgcgttcgcaaacatccacaaccgcacctgcaaccgttgcrgttacatcactcggaccctaagtctacaaggttgaaagtttattataaatctacaaatta

SEQ ID NO: 69 - DNA, 121 bp, synthetic construct; identical to SEQ ID NO: 68
  misc_feature 1..121: note = synthesized oligonucleotide
  source 1..121: mol_type = other DNA

SEQ ID NO: 70 - DNA, 401 bp, synthetic construct
  misc_feature 1..401: note = synthesized oligonucleotide
  source 1..401: mol_type = other DNA
atattttataattatataaaatgtaaatattgttaatttattatttaaccactgatgcgttyggtagttagctagtcataagtatcctgcaaatgtatcaatttctaaccgctataccagtcatacaaatcttttagtaacgattaaaaccgcaaacatccgcgttcgcaaacatccacaaccgcacctgcaaccgttgcrgttacatcactcggaccctaagtctacaaggttgaaagtttattataaatctacaaattatttyaacctggttctgctcaactaagagattttagataaacagttcaatcaaaaggattagataatcatcacttaagacctgcaattcaactaagaaattttaggaaaactcgcttgggttagtgatattaatcaaatya

SEQ ID NO: 71 - DNA, 201 bp, synthetic construct
  misc_feature 1..201: note = synthesized oligonucleotide
  source 1..201: mol_type = other DNA
gtgggccaaatagaagtttctttctcaaaagcccattgataaacactatttttaaagtaaaaccctaaagtgggttaatgttgatggttgacttctggtgrggaatgtggaggcccatgtatagctagagtacaggaaactctttcagtgtttgcgagcttctacttaattcatcatattcgttatcttaacaaacaaaac

SEQ ID NO: 72 - DNA, 201 bp, synthetic construct
  misc_feature 1..201: note = synthesized oligonucleotide
  source 1..201: mol_type = other DNA
aaacggaraactaaccaaccaataggaggaggaggagrttgctccaaatgaaccaaagttkgggttttgacagcgaagattgaagctgggmgctgtgaagmaattgattgagcaaggggggagaggaagacgaaaccaatttagaagaagaagcaaagagtctctctctctctttctctctcttttggggytktctacttt

SEQ ID NO: 73 - DNA, 121 bp, synthetic construct
  misc_feature 1..121: note = synthesized oligonucleotide
  source 1..121: mol_type = other DNA
ctactttactatttagcccctgtaaagtaaatgattccagggatgaaactagagggcaaartagkgaatatattayagagcagwaaaggttaagattttcttcaaaccaggagaagwgtgt

SEQ ID NO: 74 - DNA, 121 bp, synthetic construct
  misc_feature 1..121: note = synthesized oligonucleotide
  source 1..121: mol_type = other DNA
acaataggacagtaataatatttacctgcagtgttagcagttccatcctcaawgtcaacamttttatcagtaggttcagtttctgttggttggatagaattgtcaggttccttgcttatat

SEQ ID NO: 75 - DNA, 121 bp, synthetic construct
  misc_feature 1..121: note = synthesized oligonucleotide
  source 1..121: mol_type = other DNA
aacttcaaaaggtkatattattgacaattgccctttacatatcacatgtttatttattaaytggttgaatatttatattcttaacttaccactgctaattattgatgaacaattgagaaag

SEQ ID NO: 76 - DNA, 401 bp, synthetic construct
  misc_feature 1..401: note = synthesized oligonucleotide
  misc_feature 8: note = n is a, t, c, or g
  misc_feature 14: note = n is a, t, c, or g
  source 1..401: mol_type = other DNA
aaawttcntttttnttttwttttwttttatttawttttatttatttatttaattttaaaccaagagtattagggatattttaccctttaatgaatgtcatttttgtgactttctccttctartrttatttttgagacataaacttcaaaaggtkatattattgacaattgccctttacatatcacatgtttatttattaaytggttgaatatttatattcttaacttaccactgctaattattgatgaacaattgagaaagtatcaatatggagctggaacaagaaaaatcaagatatgcgatacttgytgcattatattctacaatacttgcacttacaaaaatttaatatattctccaaattttctcagcatgtagcattttgtttttycagcatgtag

SEQ ID NO: 77 - DNA, 121 bp, synthetic construct
  misc_feature 1..121: note = synthesized oligonucleotide
  source 1..121: mol_type = other DNA
ctttctcaattgttcatcaataattagcagtggtaagttaagaatataaatattcaaccarttaataaataaacatgtgatatgtaaagggcaattgtcaataatatmaccttttgaagtt

SEQ ID NO: 78 - DNA, 121 bp, synthetic construct
  misc_feature 1..121: note = synthesized oligonucleotide
  source 1..121: mol_type = other DNA
ggtttagttttatcttctgccaaaaatatgttggctggacggtagctgactgtgcattgtrgttgcgttacaaaaaagaatacgtctcattaactttccacaatggtgtctttttcacaac

SEQ ID NO: 79 - DNA, 201 bp, synthetic construct
  misc_feature 1..201: note = synthesized oligonucleotide
  source 1..201: mol_type = other DNA
aaagttgtcaagagaacattcaaaagctattaagcagcgctttaactgaagggactgtatgtataagttttcttggttttaccctctatgtgaatctgtargtttaccgacaaataatattttgttatttcgtattccatatctttaaaaaaataagaaaagttatattatattttaaaaataaaaaataaaaataaataa

SEQ ID NO: 80 - DNA, 301 bp, synthetic construct
  misc_feature 1..301: note = synthesized oligonucleotide
  source 1..301: mol_type = other DNA
aaataattataagatagttagtgctaatgatgattagatgataagttaaatagagaaaagtgagtctgcagttaatttgcatgggcttagggatattttttcaatataagtaaaagataattcaagggaattcgtgagcactcccacattmcactcaccaccttttcgcagcgacaccgacatatgccacattttcattttaccttttttttcttgcaaaagatcgttaattaaagaactggatttaagattaatctctacatatatacatatttacggatatgtacgttttttttgaaca

SEQ ID NO: 81 - DNA, 301 bp, synthetic construct
  misc_feature 1..301: note = synthesized oligonucleotide
  source 1..301: mol_type = other DNA
cattttcattttaccttttttttcttgcaaaagatcgttaattaaagaactggatttaagattaatctctacatatatacatatttacggatatgtacgttttttttgaacaacaagtgcttatgctaaaattaaacgcacatgagttaartcagttaaatccaaagagacactaaattgataaactaacataactaaaacataaatatacatattcattctcgtaaaatatactcaatattacagagatatatgtgccagctgtttagatacatacatatgcatgaataataaaacccac

SEQ ID NO: 82 - DNA, 121 bp, synthetic construct
  misc_feature 1..121: note = synthesized oligonucleotide
  source 1..121: mol_type = other DNA
tggttgtwcagacagacggacaggtctttaattactacctagtcttttwctttccaaatamagaagagtctctccctgagagggtattctgaatctaatggcgaagaaggtgttgatgayc

SEQ ID NO: 83 - DNA, 121 bp, synthetic construct
  misc_feature 1..121: note = synthesized oligonucleotide
  source 1..121: mol_type = other DNA
gcaaaacacttggttaccaaatagccctastagtacaaaaaraagataacaraaaagttcraaggctttttcatttgatgattgcattctactctcttagcctttgttaaagctggaaaac

SEQ ID NO: 84 - DNA, 201 bp, synthetic construct
  misc_feature 1..201: note = synthesized oligonucleotide
  misc_feature 137..140: note = n is a, t, c, or g
  source 1..201: mol_type = other DNA
acattgatgtgtttttctgtttcagttctcacttacggtgttatatgtctttgccagttcgttgttggtgttggtatgtatagaatgtagcacaattatcrcaagtaacagtatcaaagacatgtttgtgcgagtgnnnnagagacgtgcacaaggttgtataacaagtaaagcaaatctatgctaacactaataaatatc

SEQ ID NO: 85 - DNA, 121 bp, synthetic construct
  misc_feature 1..121: note = synthesized oligonucleotide
  source 1..121: mol_type = other DNA
twatattttataatgggattagaattaacaaaagaaatkatatattggcatttgtagttgragagaactttccaaacttgaaattcaaggtaaaggaggttggagatgagtcaattttgta

SEQ ID NO: 86 - DNA, 121 bp, synthetic construct
  misc_feature 1..121: note = synthesized oligonucleotide
  source 1..121: mol_type = other DNA
tttgcagcgtaacaaagctgtaatgacaacaagttgaaggtttctcagacctccaaggctktaaggaggaacaakaaggtttaaaagaaaactgtggcactgaaatactcacctggaatcc

SEQ ID NO: 87 - DNA, 121 bp, synthetic construct
  misc_feature 1..121: note = synthesized oligonucleotide
  source 1..121: mol_type = other DNA
ggattccaggtgagtatttcagtgccacagttttcttttaaaccttmttgttcctccttamagccttggaggtctgagaaaccttcaacttgttgtcattacagctttgttacgctgcaaa

SEQ ID NO: 88 - DNA, 121 bp, synthetic construct; identical to SEQ ID NO: 87
  misc_feature 1..121: note = synthesized oligonucleotide
  source 1..121: mol_type = other DNA

SEQ ID NO: 89 - DNA, 301 bp, synthetic construct
  misc_feature 1..301: note = synthesized oligonucleotide
  source 1..301: mol_type = other DNA
tctgtccttcaagtgagaagcgaccacacttgtgatgctttgatcacgacagctgaacatccctccggctgaaaccamccgtctgaacaggagatcagacgttggagactgcggcttatgcctgatgagatcaagcggagtcataccagargcatccttgatgttgatatcaatcgatttcacagacatgagmatctcaacgaackcgagaggaacgtttccggagattgccaagtgaagagccgtacgtccctcgttgttcctgcagttgacgacatcgcmgggggagttggaggcagcg

SEQ ID NO: 90 - DNA, 121 bp, synthetic construct
  misc_feature 1..121: note = synthesized oligonucleotide
  source 1..121: mol_type = other DNA
cagcaagagttggtatccacccacctagagaagagtgatgatgcaatatcaataatactammggtcccatctaatgctaagacagtaaacagtacgtytgatagctaaaactgaacaggta

SEQ ID NO: 91 - DNA, 301 bp, synthetic construct
  misc_feature 1..301: note = synthesized oligonucleotide
  source 1..301: mol_type = other DNA
ctgcccttagtgagctcaggaggaagaacgtcgagactcttccctacgatgacctsaaacgttttgtcaagctygacmcccgtgccaaaatcaaggagaacaactccatcaaggccaagaactgatctgtctccggtttagagagatctarattgattttgcttcttactgcttaatgttttttkgrgtgtggttaagattatctatcatttaagttatgttacattttcagagtttataaktttcctagtaaacccaataagaattgctgcttcatctattgctttttttttagaccaaa

SEQ ID NO: 92 - DNA, 301 bp, synthetic construct
  misc_feature 1..301: note = synthesized oligonucleotide
  source 1..301: mol_type = other DNA
tagtctgtggcgtactgttgcmaaagcagcaaacattcagatcgagatggtttatgttggaaaaaggaaccctaagaacgggatccagcsgataatcaacacaatcagagatgaaaacctcagccatacattgccggacttgtttcagatmtggttcttctgggcgagggttgagagcatgtgggagtcgaaacaaaggatgctaaaagcccaaagaacaaaaggagggcggcaagggtttaaagaagaagaggaaaaggaccttgtgctgcaagaaatcgttgctttgttaggttttgga

SEQ ID NO: 93 - DNA, 301 bp, synthetic construct
  misc_feature 1..301: note = synthesized oligonucleotide
  source 1..301: mol_type = other DNA
ctccacacggcgtcaaggctcgcttccataccaatcttcgaacggatwcgccgcgagctcttctcgagcttcttcacctctatgagaaccccatcgtcgcctaccaccaccaattcctcactcttctcatctgtcaagcaatcaaccaaayaatcatcttcatcggtttcactttcatcctcgttacaataatcatcgtcgccgattcggcatgtgaaacatctacgtgmgggaggggaaggtagatgtagtgtcgaggaaggtggaaagtagggtgacaatacgtttgtagagtggaatt

SEQ ID NO: 94 - DNA, 121 bp, synthetic construct
  misc_feature 1..121: note = synthesized oligonucleotide
  source 1..121: mol_type = other DNA
tagggctttaggcaacatcctaactcctccaatgtaatcctaaactcctaacgctgaatcrtacttgcaaagatcttggacagagaagacaatacaaagtttcggaaatggtgacttttac

SEQ ID NO: 95 - DNA, 121 bp, synthetic construct
  misc_feature 1..121: note = synthesized oligonucleotide
  source 1..121: mol_type = other DNA
caggcaacggatgaagatgacggagagagaaaatcwtagggtgtgagaatcttmtttcagractattaatgggtctaaacatgggtccattaatagaataatctgtttatcaaacgtgcat

SEQ ID NO: 96 - DNA, 121 bp, synthetic construct
  misc_feature 1..121: note = synthesized oligonucleotide
  source 1..121: mol_type = other DNA
gtatagtgtataacatgaaaaagataagggaaaagaagatracacagtgatcggggagctrgcagcccacattatcatcacctttaaagaaataaataagcaaagcatgcacatgattatc

SEQ ID NO: 97 - DNA, 201 bp, synthetic construct
  misc_feature 1..201: note = synthesized oligonucleotide
  source 1..201: mol_type = other DNA
atgtygtakatmacayagcaagttccaaattcctcttgctcttttacttgattaataattgtacacagctcctgattatctccttcaaaccacactcttcrccagccacaaatccaaacttgttgaagagctataaggaaacttaaagcttccccttctaawgatgtctgcatcttttttmacttggmcacttcccgctcc

SEQ ID NO: 98 - DNA, 301 bp, synthetic construct
  misc_feature 1..301: note = synthesized oligonucleotide
  source 1..301: mol_type = other DNA
atagggagtggttgcacaaggaatgtgyacggtgggaatatcatggtgtattaaacaacgaggacctgtctttacctcagcatttactcctgtcatgcttatgtctgcaaccttgttcgatttcttgatatttcatcgtatgatttatttrggaaggtaaggatgtattctctaaaagaaaacaatgcatatttgatatatagaactttcatattgraaagaatatacttagtttatcaaatacgaaacattgatcattgcattaactttcaactgatattaatatggaactcttctagaa

SEQ ID NO: 99 - DNA, 121 bp, synthetic construct
  misc_feature 1..121: note = synthesized oligonucleotide
  source 1..121: mol_type = other DNA
gaagggatgtaatctatatgggcatattaaaagaaaaaaataataagcatattgcgtatgmttgaaaaagttagtaatataaactcgtttggatgccagagttaaacaaagtttaagatgt

SEQ ID NO: 100 - DNA, 121 bp, synthetic construct
  misc_feature 1..121: note = synthesized oligonucleotide
  source 1..121: mol_type = other DNA
ctcaccygatttggtcccataggacattattttgggttgtacggtcaaggaatgttcgagraaggcaaaaagccgcwgtycggaaacgcgttaggtrttaagcgctagtcrgayggtyggr

SEQ ID NO: 101 - DNA, 121 bp, synthetic construct
  misc_feature 1..121: note = synthesized oligonucleotide
  source 1..121: mol_type = other DNA
tgccttyctcgaacattccttgaccgtacaacccaaaataatgtcctatgggaccaaatcrggtgagcaaccrtaagttttctattatattttaaatttacaagttcctagaaatttatgt

SEQ ID NO: 102 - DNA, 121 bp, synthetic construct
  misc_feature 1..121: note = synthesized oligonucleotide
  source 1..121: mol_type = other DNA
tcakcstcaacacmacaatgatgcagwgactaagtcaggctttgaagtttcctcataaaarcacaaagtttcagtaatatcactagcttggacagaagcttataacccctaagrttwacat

SEQ ID NO: 103 - DNA, 121 bp, synthetic construct; identical to SEQ ID NO: 102
  misc_feature 1..121: note = synthesized oligonucleotide
  source 1..121: mol_type = other DNA

SEQ ID NO: 104 - DNA, 121 bp, synthetic construct; identical to SEQ ID NO: 102
  misc_feature 1..121: note = synthesized oligonucleotide
  source 1..121: mol_type = other DNA

SEQ ID NO: 105 - DNA, 301 bp, synthetic construct
  misc_feature 1..301: note = synthesized oligonucleotide
  source 1..301: mol_type = other DNA
tgatacaataaacaagagctttaaatctcaagaaaagcagcagcaaaggtaagtcggaggatgaagaaatacctcagaatgcgattagaagtggcgccgcttaaacacacagattagggttttgcaatgcagccaaggagtggatcgtcarttataaatagagcaaatctaccgaaactttgttgggcctatggatgaaggcccattttggaaacctgacgtttcggccttatttttcgcgtggcttttctacaatatatctctctatgcgaacatgcatcggccattgtttcactttcag

SEQ ID NO: 106 - DNA, 121 bp, synthetic construct
  misc_feature 1..121: note = synthesized oligonucleotide
  source 1..121: mol_type = other DNA
ttagccttttgtctcaacttctccatcttctatgtcattcactccgcagctgcagtcacartcaatttagctggaaaccttacggttgaaggttgcagtggctgtactggtgttgtgkttg

SEQ ID NO: 107 - DNA, 301 bp, synthetic construct
  misc_feature 1..301: note = synthesized oligonucleotide
  source 1..301: mol_type = other DNA
atgaagaggaagaagacgatgatgacaatcaagatgtggatagggaagaaggtcaggaggagttagctgaagatgaagacaacaatattgatattgatattgagacagcagaagatgaagagaaatcaccctctcctattgacgaagaaarggaagagtattcctttcttctctcacttcctcctcatggttctgaagttttcattggcggcctccctagggatgttggagaagaggacctgagggatctatgtgaaccaatcggcgmcatmtttgaggttggaacatcaataatattttg

SEQ ID NO: 108 - DNA, 121 bp, synthetic construct
  misc_feature 1..121: note = synthesized oligonucleotide
  source 1..121: mol_type = other DNA
gtcttcttctgtccaatatgggcaggractcagcttcatcgccttgttcaacgyaattttrtaaaacttgctgactcattagacggtaagggtgataacaaaataggaaacttgtcacgca

SEQ ID NO: 109 - DNA, 301 bp, synthetic construct
  misc_feature 1..301: note = synthesized oligonucleotide
  source 1..301: mol_type = other DNA
tttatttcaacctacaagagaaaggsatgtgttaatgagtatacttctcaaatattgtagaagagagagaatggagttgactgacctcacaccagtcaaaacagtctaaaactctcacgcttttcccatctcctttgcatttaacttttgrattcatttccctgcaasccctccacataaccattggatcccaacttagtccagatgtcttatcgagaacatttctgacaatcacaggttctgccttcatccaatgcgactgaaaatgaacaatatcatcttcagccagatcaacagcatt

SEQ ID NO: 110 - DNA, 301 bp, synthetic construct
  misc_feature 1..301: note = synthesized oligonucleotide
  source 1..301: mol_type = other DNA
aatagtttaccatctcttgatgcagtcatagcaatagcgcttcttaatgcatttagaacagaagacragatcaaccttatacgttaagttcttrcactgatgacaagtcaaacccgcatacttttttgttccatcagagttatcatcctgmaaaaggaaaacagtgttcacatttatccaaaaaaaaaaacatttccacaaatagaagtaaacaaagatgattcaagcttgttgacaaactgaacaattcaattacaaaaagaaaatamccttgtkgggcgtagacatgcctctctctctc

SEQ ID NO: 111 - DNA, 301 bp, synthetic construct
  misc_feature 1..301: note = synthesized oligonucleotide
  source 1..301: mol_type = other DNA
tgagaagcaattagattttctgaaagacatcattatgaattgaattttgcatatgagaagaatgaatctattacatacctccatatcttttgctgaactgaagtcakcaaagttaagtggttcaacacgtttgcaacacaytgcatcaacmgtctctgtttctccagcaactccttcttgcttaacttcagaatcattgccttgattcttaacatctacaaccgtttcacaagctacttccaccagattcttttctgcgtctttatataaattatcttccttttcagtctgaaccattgca

SEQ ID NO: 112 - DNA, 301 bp, synthetic construct
  misc_feature 1..301: note = synthesized oligonucleotide
  source 1..301: mol_type = other DNA
ctccgcagatcagccgctggagatgcgtcgggatgtgagtgtggtcgaagatccgctgcttgatctggtcgccataggctaagggagaggagaaagctacggttatcgattttccgttgagtagcctaacgaataactgcaacggyttagrgctctgatccgccattgttagcaaagttcagtgatcgaagaagaagaaaatcgattttgtggaaatcacaacgaaatgtaaaaatcttgggatgggcaataaaccgagccgaacttatggtatttccggttaacgaaactcgaaccttaa

SEQ ID NO: 113 - DNA, 301 bp, synthetic construct
  misc_feature 1..301: note = synthesized oligonucleotide
  source 1..301: mol_type = other DNA
caacaatattgatctgaacagtctataacgccgtgagaaacaaccgcacaactgggaattcataaaattaatctacaacaaatactagataacctcacaggagaagaacgcgagaaagaacaaaaacacatagatggtgacggttcaagargaaaatatggrgagatgacaaaaacatcttacaaataatgtttaaaattttaaaaataatatcccgcgtgaagcgcggctacacccttagttcttaataaaaaaacgttgcttctagctgctgtatcaacagtttgatcattcaccctta

SEQ ID NO: 114 - DNA, 121 bp, synthetic construct
  misc_feature 1..121: note = synthesized oligonucleotide
  source 1..121: mol_type = other DNA
gccatttaaattaaatcaaagtggtgatcagatgattttcctaggagtttattactctggrtctgatttatatgagaaaacatgtagcatttagttcttgcttctcttaacgtctgaccat

SEQ ID NO: 115 - DNA, 301 bp, synthetic construct
  misc_feature 1..301: note = synthesized oligonucleotide
  source 1..301: mol_type = other DNA
gcgtaaatcaacgttaggcataacgggaagtgaaaatagtgaggaaggacatctgagagtacaggtttgttttgggaattgtggtgagggctgcaggtgttgttgtaagccacagatttatagtagtcgttcgattgatacgtgaaacgcmaggagagggaacctgttgagtagtagttaagagtaataataagaattcatgtagatgtggaaggaataaatttattaacaacgattgtgtatgtaacggaggctgattgaactccttaagaactaaattagggttcaccttaagcgtcac

SEQ ID NO: 116 - DNA, 301 bp, synthetic construct
  misc_feature 1..301: note = synthesized oligonucleotide
  source 1..301: mol_type = other DNA
tcgaacgtttgtagatgccttctacattattcacattgtgtttcagtttaggacagcktatgtctctcctttgtcccgrgttttcggkagaggcgagttggttgaagatcctaaagccattgctttaaagtacatctcctcttacttcatmatcgatgttctctctatccttccactcccacaggtaatattaagtgactgaaccttttaattaagttgaaatggtttcttgataacttcrttcagtgatgttctgtttgtttcttctccagcttgtagtcttggctgttatcccgaatgt

SEQ ID NO: 117 - DNA, 121 bp, synthetic construct
  misc_feature 1..121: note = synthesized oligonucleotide
  source 1..121: mol_type = other DNA
tcaaatttttttcttgttttgtcagatcaaatatggagtaatttatggataaaagttatargttattcgaataatttggttatttgagtaatttggatcattttcaatataaaaactgctt

SEQ ID NO: 118 - DNA, 301 bp, synthetic construct
  misc_feature 1..301: note = synthesized oligonucleotide
  source 1..301: mol_type = other DNA
atcctttagagttcctacacatcatttgcatcaaatataataagaccaattctcatgtgaaactgtttccaagaaactatgagaaaccaaccacggcgtaatgtaagccaacactggataatcataatgcctagctgatgaattctctgamgcctttgaatgttcctgaaccaaaactcaaatcccctaaacctttcagctttttttttcaaaggattctcaaacattgaaacgaaaaaaagtaaggcaacactcacagtgaggatctcttggaaatgaatatcggttttgattattccct

SEQ ID NO: 119 - DNA, 121 bp, synthetic construct
  misc_feature 1..121: note = synthesized oligonucleotide
  source 1..121: mol_type = other DNA
ttcataggtctataaagtcagtctttagaaattacagtatgatttaaataataacaattcrtattaaaartggtttgaatctgtggcacgaaggaaccaatccatcgcataaccactagac

SEQ ID NO: 120 - DNA, 121 bp, synthetic construct
  misc_feature 1..121: note = synthesized oligonucleotide
  source 1..121: mol_type = other DNA
tggtgagtttccatgtgcaatatattgtttttgcaattgtccaaaagaggaaagtttttcrgaactgatatgaatgcaccttaytgcagtcaggtaaaggaaggagacctagrakggatga

SEQ ID NO: 121 - DNA, 121 bp, synthetic construct
  misc_feature 1..121: note = synthesized oligonucleotide
  source 1..121: mol_type = other DNA
attycagmtraaaaacattattatcaggacaagaagsttatattcagacagcttcactacrttttccacaaaagaaaccaatcccaaataaaaagcacaaataatgtgaaattaatamaaa

SEQ ID NO: 122 - DNA, 121 bp, synthetic construct
  misc_feature 1..121: note = synthesized oligonucleotide
  source 1..121: mol_type = other DNA
tcggcgtcgccttttgtaatttctgttgataaggatttctcgtttaacccttgtaaaagtmraccttyccgttaaagccccaaaattaatttttgaagttcaaattaacccaaaagaggga

SEQ ID NO: 123 - DNA, 121 bp, synthetic construct
  misc_feature 1..121: note = synthesized oligonucleotide
  source 1..121: mol_type = other DNA
gacaacaatcacaggatcatccacgggcagcgacacatcctccagcagctggtgcgactcrgattttacgtcagagtttttaccgtcagcatggggaggtaacgttgaaaaagaggaaggt

SEQ ID NO: 124 - DNA, 121 bp, synthetic construct
  misc_feature 1..121: note = synthesized oligonucleotide
  source 1..121: mol_type = other DNA
wtttgttcacattctgtgttacttaattataggctccactcsgccgatgcattcgacttamacttttcgggtaaggtttgaagtaccttgtttatcacatgatatcatgaataacacatag

SEQ ID NO: 125 - DNA, 121 bp, synthetic construct
  misc_feature 1..121: note = synthesized oligonucleotide
  source 1..121: mol_type = other DNA
aacatgatcgtaaattcatctcmcgtctttcttatataatattgtagcaagtcyaagtccrgggtgtgtagagatagtttccctttcaaagtatttttttttcataattttaaccccctta

SEQ ID NO: 126 - DNA, 121 bp, synthetic construct
  misc_feature 1..121: note = synthesized oligonucleotide
  source 1..121: mol_type = other DNA
ttwtttgmtaataaggacaaactaaaaaccttattgctaagctcacattaattacatatamttttcaatwcgctwmtatataatcaaaaagaattgtgcgttaatgacaaaaggcacaaca

SEQ ID NO: 127 - DNA, 121 bp, synthetic construct
  misc_feature 1..121: note = synthesized oligonucleotide
  source 1..121: mol_type = other DNA
atgctctagatatggccttgttacttaccgtaagtccttgccttttccctgattgagaatraccctctccgagcattgatytcaaygttgttttcagggagctacgatcgttgatcaaacc

SEQ ID NO: 128 - DNA, 121 bp, synthetic construct
  misc_feature 1..121: note = synthesized oligonucleotide
  source 1..121: mol_type = other DNA
kacgragaggaaggagaagaaatgataacgagtccaaggtttttggaatgacctggatatrgaatwtatattcaatyycgrrttagaaaatgcgartarraaaatctckaaaagaaagaca

SEQ ID NO: 129 - DNA, 121 bp, synthetic construct
  misc_feature 1..121: note = synthesized oligonucleotide
  source 1..121: mol_type = other DNA
tctcgcstttcacttttagttgtgtctcccataacgtttctgacttcttgtctttcttttmgagattttyytaytcgcattttctaayycgrrattgaatatawattcyatatccaggtca

SEQ ID NO: 130 - DNA, 201 bp, synthetic construct
  misc_feature 1..201: note = synthesized oligonucleotide
  source 1..201: mol_type = other DNA
ttagtgctttcttcattggatggaggatataacttaaacgaatgattttacaggcttttcaaatatataaagatatttatcatttttaaaaatagaaactrtgtggtccatttgctatcaattcaactagaggtgtaagcccttgtataggagataaaggattgaaattattagtttacaaaaatcaaatgtttacactat

SEQ ID NO: 131 - DNA, 121 bp, synthetic construct
  misc_feature 1..121: note = synthesized oligonucleotide
  source 1..121: mol_type = other DNA
caccaatgatcaaacatgaatcattgtctctaaatataaattgctracatctcaaaaccgrattctgtttttattacctgagctgggacagcagcagcaagcacacggggaatattcttag

SEQ ID NO: 132 - DNA, 201 bp, synthetic construct
  misc_feature 1..201: note = synthesized oligonucleotide
  source 1..201: mol_type = other DNA
gaaccaytgtttcctcgtcaaacatggtagatgttttaatatttatttatgtgyataataattaataagcatatacatgcaaatcagctaactttttaaastccctaccaagctaccaaccctaaacttgtgaccttaatttcaacatattaaaattcacattagaacttgaacgttttcakaccgtcctggtttgtttgt

SEQ ID NO: 133 - DNA, 121 bp, synthetic construct
  misc_feature 1..121: note = synthesized oligonucleotide
  source 1..121: mol_type = other DNA
gccctatgatcagcttgcgaaacttcaaaatattgacgaccatgcacggccgtggatgttratggtggcatctggttaggaaatgagtctggtcgttgckgttggacaaggctggtctgac

SEQ ID NO: 134 - DNA, 121 bp, synthetic construct; identical to SEQ ID NO: 133
  misc_feature 1..121: note = synthesized oligonucleotide
  source 1..121: mol_type = other DNA

SEQ ID NO: 135 - DNA, 121 bp, synthetic construct; identical to SEQ ID NO: 133
  misc_feature 1..121: note = synthesized oligonucleotide
  source 1..121: mol_type = other DNA

SEQ ID NO: 136 - DNA, 121 bp, synthetic construct
  misc_feature 1..121: note = synthesized oligonucleotide
  source 1..121: mol_type = other DNA
ggacttggaggaagacaaacatcagagaaacttatcattcktggctmaaagcggtaaagcrtcagagaaatttgtaatgtttttgagtttatgatggcaaatccttgagtttgmtttgtgg

SEQ ID NO: 137 - DNA, 121 bp, synthetic construct
  misc_feature 1..121: note = synthesized oligonucleotide
  source 1..121: mol_type = other DNA
tggttaatgagagattaattaatcttgaagcacactagtagagttgaaatattgttatacrttttattgactcttaacatgttytacgtgattgattcaaataaacaatataaatgctccc

SEQ ID NO: 138 - DNA, 121 bp, synthetic construct; identical to SEQ ID NO: 137
  misc_feature 1..121: note = synthesized oligonucleotide
  source 1..121: mol_type = other DNA

SEQ ID NO: 139 - DNA, 121 bp, synthetic construct; identical to SEQ ID NO: 137
  misc_feature 1..121: note = synthesized oligonucleotide
  source 1..121: mol_type = other DNA

SEQ ID NO: 140 - DNA, 301 bp, synthetic construct
  misc_feature 1..301: note = synthesized oligonucleotide
  source 1..301: mol_type = other DNA
taaataaactaaaccacgtatcatatatgagtatttagtgttgctaaatcctatgaagaagctatgcatacacagatgacaaaattcaaaacacatgaatgctaatatgctaattcataaattcacgagttaacgtttaacttgtatagaratatwtcaggaaaacatagtgcaaaatttaactaccttattatttttgttttgtgtcccctctgttgcgaacacatcgaacatttcattgtttcaataatctaatyacatgggattatcacttcctctactagtctggttgagttggact

SEQ ID NO: 141 - DNA, 121 bp, synthetic construct
  misc_feature 1..121: note = synthesized oligonucleotide
  source 1..121: mol_type = other DNA
ttactagctaaaagaatttggcaagaatatgcaccatttggtgaggttgcggttgatcacmcactgaattctgatcttatggcaccaacggctctggttttacttgtygagttttcacctt

SEQ ID NO: 142 - DNA, 121 bp, synthetic construct
  misc_feature 1..121: note = synthesized oligonucleotide
  source 1..121: mol_type = other DNA
ttttttggcaattcatcataatagggtctgatgttttgtcatctgttaggtgccaaagtaragaactttgtgtgaattgagctgctttttccctcgagaaatggttctacatgttatgttg

SEQ ID NO: 143 - DNA, 121 bp, synthetic construct; identical to SEQ ID NO: 142
  misc_feature 1..121: note = synthesized oligonucleotide
  source 1..121: mol_type = other DNA

SEQ ID NO: 144 - DNA, 121 bp, synthetic construct; identical to SEQ ID NO: 142
  misc_feature 1..121: note = synthesized oligonucleotide
  source 1..121: mol_type = other DNA

SEQ ID NO: 145 - DNA, 301 bp, synthetic construct
  misc_feature 1..301: note = synthesized oligonucleotide
  source 1..301: mol_type = other DNA
tctgctccaacctcagagaaactaggagcacctcttttctctttattaattgggaaacggggaagagcataaacaaacggcacctcctccrcttctcctgtataacgaataacatccttgactataccagaatcaccaagacaacactcamcgtctgaaacaccagaaccagatcccaaatcctctaatgcctgaacaatatccagagaattaccctctactctaccggccacgttagcataaamctgagcagamttyccaatgctctggatatactgaaccgctacgcaggtwagcgtat

SEQ ID NO: 146 - DNA, 201 bp, synthetic construct
  misc_feature 1..201: note = synthesized oligonucleotide
  misc_feature 124: note = n is a, t, c, or g
  source 1..201: mol_type = other DNA
gaaccttagtagacggcctctcctgctctcatcatcatccaaaccaamccaatcaaacaacacacattttcatcagtttctaaatcaaaagtttcaatctraaaatgtaacaaagtctawtccntttaccgtgaggaagttgccacagttttgrccttctgatctgaagtagttattaggacttcctctaacacctgcagg

SEQ ID NO: 147 - DNA, 201 bp, synthetic construct
  misc_feature 1..201: note = synthesized oligonucleotide
  source 1..201: mol_type = other DNA
taytctagyctatcakycawcggttaacgatcagatcttaaattgarawggctaagaatggtacctcgggaccagcatcagcccaggcgagctgggaagtraatcggacgacggcgagaaacggatcttcttcggargtgggaacaatcgtcgtcatagcttagctcgaagctcttctctcggcggcgctgatgaacaaaa

SEQ ID NO: 148 - DNA, 201 bp, synthetic construct
  misc_feature 1..201: note = synthesized oligonucleotide
  source 1..201: mol_type = other DNA
cctcagcctcyctacatagcctgrtgatctctggctctgcagcctakaagagtagctcaattagagtaatttgaatttgtcatatgcatcagaatcatgarcgattacaractaytctagyctatcakycawcggttaacgatcagatcttaaattgarawggctaagaatggtacctcgggaccagcatcagcccaggcg

SEQ ID NO: 149 - DNA, 121 bp, synthetic construct
  misc_feature 1..121: note = synthesized oligonucleotide
  source 1..121: mol_type = other DNA
atgcttacatggaattccaagctgcaaatkcagagtgtctgcaaaccyatggtactaaatygtaaatcagtttactgctttttcgtaaatacaatgtcctgaagaatccttcatttctata

SEQ ID NO: 150 - DNA, 121 bp, synthetic construct
  misc_feature 1..121: note = synthesized oligonucleotide
  source 1..121: mol_type = other DNA
tatagaaatgaaggattcttcaggacattgtatttacgaaaaagcagtaaactgatttacratttagtaccatrggtttgcagacactctgmatttgcagcttggaattccatgtaagcat

SEQ ID NO: 151 - DNA, 121 bp, synthetic construct; identical to SEQ ID NO: 150
  misc_feature 1..121: note = synthesized oligonucleotide
  source 1..121: mol_type = other DNA

SEQ ID NO: 152 - DNA, 121 bp, synthetic construct
  misc_feature 1..121: note = synthesized oligonucleotide
  source 1..121: mol_type = other DNA
attcattacgaagaggaaaagcccatgggcctgcaaagtttattgcatttcaggttcagcrtcgtttcttcgttcgttcgattttgccaaatccgaaacgattcaaattccctccgactct

SEQ ID NO: 153 - DNA, 121 bp, synthetic construct
  misc_feature 1..121: note = synthesized oligonucleotide
  source 1..121: mol_type = other DNA
tgycgagtgttaattaaaccttgtaatttgcaaagtaagtgactcttggttccggatttgrtgagatcaywatctacttaatcatgcttttgttcagtgcaagttttggcctgaagttata

SEQ ID NO: 154 - DNA, 301 bp, synthetic construct
  misc_feature 1..301: note = synthesized oligonucleotide
  source 1..301: mol_type = other DNA
cattctgggttytccgaaagtcatgatttttatgtaaatggtgctgagatcgctacaattgaagcttctgaagrwggatccgttgcagaagccgaggaagctcaatctgaggaagtgactcaatttgacgaggaaaagtttgmatctttgmtatctctgttacgaagtgaagaggagtctttggagtttagtctgaagacgctcgacgtcgatctaaactcagaccttgtcgtcaaagtgtttgagtcctctccaggaatctcaggtaagaacttgattaagtttctgaagtgggcaatta

SEQ ID NO: 155 - DNA, 121 bp, synthetic construct
  misc_feature 1..121: note = synthesized oligonucleotide
  source 1..121: mol_type = other DNA
aagcatgtctatagccttgagccggcatgattcaaatcttgtgtctcctactcctgaggtrgctactgaagatgacaagtctagtacatcatcagatgaarawtcatctryggacaatgaa

SEQ ID NO: 156 - DNA, 121 bp, synthetic construct
  misc_feature 1..121: note = synthesized oligonucleotide
  source 1..121: mol_type = other DNA
attaaacttcaaaatacactactaatcaaattcagccgacgtcctgaaaagtacaattacmtcttcatttacacgkccaattgttttstrtsttttcttttaaawatcgtttcgttatatc

SEQ ID NO: 157 - DNA, 121 bp, synthetic construct
  misc_feature 1..121: note = synthesized oligonucleotide
  source 1..121: mol_type = other DNA
ttgaattagrtggggsaatactcctttrgatgaagccctaggttgtggtaacargatgttrataaagttactagaagacgctaagagttcacagatgtcttcgcttccaagtagctccaaa

SEQ ID NO: 158 - DNA, 121 bp, synthetic construct
  misc_feature 1..121: note = synthesized oligonucleotide
  source 1..121: mol_type = other DNA
aaaaaamatattagatatgtttcaagaaaccaagaaggttttaaaaaagcataccaaaggmgatcttccatcataatctgttttattsgggtcacctccrgctcgaatcaagctcttaagc

SEQ ID NO: 159 - DNA, 301 bp, synthetic construct
  misc_feature 1..301: note = synthesized oligonucleotide
  source 1..301: mol_type = other DNA
tgagaagcaaataaatttattatttaagctattttttgacatcaaaaggtggtttcttgttcttctggatctaatcgtcaatgatgcaaaaagagctattttcaacgaggttgactattttaagatgggtggtgaaacttccaacactctrttaacacgtaatgatgatgactgatgaccatatctaaccgaaccaaacaaaacgctaatagagttcasaagacacaagaatagcaaaaatgaatgtcaaatgttttaaagcattttttttttttgtcaaacttttttttttaccttgtga